SEQUENCE LISTING (35 sequences)

SEQ ID NO: 1 - protein, 256 aa, synthetic construct
GGPCRSHIDVAKPICVGGGGSGGLSGRSDAGSPLGLAGSGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 653 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 3 - protein, 221 aa, synthetic construct
LAGSGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 229 aa, synthetic construct
SDAGSPLGLAGSGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 484 aa, synthetic construct
LAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 6 - protein, 492 aa, synthetic construct
SDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 7 - protein, 663 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHH

SEQ ID NO: 8 - protein, 256 aa, synthetic construct; identical to SEQ ID NO: 1

SEQ ID NO: 9 - protein, 655 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGSGGGGSGGASSGAGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGHHHHHHHH

SEQ ID NO: 10 - protein, 256 aa, synthetic construct
GGPCRSHIDVAKPICVGGGGSGGGGGSGGGGSGGASSGAGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 487 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGHHHHHHHH

SEQ ID NO: 12 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 5 aa, synthetic construct
  DISULFID 4: note = Interchain disulfide bond with another sequence
GGPCR

SEQ ID NO: 14 - protein, 15 aa, synthetic construct
  DISULFID 3: note = Interchain disulfide bond with another sequence
PICVGGGGSGGLSGR

SEQ ID NO: 15 - protein, 22 aa, synthetic construct
  DISULFID 10: note = Interchain disulfide bond with another sequence
SHIDVAKPICVGGGGSGGLSGR

SEQ ID NO: 16 - protein, 6 aa, synthetic construct
  DISULFID 5: note = Interchain disulfide bond with another sequence
VSFSCR

SEQ ID NO: 17 - protein, 42 aa, synthetic construct
  DISULFID 27: note = Interchain disulfide bond with another sequence
FSGSGSGTDFTLSINSVESEDIADYYCQQNNNWPTTFGAGTK

SEQ ID NO: 18 - protein, 16 aa, synthetic construct
  DISULFID 8: note = Interchain disulfide bond with another sequence
SGTASVVCLLNNFYPR

SEQ ID NO: 19 - protein, 17 aa, synthetic construct
  DISULFID 4: note = Interchain disulfide bond with another sequence
VYACEVTHQGLSSPVTK

SEQ ID NO: 20 - protein, 7 aa, synthetic construct
  DISULFID 7: note = Interchain disulfide bond with another sequence
SFNRGEC

SEQ ID NO: 21 - protein, 19 aa, synthetic construct
  DISULFID 3: note = Interchain disulfide bond with another sequence
LSCAASGSTFYTAVMGWVR

SEQ ID NO: 22 - protein, 23 aa, synthetic construct
  DISULFID 20: note = Interchain disulfide bond with another sequence
TTLYLQMNSLRPEDTAVYYCAAR

SEQ ID NO: 23 - protein, 62 aa, synthetic construct
  DISULFID 39..49: note = Intrachain disulfide bond
GTLGLFTTADSYDYWGQGTLVTVSSGGGGSGGGSGGVYCGPEFDESVGCMGGGGSGGGLSGR

SEQ ID NO: 24 - protein, 12 aa, synthetic construct
  DISULFID 3: note = Interchain disulfide bond with another sequence
LSCAASGFTFNK

SEQ ID NO: 25 - protein, 11 aa, synthetic construct
  DISULFID 9: note = Interchain disulfide bond with another sequence
TEDTAVYYCVR

SEQ ID NO: 26 - protein, 17 aa, synthetic construct
  DISULFID 2: note = Interchain disulfide bond with another sequence
TCGSSTGAVTSGNYPNW

SEQ ID NO: 27 - protein, 4 aa, synthetic construct
  DISULFID 1: note = Interchain disulfide bond with another sequence
CVLW

SEQ ID NO: 28 - protein, 5 aa, synthetic construct
  DISULFID 2: note = Interchain disulfide bond with another sequence
YCVLW

SEQ ID NO: 29 - protein, 21 aa, synthetic construct
  DISULFID 2: note = Interchain disulfide bond with another sequence
TCGSSTGAVTSGNYPNWVQQK

SEQ ID NO: 30 - protein, 5 aa, synthetic construct
  DISULFID 1: note = Interchain disulfide bond with another sequence
CVLWY

SEQ ID NO: 31 - protein, 33 aa, synthetic construct
  DISULFID 17: note = Interchain disulfide bond with another sequence
QSGPGLVQPSQSLSITCTVSGFSLTNYGVHWVR

SEQ ID NO: 32 - protein, 16 aa, synthetic construct
  DISULFID 14: note = Interchain disulfide bond with another sequence
MNSLQSNDTAIYYCAR

SEQ ID NO: 33 - protein, 14 aa, synthetic construct
  DISULFID 11: note = Interchain disulfide bond with another sequence
STSGGTAALGCLVK

SEQ ID NO: 34 - protein, 58 aa, synthetic construct
  DISULFID 53: note = Interchain disulfide bond with another sequence
DYFPEPVTVSWNSGALTSGVHTFPAVLQSSGLYSLSSVVTVPSSSLGTQTYICNVNHK

SEQ ID NO: 35 - protein, 63 aa, synthetic construct
  DISULFID 53: note = Interchain disulfide bond with another sequence
DYFPEPVTVSWNSGALTSGVHTFPAVLQSSGLYSLSSVVTVPSSSLGTQTYICNVNHKPSNTK

(19) United States
(12) Patent Application Publication
(10) Pub. No.: US 2025/0297017 A1
(43) Pub. Date: Sep. 25, 2025

(54) COMPOSITIONS AND USES OF TUMOR ACTIVATED ANTIBODIES TARGETING EGFR AND EFFECTOR CELL ANTIGENS

(71) Applicant: Janux Therapeutics, Inc., San Diego, CA (US)

(72) Inventors: David CAMPBELL, San Diego, CA (US); Thomas R. DIRAIMONDO, San Diego, CA (US); Carolina CAFFARO, San Diego, CA (US); Hans AERNI, San Diego, CA (US)

(21) Appl. No.: 19/187,587

(22) Filed: Apr. 23, 2025

Related U.S. Application Data
(63) Continuation of application No. PCT/IB2023/000677, filed on Nov. 10, 2023.

(30) Foreign Application Priority Data
Nov. 11, 2022 (WO) PCT/IB2022/000650
Mar. 9, 2023 (WO) PCT/IB2023/000137

(51) Int. Cl. C07K 16/28; A61K 39/00; A61K 47/22; A61K 47/26

(52) U.S. Cl. CPC C07K 16/2863; A61K 47/22; A61K 47/26; C07K 16/2809; A61K 2039/505; C07K 2317/31; C07K 2317/55; C07K 2317/622; C07K 2317/94

(57) ABSTRACT

Disclosed herein are compositions and uses of tumor activated antibodies targeting EGFR and effector cell antigens. In particular, the present application provides isolated recombinant polypeptide complexes that comprise binding domains that selectively bind to an effector cell antigen and EGFR, in which one or more of the binding domains is selectively activated in the tumor microenvironment and the isolated polypeptide or polypeptide complex comprises a half-life extending molecule.

### CROSS-REFERENCE

This application is a continuation of PCT International Application No. PCT/IB2023/000677, filed Nov. 10, 2023, which claims the benefit of PCT International Application No. PCT/IB2022/000650, filed Nov. 11, 2022, and PCT International Application No. PCT/IB2023/000137, filed Mar. 9, 2023, each of which is incorporated herein by reference in its entirety.

### SEQUENCE LISTING

The instant application contains a Sequence Listing which has been submitted electronically in XML file format and is hereby incorporated by reference in its entirety. The XML copy, created on Oct. 26, 2023, is named 52426-750_301SL.xml and is 45,501 bytes in size.

### SUMMARY

In one aspect, disclosed herein is an isolated recombinant polypeptide complex comprising a first chain with an amino acid sequence having at least 70% sequence identity to SEQ ID NO: 1 and a second chain with an amino acid sequence having at least 70% sequence identity to SEQ ID NO: 2 wherein the isolated recombinant polypeptide complex comprises at least one of the following characteristics: (a) at least one N-glycan moiety; (b) at least one disulfide bond; (c) a melting onset temperature (T_{Onset}) between about 60° C. to about 65° C. and a transition mid-point temperature (Tₘₗ) between about 70° C. and about 75° C. when the isolated recombinant polypeptide complex is formulated at a concentration of about 1.0 mg/mL in about 10 mM histidine, about 8% (w/v) sucrose, about 0.01% (w/v) polysorbate 20 (PS20) pH of about 5.3, wherein the T_{Onset} and the Tₘₗ are measured using differential scanning calorimetry (DSC); (d) a far UV circular dichroism dip at a wavelength between 210 nm and 230 nm when the isolated recombinant polypeptide complex is formulated at a concentration of about 0.1 mg/mL in water; or (e) a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3. In some embodiments, the polypeptide complex comprises at least two of the characteristics. In some embodiments, the polypeptide complex comprises at least three of the characteristics. In some embodiments, the polypeptide complex comprises at least four of the characteristics. In some embodiments, the polypeptide complex comprises at least five of the characteristics. In some embodiments, the first chain comprises at least 75% sequence identity to SEQ ID NO: 1. In some embodiments, the first chain comprises at least 80% sequence identity to SEQ ID NO: 1. In some embodiments, the first chain comprises at least 85% sequence identity to SEQ ID NO: 1. In some embodiments, the first chain comprises at least 90% sequence identity to SEQ ID NO: 1. In some embodiments, the first chain comprises at least 95% sequence identity to SEQ ID NO: 1. In some embodiments, the first chain comprises at least 99% sequence identity to SEQ ID NOs: 1. In some embodiments, the first chain comprises the amino acid sequence according to SEQ ID NO: 1. In some embodiments, the second chain comprises at least 75% sequence identity to SEQ ID NO: 2. In some embodiments, the second chain comprises at least 80% sequence identity to SEQ ID NO: 2. In some embodiments, the second chain comprises at least 85% sequence identity to SEQ ID NO: 2. In some embodiments, the second chain comprises at least 90% sequence identity to SEQ ID NO: 2. In some embodiments, the second chain comprises at least 95% sequence identity to SEQ ID NO: 2. In some embodiments, the second chain comprises at least 99% sequence identity to SEQ ID NO: 2. In some embodiments, the second chain comprises the amino acid sequence according to SEQ ID NO: 2. In some embodiments, the at least one N-glycan moiety is located on the first chain. In some embodiments, the at least one N-glycan moiety is located on the second chain. In some embodiments, the at least one N-glycan moiety comprises G2F, G2FS1, or G2FS2. In some embodiments, the at least one N-glycan moiety comprises G2F. In some embodiments, the at least one N-glycan moiety comprises G2FS1. In some embodiments, the at least one N-glycan moiety comprises G2FS2. In some embodiments, at least one asparagine deamidation moiety is located at Asparagine 83 of SEQ ID NO: 1. In some embodiments, the at least one N-glycan moiety is located at Asparagine 519 of SEQ ID NO: 2. In some embodiments, the isolated recombinant polypeptide complex further comprises O-xylosylation, asparagine deamidation, or succinimide formation. In some embodiments, the succinimide formation is located at Asparagine 83 of SEQ ID NO: 1. In some embodiments, the polypeptide complex comprises at least two disulfide bonds formed by pairs of cysteine residues. In some embodiments, the polypeptide complex comprises at least three disulfide bonds formed by pairs of cysteine residues. In some embodiments, the polypeptide complex comprises at least four disulfide bonds formed by pairs of cysteine residues. In some embodiments, the polypeptide complex comprises at least five disulfide bonds formed by pairs of cysteine residues. In some embodiments, the polypeptide complex comprises at least six disulfide bonds formed by pairs of cysteine residues. In some embodiments, the polypeptide complex comprises at least seven disulfide bonds formed by pairs of cysteine residues. In some embodiments, the polypeptide complex comprises at least eight disulfide bonds formed by pairs of cysteine residues. In some embodiments, the polypeptide complex comprises at least nine disulfide bonds formed by pairs of cysteine residues. In some embodiments, the polypeptide complex comprises at least ten disulfide bonds formed by pairs of cysteine residues. In some embodiments, the pair of cysteine residues comprises Cysteine 4 and Cysteine 15 of SEQ ID NO: 1. In some embodiments, the pair of cysteine residues comprises Cysteine 65 and Cysteine 130 of SEQ ID NO: 1. In some embodiments, the pair of cysteine residues comprises Cysteine 176 and Cysteine 236 of SEQ ID NO: 1. In some embodiments, the pair of cysteine residues comprises Cysteine 256 of SEQ ID NO: 1 and Cysteine 653 of SEQ ID NO: 2. In some embodiments, the pair of cysteine residues comprises Cysteine 22 and Cysteine 96 of SEQ ID NO: 2. In some embodiments, the pair of cysteine residues comprises Cysteine 138 and Cysteine 148 of SEQ ID NO: 2. In some embodiments, the pair of cysteine residues comprises Cysteine 199 and Cysteine 275 of SEQ ID NO: 2. In some embodiments, the pair of cysteine residues comprises Cysteine 339 and Cysteine 407 of SEQ ID NO: 2. In some embodiments, the pair of cysteine residues comprises Cysteine 453 and Cysteine 526 of SEQ ID NO: 2. In some embodiments, the pair of cysteine residues comprises Cysteine 577 and Cysteine 633 of SEQ ID NO: 2. In some embodiments, the polypeptide complex comprises 1 inter-chain disulfide bonds between the first chain and the second chain and 9 intra-chain disulfide bonds. In some embodiments, the polypeptide complex comprises 1 inter-chain disulfide bonds between the first chain and the second chain and the second chain comprises 6 intra-chain disulfide bonds and the first chain comprises 3 intra-chain disulfide bonds. In some embodiments, the isolated recombinant polypeptide complex has a T_{Onset} between about 61° C. to about 64.5° C. In some embodiments, the isolated recombinant polypeptide complex has a T_{Onset} between about 62° C. to about 64° C. In some embodiments, the isolated recombinant polypeptide complex has a T_{Onset} of about 62.5° C. In some embodiments, the isolated recombinant polypeptide complex has a T_{Onset} of about 63.2° C. In some embodiments, the isolated recombinant polypeptide complex has a Tₘₗ between about 71° C. to about 75° C. In some embodiments, the isolated recombinant polypeptide complex has a Tₘₗ between about 72.5° C. to about 74.5° C. In some embodiments, the isolated recombinant polypeptide complex has a Tₘₗ of about 73.8° C. In some embodiments, the isolated recombinant polypeptide complex has a Tₘₗ of about 74.0° C. In some embodiments, the secondary structure composition comprises a β-sheet and a random coil. In some embodiments, the isolated recombinant polypeptide complex has a far UV circular dichroism dip at a wavelength between 215 nm and 225 nm. In some embodiments, the isolated recombinant polypeptide complex has a far UV circular dichroism dip at a wavelength between 215 nm and 220 nm. In some embodiments, the isolated recombinant polypeptide complex has a near UV circular dichroism dip at a wavelength between 280 nm and 290 nm. In some embodiments, the isolated recombinant polypeptide complex has a near UV circular dichroism dip at a wavelength between 280 nm and 285 nm. In some embodiments, the isolated recombinant polypeptide complex has a near UV circular dichroism peak at a wavelength between 270 nm and 275 nm. In some embodiments, the isolated recombinant polypeptide complex has a near UV circular dichroism peak at a wavelength between 285 nm and 290 nm. In some embodiments, the first chain comprises the amino acid sequence according to SEQ ID NO: 1, and the second chain comprises the amino acid sequence according to SEQ ID NO: 2, and the at least one N-glycan moiety comprises G2F, G2FS1, or G2FS2, and the recombinant polypeptide complex comprises disulfide bonds formed by pairs of cysteine residues Cysteine 4 and Cysteine 15 of SEQ ID NO: 1, Cysteine 65 and Cysteine 130 of SEQ ID NO: 1, Cysteine 176 and Cysteine 236 of SEQ ID NO: 1, Cysteine 256 of SEQ ID NO: 1 and Cysteine 653 of SEQ ID NO: 2, Cysteine 138 and Cysteine 148 of SEQ ID NO: 2, Cysteine 22 and Cysteine 96 of SEQ ID NO: 2, Cysteine 199 and Cysteine 275 of SEQ ID NO: 2, Cysteine 339 and Cysteine 407 of SEQ ID NO: 2, Cysteine 453 and Cysteine 526 of SEQ ID NO: 2, and Cysteine 577 and Cysteine 633 of SEQ ID NO: 2, and the T_{Onset} is between about 60° C. to about 65° C. and a transition mid-point temperature (Tₘₗ) between about 70° C. and about 75° C., wherein the T_{Onset} and the Tₘₗ are measured using differential scanning calorimetry (DSC), when the isolated recombinant polypeptide complex is formulated at a concentration of about 1.0 mg/mL in about 10 mM histidine, about 8% (w/v) sucrose, about 0.01% (w/v) PS20 pH of about 5.3, and a far UV circular dichroism dip at a wavelength between 210 nm and 230 nm when the isolated recombinant polypeptide complex is formulated at a concentration of 0.1 mg/mL in water, and a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

In another aspect, disclosed herein is an isolated recombinant polypeptide complex comprising a first chain with an amino acid sequence having at least 80% sequence identity to SEQ ID NO: 1 and a second chain with an amino acid sequence having at least 80% sequence identity to SEQ ID NO: 2 wherein the isolated recombinant polypeptide complex comprises the following characteristics: (a) at least one N-glycan moiety; (b) at least one disulfide bond; (c) a melting onset temperature (T_{Onset}) between about 60° C. to about 65° C. and a transition mid-point temperature (Tₘₗ) between about 70° C. and about 75° C., wherein the T_{Onset} and the Tₘₗ are measured using differential scanning calorimetry (DSC), when the isolated recombinant polypeptide complex is formulated at a concentration of about 1.0 mg/mL in about 10 mM histidine, about 8% (w/v) sucrose, about 0.01% (w/v) PS20 pH of about 5.3; (d) a far UV circular dichroism dip at a wavelength between 210 nm and 230 nm when the isolated recombinant polypeptide complex is formulated at a concentration of about 0.1 mg/mL in water; or (e) a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

In another aspect, disclosed herein is a pharmaceutical composition comprising: (a) the isolated recombinant polypeptide complex disclosed herein; and (b) a pharmaceutically acceptable carrier. In some embodiments, the pharmaceutically acceptable carrier comprises a buffer, a stabilizing agent, a tonicity agent, a surfactant, or combinations thereof. In some embodiments, the buffer comprises an amino acid or a derivative thereof. In some embodiments, the amino acid or derivative thereof comprises L-histidine, L-histidine hydrochloride monohydrate, or a combination thereof. In some embodiments, the surfactant is polysorbate 20. In some embodiments, the stabilizing agent is sucrose. In some embodiments, the pharmaceutical composition has a pH less than 6.0.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the disclosure are set forth with particularity in the appended claims. A better understanding of the features and advantages of the present invention will be obtained by reference to the following detailed description that sets forth illustrative embodiments, in which the principles of the disclosure are utilized, and the accompanying drawings (also “Figure” and “FIG.” herein), of which:

FIG. 1 illustrates design, structure and mechanism of action of a polypeptide complex 1 (PC-1) disclosed herein. PC-1 is a tumor-activated T cell engager with EGFR- and CD3-binding domains, an albumin-binding domain to extend circulating half-life, a peptide mask that inhibits CD3 engagement on T cells, and a tumor protease cleavable linker. Tumor-specific proteolysis of the cleavable linker in the tumor microenvironment (TME) separates the tandem mask and albumin-binding domain from PC-1. It enables TME restricted CD3 binding and subsequent T cell activation against EGFR expressing cancer cells. Loss of the albumin-binding domain likely ensures that any activated PC-1 that migrates out of the tumor will be cleared rapidly and reduces its potential accumulation in healthy tissues that can contribute to safety risks.

FIG. 2 illustrates design and structure of polypeptide complex 1 (PC-1). PC-1 is a tumor-activated T cell engager with EGFR- and CD3-binding domains (VH2 and VL1), an albumin-binding domain to extend circulating half-life (VH1), a peptide mask that inhibits CD3 engagement on T cells, a peptide mask that inhibits binding to EGFR, and a tumor protease cleavable linker.

FIG. 3 illustrates a flow diagram of an upstream cell culture process used in the production of PC-1.

FIG. 4 illustrates a flow diagram of a downstream purification process used in the production of PC-1.

FIG. 5 illustrates circular dichroism (CD) spectra of PC-1 in the far-UV region.

FIG. 6 illustrates CD spectra of PC-1 in the near-UV region.

FIG. 7 illustrates differential scanning calorimetry data for PC-1.

FIGS. 8A, 8B, 8C and 8D illustrate SEC-MALS chromatograms of PC-1.

FIGS. 9A and 9B illustrate Dynamic Light Scattering chromatograms of PC-1.

FIG. 10 illustrates symbol structures of major N-glycans.

FIG. 11 illustrates the light chain (LC) and heavy chain (HC) arrangement in PC-1.

FIGS. 12A and 12B illustrate the binding to human EGFR and cynomolgus monkey EGFR by PC-1, PC-1-MMP9 Cleaved, PC-1-SP Cleaved, or PC-1-TCE.

FIGS. 13A and 13B illustrate the binding to human CD3 and cynomolgus monkey CD3 by PC-1, PC-1-MMP Cleaved, PC-1-SP Cleaved, or PC-1-TCE.

FIGS. 14A and 14B illustrate PC-1 binding to human albumin and cynomolgus monkey albumin.

FIGS. 15A, 15B, 15C, and 15D illustrate HCT116 tumor cell killing by donor PBMCs stimulated by PC-1-TCE, PC-1, PC-1-SP Cleaved, or PC-1-MMP Cleaved.

FIGS. 16A, 16B, 16C, and 16D illustrate A549 tumor cell killing by donor PBMCs stimulated by PC-1-TCE, PC-1, PC-1-SP Cleaved, or PC-1-MMP Cleaved.

FIGS. 17A, 17B, 17C, and 17D illustrate Cal27 tumor cell killing by donor PBMCs stimulated by PC-1-TCE, PC-1, PC-1-SP Cleaved, or PC-1-MMP Cleaved.

FIGS. 18A and 18B illustrate A549 EGFR-KO tumor cell killing by donor PBMCs stimulated by PC-1-TCE, PC-1, PC-1-SP Cleaved, or PC-1-MMP Cleaved.

FIGS. 19A, 19B, 19C, 19D, 19E, and 19F illustrate release of IFNγ, TNF, and IL-6 by healthy donor PBMCs induced by PC-1-TCE, PC-1, PC-1-SP Cleaved, or PC-1-MMP Cleaved in presence of HCT116 cells.

FIGS. 20A, 20B, 20C, 20D, 20E, and 20F illustrate release of IFNγ, TNF, and IL-6 by healthy donor PBMCs induced by PC-1-TCE, PC-1, PC-1-SP Cleaved, or PC-1-MMP Cleaved in presence of A549 cells.

FIGS. 21A, 21B, 21C, 21D, 21E, and 21F illustrate release of IFNγ, TNF, and IL-6 by healthy donor PBMCs induced by PC-1-TCE, PC-1, PC-1-SP Cleaved, or PC-1-MMP Cleaved in presence of Cal27 cells.

FIGS. 22A, 22B, 22C, 22D, 22E, and 22F illustrate lack of release of IFNγ, TNF, and IL-6 by healthy donor PBMCs induced by PC-1-TCE, PC-1, PC-1-SP Cleaved, or PC-1-MMP Cleaved in presence of EGFR-KO A549 cells.

FIG. 23 illustrates cleavage dependent PC-1-Histag activity in HCT116 tumor-bearing mice co-engrafted with human PBMCs using Vehicle, PC-1-NC (0.5 mg/kg), PC-1-TCE (0.5 mg/kg), PC-1-Histag (0.15 mg/kg), PC-1-Histag (0.5 mg/kg), and PC-1-Histag (1.5 mg/kg).

FIG. 24 illustrates the structure of PC-1.

FIG. 25 illustrates the structure of PC-1-SP Cleaved.

FIG. 26 illustrates the structure of PC-1-MMP Cleaved.

FIG. 27 illustrates the structure of PC-1-TCE.

FIG. 28 illustrates the structure of PC-1-HisTag.

FIG. 29 illustrates the structure of PC-1-NC.

### DETAILED DESCRIPTION OF THE INVENTION

Multispecific antibodies combine the benefits of different binding specificities derived from two or more antibodies into a single composition. Multispecific antibodies for redirecting T cells to cancers have shown promise in both pre-clinical and clinical studies. This approach relies on binding of one antigen interacting portion of the antibody to a tumor-associated antigen or marker, while a second antigen interacting portion can bind to an effector cell antigen on a T cell, such as CD3, which then triggers cytotoxic activity. One such tumor-associated antigen is epidermal growth factor receptor (EGFR). EGFR is a transmembrane protein that is a receptor for members of the epidermal growth factor family of extracellular protein ligands. EGFR is the most commonly overexpressed membrane protein in cancer. However, EGFR expression is not limited to tumors and is widely expressed throughout the body, resulting in systemic toxicities with EGFR-directed therapies.

T cell engagers (TCEs) therapeutics have several benefits including they are not cell therapies and thus can be offered as off-the-shelf therapies as opposed to chimeric antigen receptor T cell (CAR T cell) therapies. While TCE therapeutics have displayed potent anti-tumor activity in hematological cancers, developing TCEs to treat solid tumors has faced challenges due to the limitations of prior TCE technologies, namely (i) overactivation of the immune system leading to cytokine release syndrome (CRS), (ii) on-target, healthy tissue toxicities and (iii) poor pharmacokinetics (PK) leading to short half-life. CRS arises from the systemic activation of T cells and can result in life-threatening elevations in inflammatory cytokines such as interleukin-6 (IL-6). Severe and acute CRS leading to dose limited toxicities and deaths have been observed upon the dosing of T cell engagers developed using other platforms to treat cancer patients in poor clinical studies. This toxicity restricts the maximum blood levels of T cell engagers that can be safely dosed. T cell engager effectiveness has also been limited because of on-target, healthy tissue toxicity. T cell engagers developed using a platform not designed for tumor-specification activation have resulted in clinical holds and dose-limiting toxicities resulting from target expression in healthy tissues. T cell engagers have also been limited by short half-lives. T cell engagers quickly reach sub-therapeutic levels after being administered as they are quickly eliminated from the body due to their short exposure half-lives. For this reason, T cell engagers such as blinatumomab are typically administered by a low-dose, continuous infusion pump over a period of weeks to overcome the challenge of a short half-life and to maintain therapeutic levels of drug in the body. A continuous dosing regimen represents a significant burden for patients.

To overcome these challenges associated with the effectiveness of T cell engagers, described herein, are recombinant polypeptide complexes that comprise binding domains that selectively bind to an effector cell antigen and EGFR, in which one or more of the binding domains is selectively activated in the tumor microenvironment and the isolated polypeptide or polypeptide complex comprises a half-life extending molecule. Such modifications reduce CRS and on-target healthy tissue toxicity risk, and improves stability in the bloodstream and serum half-life prior to activation. The recombinant polypeptide complexes described herein have activity at low levels of target expression, and can be easily manufactured and formulated.

In some embodiments, the recombinant polypeptide complexes described herein are used in a method of treating cancer. In some embodiments, the cancer has cells that express EGFR. In some embodiments, the recombinant polypeptide complexes described herein are used in a method of treating renal cell carcinoma, colorectal cancer (CRC), squamous cell carcinoma of the head and Neck (SCCHN), non-small cell lung cancer (NSCLC), prostate cancer, breast cancer, colon/rectum cancer, head and neck cancer, esophagogastric cancer, liver cancer, glioblastoma, cervical cancer, ovarian cancer, bladder cancer, kidney cancer, or pancreatic cancer. In some embodiments, the polypeptides or polypeptide complexes described herein are used in a method of treating subjects who are resistant to EGFR inhibitor treatment. In some embodiments, the recombinant polypeptide complexes described herein are used in a method of treating subjects who harbor KRAS mutations. In some embodiments, the recombinant polypeptide complex described herein are used in a method of treating subjects who are resistant to EGFR inhibitor treatment and harbor KRAS mutations.

### One or More Characteristics

An isolated polypeptide or recombinant polypeptide complex described herein may have one or more characteristics as described hereinabove in this section.

In some embodiments, the isolated recombinant polypeptide complex comprises a first chain with an amino acid sequence having at least 70% sequence identity to SEQ ID NO: 1 and a second chain with an amino acid sequence having at least 70% sequence identity to SEQ ID NO: 2. In some embodiments, the recombinant polypeptide complex has at least one, at least two, at least three, at least four, or all five of the characteristics (a)-(e).:
(a) at least one N-glycan moiety;
(b) at least one disulfide bond;
(c) a melting onset temperature (T_{Onset}) between about 60° C. to about 65° C. and a transition mid-point temperature (Tₘₗ) between about 71° C. and about 76° C. when the isolated recombinant polypeptide complex is formulated at a concentration of about 1.0 mg/mL in about 10 mM histidine, about 8% (w/v) sucrose, about 0.01% (w/v) PS20 pH of about 5.3;
(d) a far UV circular dichroism negative peak at a wavelength between 210 nm and 230 nm when the isolated recombinant polypeptide complex is formulated at a concentration of 0.1 mg/mL in water; or
(e) a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

In some embodiments, the isolated recombinant polypeptide complex comprises a first chain with an amino acid sequence having at least 70% sequence identity to SEQ ID NO: 1 and a second chain with an amino acid sequence having at least 70% sequence identity to SEQ ID NO: 2. In some embodiments, the recombinant polypeptide complex has at least one, at least two, at least three, at least four, or all five of the characteristics (a)-(e).:
(a) at least one N-glycan moiety;
(b) at least one disulfide bond;
(c) a melting onset temperature (T_{Onset}) between about 60° C. to about 65° C. and a transition mid-point temperature (Tₘₗ) between about 70° C. and about 75° C. when the isolated recombinant polypeptide complex is formulated at a concentration of about 1.0 mg/mL in about 10 mM histidine, about 8% (w/v) sucrose, about 0.01% (w/v) PS20 pH of about 5.3, wherein the T_{Onset} and the Tₘₗ are measured using Differential Scanning Calorimetry (DSC);
(d) a far UV circular dichroism dip at a wavelength between 210 nm and 230 nm when the isolated recombinant polypeptide complex is formulated at a concentration of about 0.1 mg/mL in water; or
(e) a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

In some embodiments, the isolated recombinant polypeptide complex comprises a first chain with an amino acid sequence having at least 80% sequence identity to SEQ ID NO: 1 and a second chain with an amino acid sequence having at least 80% sequence identity to SEQ ID NO: 2 wherein the isolated recombinant polypeptide complex comprises the following characteristics:
(a) at least one N-glycan moiety;
(b) at least one disulfide bond;
(c) a melting onset temperature (T_{Onset}) between about 60° C. to about 65° C. and a transition mid-point temperature (Tₘₗ) between about 70° C. and about 75° C., wherein the T_{Onset} and the Tₘₗ are measured using differential scanning calorimetry (DSC), when the isolated recombinant polypeptide complex is formulated at a concentration of about 1.0 mg/mL in about 10 mM histidine, about 8% (w/v) sucrose, about 0.01% (w/v) PS20 pH of about 5.3;
(d) a far UV circular dichroism dip at a wavelength between 210 nm and 230 nm when the isolated recombinant polypeptide complex is formulated at a concentration of about 0.1 mg/mL in water; or
(e) a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

In some embodiments, the recombinant polypeptide complex comprises at least one (e.g., one, two, three, four, five, six, or seven) of the characteristics (a)-(e). In some embodiments, the recombinant polypeptide complex comprises at least two (e.g., two, three, four, five, six, or seven) of the characteristics (a)-(e). In some embodiments, the recombinant polypeptide complex comprises at least three (e.g., three, four, five, six, or seven) of the characteristics (a)-(e). In some embodiments, the recombinant polypeptide complex comprises at least four (e.g., four, five, six or seven) of the characteristics (a)-(e). In some embodiments, the isolated recombinant polypeptide complex comprises at least five (e.g., five, six or seven) of the characteristics (a)-(e).

### Isolated Recombinant Polypeptide Complex Compositions

In some embodiments, the isolated recombinant polypeptide complex comprises a tumor-activated T-cell engager with EGFR and CD3 binding domains, an albumin binding domain to extend circulating half-life, a peptide mask that inhibits CD3 engagement on T-cells, and a tumor protease cleavable linker. Tumor specific proteolysis of the cleavable linker in the tumor microenvironment can separate the tandem mask and albumin-binding domain from the isolated recombinant polypeptide complex. The isolated recombinant polypeptide complex can comprise chain 1 and chain 2 as described herein. In some embodiments, chain 1 comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1. In some embodiments, chain 2 comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 2.

In some embodiments, the isolated recombinant polypeptide complex comprises an amino acid sequence having at least 80% sequence identity to SEQ ID NO: 1. In some embodiments, the isolated recombinant polypeptide complex comprises an amino acid sequence having at least 85% sequence identity to SEQ ID NO: 1. In some embodiments, the isolated recombinant polypeptide complex comprises an amino acid sequence having at least 90% sequence identity to SEQ ID NO: 1. In some embodiments, the isolated recombinant polypeptide complex comprises an amino acid sequence having at least 91% sequence identity to SEQ ID NO: 1. In some embodiments, the isolated recombinant polypeptide complex comprises an amino acid sequence having at least 92% sequence identity to SEQ ID NO: 1. In some embodiments, the isolated recombinant polypeptide complex comprises an amino acid sequence having at least 93% sequence identity to SEQ ID NO: 1. In some embodiments, the isolated recombinant polypeptide complex comprises an amino acid sequence having at least 94% sequence identity to SEQ ID NO: 1. In some embodiments, the isolated recombinant polypeptide complex comprises an amino acid sequence having at least 95% sequence identity to SEQ ID NO: 1. In some embodiments, the isolated recombinant polypeptide complex comprises an amino acid sequence having at least 96% sequence identity to SEQ ID NO: 1. In some embodiments, the isolated recombinant polypeptide complex comprises an amino acid sequence having at least 97% sequence identity to SEQ ID NO: 1. In some embodiments, the isolated recombinant polypeptide complex comprises an amino acid sequence having at least 98% sequence identity to SEQ ID NO: 1. In some embodiments, the isolated recombinant polypeptide complex comprises an amino acid sequence having at least 99% sequence identity to SEQ ID NO: 1.

In some embodiments, the isolated recombinant polypeptide complex comprises an amino acid sequence according to SEQ ID NO: 1.

In some embodiments, the isolated recombinant polypeptide complex comprises an amino acid sequence having at least 80% sequence identity to SEQ ID NO: 2. In some embodiments, the isolated recombinant polypeptide complex comprises an amino acid sequence having at least 85% sequence identity to SEQ ID NO: 2. In some embodiments, the isolated recombinant polypeptide complex comprises an amino acid sequence having at least 90% sequence identity to SEQ ID NO: 2. In some embodiments, the isolated recombinant polypeptide complex comprises an amino acid sequence having at least 91% sequence identity to SEQ ID NO: 2. In some embodiments, the isolated recombinant polypeptide complex comprises an amino acid sequence having at least 92% sequence identity to SEQ ID NO: 2. In some embodiments, the isolated recombinant polypeptide complex comprises an amino acid sequence having at least 93% sequence identity to SEQ ID NO: 2. In some embodiments, the isolated recombinant polypeptide complex comprises an amino acid sequence having at least 94% sequence identity to SEQ ID NO: 2. In some embodiments, the isolated recombinant polypeptide complex comprises an amino acid sequence having at least 95% sequence identity to SEQ ID NO: 2. In some embodiments, the isolated recombinant polypeptide complex comprises an amino acid sequence having at least 96% sequence identity to SEQ ID NO: 2. In some embodiments, the isolated recombinant polypeptide complex comprises an amino acid sequence having at least 97% sequence identity to SEQ ID NO: 2. In some embodiments, the isolated recombinant polypeptide complex comprises an amino acid sequence having at least 98% sequence identity to SEQ ID NO: 2. In some embodiments, the isolated recombinant polypeptide complex comprises an amino acid sequence having at least 99% sequence identity to SEQ ID NO: 2.

In some embodiments, the isolated recombinant polypeptide complex comprises an amino acid sequence according to SEQ ID NO: 2.

In some embodiments, the LC comprises at least 85% sequence identity to SEQ ID NO: 1. In some embodiments, the LC comprises at least 90% sequence identity to SEQ ID NO: 1. In some embodiments, the LC comprises at least 95% sequence identity to SEQ ID NO: 1. In some embodiments, the LC comprises at least 99% sequence identity to SEQ ID NO: 1. In some embodiments, the LC comprises the amino acid sequence according to SEQ ID NO: 1. In some embodiments, the HC comprises at least 85% sequence identity to SEQ ID NO: 2. In some embodiments, the HC comprises at least 90% sequence identity to SEQ ID NO: 2. In some embodiments, the HC comprises at least 95% sequence identity to SEQ ID NO: 2. In some embodiments, the HC comprises at least 99% sequence identity to SEQ ID NO: 2. In some embodiments, the HC comprises the amino acid sequence according to SEQ ID NO: 2. In some embodiments, the LC comprises at least 99% sequence identity to SEQ ID NO: 1, the HC comprises at least 99% sequence identity to SEQ ID NO: 2. In some embodiments, the LC comprises the amino acid sequence according to SEQ ID NO: 1, the HC comprises the amino acid sequence according to SEQ ID NO: 2.

TABLE 1
Exemplary Amino Acid Sequences of an Isolated Recombinant
Polypeptide Complex (PC-1) that selectively binds to EGFR
and effector cell antigens such as CD3 and its Metabolites
and Derivatives

| Construct | Amino Acid Sequence | SEQ ID |
|---|---|---|
| Description | (N to C) | NO: |
| PC-1 Light | GGPCRSHIDVAKPICVGGGGSGGLSGRSDAGSPLGLAGSG | 1 |
| Chain (LC) of | GSDILLTQSPVILSVSPGERVSFSCRASQSIGTNIHWYQQ | |
| anti-EGFR Fab | RTNGSPRLLIKYASESISGIPSRFSGSGSGTDFTLSINSV | |
| | ESEDIADYYCQQNNNWPTTFGAGTKLELKRTVAAPSVFIF | |
| | PPSDEQLKSGTASVVCLLNNFYPREAKVQWKVDNALQSGN | |
| | SQESVTEQDSKDSTYSLSSTLTLSKADYEKHKVYACEVTH | |
| | QGLSSPVTKSFNRGEC | |
| PC-1 Heavy | EVQLVESGGGLVQPGGSLRLSCAASGSTFYTAVMGWVRQA | 2 |
| Chain | PGKGLEWVAAIRWTALTTSYADSVKGRFTISRDGAKTTLY | |
| (HC) of anti- | LQMNSLRPEDTAVYYCAARGTLGLFTTADSYDYWGQGTLV | |
| EGFR Fab | TVSSGGGGSGGGSGGVYCGPEFDESVGCMGGGGSGGGLSG | |
| linked to an | RSDAGSPLGLAGSGGGSEVQLVESGGGLVQPGGSLKLSCA | |
| anti-CD3 scFv | ASGFTFNKYAMNWVRQAPGKGLEWVARIRSKYNNYATYYA | |
| | DSVKDRFTISRDDSKNTAYLQMNNLKTEDTAVYYCVRHGN | |
| | FGNSYISYWAYWGQGTLVTVSSGGGGSGGGGSGGGGSQTV | |
| | VTQEPSLTVSPGGTVTLTCGSSTGAVTSGNYPNWVQQKPG | |
| | QAPRGLIGGTKFLAPGTPARFSGSLLGGKAALTLSGVQPE | |
| | DEAEYYCVLWYSNRWVFGGGTKLTVLGGGGSQVQLKQSGP | |
| | GLVQPSQSLSITCTVSGFSLTNYGVHWVRQSPGKGLEWLG | |
| | VIWSGGNTDYNTPFTSRLSINKDNSKSQVFFKMNSLQSND | |
| | TAIYYCARALTYYDYEFAYWGQGTLVTVSAASTKGPSVFP | |
| | LAPSSKSTSGGTAALGCLVKDYFPEPVTVSWNSGALTSGV | |
| | HTFPAVLQSSGLYSLSSVVTVPSSSLGTQTYICNVNHKPS | |
| | NTKVDKKVEPKSC | |
| PC-1 LC Matrix | LAGSGGSDILLTQSPVILSVSPGERVSFSCRASQSIGTNI | 3 |
| Metallopeptidase | HWYQQRTNGSPRLLIKYASESISGIPSRFSGSGSGTDFTL | |
| 9 (MMP9) | SINSVESEDIADYYCQQNNNWPTTFGAGTKLELKRTVAAP | |
| Cleaved | SVFIFPPSDEQLKSGTASVVCLLNNFYPREAKVQWKVDNA | |
| | LQSGNSQESVTEQDSKDSTYSLSSTLTLSKADYEKHKVYA | |
| | CEVTHQGLSSPVTKSFNRGEC | |
| PC-1 LC Serine | SDAGSPLGLAGSGGSDILLTQSPVILSVSPGERVSFSCRA | 4 |
| Protease (SP) | SQSIGTNIHWYQQRTNGSPRLLIKYASESISGIPSRFSGS | |
| Cleaved | GSGTDFTLSINSVESEDIADYYCQQNNNWPTTFGAGTKLE | |
| | LKRTVAAPSVFIFPPSDEQLKSGTASVVCLLNNFYPREAK | |
| | VQWKVDNALQSGNSQESVTEQDSKDSTYSLSSTLTLSKAD | |
| | YEKHKVYACEVTHQGLSSPVTKSFNRGEC | |
| PC-1 HC | LAGSGGGSEVQLVESGGGLVQPGGSLKLSCAASGFTFNKY | 5 |
| MMP9 Cleaved | AMNWVRQAPGKGLEWVARIRSKYNNYATYYADSVKDRFTI | |
| | SRDDSKNTAYLQMNNLKTEDTAVYYCVRHGNFGNSYISYW | |
| | AYWGQGTLVTVSSGGGGSGGGGSGGGGSQTVVTQEPSLTV | |
| | SPGGTVTLTCGSSTGAVTSGNYPNWVQQKPGQAPRGLIGG | |
| | TKFLAPGTPARFSGSLLGGKAALTLSGVQPEDEAEYYCVL | |
| | WYSNRWVFGGGTKLTVLGGGGSQVQLKQSGPGLVQPSQSL | |
| | SITCTVSGFSLTNYGVHWVRQSPGKGLEWLGVIWSGGNTD | |
| | YNTPFTSRLSINKDNSKSQVFFKMNSLQSNDTAIYYCARA | |
| | LTYYDYEFAYWGQGTLVTVSAASTKGPSVFPLAPSSKSTS | |
| | GGTAALGCLVKDYFPEPVTVSWNSGALTSGVHTFPAVLQS | |
| | SGLYSLSSVVTVPSSSLGTQTYICNVNHKPSNTKVDKKVE | |
| | PKSC | |
| PC-1 HC SP | SDAGSPLGLAGSGGGSEVQLVESGGGLVQPGGSLKLSCAA | 6 |
| Cleaved | SGFTFNKYAMNWVRQAPGKGLEWVARIRSKYNNYATYYAD | |
| | SVKDRFTISRDDSKNTAYLQMNNLKTEDTAVYYCVRHGNF | |
| | GNSYISYWAYWGQGTLVTVSSGGGGSGGGGSGGGGSQTVV | |
| | TQEPSLTVSPGGTVTLTCGSSTGAVTSGNYPNWVQQKPGQ | |
| | APRGLIGGTKFLAPGTPARFSGSLLGGKAALTLSGVQPED | |
| | EAEYYCVLWYSNRWVFGGGTKLTVLGGGGSQVQLKQSGPG | |
| | LVQPSQSLSITCTVSGFSLTNYGVHWVRQSPGKGLEWLGV | |
| | IWSGGNTDYNTPFTSRLSINKDNSKSQVFFKMNSLQSNDT | |
| | AIYYCARALTYYDYEFAYWGQGTLVTVSAASTKGPSVFPL | |
| | APSSKSTSGGTAALGCLVKDYFPEPVTVSWNSGALTSGVH | |
| | TFPAVLQSSGLYSLSSVVTVPSSSLGTQTYICNVNHKPSN | |
| | TKVDKKVEPKSC | |
| PC-1 HisTag | EVQLVESGGGLVQPGGSLRLSCAASGSTFYTAVMGWVRQA | 7 |
| HC | PGKGLEWVAAIRWTALTTSYADSVKGRFTISRDGAKTTLY | |
| | LQMNSLRPEDTAVYYCAARGTLGLFTTADSYDYWGQGTLV | |
| | TVSSGGGGSGGGSGGVYCGPEFDESVGCMGGGGSGGGLSG | |
| | RSDAGSPLGLAGSGGGSEVQLVESGGGLVQPGGSLKLSCA | |
| | ASGFTFNKYAMNWVRQAPGKGLEWVARIRSKYNNYATYYA | |
| | DSVKDRFTISRDDSKNTAYLQMNNLKTEDTAVYYCVRHGN | |
| | FGNSYISYWAYWGQGTLVTVSSGGGGSGGGGSGGGGSQTV | |
| | VTQEPSLTVSPGGTVTLTCGSSTGAVTSGNYPNWVQQKPG | |
| | QAPRGLIGGTKFLAPGTPARFSGSLLGGKAALTLSGVQPE | |
| | DEAEYYCVLWYSNRWVFGGGTKLTVLGGGGSQVQLKQSGP | |
| | GLVQPSQSLSITCTVSGFSLTNYGVHWVRQSPGKGLEWLG | |
| | VIWSGGNTDYNTPFTSRLSINKDNSKSQVFFKMNSLQSND | |
| | TAIYYCARALTYYDYEFAYWGQGTLVTVSAASTKGPSVFP | |
| | LAPSSKSTSGGTAALGCLVKDYFPEPVTVSWNSGALTSGV | |
| | HTFPAVLQSSGLYSLSSVVTVPSSSLGTQTYICNVNHKPS | |
| | NTKVDKKVEPKSCAAAHHHHHHH | |
| PC-1 HisTag | GGPCRSHIDVAKPICVGGGGSGGLSGRSDAGSPLGLAGSG | 8 |
| LC | GSDILLTQSPVILSVSPGERVSFSCRASQSIGTNIHWYQQ | |
| | RTNGSPRLLIKYASESISGIPSRFSGSGSGTDFTLSINSV | |
| | ESEDIADYYCQQNNNWPTTFGAGTKLELKRTVAAPSVFIF | |
| | PPSDEQLKSGTASVVCLLNNFYPREAKVQWKVDNALQSGN | |
| | SQESVTEQDSKDSTYSLSSTLTLSKADYEKHKVYACEVTH | |
| | QGLSSPVTKSFNRGEC | |
| PC-1 Non- | EVQLVESGGGLVQPGNSLRLSCAASGFTFSKFGMSWVRQA | 9 |
| Cleavable (NC) | PGKGLEWVSSISGSGRDTLYADSVKGRFTISRDNAKTTLY | |
| HC | LQMNSLRPEDTAVYYCTIGGSLSVSSQGTLVTVSSGGGGS | |
| | GGGSGGVYCGPEFDESVGCMGGGGSGGGSGGGGSGGASSG | |
| | AGGSGGGSEVQLVESGGGLVQPGGSLKLSCAASGFTFNKY | |
| | AMNWVRQAPGKGLEWVARIRSKYNNYATYYADSVKDRFTI | |
| | SRDDSKNTAYLQMNNLKTEDTAVYYCVRHGNFGNSYISYW | |
| | AYWGQGTLVTVSSGGGGSGGGGSGGGGSQTVVTQEPSLTV | |
| | SPGGTVTLTCGSSTGAVTSGNYPNWVQQKPGQAPRGLIGG | |
| | TKFLAPGTPARFSGSLLGGKAALTLSGVQPEDEAEYYCVL | |
| | WYSNRWVFGGGTKLTVLGGGGSQVQLKQSGPGLVQPSQSL | |
| | SITCTVSGFSLTNYGVHWVRQSPGKGLEWLGVIWSGGNTD | |
| | YNTPFTSRLSINKDNSKSQVFFKMNSLQSQDTAIYYCARA | |
| | LTYYDYEFAYWGQGTLVTVSAASTKGPSVFPLAPSSKSTS | |
| | GGTAALGCLVKDYFPEPVTVSWNSGALTSGVHTFPAVLQS | |
| | SGLYSLSSVVTVPSSSLGTQTYICNVNHKPSNTKVDKKVE | |
| | PKSCGGGHHHHHHHH | |
| PC-1 NC LC | GGPCRSHIDVAKPICVGGGGSGGGGGSGGGGSGGASSGAG | 10 |
| | GSDILLTQSPVILSVSPGERVSFSCRASQSIGTNIHWYQQ | |
| | RINGSPRLLIKYASESISGIPSRFSGSGSGTDFTLSINSV | |
| | ESEDIADYYCQQNNNWPTTFGAGTKLELKRTVAAPSVFIF | |
| | PPSDEQLKSGTASVVCLLNNFYPREAKVQWKVDNALQSGN | |
| | SQESVTEQDSKDSTYSLSSTLTLSKADYEKHKVYACEVTH | |
| | QGLSSPVTKSFNRGEC | |
| PC-1 T Cell | EVQLVESGGGLVQPGGSLKLSCAASGFTFNKYAMNWVRQA | 11 |
| Engager (TCE) | PGKGLEWVARIRSKYNNYATYYADSVKDRFTISRDDSKNT | |
| HC | AYLQMNNLKTEDTAVYYCVRHGNFGNSYISYWAYWGQGTL | |
| | VTVSSGGGGSGGGGSGGGGSQTVVTQEPSLTVSPGGTVTL | |
| | TCGSSTGAVTSGNYPNWVQQKPGQAPRGLIGGTKFLAPGT | |
| | PARFSGSLLGGKAALTLSGVQPEDEAEYYCVLWYSNRWVF | |
| | GGGTKLTVLGGGGSQVQLKQSGPGLVQPSQSLSITCTVSG | |
| | FSLTNYGVHWVRQSPGKGLEWLGVIWSGGNTDYNTPFTSR | |
| | LSINKDNSKSQVFFKMNSLQSNDTAIYYCARALTYYDYEF | |
| | AYWGQGTLVTVSAASTKGPSVFPLAPSSKSTSGGTAALGC | |
| | LVKDYFPEPVTVSWNSGALTSGVHTFPAVLQSSGLYSLSS | |
| | VVTVPSSSLGTQTYICNVNHKPSNTKVDKKVEPKSCGGGH | |
| | HHHHHHH | |
| PC-1 TCE LC | DILLTQSPVILSVSPGERVSFSCRASQSIGTNIHWYQQRT | 12 |
| | NGSPRLLIKYASESISGIPSRFSGSGSGTDFTLSINSVES | |
| | EDIADYYCQQNNNWPTTFGAGTKLELKRTVAAPSVFIFPP | |
| | SDEQLKSGTASVVCLLNNFYPREAKVQWKVDNALQSGNSQ | |
| | ESVTEQDSKDSTYSLSSTLTLSKADYEKHKVYACEVTHQG | |
| | LSSPVTKSFNRGEC | |

PC-1 = polypeptide complex 1, EGFR-Epidermal Growth Factor Receptor, HC = Heavy Chain, LC = Light Chain, CD3 = Cluster of Differentiation 3, FAB = Fragment Antigen-Binding Region, scFv = Single Chain Variable Fragment, MMP9 = Matrix Metallopeptidase 9, SP = Serine Protease, HisTag-Histidine Tag, NC = Non-Cleavable, TCE = T Cell Engager

### Disulfide Bond(s)

In some embodiments, the at least one disulfide bond is an intrachain disulfide bond. In some embodiments, the at least one disulfide bond is an interchain disulfide bond. In some embodiments, the interchain disulfide bond is between the LC and the HC. In some embodiments, isolated recombinant antibody comprises at least two disulfide bonds formed by pairs of cysteine residues. In some embodiments, isolated recombinant antibody comprises at least three disulfide bonds formed by pairs of cysteine residues. In some embodiments, isolated recombinant antibody comprises at least four disulfide bonds formed by pairs of cysteine residues. In some embodiments, isolated recombinant antibody comprises at least five disulfide bonds formed by pairs of cysteine residues. In some embodiments, isolated recombinant antibody comprises at least six disulfide bonds formed by pairs of cysteine residues. In some embodiments, isolated recombinant antibody comprises at least seven disulfide bonds formed by pairs of cysteine residues. In some embodiments, isolated recombinant antibody comprises at least eight disulfide bonds formed by pairs of cysteine residues. In some embodiments, isolated recombinant antibody comprises at least nine disulfide bonds formed by pairs of cysteine residues. In some embodiments, isolated recombinant antibody comprises at least ten disulfide bonds formed by pairs of cysteine residues.

In some embodiments, the pair of cysteine residues comprises Cysteine 4 and Cysteine 15 of SEQ ID NO: 1. In some embodiments, the pair of cysteine residues comprises Cysteine 65 and Cysteine 130 of SEQ ID NO: 1. In some embodiments, the pair of cysteine residues comprises Cysteine 176 of SEQ ID NO:1 and Cysteine 236 of SEQ ID NO: 1. In some embodiments, the pair of cysteine residues comprises Cysteine 256 of SEQ ID NO: 1 and Cysteine 653 of SEQ ID NO: 2. In some embodiments, the pair of cysteine residues comprises Cysteine 22 of SEQ ID NO: 2 and Cysteine 96 of SEQ ID NO: 2. In some embodiments, the pair of cysteine residues comprises Cysteine 138 of SEQ ID NO: 2 and Cysteine 148 of SEQ ID NO: 2. In some embodiments, the pair of cysteine residues comprises Cysteine 199 of SEQ ID NO:2 and Cysteine 275 of SEQ ID NO: 2. In some embodiments, the pair of cysteine residues comprises Cysteine 339 of SEQ ID NO:2 and Cysteine 407 of SEQ ID NO: 2. In some embodiments, the pair of cysteine residues comprises Cysteine 453 of SEQ ID NO:2 and Cysteine 526 of SEQ ID NO: 2. In some embodiments, the pair of cysteine residues comprises Cysteine 577 of SEQ ID NO:2 and Cysteine 633 of SEQ ID NO: 2.

In some embodiments, the at least one disulfide bond formed by a pair of cysteine residues is selected from Cysteine 4 of SEQ ID NO: 1 and Cysteine 15 of SEQ ID NO:1; Cysteine 130 of SEQ ID NO:1 and Cysteine 176 of SEQ ID NO:1; Cysteine 236 of SEQ ID NO:2 and Cysteine 256 of SEQ ID NO: 1; Cysteine 653 of SEQ ID NO:2 and Cysteine 22 of SEQ ID NO:2; Cysteine 96 of SEQ ID NO:2 and Cysteine 199 of SEQ ID NO:2; Cysteine 275 of SEQ ID NO:2 and Cysteine 339 of SEQ ID NO:2; Cysteine 407 of SEQ ID NO:2 and Cysteine 453 of SEQ ID NO:2; Cysteine 526 of SEQ ID NO:2 and Cysteine 577 of SEQ ID NO:2; Cysteine 633 of SEQ ID NO:2.

In some embodiments, the isolated recombinant polypeptide complex comprises at least one cysteine residue that is a free sulfhydryl. The presence of free sulfhydryl(s) may be determined by mass spectrometry (MS).

### Amino Acid Modifications

In some embodiments, the isolated recombinant polypeptide complex further comprises O-xylosylation, asparagine deamidation, or succinimide formation. Asparagine 83 of SEQ ID NO: 1 can be deamidated. Asparagine 83 of SEQ ID NO: 1 can comprise a succinimide formation. Asparagine 179 of SEQ ID NO: 1 can be deamidated. Asparagine 233 of SEQ ID NO: 2 can be deamidated. Serine 110 of SEQ ID NO: 2 can comprise a o-xylosylation. Serine 123 of SEQ ID NO: 2 can comprise a o-xylosylation. Serine 124 of SEQ ID NO: 2 can comprise a o-xylosylation. Serine 129 of SEQ ID NO: 2 can comprise a o-xylosylation. Serine 133 of SEQ ID NO: 2 can comprise a o-xylosylation. Serine 154 of SEQ ID NO: 2 can comprise a o-xylosylation. In some embodiments, the succinimide formation is located at Asparagine 83 of SEQ ID NO: 1.

### N-glycan Moiety

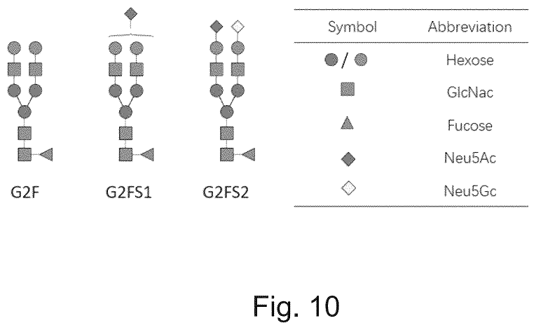

In some embodiments, the isolated recombinant polypeptide complex comprises at least one N-glycan moiety. The N-glycan moiety can comprise a fucose residue, four N-acetylglucosamine (GlcNAc) residues, and five hexose residues as can be seen in G2F of FIG. 10. The N-glycan moiety can comprise a fucose residue, four GlcNac residues, five hexose residues and a N-Acetylneuraminic acid (Neu5Ac) residue as can be seen in G2FS1 of FIG. 10. The N-glycan moiety can comprise a fucose residue, four GlcNac residues, five hexose residues, a Neu5Ac residue, and a Neu5Gc residue as can be seen in G2FS2 of FIG. 10. In some embodiments, the isolated recombinant antibody comprises at least two N-glycan moieties. In some embodiments the heavy chain sequence comprises at least one N-glycan moiety. In some embodiments the heavy chain sequence comprises a N-glycan moiety at Asparagine 83. In some embodiments, the light chain sequence comprises a N-glycan moiety at Asparagine 83. In some embodiments, a N-glycan moiety is located at Asparagine 83 of SEQ ID NO: 1. In some embodiments, at least one asparagine deamidation moiety is located at Asparagine 83 of SEQ ID NO: 1. In some embodiments the heavy chain sequence comprises a N-glycan moiety at Asparagine 519.

In some embodiments, the at least one N-glycan moiety comprises N-acetylglucosamine (GlcNAc), hexose, fucose, N-Acetylneuraminic acid (Neu5Ac), or N-Glycolylneuraminic acid (Neu5Gc). In some embodiments, the at least one N-glycan moiety comprises GlcNAc, hexose, fucose or Neu5Ac. In some embodiments, the at least one N-glycan moiety comprises GlcNAc and hexose. In some embodiments, the at least one N-glycan moiety comprises GlcNAc, hexose, and Neu5Ac. In some embodiments, the at least one N-glycan moiety comprise GlcNac, hexose and fucose. In some embodiments, the at least one N-glycan moiety comprises at least two GlcNAc moieties and at least two hexose moieties. In some embodiments, the at least one N-glycan moiety comprises at least three GlcNAc moieties and at least three hexose moieties. In some embodiments, the at least one N-glycan moiety comprises three GlcNAc moieties and three hexose moieties. In some embodiments, the at least one N-glycan moiety comprises four GlcNAc moieties and three hexose moieties. In some embodiments, the at least one N-glycan moiety comprises three GlcNAc moieties and four hexose moieties. In some embodiments, the at least one N-glycan moiety comprises two GlcNAc moieties and five hexose moieties. In some embodiments, the at least one N-glycan moiety comprises five GlcNAc moieties and three hexose moieties. In some embodiments, the at least one N-glycan moiety comprises four GlcNAc moieties and four hexose moieties. In some embodiments, the at least one N-glycan moiety comprises three GlcNAc moieties and five hexose moieties. In some embodiments, the at least one N-glycan moiety comprises five GlcNAc moieties and four hexose moieties. In some embodiments, the at least one N-glycan moiety comprises four GlcNAc moieties and five hexose moieties. In some embodiments, the at least one N-glycan moiety comprises four GlcNAc moieties, five hexose moieties, one Neu5Ac moiety and one fucose moiety. In some embodiments, the at least one N-glycan moiety comprises four GlcNAc moieties, six hexose moieties, and one fucose moiety.

### Tertiary and Secondary Structure

In some embodiments the isolated recombinant polypeptide complex has a secondary structure composition comprising a β-sheet or random coil. The secondary structure composition can comprise a β-sheet. The secondary structure composition can comprise a random coil.

In some embodiments, the isolated recombinant polypeptide complex is characterized by a far UV circular dichroism peak at a wavelength less than or equal to 220 nm, 210 nm, or 205 nm. In some embodiments, the isolated recombinant polypeptide complex is characterized by a far UV circular dichroism peak at a wavelength greater than or equal to 205 nm, 210 nm, or 220 nm. In some embodiments, the isolated recombinant polypeptide complex is characterized by a far UV circular dichroism peak at a wavelength between 200 nm and 210 nm or between 205 nm and 220 nm.

In some embodiments, the far UV circular dichroism peak is at a wavelength between 200 nm and 235 nm. In some embodiments, the far UV circular dichroism peak is at a wavelength between 210 nm and 230 nm.

In some embodiments, the isolated recombinant polypeptide complex has a far UV circular dichroism dip at a wavelength between about 215 nm and about 225 nm, e.g., about 215 nm, 216 nm, 217 nm, 218 nm, 219 nm, 220 nm, 221 nm, 222 nm, 223 nm, 224 nm, or about 225 nm, or any wavelength therebetween.

In some embodiments, the isolated recombinant polypeptide complex has a far UV circular dichroism dip at a wavelength between about 215 nm and about 220 nm, e.g., about 215 nm, 216 nm, 217 nm, 218 nm, 219 nm, or about 220 nm, or any wavelength therebetween.

In some embodiments, the isolated recombinant polypeptide complex has a near UV circular dichroism dip at a wavelength between about 280 nm and about 290 nm, e.g., about 280 nm, 281 nm, 282 nm, 283 nm, 284 nm, 285 nm, 286 nm, 287 nm, 288 nm, 289 nm, or about 290 nm, or any wavelength therebetween.

In some embodiments, the isolated recombinant polypeptide complex has a near UV circular dichroism dip at a wavelength between about 280 nm and about 285 nm, e.g., about 280 nm, 281 nm, 282 nm, 283 nm, 284 nm, or 285 nm, or any wavelength therebetween.

In some embodiments, the isolated recombinant polypeptide complex has a near UV circular dichroism peak at a wavelength between about 270 nm and about 275 nm, e.g., about 270 nm, 271 nm, 272 nm, 273 nm, 274 nm, or about 275 nm, or any wavelength therebetween.

In some embodiments, the isolated recombinant polypeptide complex has a near UV circular dichroism peak at a wavelength between about 285 nm and about 290 nm, e.g., about 285 nm, 286 nm, 284 nm, 288 nm, 289 nm, or about 290 nm, or any wavelength therebetween.

In some embodiments, the isolated recombinant polypeptide complex is characterized by a near UV circular dichroism peak at a wavelength less than or equal to 300 nm, 295 nm, 290 nm, 285 nm, 280 nm, 275 nm, or 270 nm. In some embodiments, the isolated recombinant polypeptide complex is characterized by a near UV circular dichroism peak at a wavelength greater than or equal to 270 nm, 275 nm, 280 nm, 285 nm, 290 nm, or 300 nm. In some embodiments, the isolated recombinant polypeptide complex is characterized by a near UV circular dichroism peak at a wavelength between 270 nm and 275 nm, 275 nm and 285 nm, between 280 nm and 290 nm, between 285 nm and 295 nm, or between 290 nm and 300 nm.

In some embodiments, the near UV circular dichroism peak is at a wavelength between 270 nm and 300 nm. In some embodiments, the near UV circular dichroism peak is at a wavelength between 275 nm and 290 nm.

### Metabolic Products

Disclosed herein are metabolic products of an isolated recombinant polypeptide complex disclosed herein. In some embodiments, the isolated recombinant polypeptide complex is cleaved by a protease to generate an enzymatic product of the isolated recombinant polypeptide complex after the administering. In some embodiments, the isolated recombinant polypeptide complex is cleaved by a tumor specific protease to generate the enzymatic product of the isolated recombinant polypeptide complex after the administering. In some embodiments, the tumor specific protease comprises two or more proteases. In some embodiments, the isolated recombinant polypeptide complex is cleaved by a first protease of the two or more proteases to generate a first metabolic product of the isolated recombinant polypeptide complex. In some embodiments, the isolated recombinant polypeptide complex is cleaved by a second protease of the two or more proteases to generate a second metabolic product of the isolated recombinant polypeptide complex. In some embodiments, the first protease comprises a serine protease. In some embodiments, the second protease comprises a matrix metalloprotease. In some embodiments, the serine protease comprises human matriptase (MTSP1). In some embodiments, the matrix metalloprotease comprises human matrix metalloprotease 9 (MMP9).

In some embodiments, the enzymatic product of the isolated recombinant polypeptide comprises the first metabolic product. In some embodiments, the enzymatic product of the isolated recombinant polypeptide comprises the second metabolic product. In some embodiments, the enzymatic product of the isolated recombinant polypeptide comprises the first metabolic product and the second metabolic product.

Disclosed herein is an isolated polypeptide that is an enzymatic product of an isolated recombinant polypeptide complex disclosed herein. Disclosed herein is an isolated polypeptide comprising an amino acid sequence with at least 90% sequence identity to the amino acid sequence of SEQ ID NO: 3 and wherein the isolated polypeptide is 221 amino acids in length. In some embodiments, the isolated polypeptide comprises an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 3. In some embodiments, the isolated polypeptide comprises an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 3 and wherein the isolated polypeptide is 221 amino acids in length. In some embodiments, the isolated polypeptide comprises the amino acid sequence of SEQ ID NO: 3.

Disclosed herein is an isolated polypeptide comprising an amino acid sequence with at least 90% sequence identity to the amino acid sequence of SEQ ID NO: 4 and wherein the isolated polypeptide is 229 amino acids in length. In some embodiments, the isolated polypeptide comprises an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 4. In some embodiments, the isolated polypeptide comprises an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 4 and wherein the isolated polypeptide is 229 amino acids in length. In some embodiments, the isolated polypeptide comprises the amino acid sequence of SEQ ID NO: 4.

Disclosed herein is an isolated polypeptide comprising an amino acid sequence with at least 90% sequence identity to the amino acid sequence of SEQ ID NO: 5 and wherein the isolated polypeptide is 484 amino acids in length. In some embodiments, the isolated polypeptide comprises an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 5. In some embodiments, the isolated polypeptide comprises an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 5 and wherein the isolated polypeptide is 484 amino acids in length. In some embodiments, the isolated polypeptide comprises the amino acid sequence of SEQ ID NO: 5.

Disclosed herein is an isolated polypeptide comprising an amino acid sequence with at least 90% sequence identity to the amino acid sequence of SEQ ID NO: 6 and wherein the isolated polypeptide is 492 amino acids in length. In some embodiments, the isolated polypeptide comprises an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 6. In some embodiments, the isolated polypeptide comprises an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 6 and wherein the isolated polypeptide is 492 amino acids in length. In some embodiments, the isolated polypeptide comprises the amino acid sequence of SEQ ID NO: 6.

Disclosed herein is an isolated polypeptide comprising a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 3 and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 2. In some embodiments, the isolated polypeptide comprises a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 3 and 221 amino acids in length and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 2 and 653 amino acids in length. In some embodiments, the isolated polypeptide comprises a first amino acid sequence having at least 90% sequence identity to the amino acid sequence of SEQ ID NO: 3 and 221 amino acids in length and a second amino acid sequence having at least 90% sequence identity to the amino acid sequence of SEQ ID NO: 2 and 653 amino acids in length. In some embodiments, the isolated polypeptide comprises a first amino acid sequence having the amino acid sequence of SEQ ID NO: 3 and a second amino acid sequence having the amino acid sequence of SEQ ID NO: 2.

Disclosed herein is an isolated polypeptide comprising a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 3 and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 5. In some embodiments, the isolated polypeptide comprises a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 3 and 221 amino acids in length and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 5 and 484 amino acids in length. In some embodiments, the isolated polypeptide comprises a first amino acid sequence having at least 90% sequence identity to the amino acid sequence of SEQ ID NO: 3 and 221 amino acids in length and a second amino acid sequence having at least 90% sequence identity to the amino acid sequence of SEQ ID NO: 5 and 484 amino acids in length. In some embodiments, the isolated polypeptide comprises a first amino acid sequence having the amino acid sequence of SEQ ID NO: 3 and a second amino acid sequence having the amino acid sequence of SEQ ID NO: 5.

Disclosed herein is an isolated polypeptide comprising a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 3 and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 6. In some embodiments, the isolated polypeptide comprises a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 3 and 221 amino acids in length and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 6 and 492 amino acids in length. In some embodiments, the isolated polypeptide comprises a first amino acid sequence having at least 90% sequence identity to the amino acid sequence of SEQ ID NO: 3 and 221 amino acids in length and a second amino acid sequence having at least 90% sequence identity to the amino acid sequence of SEQ ID NO: 6 and 492 amino acids in length. In some embodiments, the isolated polypeptide comprises a first amino acid sequence having the amino acid sequence of SEQ ID NO: 3 and a second amino acid sequence having the amino acid sequence of SEQ ID NO: 6.

Disclosed herein is an isolated polypeptide comprising a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 4 and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 2. In some embodiments, the isolated polypeptide comprises a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 4 and 229 amino acids in length and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 2 and 653 amino acids in length. In some embodiments, the isolated polypeptide comprises a first amino acid sequence having at least 90% sequence identity to the amino acid sequence of SEQ ID NO: 4 and 229 amino acids in length and a second amino acid sequence having at least 90% sequence identity to the amino acid sequence of SEQ ID NO: 2 and 653 amino acids in length. In some embodiments, the isolated polypeptide comprises a first amino acid sequence having the amino acid sequence of SEQ ID NO: 4 and a second amino acid sequence having the amino acid sequence of SEQ ID NO: 2.

Disclosed herein is an isolated polypeptide comprising a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 4 and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 5. In some embodiments, the isolated polypeptide comprises a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 4 and 229 amino acids in length and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 5 and 484 amino acids in length. In some embodiments, the isolated polypeptide comprises a first amino acid sequence having at least 90% sequence identity to the amino acid sequence of SEQ ID NO: 4 and 229 amino acids in length and a second amino acid sequence having at least 90% sequence identity to the amino acid sequence of SEQ ID NO: 5 and 484 amino acids in length. In some embodiments, the isolated polypeptide comprises a first amino acid sequence having the amino acid sequence of SEQ ID NO: 4 and a second amino acid sequence having the amino acid sequence of SEQ ID NO: 5.

Disclosed herein is an isolated polypeptide comprising a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 4 and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 6. In some embodiments, the isolated polypeptide comprises a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 4 and 229 amino acids in length and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 6 and 492 amino acids in length. In some embodiments, the isolated polypeptide comprises a first amino acid sequence having at least 90% sequence identity to the amino acid sequence of SEQ ID NO: 4 and 229 amino acids in length and a second amino acid sequence having at least 90% sequence identity to the amino acid sequence of SEQ ID NO: 6 and 492 amino acids in length. In some embodiments, the isolated polypeptide comprises a first amino acid sequence having the amino acid sequence of SEQ ID NO: 4 and a second amino acid sequence having the amino acid sequence of SEQ ID NO: 6.

Disclosed herein is an isolated polypeptide comprising a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 1 and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 5. In some embodiments, the isolated polypeptide comprises a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 1 and 256 amino acids in length and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 5 and 484 amino acids in length. In some embodiments, the isolated polypeptide comprises a first amino acid sequence having at least 90% sequence identity to the amino acid sequence of SEQ ID NO: 1 and 256 amino acids in length and a second amino acid sequence having at least 90% sequence identity to the amino acid sequence of SEQ ID NO: 5 and 484 amino acids in length. In some embodiments, the isolated polypeptide comprises a first amino acid sequence having the amino acid sequence of SEQ ID NO: 1 and a second amino acid sequence having the amino acid sequence of SEQ ID NO: 5.

Disclosed herein is an isolated polypeptide comprising a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 1 and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 6. In some embodiments, the isolated polypeptide comprises a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 1 and 256 amino acids in length and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 6 and 492 amino acids in length. In some embodiments, the isolated polypeptide comprises a first amino acid sequence having at least 90% sequence identity to the amino acid sequence of SEQ ID NO: 1 and 256 amino acids in length and a second amino acid sequence having at least 90% sequence identity to the amino acid sequence of SEQ ID NO: 6 and 492 amino acids in length. In some embodiments, the isolated polypeptide comprises a first amino acid sequence having the amino acid sequence of SEQ ID NO: 1 and a second amino acid sequence having the amino acid sequence of SEQ ID NO: 6.

Disclosed herein is a pharmaceutical composition comprising the isolated polypeptide disclosed herein, and a pharmaceutically acceptable excipient disclosed herein.

### Thermal Stability

In some embodiments, the isolated recombinant polypeptide complex is characterized by a melting temperature (Tₘ) of about 67° C., 68° C., 69° C., 70° C., 71° C., 72° C., 73° C., 74° C., 75° C., 76° C., 77° C., 78° C., 79° C., or 80° C. or a range between any two of the foregoing values. In some embodiments, the isolated recombinant polypeptide complex is characterized by a Tₘ from about 69° C. to about 76° C. In some embodiments, the isolated recombinant polypeptide complex is characterized by a Tₘ from about 73° C. to about 74° C. In some embodiments, the isolated recombinant polypeptide complex is characterized by a melting temperature (Tₘ) from about 73.8° C. In some embodiments, the isolated recombinant polypeptide complex is characterized by a melting onset temperature (T_{Onset}) from about 60° C. to about 65° C. In some embodiments, the isolated recombinant polypeptide complex is characterized by a melting onset temperature (T_{Onset}) from about 62° C. to about 64° C. In some embodiments, the isolated recombinant polypeptide complex is characterized by a melting onset temperature (T_{Onset}) from about 63.2° C. The melting temperature (Tₘ) may be determined by Differential Scanning Calorimetry (DSC). The melting onset temperature (T_{Onset}) may be determined by Differential Scanning Calorimetry (DSC).

In some embodiments, the isolated recombinant polypeptide complex is characterized by one or more of the following: the first chain comprises the amino acid sequence according to SEQ ID NO: 1; the second chain comprises the amino acid sequence according to SEQ ID NO: 2; the at least one N-glycan moiety comprises G2F, G2FS1, or G2FS2; the recombinant polypeptide complex comprises disulfide bonds formed by pairs of cysteine residues Cysteine 4 and Cysteine 15 of SEQ ID NO: 1, Cysteine 65 and Cysteine 130 of SEQ ID NO: 1, Cysteine 176 and Cysteine 236 of SEQ ID NO: 1, Cysteine 256 of SEQ ID NO: 1 and Cysteine 653 of SEQ ID NO: 2, Cysteine 138 and Cysteine 148 of SEQ ID NO: 2, Cysteine 22 and Cysteine 96 of SEQ ID NO: 2, Cysteine 199 and Cysteine 275 of SEQ ID NO: 2, Cysteine 339 and Cysteine 407 of SEQ ID NO: 2, Cysteine 453 and Cysteine 526 of SEQ ID NO: 2, and Cysteine 577 and Cysteine 633 of SEQ ID NO: 2; the T_{Onset} is between about 60° C. to about 65° C. and a transition mid-point temperature (Tₘₗ) between about 70° C. and about 75° C., wherein the T_{Onset} and the Tₘₗ are measured using differential scanning calorimetry (DSC), when the isolated recombinant polypeptide complex is formulated at a concentration of about 1.0 mg/mL in about 10 mM histidine, about 8% (w/v) sucrose, about 0.01% (w/v) PS20 pH of about 5.3, and a far UV circular dichroism dip at a wavelength between 210 nm and 230 nm when the isolated recombinant polypeptide complex is formulated at a concentration of about 0.1 mg/mL in water, and a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

In some embodiments, the isolated recombinant polypeptide complex is characterized by all of the following: wherein the first chain comprises the amino acid sequence according to SEQ ID NO: 1, and the second chain comprises the amino acid sequence according to SEQ ID NO: 2, and the at least one N-glycan moiety comprises G2F, G2FS1, or G2FS2, and the recombinant polypeptide complex comprises disulfide bonds formed by pairs of cysteine residues Cysteine 4 and Cysteine 15 of SEQ ID NO: 1, Cysteine 65 and Cysteine 130 of SEQ ID NO: 1, Cysteine 176 and Cysteine 236 of SEQ ID NO: 1, Cysteine 256 of SEQ ID NO: 1 and Cysteine 653 of SEQ ID NO: 2, Cysteine 138 and Cysteine 148 of SEQ ID NO: 2, Cysteine 22 and Cysteine 96 of SEQ ID NO: 2, Cysteine 199 and Cysteine 275 of SEQ ID NO: 2, Cysteine 339 and Cysteine 407 of SEQ ID NO: 2, Cysteine 453 and Cysteine 526 of SEQ ID NO: 2, and Cysteine 577 and Cysteine 633 of SEQ ID NO: 2, and the T_{Onset} is between about 60° C. to about 65° C. and a transition mid-point temperature (Tₘₗ) between about 70° C. and about 75° C., wherein the T_{Onset} and the Tₘₗ are measured using differential scanning calorimetry (DSC), when the isolated recombinant polypeptide complex is formulated at a concentration of about 1.0 mg/mL in about 10 mM histidine, about 8% (w/v) sucrose, about 0.01% (w/v) PS20 pH of about 5.3, and a far UV circular dichroism dip at a wavelength between 210 nm and 230 nm when the isolated recombinant polypeptide complex is formulated at a concentration of about 0.1 mg/mL in water, and a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

Formulations of Isolated Recombinant Antibodies that Target EGFR and CD3

Disclosed herein includes formulation(s) comprising a population of antibodies or recombinant antibodies, such as comprising any one or a combination the antibodies or recombinant antibodies as described herein.

Disclosed herein is a pharmaceutical composition comprising one or more pharmaceutically acceptable excipients, wherein the one or more pharmaceutically acceptable excipients comprise histidine, sucrose, polysorbate-20, sodium phosphate, citrate, acetate, sodium chloride, potassium chloride, magnesium chloride, and calcium chloride. Disclosed herein is a pharmaceutical composition, wherein the pharmaceutical composition comprises the isolated recombinant polypeptide complex (such as any described herein), sodium phosphate monobasic monohydrate, sodium phosphate dibasic, citrate, acetate, histidine, heptahydrate, sodium chloride, potassium chloride, histidine, citrate, acetate, sucrose, polysorbate-20, polysorbate 80, magnesium chloride hexahydrate and calcium chloride dihydrate. The pharmaceutical composition can have a pH less than or equal to 6.5, 6.0, 5.5, 5.0, or 4.5. The pharmaceutical composition can have a pH greater than or equal to 4.5, 5.0, 5.5, 6.0 or 6.5. The pharmaceutical composition can have a pH between 4.0 and 5.0, between 4.5 and 5.5, or between 5.0 and 6.0. The pharmaceutical composition can comprise greater than or equal to 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM Histidine. The pharmaceutical composition can comprise less than or equal to 20 mM, 19 mM, 18 mM, 17 mM, 16 mM, 15 mM, 14 mM, 13 mM, 12 mM, 11 mM, 10 mM, 9 mM, 8 mM, 7 mM, 6 mM, 5 mM, 4 mM, 3 mM, 2 mM, or 1 mM Histidine. The pharmaceutical composition can comprise greater than or equal to 5% (w/v), 6% (w/v), 7% (w/v), 8% (w/v), 9% (w/v), or 10% (w/v) sucrose. The pharmaceutical composition can comprise less than or equal to 10% (w/v), 9% (w/v), 8% (w/v), 7% (w/v), 6% (w/v), or 5% (w/v) sucrose. The pharmaceutical composition can comprise less than or equal to 0.05% (w/v) polysorbate 20, 0.04% (w/v) polysorbate 20, 0.03% (w/v) polysorbate 20, 0.02% (w/v) polysorbate 20, or 0.010% polysorbate 20. The pharmaceutical composition can comprise greater than or equal to 0.01% (w/v) polysorbate 20, 0.02% (w/v) polysorbate 20, 0.03% (w/v) polysorbate 20, 0.04% (w/v) polysorbate 20, or 0.05% polysorbate 20. Disclosed herein is a pharmaceutical composition, wherein the pharmaceutical composition comprises the isolated recombinant polypeptide complex (such as any described herein), about 10 mM Histidine, about 8% (w/v) sucrose, about 0.01% (w/v) polysorbate 20, pH of about 5.3.

In some embodiments of the formulation, at least 90%, 91%, 92%, 93%, 94%, 95%, 96%, 97%, 98%, or 99% (e.g., by mole or by mass) of the antibodies of the population is monomeric. In some embodiments of the formulation, less than or equal to 5%, 4%, 3%, 2%, 1%, 0.5%, or 0.1% (e.g., by mole or by mass) of the antibodies of the population is aggregated.

Disclosed herein are a plurality of isolated recombinant antibodies that target EGFR and CD3 wherein the isolated recombinant antibodies comprise a light chain (LC) amino acid sequence that has at least 80% sequence identity to SEQ ID NO: 1, a heavy chain (HC) amino acid sequence that has at least 80% sequence identity to SEQ ID NO: 2, wherein the plurality comprises greater than 90% monomer of the isolated recombinant antibodies.

In some embodiments, the plurality comprises greater than 910% monomer. In some embodiments, the plurality comprises greater than 92% monomer. In some embodiments, the plurality comprises greater than 93% monomer. In some embodiments, the plurality comprises greater than 94% monomer. In some embodiments, the plurality comprises greater than 95% monomer. In some embodiments, the plurality comprises greater than 96% monomer. In some embodiments, the plurality comprises greater than 97% monomer. In some embodiments, the plurality comprises greater than 98% monomer. In some embodiments, the plurality comprises greater than 99% monomer.

In some embodiments, the LC comprises at least 85% sequence identity to SEQ ID NO: 1. In some embodiments, the LC comprises at least 90% sequence identity to SEQ ID NO: 1. In some embodiments, the LC comprises at least 95% sequence identity to SEQ ID NO: 1. In some embodiments, the LC comprises at least 99% sequence identity to SEQ ID NO: 1. In some embodiments, the LC comprises the amino acid sequence according to SEQ ID NO: 1. In some embodiments, the HC comprises at least 85% sequence identity to SEQ ID NO: 2. In some embodiments, the HC comprises at least 90% sequence identity to SEQ ID NO: 2. In some embodiments, the HC comprises at least 95% sequence identity to SEQ ID NO: 2. In some embodiments, the HC comprises at least 99% sequence identity to SEQ ID NO: 2. In some embodiments, the HC comprises the amino acid sequence according to SEQ ID NO: 2.

In some embodiments, the LC comprises at least 99% sequence identity to SEQ ID NO: 1, the HC comprises at least 99% sequence identity to SEQ ID NO: 2. In some embodiments, the LC comprises the amino acid sequence according to SEQ ID NO: 1, the HC comprises the amino acid sequence according to SEQ ID NO: 2.

In some embodiments, the concentration of the isolated recombinant antibodies is greater than or equal to 1.0 mg/mL. In some embodiments, the concentration of the isolated recombinant antibodies is at least 2.0 mg/mL. In some embodiments, the concentration of the isolated recombinant antibodies is at least 5.0 mg/mL. In some embodiments, the concentration of the isolated recombinant antibodies is at least 10.0 mg/mL. In some embodiments, the concentration of the isolated recombinant antibodies is at least 15.0 mg/mL. In some embodiments, the concentration of the isolated recombinant antibodies is at least 20.0 mg/mL. In some embodiments, the plurality is in a buffered solution having a pH less than or equal to 5.5. In some embodiments, the buffered solution comprises one or more of acetate, phosphate, or histidine. In some embodiments, the buffered solution comprises histidine at a concentration greater than 5 mM. In some embodiments, the buffered solution comprises sucrose. In some embodiments, the sucrose is at a concentration greater than or equal to 5% w/v. In some embodiments, the buffered solution comprises polysorbate 20. In some embodiments, the polysorbate 20 is at a concentration greater than or equal to 0.010% w/v. In some embodiments, the plurality comprises greater than 90% monomer at a concentration of greater than or equal to about 20.0 mg/mL in about 10 mM histidine, about 8% (w/v) sucrose, about 0.01% (w/v) PS20 pH of about 5.3.

Disclosed herein is a pharmaceutical composition comprising an isolated recombinant polypeptide complex disclosed herein and a pharmaceutically acceptable excipient. In some embodiments, the isolated recombinant polypeptide complex comprises a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NO: 1. In some embodiments, the isolated recombinant polypeptide complex comprises a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NO: 2. In some embodiments, the isolated recombinant polypeptide complex comprises a first amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NO: 1 and a second amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NO: 2. In some embodiments, the isolated recombinant polypeptide complex comprises a first amino acid sequence having at least 70% sequence identity to SEQ ID NO: 1 and a second amino acid sequence having at least 70% sequence identity to SEQ ID NO: 2. In some embodiments, the isolated recombinant polypeptide complex comprises a first amino acid sequence having at least 90% sequence identity to SEQ ID NO: 1 and a second amino acid sequence having at least 90% sequence identity to SEQ ID NO: 2. In some embodiments, the isolated recombinant polypeptide complex comprises a first amino acid sequence having at least 90% sequence identity to SEQ ID NO: 1. In some embodiments, the isolated recombinant polypeptide complex comprises a second amino acid sequence having at least 90% sequence identity to SEQ ID NO: 2. In some embodiments, the isolated recombinant polypeptide complex comprises a first amino acid sequence having the amino acid sequence of SEQ ID NO: 1 and a second amino acid sequence having the amino acid sequence of SEQ ID NO: 2. In some embodiments, the isolated recombinant polypeptide complex comprises a first amino acid sequence having the amino acid sequence of SEQ ID NO: 1. In some embodiments, the isolated recombinant polypeptide complex comprises a second amino acid sequence having the amino acid sequence of SEQ ID NO: 2.

In some embodiments, the pharmaceutical composition comprises the isolated recombinant polypeptide complex at a concentration of about 2 mg/ml. In some embodiments, the pharmaceutically acceptable excipient comprises a buffer, a stabilizing agent, a tonicity agent, a surfactant, or combinations thereof. In some embodiments, the pharmaceutically acceptable excipient comprises a buffer. In some embodiments, the pharmaceutically acceptable excipient comprises a tonicity agent. In some embodiments, the pharmaceutically acceptable excipient comprises a surfactant. In some embodiments, the pharmaceutically acceptable excipient comprises a buffer, a stabilizing agent, a tonicity agent, and a surfactant. In some embodiments, the buffer comprises an amino acid or a derivative thereof. In some embodiments, the amino acid or the derivative thereof comprises L-histidine, L-histidine monohydrochloride monohydrate, or combinations thereof. In some embodiments, the stabilizing agent comprises sugar. In some embodiments, the sugar comprises sucrose. In some embodiments, the tonicity agent comprises sugar. In some embodiments, the sugar comprises sucrose. In some embodiments, the surfactant comprises a polysorbate. In some embodiments, the surfactant comprises polysorbate 20 (PS20).

In some embodiments, the pharmaceutical composition comprises about 1 millimolar (mM) to about 50 mM L-histidine in the form of L-histidine and/or L-histidine monohydrochloride monohydrate, e.g., about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, or about 50 mM, or any concentration therebetween. In some embodiments, the pharmaceutical composition comprises about 1 to about 50 mM L-histidine in the form of L-histidine and L-histidine monohydrochloride monohydrate, e.g., about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, or about 50 mM, or any concentration therebetween. In some embodiments, the pharmaceutical composition comprises about 10 mM L-histidine in the form of L-histidine and/or L-histidine monohydrochloride monohydrate. In some embodiments, the pharmaceutical composition comprises about 10 mM L-histidine in the form of L-histidine or L-histidine monohydrochloride monohydrate. In some embodiments, the pharmaceutical composition comprises about 10 mM L-histidine in the form of L-histidine and L-histidine monohydrochloride monohydrate.

In some embodiments, the pharmaceutical composition comprises about 1% weight/volume (w/v) to about 20% (w/v) sucrose, e.g., about 1%, 2%, 3%, 4%, 5%, 6%, 7%, 8%, 9%, 10%, 11%, 12%, 13%, 14%, 15%, 16%, 17%, 18%, 19%, or about 20%, or any concentration therebetween. In some embodiments, the pharmaceutical composition comprises about 8% (w/v) sucrose.

In some embodiments, the pharmaceutical composition comprises about 0.001% (w/v) to about 0.1% (w/v) polysorbate 20 (PS20), e.g., about 0.001%, 0.002%, 0.003%, 0.004%, 0.005%, 0.006%, 0.007%, 0.008%, 0.009%, 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.031%, 0.032%, 0.033%, 0.034%, 0.035%, 0.036%, 0.037%, 0.038%, 0.039%, 0.04%, 0.041%, 0.042%, 0.043%, 0.044%, 0.045%, 0.046%, 0.047%, 0.048%, 0.049%, 0.05%, 0.051%, 0.052%, 0.053%, 0.054%, 0.055%, 0.056%, 0.057%, 0.058%, 0.059%, 0.06%, 0.061%, 0.062%, 0.063%, 0.064%, 0.065%, 0.066%, 0.067%, 0.068%, 0.069%, 0.07%, 0.071%, 0.072%, 0.073%, 0.074%, 0.075%, 0.076%, 0.077%, 0.078%, 0.079%, 0.08%, 0.081%, 0.082%, 0.083%, 0.084%, 0.085%, 0.086%, 0.087%, 0.088%, 0.089%, 0.09%, 0.091%, 0.092%, 0.093%, 0.094%, 0.095%, 0.096%, 0.097%, 0.098%, 0.099%, or about 0.1%, or any concentration therebetween. In some embodiments, the pharmaceutical composition comprises about 0.01% (w/v) polysorbate 20 (PS20).

In some embodiments, the pharmaceutical composition comprises about 10 mM L-histidine in the form of L-histidine and L-histidine monohydrochloride monohydrate, about 8% (w/v) sucrose, and about 0.01% (w/v) polysorbate 20. In some embodiments, the pharmaceutical composition comprises a pH of about 5.3. In some embodiments, the pharmaceutical composition comprises an osmolality of about 276 mOsmol/kg.

Disclosed herein is a pharmaceutical composition comprising an isolated polypeptide disclosed herein, and a pharmaceutically acceptable excipient disclosed herein. In some embodiments, the isolated polypeptide is an enzymatic product of the isolated recombinant polypeptide complex disclosed herein.

### Dosing and Pharmacology

In some embodiments, the isolated recombinant polypeptide complex disclosed herein provides a maximum plasma concentration (Cmax) in a subject after a single intravenous bolus administration to the subject of a dose of about 0.1 milligram per kilogram of the body weight (mg/kg) to about 1 mg/kg, e.g., about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, or about 1 mg/kg, or any dose therebetween. In some embodiments, the Cmax increases when the dose increases. In some embodiments, an increase of the Cmax is proportional to an increase of the dose. In some embodiments, an increase of the Cmax is more than a value that is proportional to an increase of the dose. In some embodiments, an increase of the area under the drug concentration versus time curve between 0 hour (h) and 216 h after the administration (AUC₀₋₂₁₆ₕ) is more than a value that is proportional to an increase of the dose. In some embodiments, an increase of AUC₀₋₂₁₆ₕ is less than a value that is proportional to an increase of the dose being administered. In some embodiments, the subject has the highest Cmax within 24 h after the administration. In some embodiments, the subject has the highest AUC₀₋₂₄ₕ at 24 h after the administration.

In some embodiments, the dose is about 0.1 mg/kg. In some embodiments, the dose is about 0.3 mg/kg. In some embodiments, the dose is about 1 mg/kg. In some embodiments, the dose is about 0.3 mg/kg to about 1 mg/kg. In some embodiments, the dose is about 0.1 mg/kg to about 0.3 mg/kg. In some embodiments, the dose is about 0.1 mg/kg to about 0.3 mg/kg. In some embodiments, the dose is about 0.3 mg/kg to about 1 mg/kg.

In some embodiments, the isolated recombinant polypeptide complex provides a half maximum plasma concentration (T_{1/2}) in the subject at about 88.8 h to about 101 h, e.g. about 88.8 h, 88.9 h, 89 h, 89.1 h, 89.2 h, 89.3 h, 89.4 h, 89.5 h, 89.6 h, 89.7 h, 89.8 h, 89.9 h, 90 h, 90.1 h, 90.2 h, 90.3 h, 90.4 h, 90.5 h, 90.6 h, 90.7 h, 90.8 h, 90.9 h, 91 h, 91.1 h, 91.2 h, 91.3 h, 91.4 h, 91.5 h, 91.6 h, 91.7 h, 91.8 h, 91.9 h, 92 h, 92.1 h, 92.2 h, 92.3 h, 92.4 h, 92.5 h, 92.6 h, 92.7 h, 92.8 h, 92.9 h, 93 h, 93.1 h, 93.2 h, 93.3 h, 93.4 h, 93.5 h, 93.6 h, 93.7 h, 93.8 h, 93.9 h, 94 h, 94.1 h, 94.2 h, 94.3 h, 94.4 h, 94.5 h, 94.6 h, 94.7 h, 94.8 h, 94.9 h, 95 h, 95.1 h, 95.2 h, 95.3 h, 95.4 h, 95.5 h, 95.6 h, 95.7 h, 95.8 h, 95.9 h, 96 h, 96.1 h, 96.2 h, 96.3 h, 96.4 h, 96.5 h, 96.6 h, 96.7 h, 96.8 h, 96.9 h, 97 h, 97.1 h, 97.2 h, 97.3 h, 97.4 h, 97.5 h, 97.6 h, 97.7 h, 97.8 h, 97.9 h, 98 h, 98.1 h, 98.2 h, 98.3 h, 98.4 h, 98.5 h, 98.6 h, 98.7 h, 98.8 h, 98.9 h, 99 h, 99.1 h, 99.2 h, 99.3 h, 99.4 h, 99.5 h, 99.6 h, 99.7 h, 99.8 h, 99.9 h, 100 h, 100.1 h, 100.2 h, 100.3 h, 100.4 h, 100.5 h, 100.6 h, 100.7 h, 100.8 h, 100.9 h, or about 101 h, or any time therebetween.

In some embodiments, the isolated recombinant polypeptide complex is administered to a subject through a single intravenous infusion over about 30 minutes (min). In some embodiments, an increase of the Cmax is proportional to an increase of the dose. In some embodiments, an increase of the Cmax is proportional to an increase of the dose when the isolated recombinant polypeptide complex is administered to a subject through a single intravenous infusion over about 30 min. In some embodiments, an increase of the area under the curve between 0 h and 168 h after the administration (AUC₀₋₁₆₈ₕ) is less than a value that is proportional to an increase of the dose. In some embodiments, an increase of the area under the curve between 0 h and 168 h after the administration (AUC₀₋₁₆₈ₕ) is less than a value that is proportional to an increase of the dose when the isolated recombinant polypeptide complex is administered to a subject through a single intravenous infusion over about 30 min. In some embodiments, an increase of AUC₀₋₁₆₈ₕ is proportional to an increase of the dose when the isolated recombinant polypeptide complex is administered to a subject through a single intravenous infusion over about 30 min. In some embodiments, the dose is about 0.05 mg/kg. In some embodiments, the dose is about 0.2 mg/kg. In some embodiments, the dose is about 0.6 mg/kg. In some embodiments, the dose is about 0.05 mg/kg to about 0.2 mg/kg. In some embodiments, the dose is about 0.2 mg/kg to about 0.6 mg/kg, e.g., about 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, 0.3 mg/kg, 0.31 mg/kg, 0.32 mg/kg, 0.33 mg/kg, 0.34 mg/kg, 0.35 mg/kg, 0.36 mg/kg, 0.37 mg/kg, 0.38 mg/kg, 0.39 mg/kg, 0.4 mg/kg, 0.41 mg/kg, 0.42 mg/kg, 0.43 mg/kg, 0.44 mg/kg, 0.45 mg/kg, 0.46 mg/kg, 0.47 mg/kg, 0.48 mg/kg, 0.49 mg/kg, 0.5 mg/kg, 0.51 mg/kg, 0.52 mg/kg, 0.53 mg/kg, 0.54 mg/kg, 0.55 mg/kg, 0.56 mg/kg, 0.57 mg/kg, 0.58 mg/kg, 0.59 mg/kg, or about 0.6 mg/kg, or any dose therebetween. In some embodiments, the isolated recombinant polypeptide complex provides a T_{1/2} at about 68.2 h to about 96.5 h, e.g. about 68.2 h, 68.3 h, 68.4 h, 68.5 h, 68.6 h, 68.7 h, 68.8 h, 68.9 h, 69 h, 69.1 h, 69.2 h, 69.3 h, 69.4 h, 69.5 h, 69.6 h, 69.7 h, 69.8 h, 69.9 h, 70 h, 70.1 h, 70.2 h, 70.3 h, 70.4 h, 70.5 h, 70.6 h, 70.7 h, 70.8 h, 70.9 h, 71 h, 71.1 h, 71.2 h, 71.3 h, 71.4 h, 71.5 h, 71.6 h, 71.7 h, 71.8 h, 71.9 h, 72 h, 72.1 h, 72.2 h, 72.3 h, 72.4 h, 72.5 h, 72.6 h, 72.7 h, 72.8 h, 72.9 h, 73 h, 73.1 h, 73.2 h, 73.3 h, 73.4 h, 73.5 h, 73.6 h, 73.7 h, 73.8 h, 73.9 h, 74 h, 74.1 h, 74.2 h, 74.3 h, 74.4 h, 74.5 h, 74.6 h, 74.7 h, 74.8 h, 74.9 h, 75 h, 75.1 h, 75.2 h, 75.3 h, 75.4 h, 75.5 h, 75.6 h, 75.7 h, 75.8 h, 75.9 h, 76 h, 76.1 h, 76.2 h, 76.3 h, 76.4 h, 76.5 h, 76.6 h, 76.7 h, 76.8 h, 76.9 h, 77 h, 77.1 h, 77.2 h, 77.3 h, 77.4 h, 77.5 h, 77.6 h, 77.7 h, 77.8 h, 77.9 h, 78 h, 78.1 h, 78.2 h, 78.3 h, 78.4 h, 78.5 h, 78.6 h, 78.7 h, 78.8 h, 78.9 h, 79 h, 79.1 h, 79.2 h, 79.3 h, 79.4 h, 79.5 h, 79.6 h, 79.7 h, 79.8 h, 79.9 h, 80 h, 80.1 h, 80.2 h, 80.3 h, 80.4 h, 80.5 h, 80.6 h, 80.7 h, 80.8 h, 80.9 h, 81 h, 81.1 h, 81.2 h, 81.3 h, 81.4 h, 81.5 h, 81.6 h, 81.7 h, 81.8 h, 81.9 h, 82 h, 82.1 h, 82.2 h, 82.3 h, 82.4 h, 82.5 h, 82.6 h, 82.7 h, 82.8 h, 82.9 h, 83 h, 83.1 h, 83.2 h, 83.3 h, 83.4 h, 83.5 h, 83.6 h, 83.7 h, 83.8 h, 83.9 h, 84 h, 84.1 h, 84.2 h, 84.3 h, 84.4 h, 84.5 h, 84.6 h, 84.7 h, 84.8 h, 84.9 h, 85 h, 85.1 h, 85.2 h, 85.3 h, 85.4 h, 85.5 h, 85.6 h, 85.7 h, 85.8 h, 85.9 h, 86 h, 86.1 h, 86.2 h, 86.3 h, 86.4 h, 86.5 h, 86.6 h, 86.7 h, 86.8 h, 86.9 h, 87 h, 87.1 h, 87.2 h, 87.3 h, 87.4 h, 87.5 h, 87.6 h, 87.7 h, 87.8 h, 87.9 h, 88 h, 88.1 h, 88.2 h, 88.3 h, 88.4 h, 88.5 h, 88.6 h, 88.7 h, 88.8 h, 88.9 h, 89 h, 89.1 h, 89.2 h, 89.3 h, 89.4 h, 89.5 h, 89.6 h, 89.7 h, 89.8 h, 89.9 h, 90 h, 90.1 h, 90.2 h, 90.3 h, 90.4 h, 90.5 h, 90.6 h, 90.7 h, 90.8 h, 90.9 h, 91 h, 91.1 h, 91.2 h, 91.3 h, 91.4 h, 91.5 h, 91.6 h, 91.7 h, 91.8 h, 91.9 h, 92 h, 92.1 h, 92.2 h, 92.3 h, 92.4 h, 92.5 h, 92.6 h, 92.7 h, 92.8 h, 92.9 h, 93 h, 93.1 h, 93.2 h, 93.3 h, 93.4 h, 93.5 h, 93.6 h, 93.7 h, 93.8 h, 93.9 h, 94 h, 94.1 h, 94.2 h, 94.3 h, 94.4 h, 94.5 h, 94.6 h, 94.7 h, 94.8 h, 94.9 h, 95 h, 95.1 h, 95.2 h, 95.3 h, 95.4 h, 95.5 h, 95.6 h, 95.7 h, 95.8 h, 95.9 h, 96 h, 96.1 h, 96.2 h, 96.3 h, 96.4 h, or about 96.5 h, or any duration therebetween. In some embodiments, the isolated recombinant polypeptide complex provides a T_{1/2} at about 81.3 h at a dose of about 0.05 mg/kg. In some embodiments, the isolated recombinant polypeptide complex provides a T_{1/2} at about 68.2 h at a dose of about 0.2 mg/kg being administered. In some embodiments, the isolated recombinant polypeptide complex provides a T_{1/2} at about 96.5 h at a dose of about 0.6 mg/kg.

In some embodiments, the isolated recombinant polypeptide complex is administered at least once weekly. In some embodiments, the isolated recombinant polypeptide complex is administered once weekly. In some embodiments, the isolated recombinant polypeptide complex is administered at least twice weekly. In some embodiments, the isolated recombinant polypeptide complex is administered for at least 1 week, 2 weeks, 3 weeks, 4 weeks, 5 weeks, 6 weeks, 7 weeks, 8 weeks, 9 weeks, or 10 weeks. In some embodiments, the isolated recombinant polypeptide complex is administered for at least 1 month, 2 months, 3 months, 4 months, 5 months, 6 months, 7 months, 8 months, 9 months, 10 months, 11 months, or 12 months. In some embodiments, the isolated recombinant polypeptide complex is administered for at least 1 year, 2 years, 3 years, 4 years, or 5 years. In some embodiments, the isolated recombinant polypeptide complex is administered for at most 1 week, 2 weeks, 3 weeks, 4 weeks, 5 weeks, 6 weeks, 7 weeks, 8 weeks, 9 weeks, or 10 weeks. In some embodiments, the isolated recombinant polypeptide complex is administered for at most 1 month, 2 months, 3 months, 4 months, 5 months, 6 months, 7 months, 8 months, 9 months, 10 months, 11 months, or 12 months. In some embodiments, the isolated recombinant polypeptide complex is administered for at most 1 year, 2 years, 3 years, 4 years, or 5 years. In some embodiments, an increase of the Cmax is proportional to an increase of the dose after day 1, wherein day 1 is the day of the administration. In some embodiments, an increase of the AUC₀₋₂₄ₕ is proportional to an increase of the dose after day 1, wherein day 1 is the day of the administration. In some embodiments, an increase of the AUC₀₋₁₆₈ₕ is proportional to an increase of the dose after day 1, wherein day 1 is the day of the administration. In some embodiments, the dose is about 0.05 mg/kg. In some embodiments, the dose is about 0.2 mg/kg. In some embodiments, the dose is about 0.6 mg/kg. In some embodiments, the isolated recombinant polypeptide complex is not metabolized by a cytochrome P450 (CYP) enzyme. In some embodiments, the isolated recombinant polypeptide complex is not transported by P-glycoprotein (Pgp) or a related adenosine triphosphate-binding cassette membrane transporter. In some embodiments, the isolated recombinant polypeptide complex results in release of cytokine in the subject. In some embodiments, the cytokine is interleukin 6 (IL-6), interleukin 10 (IL-10), interferon γ (IFNγ), tumor necrosis factor (TNF), or a combination thereof. In some embodiments, the cytokine release is correlated with the dose. In some embodiments, IL-10 release occurs at a dose no less than 0.2 mg/kg. In some embodiments, IL-6 release occurs at a dose no less than 0.6 mg/kg. In some embodiments, IFNγ release occurs at a dose no less than 0.6 mg/kg.

In some embodiments, the isolated recombinant polypeptide complex provides a T_{1/2} of about 79 h to about 101 h, e.g., about 79 h, 80 h, 81 h, 82 h, 83 h, 84 h, 85 h, 86 h, 87 h, 88 h, 89 h, 90 h, 91 h, 92 h, 93 h, 94 h, 95 h, 96 h, 97 h, 98 h, 99 h, 100 h, or about 101 h, or any time therebetween, after administration of a single dose at about 0.05 mg/kg to about 1 mg/kg, e.g., about 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, 0.3 mg/kg, 0.31 mg/kg, 0.32 mg/kg, 0.33 mg/kg, 0.34 mg/kg, 0.35 mg/kg, 0.36 mg/kg, 0.37 mg/kg, 0.38 mg/kg, 0.39 mg/kg, 0.4 mg/kg, 0.41 mg/kg, 0.42 mg/kg, 0.43 mg/kg, 0.44 mg/kg, 0.45 mg/kg, 0.46 mg/kg, 0.47 mg/kg, 0.48 mg/kg, 0.49 mg/kg, 0.5 mg/kg, 0.51 mg/kg, 0.52 mg/kg, 0.53 mg/kg, 0.54 mg/kg, 0.55 mg/kg, 0.56 mg/kg, 0.57 mg/kg, 0.58 mg/kg, 0.59 mg/kg, 0.6 mg/kg, 0.61 mg/kg, 0.62 mg/kg, 0.63 mg/kg, 0.64 mg/kg, 0.65 mg/kg, 0.66 mg/kg, 0.67 mg/kg, 0.68 mg/kg, 0.69 mg/kg, 0.7 mg/kg, 0.71 mg/kg, 0.72 mg/kg, 0.73 mg/kg, 0.74 mg/kg, 0.75 mg/kg, 0.76 mg/kg, 0.77 mg/kg, 0.78 mg/kg, 0.79 mg/kg, 0.8 mg/kg, 0.81 mg/kg, 0.82 mg/kg, 0.83 mg/kg, 0.84 mg/kg, 0.85 mg/kg, 0.86 mg/kg, 0.87 mg/kg, 0.88 mg/kg, 0.89 mg/kg, 0.9 mg/kg, 0.91 mg/kg, 0.92 mg/kg, 0.93 mg/kg, 0.94 mg/kg, 0.95 mg/kg, 0.96 mg/kg, 0.97 mg/kg, 0.98 mg/kg, 0.99 mg/kg, or about 1 mg/kg, or any dose therebetween. In some embodiments, the isolated recombinant polypeptide complex provides a T_{1/2} of about 79 h to about 101 h after administration of a single dose at about 0.05 mg/kg to about 1 mg/kg.

In some embodiments, the isolated recombinant polypeptide complex is administered to a subject through a single intravenous bolus administration. In some embodiments, the isolated recombinant polypeptide complex provides a Cmax, wherein an increase of the Cmax is proportional to an increase of the dose. In some embodiments, the isolated recombinant polypeptide complex provides an AUC, wherein an increase of the AUC is proportional to an increase of the dose. In some embodiments, the isolated recombinant polypeptide complex is administered for at least 4 weeks. In some embodiments, the isolated recombinant polypeptide complex provides a no observed adverse effect level (NOAEL) at about 0.6 mg/kg. In some embodiments, the isolated recombinant polypeptide complex provides a Cmax of about 16100 ng/ml at day 1 at a dose of about 0.6 mg/kg, wherein day 1 is the day of the administration. In some embodiments, the isolated recombinant polypeptide complex provides an AUC₀₋₁₆₈ₕ of about 775000 hr*ng/ml at a dose of about 0.6 mg/kg administered on day 1. In some embodiments, the dose is about 0.05 mg/kg, about 0.2 mg/kg, or about 0.6 mg/kg. In some embodiments, the dose is about 0.05 mg/kg. In some embodiments, the dose is about 0.2 mg/kg. In some embodiments, the dose is about 0.6 mg/kg. In some embodiments, the dose is administered at days 1, 8, 15, 22, and 29. In some embodiments, the isolated recombinant polypeptide complex provides a Cmax of about 1870 ng/ml at a dose of about 0.05 mg/kg administered on day 1. In some embodiments, the isolated recombinant polypeptide complex provides a Cmax of about 6180 ng/ml at a dose of about 0.2 mg/kg administered on day 1. In some embodiments, the isolated recombinant polypeptide complex provides a Cmax of about 16100 ng/ml at a dose of about 0.6 mg/kg administered on day 1. In some embodiments, the isolated recombinant polypeptide complex provides an AUC₀₋₁₆₈ₕ of about 106000 hr*ng/ml at a dose of about 0.05 mg/kg administered on day 1. In some embodiments, the isolated recombinant polypeptide complex provides an AUC₀₋₁₆₈ₕ Of about 325000 hr*ng/ml at a dose of about 0.2 mg/kg administered on day 1. In some embodiments, the isolated recombinant polypeptide complex provides an AUC₀₋₁₆₈ₕ of about 775000 hr*ng/ml at a dose of about 0.6 mg/kg administered on day 1.

In some embodiments, the isolated recombinant polypeptide complex provides an AUC at a dose administered on day 22 that is similar to or same as an AUC at the same dose administered on day 1, wherein, after the administering on day 22, the subject exhibits no detectable level of an anti-drug antibody. In some embodiments, the isolated recombinant polypeptide complex provides an AUC at a dose administered on day 29 that is similar to or same as an AUC at the same dose administered on day 1, wherein, after the administering on day 29, the subject exhibits no detectable level of an anti-drug antibody. In some embodiments, the AUC is AUC₀₋₂₄ₕ or AUC₀₋₁₆₈ₕ. In some embodiments, the isolated recombinant polypeptide complex provides a Cmax at a dose administered on day 22 that is similar to or same as a Cmax at the same dose administered on day 1, wherein, after the administering on day 22, the subject exhibits no detectable level of an anti-drug antibody. In some embodiments, the isolated recombinant polypeptide complex provides a Cmax at a dose administered on day 29 that is similar to or same as a Cmax at the same dose administered on day 1, wherein, after the administering on day 29, the subject exhibits no detectable level of an anti-drug antibody.

In some embodiments, the isolated recombinant polypeptide complex provides a Cmax of about 1620 ng/ml after a single intravenous bolus administration of a dose of about 0.1 mg/kg. In some embodiments, the isolated recombinant polypeptide complex provides a Cmax of about 8150 ng/ml after a single intravenous bolus administration of a dose of about 0.3 mg/kg. In some embodiments, the isolated recombinant polypeptide complex provides a Cmax of about 27200 ng/ml after a single intravenous bolus administration of a dose of about 1 mg/kg. In some embodiments, the isolated recombinant polypeptide complex provides a T_{1/2} of about 88.8 h after a single intravenous bolus administration of a dose of about 0.1 mg/kg. In some embodiments, the isolated recombinant polypeptide complex provides a T_{1/2} of about 101 h after a single intravenous bolus administration of a dose of about 0.3 mg/kg. In some embodiments, the isolated recombinant polypeptide complex provides a T_{1/2} of about 79 h after a single intravenous bolus administration of a dose of about 1 mg/kg.

In some embodiments, the isolated recombinant polypeptide complex provides a clearance of about 2.5 ml/h to about 4.47 ml/h after a single intravenous bolus administration, e.g., about 2.5 ml/h, 2.51 ml/h, 2.52 ml/h, 2.53 ml/h, 2.54 ml/h, 2.55 ml/h, 2.56 ml/h, 2.57 ml/h, 2.58 ml/h, 2.59 ml/h, 2.6 ml/h, 2.61 ml/h, 2.62 ml/h, 2.63 ml/h, 2.64 ml/h, 2.65 ml/h, 2.66 ml/h, 2.67 ml/h, 2.68 ml/h, 2.69 ml/h, 2.7 ml/h, 2.71 ml/h, 2.72 ml/h, 2.73 ml/h, 2.74 ml/h, 2.75 ml/h, 2.76 ml/h, 2.77 ml/h, 2.78 ml/h, 2.79 ml/h, 2.8 ml/h, 2.81 ml/h, 2.82 ml/h, 2.83 ml/h, 2.84 ml/h, 2.85 ml/h, 2.86 ml/h, 2.87 ml/h, 2.88 ml/h, 2.89 ml/h, 2.9 ml/h, 2.91 ml/h, 2.92 ml/h, 2.93 ml/h, 2.94 ml/h, 2.95 ml/h, 2.96 ml/h, 2.97 ml/h, 2.98 ml/h, 2.99 ml/h, 3 ml/h, 3.01 ml/h, 3.02 ml/h, 3.03 ml/h, 3.04 ml/h, 3.05 ml/h, 3.06 ml/h, 3.07 ml/h, 3.08 ml/h, 3.09 ml/h, 3.1 ml/h, 3.11 ml/h, 3.12 ml/h, 3.13 ml/h, 3.14 ml/h, 3.15 ml/h, 3.16 ml/h, 3.17 ml/h, 3.18 ml/h, 3.19 ml/h, 3.2 ml/h, 3.21 ml/h, 3.22 ml/h, 3.23 ml/h, 3.24 ml/h, 3.25 ml/h, 3.26 ml/h, 3.27 ml/h, 3.28 ml/h, 3.29 ml/h, 3.3 ml/h, 3.31 ml/h, 3.32 ml/h, 3.33 ml/h, 3.34 ml/h, 3.35 ml/h, 3.36 ml/h, 3.37 ml/h, 3.38 ml/h, 3.39 ml/h, 3.4 ml/h, 3.41 ml/h, 3.42 ml/h, 3.43 ml/h, 3.44 ml/h, 3.45 ml/h, 3.46 ml/h, 3.47 ml/h, 3.48 ml/h, 3.49 ml/h, 3.5 ml/h, 3.51 ml/h, 3.52 ml/h, 3.53 ml/h, 3.54 ml/h, 3.55 ml/h, 3.56 ml/h, 3.57 ml/h, 3.58 ml/h, 3.59 ml/h, 3.6 ml/h, 3.61 ml/h, 3.62 ml/h, 3.63 ml/h, 3.64 ml/h, 3.65 ml/h, 3.66 ml/h, 3.67 ml/h, 3.68 ml/h, 3.69 ml/h, 3.7 ml/h, 3.71 ml/h, 3.72 ml/h, 3.73 ml/h, 3.74 ml/h, 3.75 ml/h, 3.76 ml/h, 3.77 ml/h, 3.78 ml/h, 3.79 ml/h, 3.8 ml/h, 3.81 ml/h, 3.82 ml/h, 3.83 ml/h, 3.84 ml/h, 3.85 ml/h, 3.86 ml/h, 3.87 ml/h, 3.88 ml/h, 3.89 ml/h, 3.9 ml/h, 3.91 ml/h, 3.92 ml/h, 3.93 ml/h, 3.94 ml/h, 3.95 ml/h, 3.96 ml/h, 3.97 ml/h, 3.98 ml/h, 3.99 ml/h, 4 ml/h, 4.01 ml/h, 4.02 ml/h, 4.03 ml/h, 4.04 ml/h, 4.05 ml/h, 4.06 ml/h, 4.07 ml/h, 4.08 ml/h, 4.09 ml/h, 4.1 ml/h, 4.11 ml/h, 4.12 ml/h, 4.13 ml/h, 4.14 ml/h, 4.15 ml/h, 4.16 ml/h, 4.17 ml/h, 4.18 ml/h, 4.19 ml/h, 4.2 ml/h, 4.21 ml/h, 4.22 ml/h, 4.23 ml/h, 4.24 ml/h, 4.25 ml/h, 4.26 ml/h, 4.27 ml/h, 4.28 ml/h, 4.29 ml/h, 4.3 ml/h, 4.31 ml/h, 4.32 ml/h, 4.33 ml/h, 4.34 ml/h, 4.35 ml/h, 4.36 ml/h, 4.37 ml/h, 4.38 ml/h, 4.39 ml/h, 4.4 ml/h, 4.41 ml/h, 4.42 ml/h, 4.43 ml/h, 4.44 ml/h, 4.45 ml/h, 4.46 ml/h, or about 4.47 ml/h, or any rate therebetween.

In some embodiments, the isolated recombinant polypeptide complex provides a volume of distribution of about 372 ml to about 576 ml after a single intravenous bolus administration, e.g., about 372 ml, 373 ml, 374 ml, 375 ml, 376 ml, 377 ml, 378 ml, 379 ml, 380 ml, 381 ml, 382 ml, 383 ml, 384 ml, 385 ml, 386 ml, 387 ml, 388 ml, 389 ml, 390 ml, 391 ml, 392 ml, 393 ml, 394 ml, 395 ml, 396 ml, 397 ml, 398 ml, 399 ml, 400 ml, 401 ml, 402 ml, 403 ml, 404 ml, 405 ml, 406 ml, 407 ml, 408 ml, 409 ml, 410 ml, 411 ml, 412 ml, 413 ml, 414 ml, 415 ml, 416 ml, 417 ml, 418 ml, 419 ml, 420 ml, 421 ml, 422 ml, 423 ml, 424 ml, 425 ml, 426 ml, 427 ml, 428 ml, 429 ml, 430 ml, 431 ml, 432 ml, 433 ml, 434 ml, 435 ml, 436 ml, 437 ml, 438 ml, 439 ml, 440 ml, 441 ml, 442 ml, 443 ml, 444 ml, 445 ml, 446 ml, 447 ml, 448 ml, 449 ml, 450 ml, 451 ml, 452 ml, 453 ml, 454 ml, 455 ml, 456 ml, 457 ml, 458 ml, 459 ml, 460 ml, 461 ml, 462 ml, 463 ml, 464 ml, 465 ml, 466 ml, 467 ml, 468 ml, 469 ml, 470 ml, 471 ml, 472 ml, 473 ml, 474 ml, 475 ml, 476 ml, 477 ml, 478 ml, 479 ml, 480 ml, 481 ml, 482 ml, 483 ml, 484 ml, 485 ml, 486 ml, 487 ml, 488 ml, 489 ml, 490 ml, 491 ml, 492 ml, 493 ml, 494 ml, 495 ml, 496 ml, 497 ml, 498 ml, 499 ml, 500 ml, 501 ml, 502 ml, 503 ml, 504 ml, 505 ml, 506 ml, 507 ml, 508 ml, 509 ml, 510 ml, 511 ml, 512 ml, 513 ml, 514 ml, 515 ml, 516 ml, 517 ml, 518 ml, 519 ml, 520 ml, 521 ml, 522 ml, 523 ml, 524 ml, 525 ml, 526 ml, 527 ml, 528 ml, 529 ml, 530 ml, 531 ml, 532 ml, 533 ml, 534 ml, 535 ml, 536 ml, 537 ml, 538 ml, 539 ml, 540 ml, 541 ml, 542 ml, 543 ml, 544 ml, 545 ml, 546 ml, 547 ml, 548 ml, 549 ml, 550 ml, 551 ml, 552 ml, 553 ml, 554 ml, 555 ml, 556 ml, 557 ml, 558 ml, 559 ml, 560 ml, 561 ml, 562 ml, 563 ml, 564 ml, 565 ml, 566 ml, 567 ml, 568 ml, 569 ml, 570 ml, 571 ml, 572 ml, 573 ml, 574 ml, 575 ml, or about 576 ml, or any volume therebetween.

In some embodiments, the isolated recombinant polypeptide complex provides a Cmax of about 1170 ng/ml after a single intravenous infusion of a dose of about 0.05 mg/kg over about 30 min. In some embodiments, the isolated recombinant polypeptide complex provides a Cmax of about 5950 ng/ml after a single intravenous infusion of a dose of about 0.2 mg/kg over about 30 min. In some embodiments, the isolated recombinant polypeptide complex provides a Cmax of about 17300 ng/ml after a single intravenous infusion of a dose of about 0.6 mg/kg over about 30 min. In some embodiments, the isolated recombinant polypeptide complex provides a clearance of about 0.504 ml/hr/kg after a single intravenous infusion of a dose of about 0.05 mg/kg over about 30 min. In some embodiments, the isolated recombinant polypeptide complex provides a clearance of about 0.762 ml/hr/kg after a single intravenous infusion of a dose of about 0.2 mg/kg over about 30 min. In some embodiments, the isolated recombinant polypeptide complex provides a clearance of about 0.650 ml/hr/kg after a single intravenous infusion of a dose of about 0.6 mg/kg over about 30 min. In some embodiments, the isolated recombinant polypeptide complex provides a volume of distribution of about 58.8 ml/kg after a single intravenous infusion of a dose of about 0.05 mg/kg over about 30 min. In some embodiments, the isolated recombinant polypeptide complex provides a volume of distribution of about 75.7 ml/kg after a single intravenous infusion of a dose of about 0.2 mg/kg over about 30 min. In some embodiments, the isolated recombinant polypeptide complex provides a volume of distribution of about 88.9 ml/kg after a single intravenous infusion of a dose of about 0.6 mg/kg over about 30 min. In some embodiments, the isolated recombinant polypeptide complex provides a T_{1/2} of about 81.3 h after a single intravenous infusion of a dose of about 0.05 mg/kg over about 30 min. In some embodiments, the isolated recombinant polypeptide complex provides a T_{1/2} of about 68.2 h after a single intravenous infusion of a dose of about 0.05 mg/kg over about 30 min. In some embodiments, the isolated recombinant polypeptide complex provides a T_{1/2} of about 96.5 h after a single intravenous infusion of a dose of about 0.05 mg/kg over about 30 min. In some embodiments, the subject is a primate. In some embodiments, the subject is a non-human primate. In some embodiments, the subject is a monkey. In some embodiments, the subject is a cynomolgus monkey. In some embodiments, the subject is a human. In some embodiments, the isolated recombinant polypeptide complex degrades in vitro at a rate of about 1% per day in the serum of a subject. In some embodiments, the isolated recombinant polypeptide complex degrades in vitro at a rate of about 2% per day in the serum of a subject. In some embodiments, the isolated recombinant polypeptide complex degrades in vitro at a rate of about 6.6% to about 11.5% per day in the serum of a subject, e.g., about 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, about 11.5%, or any percentage therebetween. In some embodiments, the subject is healthy.

In some embodiments, the subject has cancer. In some embodiments, the cancer comprises colorectal cancer (CRC), squamous cell carcinoma of head and neck (SCCHN), or non-small cell lung cancer (NSCL). In some embodiments, the cancer is CRC. In some embodiments, the cancer is SCCHN. In some embodiments, the cancer is NSCLC.

### Kits

Provided herein, in some embodiments, is a kit comprising a recombinant antibody (such as any described herein) or a composition (such as any described herein), a container, and a label or package insert on or associated with the container.

### Methods of Treatment

Disclosed herein are methods of treating a subject having cancer, the method comprising: administering to the subject any of the antibodies that bind specifically to EGFR and CD3 as disclosed herein. In some embodiments, the cancer comprises cancer cells that express EGFR or CD3. In some embodiments, the cancer cells that express EGFR or CD3 are lysed. In some embodiments, the antibody induces antibody-dependent cellular phagocytosis (ADCP) of the cancer cells that express EGFR or CD3.

In some embodiments, the isolated recombinant polypeptide complex described herein is used in a method of treating cancer. In some embodiments, the isolated polypeptide disclosed herein is used in a method of treating cancer and the isolated polypeptide is an enzymatic product of the isolated recombinant polypeptide complex after the administering. In some embodiments, the cancer has cells that express EGFR. In some embodiments, the polypeptides or polypeptide complexes described herein are used in a method of treating renal cell carcinoma, colorectal cancer (CRC), squamous cell carcinoma of the head and neck (SCCHN), non-small cell lung cancer (NSCLC), prostate cancer, breast cancer, colon/rectum cancer, head and neck cancer, esophagogastric cancer, liver cancer, glioblastoma, cervical cancer, ovarian cancer, bladder cancer, kidney cancer, or pancreatic cancer. In some embodiments, the polypeptides or polypeptide complexes described herein are used in a method of treating subjects who are resistant to EGFR inhibitor treatment. In some embodiments, the polypeptides or polypeptide complexes described herein are used in a method of treating subjects who harbor KRAS mutations. In some embodiments, the polypeptides or polypeptide complexes described herein are used in a method of treating subjects who are resistant to EGFR inhibitor treatment and harbor KRAS mutations.

Described herein, in some embodiments, is an isolated recombinant polypeptide complex administered as once weekly. In some embodiments, the isolated recombinant polypeptide complex is administered once weekly by intravenous, intramuscular, intralesional, topical, subcutaneous, infusion, or oral. In some embodiments, the isolated recombinant polypeptide complex is administered once weekly by bolus injection. In some embodiments, the isolated recombinant polypeptide complex is administered once weekly by continuous infusion. In some embodiments, the isolated recombinant polypeptide complex is administered to the subject once a week as a continuous infusion over a period of no more than 60 minutes. In some embodiments, the isolated recombinant polypeptide complex is administered to the subject once a week as a continuous intravenous infusion over a period of no more than 30 minutes. In some embodiments, the isolated recombinant polypeptide complex is administered to the subject once a week as a continuous intravenous infusion over a period of at least 10 minutes.

In some embodiments, the method further comprises administering to the subject an anti-cancer agent. In some embodiments, the anti-cancer agent is a chemotherapeutic agent or a biologic agent. In some embodiments, the administering is sufficient to reduce or eliminate the cancer as compared to a comparable method lacking the administering.

In some embodiments, are methods of treating cancer in a subject need in need thereof comprising administering to the subject an isolated recombinant polypeptide complex as described herein. In some embodiments, the cancer has cells that express EGFR.

For administration to a subject, the isolated recombinant polypeptide complex as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions. In some embodiments, a pharmaceutical composition disclosed herein is administered intravenously to a subject in need thereof.

Dosages of the substances of the present disclosure can vary between wide limits, depending upon the disease or disorder to be treated, the age and condition of the individual to be treated, etc. and a physician will ultimately determine appropriate dosages to be used.

In some embodiments, disclosed herein is a method for treating cancer comprising administering to a subject in need thereof an effective amount of a pharmaceutical composition. In some embodiments, the pharmaceutical composition comprises a dose of the isolated recombinant polypeptide complex disclosed herein. In some embodiments, the pharmaceutical composition comprises a pharmaceutically acceptable excipient. In some embodiments, the cancer comprises a cancer cell expressing epidermal growth factor receptor (EGFR). In some embodiments, the cancer comprises a cancer cell overexpressing EGFR.

In some embodiments, the cancer comprises CRC, SCCHN, NSCLC, renal cell carcinoma (RCC), breast cancer, pancreatic cancer, ovarian cancer, prostate cancer, brain cancer, glioblastoma multiforme, or papillary carcinoma. In some embodiments, the cancer comprises CRC. In some embodiments, the cancer comprises SCCHN. In some embodiments, the cancer comprises NSCLC. In some embodiments, the cancer comprises RCC. In some embodiments, the cancer comprises breast cancer. In some embodiments, the cancer comprises pancreatic cancer. In some embodiments, the cancer comprises ovarian cancer. In some embodiments, the cancer comprises prostate cancer. In some embodiments, the cancer comprises brain cancer. In some embodiments, the cancer comprises glioblastoma multiforme. In some embodiments, the cancer comprises papillary carcinoma. In some embodiments, the cancer is metastatic, refractory, or relapsed. In some embodiments, the cancer is metastatic. In some embodiments, the cancer is refractory. In some embodiments, the cancer is relapsed. In some embodiments, the cancer is advanced or non-advanced. In some embodiments, the cancer is advanced. In some embodiments, the cancer is non-advanced.

In some embodiments, the dose is at least about 25 g to at least about 80 mg, e.g., at least about 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, or at least about 80 mg, or any dose therebetween. In some embodiments, the dose is at most about 25 g to at most about 80 mg, e.g., at most about 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, or at most about 80 mg, or any dose therebetween. In some embodiments, the dose is about 25 μg to about 80 mg, e.g., about 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, or about 80 mg, or any dose therebetween. In some embodiments, a dose disclosed herein is any dose administered in the method. In some embodiments, a dose disclosed herein is the first dose administered to the subject in the method. In some embodiments, a dose disclosed herein is the last dose administered to the subject in the method. In some embodiments, a dose disclosed herein is a dose administered to the subject between the first dose and the last dose in the method. In some embodiments, two or more doses disclosed herein are administered to the subject in a dosing regimen. In some embodiments, three or more doses disclosed herein are administered to the subject in a dosing regimen. In some embodiments, more than three doses disclosed herein are administered to the subject in a dosing regimen.

In some embodiments, the dose is at least about 25 μg, at least about 50 μg, at least about 100 μg, at least about 150 μg, or at least about 200 μg. In some embodiments, the dose is at least about 25 μg. In some embodiments, the dose is at least about 50 μg. In some embodiments, the dose is at least about 100 μg. In some embodiments, the dose is at least about 150 μg. In some embodiments, the dose is at least about 200 μg. In some embodiments, the dose is at most about 25 μg, at most about 50 μg, at most about 100 μg, at most about 150 μg, or at most about 200 μg. In some embodiments, the dose is at most about 25 μg. In some embodiments, the dose is at most about 50 μg. In some embodiments, the dose is at most about 100 μg. In some embodiments, the dose is at most about 150 μg. In some embodiments, the dose is at most about 200 μg. In some embodiments, the dose is about 25 μg, about 50 μg, about 100 μg, about 150 μg, or about 200 μg. In some embodiments, the dose is about 25 μg. In some embodiments, the dose is about 50 μg. In some embodiments, the dose is about 100 μg. In some embodiments, the dose is about 150 μg. In some embodiments, the dose is about 200 μg. In some embodiments, the dose is at least about 300 μg, at least about 400 μg, at least about 500 μg, at least about 600 μg, at least about 700 μg, at least about 800 μg, at least about 900 μg, at least about 1 mg, at least about 5 mg, at least about 10 mg, at least about 20 mg, at least about 40 mg, at least about 60 mg, or at least about 80 mg. In some embodiments, the dose is at most about 300 μg, at most about 400 μg, at most about 500 μg, at most about 600 μg, at most about 700 μg, at most about 800 μg, at most about 900 μg, at most about 1 mg, at most about 5 mg, at most about 10 mg, at most about 20 mg, at most about 40 mg, at most about 60 mg, or at most about 80 mg. In some embodiments, the dose is about 300 μg, about 400 μg, about 500 μg, about 600 μg, about 700 μg, about 800 μg, about 900 μg, about 1 mg, about 5 mg, about 10 mg, about 20 mg, about 40 mg, about 60 mg, or about 80 mg. In some embodiments, the dose is at least about 300 μg. In some embodiments, the dose is at least about 400 μg. In some embodiments, the dose is at least about 500 μg. In some embodiments, the dose is at least about 600 μg. In some embodiments, the dose is at least about 700 μg. In some embodiments, the dose is at least about 800 μg. In some embodiments, the dose is at least about 900 μg. In some embodiments, the dose is at least about 1 mg. In some embodiments, the dose is at least about 5 mg. In some embodiments, the dose is at least about 10 mg. In some embodiments, the dose is at least about 20 mg. In some embodiments, the dose is at least about 40 mg. In some embodiments, the dose is at least about 60 mg. In some embodiments, the dose is at least about 80 mg. In some embodiments, the dose is at most about 300 μg. In some embodiments, the dose is at most about 400 μg. In some embodiments, the dose is at most about 500 μg. In some embodiments, the dose is at most about 600 μg. In some embodiments, the dose is at most about 700 μg. In some embodiments, the dose is at most about 800 μg. In some embodiments, the dose is at most about 900 μg. In some embodiments, the dose is at most about 1 mg. In some embodiments, the dose is at most about 5 mg. In some embodiments, the dose is at most about 10 mg. In some embodiments, the dose is at most about 20 mg. In some embodiments, the dose is at most about 40 mg. In some embodiments, the dose is at most about 60 mg. In some embodiments, the dose is at most about 80 mg. In some embodiments, the dose is about 300 μg. In some embodiments, the dose is about 400 μg. In some embodiments, the dose is about 500 μg. In some embodiments, the dose is about 600 μg. In some embodiments, the dose is about 700 μg. In some embodiments, the dose is about 800 μg. In some embodiments, the dose is about 900 μg. In some embodiments, the dose is about 1 mg. In some embodiments, the dose is about 5 mg. In some embodiments, the dose is about 10 mg. In some embodiments, the dose is about 20 mg. In some embodiments, the dose is about 40 mg. In some embodiments, the dose is about 60 mg. In some embodiments, the dose is about 80 mg. In some embodiments, a dose disclosed herein is any dose administered in the method. In some embodiments, a dose disclosed herein is the first dose administered to the subject in the method. In some embodiments, a dose disclosed herein is the last dose administered to the subject in the method. In some embodiments, a dose disclosed herein is a dose administered to the subject between the first dose and the last dose in the method. In some embodiments, two or more doses disclosed herein are administered to the subject in a dosing regimen. In some embodiments, three or more doses disclosed herein are administered to the subject in a dosing regimen. In some embodiments, more than three doses disclosed herein are administered to the subject in a dosing regimen.

In some embodiments, the dose comprises 1 dose, 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 18 doses, 19 doses, 20 doses, 21 doses, 22 doses, 23 doses, 24 doses, 25 doses, 26 doses, 27 doses, 28 doses, 29 doses, 30 doses, 31 doses, 32 doses, 33 doses, 34 doses, 35 doses, 36 doses, 37 doses, 38 doses, 39 doses, 40 doses, 41 doses, 42 doses, 43 doses, 44 doses, 45 doses, 46 doses, 47 doses, 48 doses, 49 doses, 50 doses, 51 doses, 52 doses, 53 doses, 54 doses, 55 doses, 56 doses, 57 doses, 58 doses, 59 doses, 60 doses, 61 doses, 62 doses, 63 doses, 64 doses, 65 doses, 66 doses, 67 doses, 68 doses, 69 doses, 70 doses, 71 doses, 72 doses, 73 doses, 74 doses, 75 doses, 76 doses, 77 doses, 78 doses, 79 doses, 80 doses, 81 doses, 82 doses, 83 doses, 84 doses, 85 doses, 86 doses, 87 doses, 88 doses, 89 doses, 90 doses, 91 doses, 92 doses, 93 doses, 94 doses, 95 doses, 96 doses, 97 doses, 98 doses, 99 doses, or 100 doses, or more than 100 doses. In some embodiments, the dose comprises at least 2 doses. In some embodiments, the dose comprises at least 3 doses. In some embodiments, the dose comprises at least 4 doses. In some embodiments, the dose comprises at least 5 doses. In some embodiments, the dose comprises at least 6 doses. In some embodiments, the dose comprises at least 7 doses. In some embodiments, the dose comprises at least 8 doses. In some embodiments, the dose comprises at least 9 doses. In some embodiments, the dose comprises at least 10 doses. In some embodiments, the dose comprises at least 9 doses. In some embodiments, the dose comprises at least 20 doses. In some embodiments, the dose comprises at least 9 doses. In some embodiments, the dose comprises at least 30 doses. In some embodiments, the dose comprises at least 9 doses. In some embodiments, the dose comprises at least 40 doses. In some embodiments, the dose comprises at least 9 doses. In some embodiments, the dose comprises at least 50 doses. In some embodiments, the dose comprises at least 9 doses. In some embodiments, the dose comprises at least 100 doses. In some embodiments, the dose comprises at least 9 doses. In some embodiments, the dose comprises at least 200 doses. In some embodiments, the dose comprises at least 9 doses. In some embodiments, the dose comprises at least 300 doses. In some embodiments, the dose comprises at least 9 doses. In some embodiments, the dose comprises at least 400 doses. In some embodiments, the dose comprises at least 500 doses.

In some embodiments, the dose comprises a first dose and a second dose, wherein the second dose is equal to or higher than the first dose, wherein the first dose or the second dose is a dose disclosed herein. In some embodiments, the dose comprises a first dose and a second dose, wherein the second dose is equal to or higher than the first dose, wherein the first dose is at least about 25 μg. In some embodiments, the dose comprises a first dose and a second dose, wherein the second dose is equal to or higher than the first dose, wherein the first dose is at least about 50 μg. In some embodiments, the dose comprises a first dose and a second dose, wherein the second dose is equal to or higher than the first dose, wherein the first dose is at least about 100 μg. In some embodiments, the dose comprises a first dose and a second dose, wherein the second dose is equal to or higher than the first dose, wherein the first dose is at least about 150 μg. In some embodiments, the dose comprises a first dose and a second dose, wherein the second dose is equal to or higher than the first dose, wherein the first dose is at least about 200 μg. In some embodiments, the dose comprises a first dose and a second dose, wherein the second dose is equal to or higher than the first dose, wherein the first dose is a dose disclosed herein. In some embodiments, the dose comprises a first dose and a second dose, wherein the second dose is equal to or higher than the first dose, wherein the first dose is at most about 25 μg. In some embodiments, the dose comprises a first dose and a second dose, wherein the second dose is equal to or higher than the first dose, wherein the first dose is at most about 50 μg. In some embodiments, the dose comprises a first dose and a second dose, wherein the second dose is equal to or higher than the first dose, wherein the first dose is at most about 100 μg. In some embodiments, the dose comprises a first dose and a second dose, wherein the second dose is equal to or higher than the first dose, wherein the first dose is at most about 150 μg. In some embodiments, the dose comprises a first dose and a second dose, wherein the second dose is equal to or higher than the first dose, wherein the first dose is at most about 200 μg. In some embodiments, the dose comprises a first dose and a second dose, wherein the second dose is equal to or higher than the first dose, wherein the first dose is about 25 μg. In some embodiments, the dose comprises a first dose and a second dose, wherein the second dose is equal to or higher than the first dose, wherein the first dose is about 50 μg. In some embodiments, the dose comprises a first dose and a second dose, wherein the second dose is equal to or higher than the first dose, wherein the first dose is about 100 μg. In some embodiments, the dose comprises a first dose and a second dose, wherein the second dose is equal to or higher than the first dose, wherein the first dose is about 150 μg. In some embodiments, the dose comprises a first dose and a second dose, wherein the second dose is equal to or higher than the first dose, wherein the first dose is about 200 μg.

In some embodiments, the dose comprises a first dose, a second dose, and a third dose, wherein the second dose is equal to or higher than the first dose, wherein the third dose is equal to or higher than the second dose, wherein the first dose, the second dose, or the third dose is a dose disclosed herein. In some embodiments, the dose comprises a first dose, a second dose, and a third dose, wherein the second dose is equal to or higher than the first dose, wherein the third dose is equal to or higher than the second dose, wherein the first dose is at least about 25 μg. In some embodiments, the dose comprises a first dose, a second dose, and a third dose, wherein the second dose is equal to or higher than the first dose, wherein the third dose is equal to or higher than the second dose, wherein the first dose is at least about 50 μg. In some embodiments, the dose comprises a first dose, a second dose, and a third dose, wherein the second dose is equal to or higher than the first dose, wherein the third dose is equal to or higher than the second dose, wherein the first dose is at least about 100 μg. In some embodiments, the dose comprises a first dose, a second dose, and a third dose, wherein the second dose is equal to or higher than the first dose, wherein the third dose is equal to or higher than the second dose, wherein the first dose is at least about 150 μg. In some embodiments, the dose comprises a first dose, a second dose, and a third dose, wherein the second dose is equal to or higher than the first dose, wherein the third dose is equal to or higher than the second dose, wherein the first dose is at least about 200 μg. In some embodiments, the dose comprises a first dose, a second dose, and a third dose, wherein the second dose is equal to or higher than the first dose, wherein the third dose is equal to or higher than the second dose, wherein the first dose is at most about 25 μg. In some embodiments, the dose comprises a first dose, a second dose, and a third dose, wherein the second dose is equal to or higher than the first dose, wherein the third dose is equal to or higher than the second dose, wherein the first dose is at most about 50 μg. In some embodiments, the dose comprises a first dose, a second dose, and a third dose, wherein the second dose is equal to or higher than the first dose, wherein the third dose is equal to or higher than the second dose, wherein the first dose is at most about 100 μg. In some embodiments, the dose comprises a first dose, a second dose, and a third dose, wherein the second dose is equal to or higher than the first dose, wherein the third dose is equal to or higher than the second dose, wherein the first dose is at most about 150 μg. In some embodiments, the dose comprises a first dose, a second dose, and a third dose, wherein the second dose is equal to or higher than the first dose, wherein the third dose is equal to or higher than the second dose, wherein the first dose is at most about 200 μg.

In some embodiments, the dose comprises a first dose, a second dose, and a third dose, wherein the second dose is equal to or higher than the first dose, wherein the third dose is equal to or higher than the second dose, wherein the first dose is about 25 μg. In some embodiments, the dose comprises a first dose, a second dose, and a third dose, wherein the second dose is equal to or higher than the first dose, wherein the third dose is equal to or higher than the second dose, wherein the first dose is about 50 μg. In some embodiments, the dose comprises a first dose, a second dose, and a third dose, wherein the second dose is equal to or higher than the first dose, wherein the third dose is equal to or higher than the second dose, wherein the first dose is about 100 μg. In some embodiments, the dose comprises a first dose, a second dose, and a third dose, wherein the second dose is equal to or higher than the first dose, wherein the third dose is equal to or higher than the second dose, wherein the first dose is about 150 μg. In some embodiments, the dose comprises a first dose, a second dose, and a third dose, wherein the second dose is equal to or higher than the first dose, wherein the third dose is equal to or higher than the second dose, wherein the first dose is about 200 μg. In some embodiments, the dose comprises a number of doses disclosed herein, wherein the first dose of the number of doses is a dose disclosed herein and at most equal to any other dose of the number of doses.

In some embodiments, a dose of the isolated recombinant polypeptide complex is administered at least once weekly. In some embodiments, a dose of the isolated recombinant polypeptide complex is administered at least twice weekly. In some embodiments, a dose of the isolated recombinant polypeptide complex is administered at most once weekly. In some embodiments, a dose of the isolated recombinant polypeptide complex is administered at most twice weekly. In some embodiments, a dose of the isolated recombinant polypeptide complex is administered once weekly. In some embodiments, a dose of the isolated recombinant polypeptide complex is administered twice weekly.

In some embodiments, a dose of the isolated recombinant polypeptide complex is administered at least once every two weeks. In some embodiments, a dose of the isolated recombinant polypeptide complex is administered at least twice every two weeks. In some embodiments, a dose of the isolated recombinant polypeptide complex is administered at least three times every two weeks. In some embodiments, a dose of the isolated recombinant polypeptide complex is administered at most once every two weeks. In some embodiments, a dose of the isolated recombinant polypeptide complex is administered at most twice every two weeks. In some embodiments, a dose of the isolated recombinant polypeptide complex is administered at most three times every two weeks. In some embodiments, a dose of the isolated recombinant polypeptide complex is administered once every two weeks. In some embodiments, a dose of the isolated recombinant polypeptide complex is administered twice every two weeks. In some embodiments, a dose of the isolated recombinant polypeptide complex is administered three times every two weeks. In some embodiments, a dose of the isolated recombinant polypeptide complex is administered at least once every three weeks. In some embodiments, a dose of the isolated recombinant polypeptide complex is administered at least twice every three weeks. In some embodiments, a dose of the isolated recombinant polypeptide complex is administered at least three times every three weeks. In some embodiments, a dose of the isolated recombinant polypeptide complex is administered at most once every three weeks. In some embodiments, a dose of the isolated recombinant polypeptide complex is administered at most twice every three weeks. In some embodiments, a dose of the isolated recombinant polypeptide complex is administered at most three times every three weeks. In some embodiments, a dose of the isolated recombinant polypeptide complex is administered once every three weeks. In some embodiments, a dose of the isolated recombinant polypeptide complex is administered twice every three weeks. In some embodiments, a dose of the isolated recombinant polypeptide complex is administered three times every three weeks. In some embodiments, a dose of the isolated recombinant polypeptide complex is administered at least once every four weeks. In some embodiments, a dose of the isolated recombinant polypeptide complex is administered at least twice every four weeks. In some embodiments, a dose of the isolated recombinant polypeptide complex is administered at most once every four weeks. In some embodiments, a dose of the isolated recombinant polypeptide complex is administered at most twice every four weeks. In some embodiments, a dose of the isolated recombinant polypeptide complex is administered once every four weeks. In some embodiments, a dose of the isolated recombinant polypeptide complex is administered twice every four weeks. In some embodiments, a dose of the isolated recombinant polypeptide complex is administered at least once every five or more weeks. In some embodiments, a dose of the isolated recombinant polypeptide complex is administered at most once every five or more weeks.

In some embodiments, the method further comprises a treatment course. In some embodiments, the method further comprises at least two of the treatment course. In some embodiments, the method further comprises a 21-day treatment course. In some embodiments, the method further comprises a 28-day treatment course. In some embodiments, the method further comprises at least one of the 21-day treatment course. In some embodiments, the method further comprises at least one of the 28-day treatment course. In some embodiments, the method further comprises at least one of the 21-day treatment course and at least one of the 28-day treatment course. In some embodiments, the method further comprises at least two of the 21-day treatment course. In some embodiments, the method further comprises at least two of the 28-day treatment course. In some embodiments, the method further comprises at least two of the 21-day treatment course and at least two of the 28-day treatment course. In some embodiments, the method further comprises one of the 21-day treatment course and at least one of the 28-day treatment course. In some embodiments, the method further comprises at least one of the 21-day treatment course and at least two of the 28-day treatment course. In some embodiments, the method further comprises at least two of the 21-day treatment course and at least one of the 28-day treatment course. In some embodiments, the method further comprises at least two of the 21-day treatment course and at least two of the 28-day treatment course. In some embodiments, the method comprises at least one of the 28-day treatment course and does not comprise any of the 21-day treatment course. In some embodiments, the method further comprises at least two of the 21-day treatment course and at least four of the 28-day treatment course. In some embodiments, the method further comprises two of the 21-day treatment course and at least four of the 28-day treatment course. In some embodiments, the method further comprises at least six of the 28-day treatment course. In some embodiments, the method further comprises one of the 21-day treatment course and at least five of the 28-day treatment course. In some embodiments, the method further comprises at least two, three, four, five, six, seven, eight, nine, ten, or more than ten of the 21-day treatment course. In some embodiments, the method further comprises at most two, three, four, five, six, seven, eight, nine, ten, or more than ten of the 21-day treatment course. In some embodiments, the method further comprises at least 20, 30, 40, 50, 60, 70, 80, 90, or 100 of the 21-day treatment course. In some embodiments, the method further comprises at most 20, 30, 40, 50, 60, 70, 80, 90, or 100 of the 21-day treatment course. In some embodiments, the method further comprises at least two, three, four, five, six, seven, eight, nine, ten, or more than ten of the 28-day treatment course. In some embodiments, the method further comprises at most two, three, four, five, six, seven, eight, nine, ten, or more than ten of the 28-day treatment course. In some embodiments, the method further comprises at least 20, 30, 40, 50, 60, 70, 80, 90, or 100 of the 28-day treatment course. In some embodiments, the method further comprises at most 20, 30, 40, 50, 60, 70, 80, 90, or 100 of the 28-day treatment course.

In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is a dose disclosed herein. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at least about 25 μg, 50 μg, 100 μg, 150 μg, or 200 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at least about 25 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at least about 50 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at least about 100 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at least about 150 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at least about 200 μg.

In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at most about 25 μg, 50 μg, 100 μg, 150 μg, or 200 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at most about 25 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at most about 50 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at most about 100 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at most about 150 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at most about 200 μg.

In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is about 25 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is about 50 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is about 100 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is about 150 μg. In some embodiments, a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is about 200 μg. In some embodiments, a first dose and a second dose are each administered to the subject biweekly during the treatment course, wherein the second dose is equal to or higher than the first dose, wherein the first dose is at least about 25 μg. In some embodiments, a first dose and a second dose are each administered to the subject biweekly during the treatment course, wherein the second dose is equal to or higher than the first dose, wherein the first dose is at least about 50 μg. In some embodiments, a first dose and a second dose are each administered to the subject biweekly during the treatment course, wherein the second dose is equal to or higher than the first dose, wherein the first dose is at least about 100 μg. In some embodiments, a first dose and a second dose are each administered to the subject biweekly during the treatment course, wherein the second dose is equal to or higher than the first dose, wherein the first dose is at least about 150 μg. In some embodiments, a first dose and a second dose are each administered to the subject biweekly during the treatment course, wherein the second dose is equal to or higher than the first dose, wherein the first dose is at least about 200 μg. In some embodiments, a first dose and a second dose are each administered to the subject biweekly during the treatment course, wherein the second dose is equal to or higher than the first dose, wherein the first dose is at most about 25 μg. In some embodiments, a first dose and a second dose are each administered to the subject biweekly during the treatment course, wherein the second dose is equal to or higher than the first dose, wherein the first dose is at most about 50 μg. In some embodiments, a first dose and a second dose are each administered to the subject biweekly during the treatment course, wherein the second dose is equal to or higher than the first dose, wherein the first dose is at most about 100 μg. In some embodiments, a first dose and a second dose are each administered to the subject biweekly during the treatment course, wherein the second dose is equal to or higher than the first dose, wherein the first dose is at most about 150 μg. In some embodiments, a first dose and a second dose are each administered to the subject biweekly during the treatment course, wherein the second dose is equal to or higher than the first dose, wherein the first dose is at most about 200 μg. In some embodiments, a first dose and a second dose are each administered to the subject biweekly during the treatment course, wherein the second dose is equal to or higher than the first dose, wherein the first dose is about 25 μg. In some embodiments, a first dose and a second dose are each administered to the subject biweekly during the treatment course, wherein the second dose is equal to or higher than the first dose, wherein the first dose is about 50 μg. In some embodiments, a first dose and a second dose are each administered to the subject biweekly during the treatment course, wherein the second dose is equal to or higher than the first dose, wherein the first dose is about 100 μg. In some embodiments, a first dose and a second dose are each administered to the subject biweekly during the treatment course, wherein the second dose is equal to or higher than the first dose, wherein the first dose is about 150 μg. In some embodiments, a first dose and a second dose are each administered to the subject biweekly during the treatment course, wherein the second dose is equal to or higher than the first dose, wherein the first dose is about 200 μg.

In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is at least about 25 μg. In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is at least about 50 μg. In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is at least about 100 μg. In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is at least about 150 μg. In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is at least about 200 μg.

In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is at most about 25 μg. In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is at most about 50 μg. In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is at most about 100 μg. In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is at most about 150 μg. In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is at most about 200 μg.

In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is about 25 μg. In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is about 50 μg. In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is about 100 μg. In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is about 150 μg. In some embodiments, a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is about 200 μg.

In some embodiments, a dose is administered on day 1 of the treatment course. In some embodiments, a dose is administered on day 4 of the treatment course. In some embodiments, a dose is administered on day 8 of the treatment course. In some embodiments, a dose is administered on day 15 of the treatment course. In some embodiments, the first dose is administered on day 1 of the treatment course, the second dose is administered on day 8 of the treatment course, and the third dose is administered on day 15 of the treatment course. In some embodiments, the first dose is administered on day 1 of the 21-day treatment course, the second dose is administered on day 8 of the 21-day treatment course, and the third dose is administered on day 15 of the 21-day treatment course. In some embodiments, the first dose is administered on day 1 of the 28-day treatment course, the second dose is administered on day 8 of the 28-day treatment course, and the third dose is administered on day 15 of the 28-day treatment course. In some embodiments, the first dose is administered on day 1 of the treatment course and the second dose is administered on day 15 of the treatment course. In some embodiments, the first dose is administered on day 1 of the 28-day treatment course and the second dose is administered on day 15 of the 28-day treatment course. In some embodiments, the first dose is administered on day 1 of the treatment course, the second dose is administered on day 4 of the treatment course, and the third dose is administered on day 8 of the treatment course. In some embodiments, the first dose is administered on day 1 of the 21-day treatment course, the second dose is administered on day 4 of the 21-day treatment course, and the third dose is administered on day 8 of the 21-day treatment course. In some embodiments, the method may comprise one or more of a dosing regimen disclosed herein. In some embodiments, a dosing regimen disclosed herein may be combined with one or more of another dosing regimen disclosed herein.

In some embodiments, the method comprises a first 21-day treatment course and a second 21-day treatment course, wherein the first 21-day treatment course predates the second 21-day treatment course, wherein a first dose of the second 21-day treatment course is equal to or higher than a first dose of the first 21-day treatment course, a second dose of the second 21-day treatment course is equal to or higher than a second dose of the first 21-day treatment course, and a third dose of the second 21-day treatment course is equal to or higher than a third dose of the 21-day first treatment course, wherein the first dose of the first 21-day treatment course is a dose disclosed herein. In some embodiments, the method comprises a first 21-day treatment course and a second 21-day treatment course, wherein the first 21-day treatment course predates the second 21-day treatment course, wherein a first dose of the second 21-day treatment course is equal to or higher than a first dose of the first 21-day treatment course, a second dose of the second 21-day treatment course is equal to or higher than a second dose of the first 21-day treatment course, and a third dose of the second 21-day treatment course is equal to or higher than a third dose of the 21-day first treatment course, wherein the first dose of the first 21-day treatment course is at least 25 μg, at least 50 μg, at least 100 μg, at least 150 μg, or at least 200 μg. In some embodiments, the method comprises a first 21-day treatment course and a second 21-day treatment course, wherein the first 21-day treatment course predates the second 21-day treatment course, wherein a first dose of the second 21-day treatment course is equal to or higher than a first dose of the first 21-day treatment course, a second dose of the second 21-day treatment course is equal to or higher than a second dose of the first 21-day treatment course, and a third dose of the second 21-day treatment course is equal to or higher than a third dose of the 21-day first treatment course, wherein the first dose of the first 21-day treatment course is at most 25 μg, at most 50 μg, at most 100 μg, at most 150 μg, or at most 200 g. In some embodiments, the method comprises a first 21-day treatment course and a second 21-day treatment course, wherein the first 21-day treatment course predates the second 21-day treatment course, wherein a first dose of the second 21-day treatment course is equal to or higher than a first dose of the first 21-day treatment course, a second dose of the second 21-day treatment course is equal to or higher than a second dose of the first 21-day treatment course, and a third dose of the second 21-day treatment course is equal to or higher than a third dose of the 21-day first treatment course, wherein the first dose of the first 21-day treatment course is about 25 μg, about 50 μg, about 100 μg, about 150 μg, or about 200 μg.

In some embodiments, the method comprises a first 28-day treatment course and a second 28-day treatment course, wherein the first 28-day treatment course predates the second 28-day treatment course, wherein a first dose of the second 28-day treatment course is equal to or higher than a first dose of the first 28-day treatment course, a second dose of the second 28-day treatment course is equal to or higher than a second dose of the first 28-day treatment course, wherein the first dose of the first 21-day treatment course is a dose disclosed herein. In some embodiments, the method comprises a first 28-day treatment course and a second 28-day treatment course, wherein the first 28-day treatment course predates the second 28-day treatment course, wherein a first dose of the second 28-day treatment course is equal to or higher than a first dose of the first 28-day treatment course, a second dose of the second 28-day treatment course is equal to or higher than a second dose of the first 28-day treatment course, wherein the first dose of the first 21-day treatment course is at least 25 μg, at least 50 μg, at least 100 μg, at least 150 μg, or at least 200 μg. In some embodiments, the method comprises a first 28-day treatment course and a second 28-day treatment course, wherein the first 28-day treatment course predates the second 28-day treatment course, wherein a first dose of the second 28-day treatment course is equal to or higher than a first dose of the first 28-day treatment course, a second dose of the second 28-day treatment course is equal to or higher than a second dose of the first 28-day treatment course, wherein the first dose of the first 21-day treatment course is at most 25 μg, at most 50 μg, at most 100 μg, at most 150 μg, or at most 200 μg. In some embodiments, the method comprises a first 28-day treatment course and a second 28-day treatment course, wherein the first 28-day treatment course predates the second 28-day treatment course, wherein a first dose of the second 28-day treatment course is equal to or higher than a first dose of the first 28-day treatment course, a second dose of the second 28-day treatment course is equal to or higher than a second dose of the first 28-day treatment course, wherein the first dose of the first 21-day treatment course is about 25 μg, about 50 μg, about 100 μg, about 150 μg, or about 200 μg.

In some embodiments, the method comprises at least one of the 21-day treatment course and at least one of the 28-day treatment course, wherein a dose is administered on day 1, day 8, and day 15 of the 21-day treatment course, and on day 1 and day 15 of the 28-day treatment course. In some embodiments, the 21-day or 28-day treatment course is repeated at least once. In some embodiments, the method comprises a first 28-day treatment course and a second 28-day treatment course, wherein a dose is administered on day 1, day 8, and day 15 of the first 28-day treatment course, and on day 1 and day 15 of the second 28-day treatment course. In some embodiments, either of the 28-day treatment course is repeated at least once. In some embodiments, the method comprises at least one of the 21-day treatment course and at least one of the 28-day treatment course, wherein a dose is administered on day 1, day 4, and day 8 of the 21-day treatment course, and on day 1 and day 15 of the 28-day treatment course. In some embodiments, the 21-day or 28-day treatment course is repeated at least once.

In some embodiments, the method comprises at least two of a treatment course disclosed herein, wherein the administering is suspended or paused for a period of time between two of the treatment course. In some embodiments, the administering continues after the suspension or pause.

In some embodiments, the administering comprises administering through intravenous infusion. In some embodiments, the administering comprises administering through intravenous infusion over about 30 min to about 2 h, e.g., about 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, 60 min, 61 min, 62 min, 63 min, 64 min, 65 min, 66 min, 67 min, 68 min, 69 min, 70 min, 71 min, 72 min, 73 min, 74 min, 75 min, 76 min, 77 min, 78 min, 79 min, 80 min, 81 min, 82 min, 83 min, 84 min, 85 min, 86 min, 87 min, 88 min, 89 min, 90 min, 91 min, 92 min, 93 min, 94 min, 95 min, 96 min, 97 min, 98 min, 99 min, 100 min, 101 min, 102 min, 103 min, 104 min, 105 min, 106 min, 107 min, 108 min, 109 min, 110 min, 111 min, 112 min, 113 min, 114 min, 115 min, 116 min, 117 min, 118 min, 119 min, or about 120 min, or any time therebetween.

In some embodiments, the administering comprises administering a mixture comprising the pharmaceutical composition and dextrose, wherein the mixture comprises about 5% (w/v) dextrose. In some embodiments, the administering comprises administering a mixture comprising the pharmaceutical composition and dextrose, wherein the mixture comprises about 5% (v/v) dextrose.

In some embodiments, the pharmaceutical composition comprises the isolated recombinant polypeptide complex at a concentration of about 2 mg/ml. In some embodiments, the pharmaceutically acceptable excipient comprises a buffer, a stabilizing agent, a tonicity agent, a surfactant, or combinations thereof. In some embodiments, the pharmaceutically acceptable excipient comprises a buffer. In some embodiments, the pharmaceutically acceptable excipient comprises a tonicity agent. In some embodiments, the pharmaceutically acceptable excipient comprises a surfactant. In some embodiments, the pharmaceutically acceptable excipient comprises a buffer, a stabilizing agent, a tonicity agent, and a surfactant. In some embodiments, the buffer comprises an amino acid or a derivative thereof. In some embodiments, the amino acid or the derivative thereof comprises L-histidine, L-histidine monohydrochloride monohydrate, or combinations thereof. In some embodiments, the stabilizing agent comprises sugar. In some embodiments, the sugar comprises sucrose. In some embodiments, the tonicity agent comprises sugar. In some embodiments, the sugar comprises sucrose. In some embodiments, the surfactant comprises a polysorbate. In some embodiments, the surfactant comprises polysorbate 20 (PS20).

In some embodiments, the pharmaceutical composition comprises about 1 millimolar (mM) to about 50 mM L-histidine in the form of L-histidine and/or L-histidine monohydrochloride monohydrate, e.g., about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, or about 50 mM, or any concentration therebetween. In some embodiments, the pharmaceutical composition comprises about 1 to about 50 mM L-histidine in the form of L-histidine and L-histidine monohydrochloride monohydrate, e.g., about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, or about 50 mM, or any concentration therebetween. In some embodiments, the pharmaceutical composition comprises about 10 mM L-histidine in the form of L-histidine and/or L-histidine monohydrochloride monohydrate. In some embodiments, the pharmaceutical composition comprises about 10 mM L-histidine in the form of L-histidine or L-histidine monohydrochloride monohydrate. In some embodiments, the pharmaceutical composition comprises about 10 mM L-histidine in the form of L-histidine and L-histidine monohydrochloride monohydrate.

In some embodiments, the pharmaceutical composition comprises about 1% weight/volume (w/v) to about 20% (w/v) sucrose, e.g., about 1%, 2%, 3%, 4%, 5%, 6%, 7%, 8%, 9%, 10%, 11%, 12%, 13%, 14%, 15%, 16%, 17%, 18%, 19%, or about 20%, or any concentration therebetween. In some embodiments, the pharmaceutical composition comprises about 8% (w/v) sucrose.

In some embodiments, the pharmaceutical composition comprises about 0.001% (w/v) to about 0.1% (w/v) polysorbate 20 (PS20), e.g., about 0.001%, 0.002%, 0.003%, 0.004%, 0.005%, 0.006%, 0.007%, 0.008%, 0.009%, 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.031%, 0.032%, 0.033%, 0.034%, 0.035%, 0.036%, 0.037%, 0.038%, 0.039%, 0.04%, 0.041%, 0.042%, 0.043%, 0.044%, 0.045%, 0.046%, 0.047%, 0.048%, 0.049%, 0.05%, 0.051%, 0.052%, 0.053%, 0.054%, 0.055%, 0.056%, 0.057%, 0.058%, 0.059%, 0.06%, 0.061%, 0.062%, 0.063%, 0.064%, 0.065%, 0.066%, 0.067%, 0.068%, 0.069%, 0.07%, 0.071%, 0.072%, 0.073%, 0.074%, 0.075%, 0.076%, 0.077%, 0.078%, 0.079%, 0.08%, 0.081%, 0.082%, 0.083%, 0.084%, 0.085%, 0.086%, 0.087%, 0.088%, 0.089%, 0.09%, 0.091%, 0.092%, 0.093%, 0.094%, 0.095%, 0.096%, 0.097%, 0.098%, 0.099%, or about 0.1%, or any concentration therebetween. In some embodiments, the pharmaceutical composition comprises about 0.01% (w/v) polysorbate 20 (PS20).

In some embodiments, the pharmaceutical composition comprises about 10 mM L-histidine in the form of L-histidine and L-histidine monohydrochloride monohydrate. In some embodiments, the pharmaceutical composition comprises about 8% weight/volume (w/v) sucrose. In some embodiments, the pharmaceutical composition comprises about 0.01% (w/v) polysorbate 20 (PS20). In some embodiments, the pharmaceutical composition comprises about 10 mM L-histidine in the form of L-histidine and L-histidine monohydrochloride monohydrate, about 8% (w/v) sucrose, and about 0.01% (w/v) polysorbate 20. In some embodiments, the pharmaceutical composition comprises a pH of about 5.3. In some embodiments, the pharmaceutical composition comprises an osmolality of about 276 mOsmol/kg.

In some embodiments, the method further comprises treating the subject with a therapy for an infusion-related reaction before or after the administering. In some embodiments, the method further comprises treating the subject with a therapy for an infusion-related reaction before the administering. In some embodiments, the method further comprises treating the subject with a therapy for an infusion-related reaction after the administering. In some embodiments, the method further comprises treating the subject with a therapy for an infusion-related reaction before the administering of each dose of the isolated recombinant polypeptide complex. In some embodiments, the method further comprises treating the subject with a therapy for an infusion-related reaction after the administering of a dose of the isolated recombinant polypeptide complex. In some embodiments, the method further comprises treating the subject with a therapy for an infusion-related reaction after the administering of a dose of the isolated recombinant polypeptide complex and before the administering of a second dose of the isolated recombinant polypeptide complex. In some embodiments, the therapy for an infusion-related reaction comprises acetaminophen, paracetamol, or an antihistamine drug. In some embodiments, the therapy for an infusion-related reaction comprises acetaminophen or paracetamol, and an antihistamine drug. In some embodiments, the therapy for an infusion-related reaction comprises a therapy for treating fever, rigors, rash, urticaria, dyspnea, hypotension, or nausea. In some embodiments, the method further comprises treating the subject with a therapy for cytokine release syndrome (CRS) before or after the administering. In some embodiments, the therapy for CRS comprises a glucocorticoid, an intravenous pre-hydration, or suspension of anti-hypertensive medication before the administering of the first dose in the method. In some embodiments, the therapy for CRS comprises a glucocorticoid, an intravenous pre-hydration, or suspension of anti-hypertensive medication after the administering of the first dose in the method. In some embodiments, the therapy for CRS comprises a glucocorticoid, an intravenous pre-hydration, or suspension of anti-hypertensive medication before the administering of a dose in the method. In some embodiments, the therapy for CRS comprises a glucocorticoid, an intravenous pre-hydration, or suspension of anti-hypertensive medication after the administering of a dose in the method. In some embodiments, the therapy for CRS comprises a therapy for fever, tachycardia, hypotension, hypoxia, fatigue, nausea, headache, dyspnea, rigors, myalgia/arthralgia, or anorexia. In some embodiments, the method further comprises treating the subject with a therapy for tumor lysis syndrome (TLS) before or after the administering. In some embodiments, the therapy for TLS comprises an intravenous hydration, a hypouricemic agent, or correction of acidosis, before or after the administering. In some embodiments, the therapy for TLS comprises a therapy for hyperuricemia, hyperkalemia, hyperphosphatemia, or hypocalcemia.

In some embodiments, the isolated recombinant polypeptide complex is cleaved by a protease to generate an enzymatic product of the isolated recombinant polypeptide complex after the administering. In some embodiments, the isolated recombinant polypeptide complex is cleaved by a tumor specific protease to generate the enzymatic product of the isolated recombinant polypeptide complex after the administering. In some embodiments, the tumor specific protease comprises two or more proteases. In some embodiments, the isolated recombinant polypeptide complex is cleaved by a first protease of the two or more proteases to generate a first metabolic product of the isolated recombinant polypeptide complex. In some embodiments, the isolated recombinant polypeptide complex is cleaved by a second protease of the two or more proteases to generate a second metabolic product of the isolated recombinant polypeptide complex. In some embodiments, the first protease comprises a serine protease. In some embodiments, the second protease comprises a matrix metalloprotease. In some embodiments, the serine protease comprises human matriptase (MTSP1). In some embodiments, the matrix metalloprotease comprises human matrix metalloprotease 9 (MMP9).

In some embodiments, the enzymatic product of the isolated recombinant polypeptide comprises the first metabolic product. In some embodiments, the enzymatic product of the isolated recombinant polypeptide comprises the second metabolic product. In some embodiments, the enzymatic product of the isolated recombinant polypeptide comprises the first metabolic product and the second metabolic product.

In some embodiments, the first metabolic product comprises a first amino acid sequence with at least 90% sequence identity to the amino acid sequence of SEQ ID NO: 4, a second amino acid sequence with at least 90% sequence identity to the amino acid sequence of SEQ ID NO: 6, or both. In some embodiments, the first metabolic product comprises a first amino acid sequence with at least 90% sequence identity to the amino acid sequence of SEQ ID NO: 4. In some embodiments, the first metabolic product comprises a first amino acid sequence with at least 90% sequence identity to the amino acid sequence of SEQ ID NO: 4 and the first amino acid is 229 amino acids in length. In some embodiments, the first metabolic product comprises a second amino acid sequence with at least 90% sequence identity to the amino acid sequence of SEQ ID NO: 6. In some embodiments, the first metabolic product comprises a second amino acid sequence with at least 90% sequence identity to the amino acid sequence of SEQ ID NO: 6 and the second amino acid sequence is 492 amino acids in length.

In some embodiments, the first metabolic product comprises a first amino acid sequence with at least 90% sequence identity to the amino acid sequence of SEQ ID NO: 4 and a second amino acid sequence with at least 90% sequence identity to the amino acid sequence of SEQ ID NO: 6. In some embodiments, the first metabolic product comprises a first amino acid sequence with at least 90% sequence identity to the amino acid sequence of SEQ ID NO: 4 and the first amino acid is 229 amino acids in length; and a second amino acid sequence with at least 90% sequence identity to the amino acid sequence of SEQ ID NO: 6 and the second amino acid sequence is 492 amino acids in length.

In some embodiments, the first metabolic product comprises a first amino acid sequence with at least 90% sequence identity to the amino acid sequence of SEQ ID NO: 4 and a second amino acid sequence with at least 90% sequence identity to the amino acid sequence of SEQ ID NO: 2. In some embodiments, the first metabolic product comprises a first amino acid sequence with at least 90% sequence identity to the amino acid sequence of SEQ ID NO: 4 and the first amino acid is 229 amino acids in length; and a second amino acid sequence with at least 90% sequence identity to the amino acid sequence of SEQ ID NO: 2 and the second amino acid sequence is 653 amino acids in length.

In some embodiments, the first metabolic product comprises a first amino acid sequence with at least 90% sequence identity to the amino acid sequence of SEQ ID NO: 1 and a second amino acid sequence with at least 90% sequence identity to the amino acid sequence of SEQ ID NO: 6. In some embodiments, the first metabolic product comprises a first amino acid sequence with at least 90% sequence identity to the amino acid sequence of SEQ ID NO: 1 and the first amino acid is 256 amino acids in length and a second amino acid sequence with at least 90% sequence identity to the amino acid sequence of SEQ ID NO: 6 and the second amino acid sequence is 492 amino acids in length.

In some embodiments, the first metabolic product comprises a first amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 4. In some embodiments, the first metabolic product comprises a first amino acid sequence comprising the amino acid sequence of SEQ ID NO: 4. In some embodiments, the first metabolic product comprises a second amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 6. In some embodiments, the first metabolic product comprises a second amino acid sequence comprising the amino acid sequence of SEQ ID NO: 6. In some embodiments, the first metabolic product comprises a first amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 1. In some embodiments, the first metabolic product comprises a first amino acid sequence comprising the amino acid sequence of SEQ ID NO: 1. In some embodiments, the first metabolic product comprises a second amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 2. In some embodiments, the first metabolic product comprises a second amino acid sequence comprising the amino acid sequence of SEQ ID NO: 2. In some embodiments, the first amino acid sequence of the first metabolic product is 256 amino acids in lengths. In some embodiments, the first amino acid sequence of the first metabolic product has less than 256 amino acids in lengths. In some embodiments, the second amino acid sequence of the first metabolic product is 653 amino acids in lengths. In some embodiments, the second amino acid sequence of the first metabolic product has less than 653 amino acids in lengths. In some embodiments, the first amino acid sequence of the first metabolic product is 229 amino acids in lengths. In some embodiments, the first amino acid sequence of the first metabolic product has less than 229 amino acids in lengths. In some embodiments, the second amino acid sequence of the first metabolic product is 492 amino acids in lengths. In some embodiments, the second amino acid sequence of the first metabolic product has less than 492 amino acids in lengths.

In some embodiments, the first metabolic product comprises a first amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 4 and a second amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 6. In some embodiments, the first metabolic product comprises a first amino acid sequence comprising the amino acid sequence of SEQ ID NO: 4 and a second amino acid sequence comprising the amino acid sequence of SEQ ID NO: 6. In some embodiments, the first metabolic product comprises a second amino acid sequence comprising the amino acid sequence of SEQ ID NO: 2. In some embodiments, the first amino acid sequence of the first metabolic product is 256 amino acids in lengths. In some embodiments, the first amino acid sequence of the first metabolic product has less than 256 amino acids in lengths. In some embodiments, the second amino acid sequence of the first metabolic product is 653 amino acids in lengths. In some embodiments, the second amino acid sequence of the first metabolic product has less than 653 amino acids in lengths. In some embodiments, the first amino acid sequence of the first metabolic product is 229 amino acids in lengths. In some embodiments, the first amino acid sequence of the first metabolic product has less than 229 amino acids in lengths. In some embodiments, the second amino acid sequence of the first metabolic product is 492 amino acids in lengths. In some embodiments, the second amino acid sequence of the first metabolic product has less than 492 amino acids in lengths.

In some embodiments, the first metabolic product comprises a first amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 4 and a second amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 2. In some embodiments, the first metabolic product comprises a first amino acid sequence comprising the amino acid sequence of SEQ ID NO: 4 and a second amino acid sequence comprising the amino acid sequence of SEQ ID NO: 2. In some embodiments, the first metabolic product comprises a second amino acid sequence comprising the amino acid sequence of SEQ ID NO: 2. In some embodiments, the first amino acid sequence of the first metabolic product is 256 amino acids in lengths. In some embodiments, the first amino acid sequence of the first metabolic product has less than 256 amino acids in lengths. In some embodiments, the second amino acid sequence of the first metabolic product is 653 amino acids in lengths. In some embodiments, the second amino acid sequence of the first metabolic product has less than 653 amino acids in lengths. In some embodiments, the first amino acid sequence of the first metabolic product is 229 amino acids in lengths. In some embodiments, the first amino acid sequence of the first metabolic product has less than 229 amino acids in lengths. In some embodiments, the second amino acid sequence of the first metabolic product is 492 amino acids in lengths. In some embodiments, the second amino acid sequence of the first metabolic product has less than 492 amino acids in lengths.

In some embodiments, the first metabolic product comprises a first amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 1 and a second amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 6. In some embodiments, the first metabolic product comprises a first amino acid sequence comprising the amino acid sequence of SEQ ID NO: 1 and a second amino acid sequence comprising the amino acid sequence of SEQ ID NO: 6. In some embodiments, the first metabolic product comprises a second amino acid sequence comprising the amino acid sequence of SEQ ID NO: 2. In some embodiments, the first amino acid sequence of the first metabolic product is 256 amino acids in lengths. In some embodiments, the first amino acid sequence of the first metabolic product has less than 256 amino acids in lengths. In some embodiments, the second amino acid sequence of the first metabolic product is 653 amino acids in lengths. In some embodiments, the second amino acid sequence of the first metabolic product has less than 653 amino acids in lengths. In some embodiments, the first amino acid sequence of the first metabolic product is 229 amino acids in lengths. In some embodiments, the first amino acid sequence of the first metabolic product has less than 229 amino acids in lengths. In some embodiments, the second amino acid sequence of the first metabolic product is 492 amino acids in lengths. In some embodiments, the second amino acid sequence of the first metabolic product has less than 492 amino acids in lengths.

In some embodiments, the second metabolic product comprises a first amino acid sequence with at least 90% sequence identity to the amino acid sequence of SEQ ID NO: 3, a second amino acid sequence with at least 90% sequence identity to the amino acid sequence of SEQ ID NO: 5, or both.

In some embodiments, the second metabolic product comprises a first amino acid sequence with at least 90% sequence identity to the amino acid sequence of SEQ ID NO: 3. In some embodiments, the second metabolic product comprises a first amino acid sequence with at least 90% sequence identity to the amino acid sequence of SEQ ID NO: 3 and the first amino acid sequence is 221 amino acids in length.

In some embodiments, the second metabolic product comprises a second amino acid sequence with at least 90% sequence identity to the amino acid sequence of SEQ ID NO: 5. In some embodiments, the second metabolic product comprises a second amino acid sequence with at least 90% sequence identity to the amino acid sequence of SEQ ID NO: 5 and the second amino acid sequence is 484 amino acids in length.

In some embodiments, the second metabolic product comprises a first amino acid sequence with at least 90% sequence identity to the amino acid sequence of SEQ ID NO: 3 and the first amino acid sequence is 221 amino acids in length; and a second amino acid sequence with at least 90% sequence identity to the amino acid sequence of SEQ ID NO: 5 and the second amino acid sequence is 484 amino acids in length.

In some embodiments, the second metabolic product comprises a first amino acid sequence with at least 90% sequence identity to the amino acid sequence of SEQ ID NO: 3 and a second amino acid sequence with at least 90% sequence identity to the amino acid sequence of SEQ ID NO: 2. In some embodiments, the second metabolic product comprises a first amino acid sequence with at least 90% sequence identity to the amino acid sequence of SEQ ID NO: 3 and the first amino acid sequence is 221 amino acids in length; and a second amino acid sequence with at least 90% sequence identity to the amino acid sequence of SEQ ID NO: 2 and the second amino acid sequence is 653 amino acids in length.

In some embodiments, the second metabolic product comprises a first amino acid sequence with at least 90% sequence identity to the amino acid sequence of SEQ ID NO: 1 and a second amino acid sequence with at least 90% sequence identity to the amino acid sequence of SEQ ID NO: 5.

In some embodiments, the second metabolic product comprises a first amino acid sequence with at least 90% sequence identity to the amino acid sequence of SEQ ID NO: 1 and the first amino acid sequence is 256 amino acids in length; and a second amino acid sequence with at least 90% sequence identity to the amino acid sequence of SEQ ID NO: 5 and the second amino acid sequence is 484 amino acids in length.

In some embodiments, the second metabolic product comprises a first amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 3. In some embodiments, the second metabolic product comprises a first amino acid sequence comprising the amino acid sequence of SEQ ID NO: 3. In some embodiments, the second metabolic product comprises a second amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 5. In some embodiments, the second metabolic product comprises a second amino acid sequence comprising the amino acid sequence of SEQ ID NO: 5. In some embodiments, the second metabolic product comprises a first amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 1. In some embodiments, the second metabolic product comprises a first amino acid sequence comprising the amino acid sequence of SEQ ID NO: 1. In some embodiments, the second metabolic product comprises a second amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 2. In some embodiments, the second metabolic product comprises a second amino acid sequence comprising the amino acid sequence of SEQ ID NO: 2. In some embodiments, the first amino acid sequence of the second metabolic product is 256 amino acids in lengths. In some embodiments, the first amino acid sequence of the second metabolic product has less than 256 amino acids in lengths. In some embodiments, the second amino acid sequence of the second metabolic product is 653 amino acids in lengths. In some embodiments, the second amino acid sequence of the first metabolic product has less than 653 amino acids in lengths. In some embodiments, the first amino acid sequence of the second metabolic product is 221 amino acids in lengths. In some embodiments, the first amino acid sequence of the second metabolic product has less than 221 amino acids in lengths. In some embodiments, the second amino acid sequence of the second metabolic product is 484 amino acids in lengths. In some embodiments, the second amino acid sequence of the second metabolic product has less than 484 amino acids in lengths.

In some embodiments, the second metabolic product comprises a first amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 3 and a second amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 5. In some embodiments, the second metabolic product comprises a first amino acid sequence comprising the amino acid sequence of SEQ ID NO: 3 and a second amino acid sequence comprising the amino acid sequence of SEQ ID NO: 5. In some embodiments, the first amino acid sequence of the second metabolic product is 256 amino acids in lengths. In some embodiments, the first amino acid sequence of the second metabolic product has less than 256 amino acids in lengths. In some embodiments, the second amino acid sequence of the second metabolic product is 653 amino acids in lengths. In some embodiments, the second amino acid sequence of the first metabolic product has less than 653 amino acids in lengths. In some embodiments, the first amino acid sequence of the second metabolic product is 221 amino acids in lengths. In some embodiments, the first amino acid sequence of the second metabolic product has less than 221 amino acids in lengths. In some embodiments, the second amino acid sequence of the second metabolic product is 484 amino acids in lengths. In some embodiments, the second amino acid sequence of the second metabolic product has less than 484 amino acids in lengths.

In some embodiments, the second metabolic product comprises a first amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 3 and a second amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 2. In some embodiments, the second metabolic product comprises a first amino acid sequence comprising the amino acid sequence of SEQ ID NO: 3 and a second amino acid sequence comprising the amino acid sequence of SEQ ID NO: 2. In some embodiments, the first amino acid sequence of the second metabolic product is 256 amino acids in lengths. In some embodiments, the first amino acid sequence of the second metabolic product has less than 256 amino acids in lengths. In some embodiments, the second amino acid sequence of the second metabolic product is 653 amino acids in lengths. In some embodiments, the second amino acid sequence of the first metabolic product has less than 653 amino acids in lengths. In some embodiments, the first amino acid sequence of the second metabolic product is 221 amino acids in lengths. In some embodiments, the first amino acid sequence of the second metabolic product has less than 221 amino acids in lengths. In some embodiments, the second amino acid sequence of the second metabolic product is 484 amino acids in lengths. In some embodiments, the second amino acid sequence of the second metabolic product has less than 484 amino acids in lengths.

In some embodiments, the second metabolic product comprises a first amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 1 and a second amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 5. In some embodiments, the second metabolic product comprises a first amino acid sequence comprising the amino acid sequence of SEQ ID NO: 1 and a second amino acid sequence comprising the amino acid sequence of SEQ ID NO: 5. In some embodiments, the first amino acid sequence of the second metabolic product is 256 amino acids in lengths. In some embodiments, the first amino acid sequence of the second metabolic product has less than 256 amino acids in lengths. In some embodiments, the second amino acid sequence of the second metabolic product is 653 amino acids in lengths. In some embodiments, the second amino acid sequence of the first metabolic product has less than 653 amino acids in lengths. In some embodiments, the first amino acid sequence of the second metabolic product is 221 amino acids in lengths. In some embodiments, the first amino acid sequence of the second metabolic product has less than 221 amino acids in lengths. In some embodiments, the second amino acid sequence of the second metabolic product is 484 amino acids in lengths. In some embodiments, the second amino acid sequence of the second metabolic product has less than 484 amino acids in lengths.

In some embodiments, the enzymatic product of the isolated recombinant polypeptide complex comprises a first amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 3 and a second amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 6. In some embodiments, the enzymatic product of the isolated recombinant polypeptide complex comprises a first amino acid sequence with at least 90% sequence identity to the amino acid sequence of SEQ ID NO: 3 and a second amino acid sequence with at least 90% sequence identity to the amino acid sequence of SEQ ID NO: 6. In some embodiments, the enzymatic product of the isolated recombinant polypeptide complex comprises a first amino acid sequence with at least 90% sequence identity to the amino acid sequence of SEQ ID NO: 3 and the first amino acid sequence is 221 amino acids in length; and a second amino acid sequence with at least 90% sequence identity to the amino acid sequence of SEQ ID NO: 6 and the second amino acid is 492 amino acids in length. In some embodiments, the second metabolic product comprises a first amino acid sequence comprising the amino acid sequence of SEQ ID NO: 3 and a second amino acid sequence comprising the amino acid sequence of SEQ ID NO: 6.

In some embodiments, the enzymatic product of the isolated recombinant polypeptide complex comprises a first amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 4 and a second amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the amino acid sequence of SEQ ID NO: 5. In some embodiments, the enzymatic product of the isolated recombinant polypeptide complex comprises a first amino acid sequence with at least 90% sequence identity to the amino acid sequence of SEQ ID NO: 4 and a second amino acid sequence with at least 90% sequence identity to the amino acid sequence of SEQ ID NO: 5. In some embodiments, the enzymatic product of the isolated recombinant polypeptide complex comprises a first amino acid sequence with at least 90% sequence identity to the amino acid sequence of SEQ ID NO: 4 and the first amino acid sequence is 229 amino acid in length; and a second amino acid sequence with at least 90% sequence identity to the amino acid sequence of SEQ ID NO: 5 and the second amino acid sequence is 484 amino acid in length. In some embodiments, the second metabolic product comprises a first amino acid sequence comprising the amino acid sequence of SEQ ID NO: 4 and a second amino acid sequence comprising the amino acid sequence of SEQ ID NO: 5.

In some embodiments, the first amino acid sequence of the first metabolic product is 256 amino acids in lengths. In some embodiments, the first amino acid sequence of the first metabolic product has less than 256 amino acids in lengths. In some embodiments, the second amino acid sequence of the first metabolic product is 653 amino acids in lengths. In some embodiments, the second amino acid sequence of the first metabolic product has less than 653 amino acids in lengths. In some embodiments, the first amino acid sequence of the first metabolic product is 229 amino acids in lengths. In some embodiments, the first amino acid sequence of the first metabolic product has less than 229 amino acids in lengths. In some embodiments, the second amino acid sequence of the first metabolic product is 492 amino acids in lengths. In some embodiments, the second amino acid sequence of the first metabolic product has less than 492 amino acids in lengths.

In some embodiments, the first amino acid sequence of the second metabolic product is 256 amino acids in lengths. In some embodiments, the first amino acid sequence of the second metabolic product has less than 256 amino acids in lengths. In some embodiments, the second amino acid sequence of the second metabolic product is 653 amino acids in lengths. In some embodiments, the second amino acid sequence of the first metabolic product has less than 653 amino acids in lengths. In some embodiments, the first amino acid sequence of the second metabolic product is 221 amino acids in lengths. In some embodiments, the first amino acid sequence of the second metabolic product has less than 221 amino acids in lengths. In some embodiments, the second amino acid sequence of the second metabolic product is 484 amino acids in lengths. In some embodiments, the second amino acid sequence of the second metabolic product has less than 484 amino acids in lengths.

In some embodiments, the isolated recombinant polypeptide complex exhibits a cancer cell killing activity that is dependent on the dose, wherein an increase in the dose of the isolated recombinant polypeptide complex results in an increase in the cancer cell killing activity. In some embodiments, the isolated recombinant polypeptide complex exhibits a cancer cell killing activity that is dependent on the dose. In some embodiments, an increase in the dose of the isolated recombinant polypeptide complex results in an increase in the cancer cell killing activity. In some embodiments, the isolated recombinant polypeptide complex exhibits a cancer cell killing activity that is dependent on the amount of the enzymatic product, wherein an increase in the amount of the enzymatic product results in an increase in the cancer cell killing activity. In some embodiments, the isolated recombinant polypeptide complex exhibits a cancer cell killing activity that is dependent on the amount of the enzymatic product. In some embodiments, an increase in the amount of the enzymatic product results in an increase in the cancer cell killing activity. In some embodiments, the isolated recombinant polypeptide complex exhibits a cancer cell killing activity that correlates with the expression level of EGFR on the cancer cell, wherein an increase in the expression level of EGFR on the cancer cell results in an increase in the cancer cell killing activity. In some embodiments, the isolated recombinant polypeptide complex exhibits a cancer cell killing activity that correlates with the expression level of EGFR on the cancer cell. In some embodiments, an increase in the expression level of EGFR on the cancer cell results in an increase in the cancer cell killing activity. In some embodiments, the enzymatic product has a binding affinity with EGFR that is more than 300-fold of the binding affinity of the isolated recombinant polypeptide complex with EGFR. In some embodiments, the enzymatic product has a binding affinity with cluster of differentiation 3 (CD3) that is more than 1000-fold of the binding affinity of the isolated recombinant polypeptide complex with CD3.

In some embodiments, the isolated recombinant polypeptide complex binds with:
(a) a human EGFR at a half-maximal effective concentration (EC₅₀) of about 109 nM;
(b) a human CD3 at an EC₅₀ of about 89 nM;
(c) a human albumin at an EC₅₀ of about 0.1 nM;
(d) a cynomolgus monkey EGFR at an EC₅₀ of about 126 nM;
(e) a cynomolgus monkey CD3 at an EC₅₀ of about 87 nM; and/or
(f) a cynomolgus monkey albumin at an EC₅₀ of about 0.3 nM.

In some embodiments, the isolated recombinant polypeptide complex binds with a mouse or rat EGFR, CD3, or albumin at an EC₅₀ that is more than 1000 fold higher than the EC₅₀ of the human or cynomolgus monkey EGFR, CD3, or albumin.

In some embodiments, the first metabolic product of the isolated recombinant polypeptide complex binds with:
(a) a human EGFR at an EC₅₀ of about 0.28 nM;
(b) a human CD3 at an EC₅₀ of about 0.08 nM;
(c) a cynomolgus monkey EGFR at an EC₅₀ of about 0.27 nM; and/or
(d) a cynomolgus monkey CD3 at an EC₅₀ of about 0.08 nM.

In some embodiments, the first metabolic product of the isolated recombinant polypeptide complex:
(a) does not bind with an albumin derived from human, cynomolgus monkey, mouse, or rat;
(b) binds with mouse EGFR at an EC₅₀ of about 29 nM;
(c) binds with mouse CD3 at an EC₅₀ that is more than 1000-fold higher than the EC₅₀ of the human or cynomolgus monkey CD3;
(d) binds with rat EGFR at an EC₅₀ of about 26 nM; and/or
(e) binds with rat CD3 at an EC₅₀ of about 542 nM.

In some embodiments, the second metabolic product of the isolated recombinant polypeptide complex binds with:
(a) a human EGFR at an EC₅₀ of about 0.27 nM;
(b) a human CD3 at an EC₅₀ of about 0.09 nM;
(c) a cynomolgus monkey EGFR at an EC₅₀ of about 0.26 nM; and/or
(d) a cynomolgus monkey CD3 at an EC₅₀ of about 0.09 nM.

In some embodiments, the second metabolic product of the isolated recombinant polypeptide complex:
(a) does not bind with an albumin derived from human, cynomolgus monkey, mouse, or rat;
(b) binds with mouse EGFR at an EC₅₀ of about 20 nM;
(c) binds with mouse CD3 at an EC₅₀ that is more than 1000-fold higher than the EC₅₀ of the human or cynomolgus monkey CD3;
(d) binds with rat EGFR at an EC₅₀ of about 18 nM; and/or
(e) binds with rat CD3 at an EC₅₀ of about 415 nM.

In some embodiments, the isolated recombinant polypeptide complex exhibits a cancer cell killing activity in an in vitro assay that is at least 100 fold weaker than the enzymatic product of the isolated recombinant polypeptide complex. In some embodiments, the isolated recombinant polypeptide complex induces cytokine release from an immune cell in an in vitro assay at an EC50 that is at least 100 fold higher than the enzymatic product of the isolated recombinant polypeptide complex. In some embodiments, the cancer cell killing activity is measured in the presence of a cancer cell and an immune cell. In some embodiments, the cytokine release is measured in the presence of a cancer cell and an immune cell. In some embodiments, the immune cell is a human peripheral blood mononuclear cell (PBMC). In some embodiments, the cytokine comprises IFNγ, TNF, or IL-6. In some embodiments, the cytokine comprises IFNγ. In some embodiments, the cytokine comprises TNF. In some embodiments, the cytokine comprises IL-6. In some embodiments, the cytokine comprises IFNγ, TNF, and IL-6. In some embodiments, the subject is human.

### Production of Antibodies

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or fragment thereof or the nucleotide sequence of an antibody or fragment thereof is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, HEK293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to blasticidin, zeocin, the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an isolated recombinant polypeptide complex are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an isolated recombinant polypeptide complex is amplifiable, an increase in the level of inhibitor present in culture of host cell will increase the number of copies of the marker gene. Since the amplified region is associated with the nucleotide sequence of the isolated recombinant polypeptide complex, production of the isolated recombinant polypeptide complex will also increase (Crouse et al., 1983, Mol. Cell Biol. 3:257).

In some instances, any method known in the art for purification of an isolated recombinant polypeptide complex is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors.

Mammalian transient expression vectors may include pcDNA, pcDNA3.1+, pcDNA 3.1, pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD 1168, SMD 1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, ExpiCHO-S cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is an antibody that specifically binds to EGFR and CD3.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

“Antibodies” and “immunoglobulins” (IGs) are glycoproteins having the same structural characteristics. The terms are used synonymously. In some instances, the antigen specificity of the immunoglobulin is known.

The term “antibody” is used in the broadest sense, and covers fully assembled antibodies, antibody fragments that can bind antigen (e.g., Fab, F(ab′)2, Fv, single chain antibodies (scFv), diabodies, antibody chimeras, hybrid antibodies, bispecific antibodies, and the like), and recombinant peptides comprising the forgoing.

As used herein, the terms “individual(s),” “subject(s),” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “individual(s)”, “subject(s)” and “patient(s)” are used interchangeably herein and refer to any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### EXAMPLES

### Example 1: Production and Purification Process

### Summary

The manufacturing process is divided into an upstream (cell culture) process, harvest and downstream (purification) process. The upstream process consists of an upstream fed-batch expression in Chinese Hamster Ovary (CHO)-K1 cell culture, through the harvest step. The downstream process includes multiple purification steps followed by final formulation and bulk filtration.

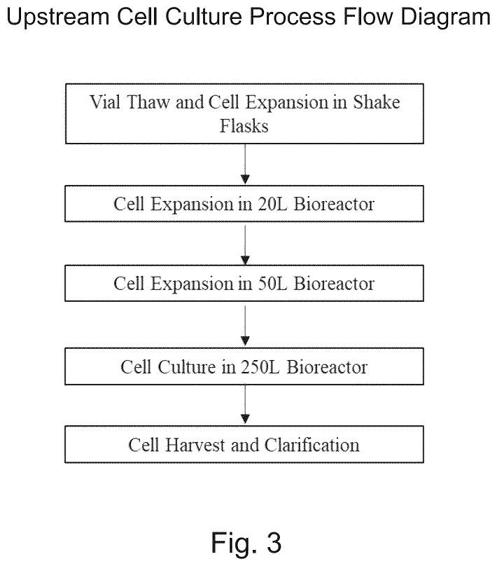

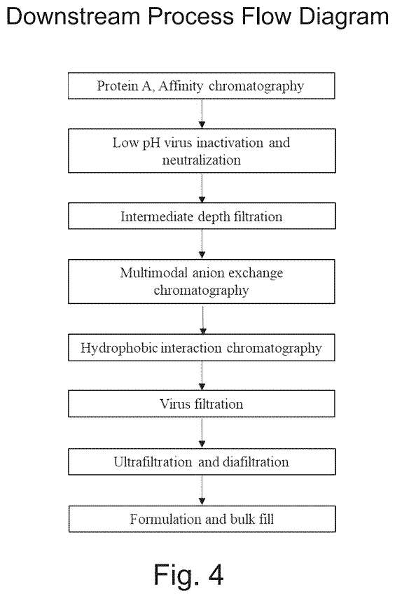

The recombinant antibody was produced and purified following the steps shown in FIGS. 3-4. The upstream process starts with the thawing of a single vial of Master Cell Bank followed by cell culture expansion and fed-batch production of the recombinant antibody in the harvested cell culture fluid (HCCF). The Basal Media 1 consisting of CD CHO media with glutamine, hypoxanthine and thymidine is inoculated with 1 vial of Master Cell Bank and expanded in shake flasks to a defined viable cell density. The culture is further expanded in 20 liter (L) and 50 L single-use bioreactors filled with Basal Media 2 consisting of ActiPro media with glutamine, hypoxanthine, and thymidine followed by transferred to a 250 L single-use bioreactor for fed-batch production. The cell culture proceeds for approximately 12 days, at which point harvest is performed by depth filtration.

The downstream process starts with the capture of the recombinant antibody from the harvested cell culture fluid (HCCF) by a Protein A affinity chromatography followed by virus inactivation at low pH, neutralization, and depth filtration steps. Host cell impurities are removed by an intermediate polishing step using multimodal anion exchange chromatography resin. The recombinant antibody is recovered in the flow-through and it is further polished by binding to a hydrophobic interaction chromatography resin with step elution. A virus-nanofiltration step is performed to remove any potential adventitious agents. The product is then subject to ultrafiltration and diafiltration with histidine buffer pH 5.3 followed by sucrose and polysorbate 20 additions to improve bulk stability. The formulated product is filtered and aseptically dispensed into single-use, sterile polycarbonate bottles and the bulk drug substance is stored at −70° C.±10° C.

### Upstream Cell Culture Process

### Vial Thaw and Cell Culture in Shake Flasks

The manufacturing process is initiated when one vial from the Master Cell Bank is thawed in a 37.0° C.±1° C. water bath, and cells are aseptically transferred into a shake flask, followed by resuspension into fresh Basal Media 1 consisting of CD CHO media with glutamine, hypoxanthine and thymidine. The cell culture is further sub-cultured into increasing size shake flasks with the addition of fresh Basal Media 1, at defined viable cell density (VCD) and viability, under controlled environmental conditions. A backup inoculum expansion is generated at the N-3 stage and maintained for up to three passages.

### Cell Expansion in 20 L Bioreactor

After two rounds of expansion in shake flasks and when the cell culture reaches the desired VCDand viability is higher than 90.0%, the cell culture is transferred to a 20 L disposable bioreactor at cell density of approximately 0.35×10⁶ cells/mL with Basal Media 2 consisting of ActiPro media with glutamine, hypoxanthine and thymidine.

### Cell Expansion in 50 L Bioreactor

Cells from the 20 L bioreactor are inoculated into a 50 L single-use bioreactor filled with Basal Media 2 at a target VCD of approximately 0.45×10⁶ cells/mL and viability higher than 90%. The temperature and agitation speed are set at approximately 36.5° C. and 150 rpm, respectively. The pH and the dissolved oxygen (DO) are controlled with CO₂ and air/oxygen sparging. The cell culture continues to be expanded to the desired VCD and viability above 90%.

### Cell Culture in 250 L Bioreactor

Cell culture is performed in a 250 L single-use bioreactor. The temperature is set at approximately 36.5° C. at inoculation and shifted to 32.0° C. when VCD reaches approximately (10.00-16.00)×10⁶ cells/mL or approximately day 5. The pH and the dissolved oxygen are monitored and controlled at approximately 6.90 and 40.0%, respectively, and the 250 L bioreactor is operated with agitation at 100 rpm and air/oxygen and CO₂ sparging. The cell culture proceeds with regular addition of feeding and glucose supplementation. The culture is harvested on day 12 or when viability drops below 85%, whichever comes first.

### Cell Harvest and Clarification

When harvest criteria are met, the cell culture is harvested and clarified by depth filtration using a 2-staged dual layered regenerated cellulose filters to remove cells and cell debris.

### Downstream Purification Process

### Affinity Chromatography

The principle of this chromatography step is affinity binding using MabSelect PrismA Protein A resin or equivalent to capture the target protein, the recombinant antibody, while allowing impurities to be removed by flowing through the packed column.

The column is rinsed, sanitized and equilibrated with 50 mM Tris-HAc, 150 mM NaCl, pH 7.4 buffer. The clarified cell culture fluid is loaded onto the column followed by high/low salt and pH washes to remove impurities. Bound recombinant antibody is eluted with 30 mM sodium acetate-acetic acid (NaAc-HAc), pH 4.3 at the same flow rate.

### Low pH Virus Inactivation and Neutralization

The pH of the protein A eluate is adjusted to pH 3.6±0.1 with 1 molar (M) acetic acid (HAc) and maintained at 18-26° C., while stirring, to achieve a robust viral inactivation. After 1-2 hours at these conditions, the solution is neutralized with Tris-base and held at ambient temperature before proceeding to the intermediate depth filtration step.

### Intermediate Depth Filtration

The intermediate depth filtration step removes precipitates that might have formed during the low pH virus inactivation and neutralization process steps. A regenerated cellulose depth filter is equilibrated with buffer before loading of the neutralized product pool. After loading, the filters are chased with equilibration buffer and the combined filtrate is further 0.5/0.2 μm filtered into a sterile mixing and storage bag.

### Multimodal Anion-Exchange Chromatography

The multimodal anion exchange chromatography step is performed using a Capto Adhere resin or equivalent in a flow-through mode. The pH and conductivity of the filtrate from the intermediate depth filtration step are adjusted and the loading proceeds at a maximum linear flow rate of 300 cm/h and a minimum residence time of 5 minutes. After loading is completed, the column is washed and the eluate is 0.5/0.2 μm filtered during collection.

### Hydrophobic Interaction Chromatography

The hydrophobic interaction (Butyl Sepharose 4 FF or equivalent) chromatography is used in a bind-elute mode as a polishing step to further remove impurities. In preparation, the conductivity of the multimodal anion exchange eluate is adjusted followed by a 0.5/0.2 μm filtration into a storage bag. The Butyl Sepharose 4 FF column is rinsed, sanitized and equilibrated before loading of adjusted pool. After loading is completed, the column is washed with equilibration/wash buffer. Bound recombinant antibody is eluted from the column and the eluate is 0.5/0.2 μm filtered during collection.

### Viral Filtration

The viral filtration step removes potential viral particles and consists of a 0.5/0.2 μm pre-filter, 20 nanometer (nm) viral-retentive filter, and a 0.5/0.2 μm filter, in tandem. During loading, the pressure differential is maintained at ≤2 bar for the pre-filter, between 0.7-1.0 bar for the viral retentive nano filter and 2 bar for the final 0.5/0.2 μm filter. After the loading has finished, the filters are chased with wash buffer. The final combined filtrate is mixed before proceeding with next steps.

### Ultrafiltration and Diafiltration

Ultrafiltration and diafiltration serve to adjust the in-process drug substance protein concentration and exchange buffer prior to final bulk formulation.

An Ultrafiltration/Diafiltration unit with a 30 kilodalton (kDa) molecular weight cut off filter cassettes is equilibrated until determined pH and conductivity are met. The filtrate from previous step is pumped along the membrane surface and is first concentrated and then diafiltered with histidine buffer until pH and conductivity criteria are met. The DF pool is recovered after circulation at a low flow rate followed by a chase with histidine buffer pH 5.3.

### Excipient Additions, Bulk Formulation, and Fill

During bulk formulation, sucrose and polysorbate 20 are added from stock solutions to the in-process material at a final concentration of 8% (w/v) and 0.01% (w/v) respectively, to improve product stability. This is followed by dilution with histidine buffer pH 5.3 to the target concentration range. The formulated product is filtered and aseptically dispensed into individual, single-use sterile polycarbonate bottles.

### Example 2: Formulation Development

### Initial Screening

Formulation development activities were performed to identify a robust formulation that stabilizes the recombinant antibody in a liquid dosage form that can be stored frozen or refrigerated. To achieve this, an initial formulation development study was conducted at a protein concentration of 2 mg/mL to identify a pH range (4.5, 5.0, 5.5, 6.0, 6.5, or 7.0), stabilizer type (8% (w/v) sucrose or 150 mM sodium chloride), buffering agent type (10 mM glutamate, 10 mM acetate, 10 mM histidine, 10 mM phosphate buffer) while the surfactant was held constant at 0.01% (w/v) polysorbate 20 (PS20). A total of 12 formulations were prepared and analyzed by differential scanning calorimetry (DSC) for thermal stability, appearance (visible particulates, color, and clarity), pH, protein concentration, size exclusion chromatography (SEC), capillary electrophoresis (reduced and non-reduced), and imaged capillary isoelectric focusing (iCIEF). A summary of the formulation matrix is shown in Table 2.

Samples were filled into 2R vials and subjected to three freeze/thaw (F/T) cycles (−70° C. to room temperature), agitation (300 rpm at 25° C. for 2 days), and at 33° C. for 4 weeks. The formulations were then assessed for appearance, aggregation, and impurities.

TABLE 2
Formulation Design Matrix
Formulation Composition

| | | | |
|---|---|---|---|
| 1 | 10 mM glutamate | 8% (w/v) sucrose, | pH 4.5 |
| 2 | 10 mM acetate | 0.01% (w/v) | pH 5.0 |
| 3 | 10 mM L-histidine | polysorbate 20 | pH 5.5 |
| 4 | | | pH 6.0 |
| 5 | | | pH 6.5 |
| 6 | 10 mM phosphate | | pH 7.0 |
| 7 | 10 mM glutamate | 150 mM NaCl, 0.01% | pH 4.5 |
| 8 | 10 mM acetate | (w/v) polysorbate 20 | pH 5.0 |
| 9 | 10 mM L-histidine | | pH 5.5 |
| 10 | | | pH 6.0 |
| 11 | | | pH 6.5 |
| 12 | 10 mM phosphate | | pH 7.0 |

Thermal stability (DSC), appearance (visible particles, color, clarity), pH, protein concentration, SEC, capillary electrophoresis (reduced and non-reduced), and iCIEF were performed.

Changes worth noting for SEC included a large decrease in purity observed for formulations 6-12 after 1 week at 33° C., and there was also a large decrease in main peak purity of formulation 7 after two days of agitation. For iCIEF, formulations 6-12 showed major degradation at 33° C. after one week.

Based on the data generated from the initial screening experiments, the formulation matrices chosen for further development in a secondary screening study were 10 mM Acetate at pH 5.0, 10 mM histidine at pH 5.5 and, 10 mM histidine at pH 6.0.

### Secondary Screening

A secondary screening study was conducted to further optimize the stabilizer, the polysorbate 20 concentration, and the pH so that a final formulation for the drug product could be selected. The study design for the secondary screening experiments is outlined in Table 3.

TABLE 3
Secondary Screening Study Design

| | | | % | |
|---|---|---|---|---|
| | | Surfactant | Surfactant | |
| Formulation Components | | Type | (w/v) | pH |
| 1 | 10 mM Acetate, 8% (w/v) sucrose | Polysorbate 20 | 0.01 | 5.0 |
| 2 | 10 mM L-Histidine, 8% (w/v) | | 0.01 | 5.5 |
| 3 | sucrose | | 0.02 | |
| 4 | | | 0.04 | |
| 5 | | Polysorbate 80 | 0.01 | |
| 6 | 10 mM L-Histidine, 8.8% (w/v) | Polysorbate 20 | 0.01 | |
| | trehalose•2 H₂O | | | |
| 7 | 10 mM L-Histidine, 4.5% (w/v) | | 0.01 | |
| | sorbitol | | | |
| 8 | 10 mM L-Histidine, 8% (w/v) | | 0.01 | 6.0 |
| | sucrose | | | |

Samples were filled into 2R vials and subjected to three or five F/T cycles (−70° C. to room temperature), agitation (300 rpm at 25° C. for 3 days) and were stored at 33° C. for 3 weeks. In addition to the testing panel used to assess samples in the initial screening study, the formulations were also tested for osmolality and subvisible particulate matter.

Appearance (visible particles, color, clarity), pH, protein concentration, SEC, particulate matter (sub-visible particles), capillary electrophoresis (reduced and non-reduced), osmolality, and iCIEF were performed on samples from the secondary screening study.

For the pH, protein concentration, iCIEF, capillary electrophoresis (reduced and non-reduced), and sub-visible particles results no substantial changes were observed after incubating at 33° C. for three weeks, after agitation for three days, or for F/T in all formulations. The appearance results for all samples were clear, colorless solutions with no visible particles.

SEC data showed that sucrose and a lower concentration of surfactant were most effective for stabilizing the protein.

To minimize the risk of aggregation, a relatively lower pH (5.0 to 5.5) was chosen to increase the surface charge of the protein.

Based on the data generated from the secondary screening experiments, the formulation matrix chosen for formulation verification was 2 mg/mL recombinant antibody, 10 mM histidine, 8% (w/v) sucrose, 0.01% (w/v) PS20, at pH 5.3.

### Formulation Verification

The formulation chosen from the secondary screening studies (2 mg/mL recombinant antibody, 10 mM L-histidine, 8% (w/v) sucrose, 0.01% (w/v) PS20, at pH 5.3) was evaluated in a verification study to assess the stability of the molecule in its final formulation.

For the formulation verification study, the final formulation of recombinant antibody was subjected to one and three FIT cycles (−20±5° C./Room Temp), stored at −20±5° C. for 12 weeks, stored at 2-8° C. for 12 weeks, and stored at 25±2° C. for 4 weeks for stability studies. The frozen samples (−20±5° C. and FIT) were initially frozen at −40±5° C. and then transferred to −20±5° C. for storage. The verification study overview is presented in Table 4.

TABLE 4
Formulation Verification Study Overview

| | | | | | | | | | | −20 ± 5° C./RT | |
|---|---|---|---|---|---|---|---|---|---|---|---|
| | | | | | | | | | | Freeze/Thaw | |
| | | −20 ± 5° C. | | | 2 − 8° C. | | | 25 ± 2° C. | | (Upright) | |
| | | (Upright) | | | (Upright) | | | (Inverted) | | 1 | 3 |
| Tests | t0 | 2 W | 4 W | 12 W | 2 W | 4 W | 12 W | 2 W | 4 W | Cycle | Cycles |
| Appearance | X | X | X | X | X | X | X | X | X | X | X |
| pH | X | X | X | X | X | X | X | X | X | X | X |
| Protein Concentration | X | X | X | X | X | X | X | X | X | X | X |
| SEC | X | X | X | X | X | X | X | X | X | X | X |
| iCIEF | X | X | X | X | X | X | X | X | X | X | X |
| Caliper-SDS (Non-Reduced) | X | X | X | X | X | X | X | X | X | X | X |
| CE-SDS (Reduced) | X | X | X | X | X | X | X | X | X | X | X |
| Particulate Matter (HIAC) | X | NT | X | X | NT | X | X | NT | X | NT | X |
| Glass Transition | X | NT | NT | NT | NT | NT | NT | NT | NT | NT | NT |
| ELISA Binding-EGFR (cleaved) | X | NT | NT | X | NT | NT | X | NT | X | NT | NT |
| ELISA Binding-CD3 (cleaved) | X | NT | NT | X | NT | NT | X | NT | X | NT | NT |

NT—not tested

There were no practically significant changes observed in the formulation verification study for appearance (visible particles, color, clarity), pH, protein concentration, SEC, iCIEF, reduced CE-SDS, non-reduced caliper-SDS, and ELISA Binding-EGFR (cleaved).

The particulate counts for particles ≤2 μm increased for all conditions. However, the particulate counts for particles ≤2 μm were all <130 particles/mL and were obtained using a HIAC, so the increase in particulate counts can be attributed to the variation of the method and instrument. These increases in particulate counts are not considered practically significant.

### Glass Transition (Tg′)

The glass transition temperature (Tg′) was tested at the initiation of the formulation verification study. Tg′ was measured to be −34.2° C., which is lower than the intended long-term storage temperature. The recombinant antibody can be frozen at −40±5° C. prior to being transferred to −20±5° C.

### Formulation Verification Conclusion

Based on the data generated from the formulation verification experiments, the formulation matrix of 2 mg/mL recombinant antibody, 10 mM L-histidine, 8% (w/v) sucrose, 0.01% (w/v) PS20, at pH 5.3 was chosen.

### Example 3: Disulfide Bond Linkage Confirmation by LC-MS/MS

Disulfide bond linkages are important in protein folding and they play a significant role in both protein structure and functions. The number of disulfide bonds and their positions are important attributes for ensuring safety and efficacy of biopharmaceuticals.

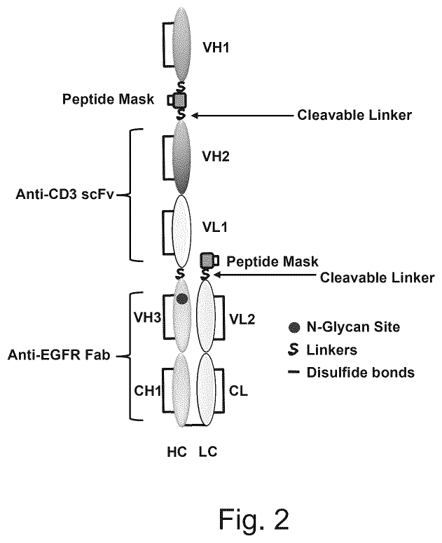

In PC-1 there are 20 cysteine residues, as shown in FIG. 2, which are cross-linked by 1 inter-chain disulfide bond between the heavy and light chain and 9 intra-chain disulfide bonds. The heavy chain contains six domains and light chain contains three domains. 10 disulfide bond related peptides (DS1 to DS10) are expected by non-reduced Lys-C/trypsin sequential digestion with PNGase F. Due to the theoretical mass of peptide DS8 is 11380.3093 Da, indicating the peptide can be high hydrophobic and hard to be ionized by MS spectrometry. In order to further elucidate the cysteine linkages, Lys-C/chymotrypsin sequential digestion was applied. DS8 is digested into smaller peptides. Liquid chromatography with tandem mass spectrometry (LC-MS/MS) results are provided in Table 5.

TABLE 5
Liquid Chromatography with tandem mass spectrometry (LC-MS/MS) Results

| | | Disulfide | | | | | PC-1 v1 | |
|---|---|---|---|---|---|---|---|---|
| | | Bond | Disulfide | Retention | | Theoretical | Measured | |
| | | Related | bond | Time | | Mass | Mass | Difference |
| No. | SEQ ID NO | Sequence | linkages | (min) | PTMs | (Da) | (Da) | (ppm) |
| DS1a | 13, | GGPCR | LC: C4- | 30.5 | / | 1758.8254 | 1758.8239 | -0.9 |
| | 14 | ═PICV | LC: C15 | | | | | |
| | Respec- | GGGGS | | | | | | |
| | tively | GGLSG | | | | | | |
| | | R | | | | | | |
| DS1b | 13, | GGPCR | | 34.9 | / | 2509.2278 | 2509.2234 | -1.8 |
| | 15 | ═SHID | | | | | | |
| | Respec- | VAKPI | | | | | | |
| | tively | CVGG | | | | | | |
| | | GGSGG | | | | | | |
| | | LSGR | | | | | | |
| DS2 | 16, | VSFSC | LC: C65- | 68.9 | / | 5203.2724 | 5203.2678 | -0.9 |
| | 17 | R═FSG | LC: C130 | | | | | |
| | Respec- | SGSGT | | | | | | |
| | tively | DFTLSI | | | | | | |
| | | NSVES | | | | | | |
| | | EDIAD | | | | | | |
| | | YYCQ | | | | | | |
| | | QNNN | | | | | | |
| | | WPTTF | | | | | | |
| | | GAGTK | | | | | | |
| DS3 | 18, | SGTAS | LC: C176- | 65.7 | / | 3555.7490 | 3555.7420 | -2.0 |
| | 19 | VVCLL | LC: C236 | | | | | |
| | Respec- | NNFYP | | | | | | |
| | tively | R═VYA | | | | | | |
| | | CEVTH | | | | | | |
| | | QGLSS | | | | | | |
| | | PVTK | | | | | | |
| DS4a | | GEC═S | LC: C256- | 4.6 | / | 513.1199 | 513.1184 | -2.9 |
| | | C | | | | | | |
| DS4b | 20 | SFNRG | HC: C653 | 19.4 | / | 1017.3644 | 1017.3635 | -0.9 |
| | | EC═SC | | | | | | |
| DS5 | 21, | LSCAA | HC: C22- | 82.2 | / | 4682.2066 | 4682.2047 | -0.4 |
| | 22 | SGSTF | HC: C96 | | | | | |
| | Respec- | YTAVM | | | | | | |
| | tively | GWVR | | | | | | |
| | | ═TTLY | | | | | | |
| | | LQMNS | | | | | | |
| | | LRPED | | | | | | |
| | | TAVYY | | | | | | |
| | | CAAR | | | | | | |
| DS6 | 23 | GTLGL | HC: C138- | 77.2 | / | 5739.4737 | 5739.4793 | 1.0 |
| | | FTTAD | HC: C148 | | | | | |
| | | SYDY | | | | | | |
| | | WGQG | | | | | | |
| | | TLVTV | | | | | | |
| | | SSGGG | | | | | | |
| | | GSGGG | | | | | | |
| | | SGGVY | | | | | | |
| | | CGPEF | | | | | | |
| | | DESVG | | | | | | |
| | | CMGG | | | | | | |
| | | GGSGG | | | | | | |
| | | GLSGR═ | | | | | | |
| DS7 | 24, | LSCAA | HC: C199- | 44.6 | / | 2561.1567 | 2561.1541 | -1.0 |
| | 25 | SGFTF | HC: C275 | | | | | |
| | Respec- | NK-TE | | | | | | |
| | tively | DTAVY | | | | | | |
| | | YCVR | | | | | | |
| DS8a | 26, | TCGSS | HC: C339- | 58.9 | / | 2217.9459 | 2217.9386 | -3.3 |
| | 27 | TGAVT | HC: C407 | | | | | |
| | Respec- | SGNYP | | | | | | |
| | tively | NW═C | | | | | | |
| | | VLW | | | | | | |
| DS8b | 26, | TCGSS | | 61.1 | / | 2381.0093 | 2381.0001 | -3.9 |
| | 28 | TGAVT | | | | | | |
| | Respec- | SGNYP | | | | | | |
| | tively | NW═Y | | | | | | |
| | | CVLW | | | | | | |
| DS8c | 29 | TCGSS | | 41.8 | / | 2515.1472 | 2515.1425 | -1.9 |
| | | TGAVT | | | | | | |
| | | SGNYP | | | | | | |
| | | NWVQ | | | | | | |
| | | QK═CV | | | | | | |
| | | L | | | | | | |
| DS8d | 29, | TCGSS | | 52.8 | / | 2701.2265 | 2701.2205 | -2.2 |
| | 27 | TGAVT | | | | | | |
| | Respec- | SGNYP | | | | | | |
| | tively | NWVQ | | | | | | |
| | | QK═CV | | | | | | |
| | | LW | | | | | | |
| DS8e | 29, | TCGSS | | 54.9 | / | 2864.2898 | 2864.2853 | -1.6 |
| | 30 | TGAVT | | | | | | |
| | Respec- | SGNYP | | | | | | |
| | tively | NWVQ | | | | | | |
| | | QK═CV | | | | | | |
| | | LWY | | | | | | |
| DS9 | 31, | QSGPG | HC: C453- | 74.1 | Deglycos- | 5352.5278 | 5352.5287 | 0.2 |
| | 32 | LVQPS | HC: C526 | | ylation | | | |
| | Respec- | QSLSIT | | | (N) | | | |
| | tively | CTVSG | | | | | | |
| | | FSLTN | | | | | | |
| | | YGVH | | | | | | |
| | | WVR═ | | | | | | |
| | | MNSLQ | | | | | | |
| | | SNDTA | | | | | | |
| | | IYYCA | | | | | | |
| | | R | | | | | | |
| DS10a | 33, | STSGG | HC: C577- | 90.5 | / | 7389.6490 | 7389.6431 | -0.8 |
| | 34 | TAALG | HC: C633 | | | | | |
| | Respec- | CLVK═ | | | | | | |
| | tively | DYFPE | | | | | | |
| | | PVTVS | | | | | | |
| | | WNSG | | | | | | |
| | | ALTSG | | | | | | |
| | | VHTFP | | | | | | |
| | | AVLQS | | | | | | |
| | | SGLYS | | | | | | |
| | | LSSVV | | | | | | |
| | | TVPSS | | | | | | |
| | | SLGTQ | | | | | | |
| | | TYICN | | | | | | |
| | | VNHK | | | | | | |
| DS10b | 33, | STSGG | | 88.6 | / | 7916.9194 | 7916.9226 | 0.4 |
| | 35 | TAALG | | | | | | |
| | Respec- | CLVK═ | | | | | | |
| | tively | DYFPE | | | | | | |
| | | PVTVS | | | | | | |
| | | WNSG | | | | | | |
| | | ALTSG | | | | | | |
| | | VHTFP | | | | | | |
| | | AVLQS | | | | | | |
| | | SGLYS | | | | | | |
| | | LSSVV | | | | | | |
| | | TVPSS | | | | | | |
| | | SLGTQ | | | | | | |
| | | TYICN | | | | | | |
| | | VNHKP | | | | | | |
| | | SNTK | | | | | | |

### Example 4: Secondary Structure Determination by Far-UV CD

Protein far ultraviolet circular dichroism (far-UV CD) spectra can reveal the characteristic secondary structures, i.e. α-helix, β-sheet, random coil, etc. Prior to measurement, protein sample was diluted with ultrapure water to a protein concentration of 0.1 mg/mL. The data collection and analysis were performed with JASCO/J-815 CD spectrometer and Spectra Manager software.

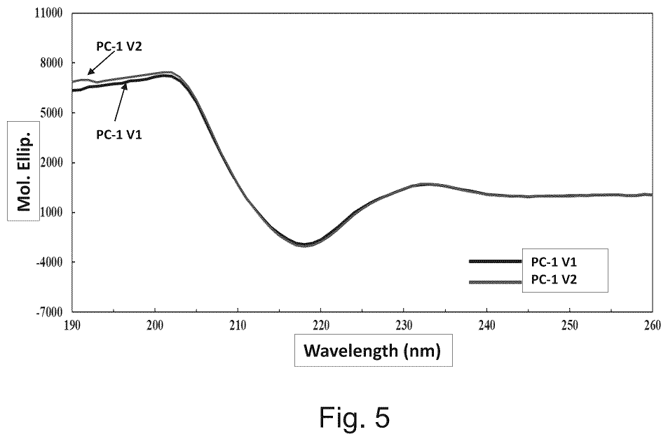

The CD spectra PC-1 in the far-UV region (190-260 nm) is shown in FIG. 5. The R-sheet and random coil were the main secondary structure composition.

### Example 5: Tertiary Structure Determination by Near-UV CD

Protein near-UV CD spectrum provides information on the protein tertiary structures. The CD spectral pattern in the 250-350 nm region is determined by the absorption, dipole orientation and the nature of the surrounding environment of the phenylalanine (250-270 nm), tyrosine (270-290 nm), and tryptophan (290-305 nm), respectively. For the measurement, the protein samples were diluted with 10 mM histidine, 8% (w/v) sucrose, 0.01% (w/v) PS20 pH 5.3 to 1.0 mg/mL. Data collection and analysis were performed by JASCO/J-815 CD spectrometer and Spectra Manager software, respectively.

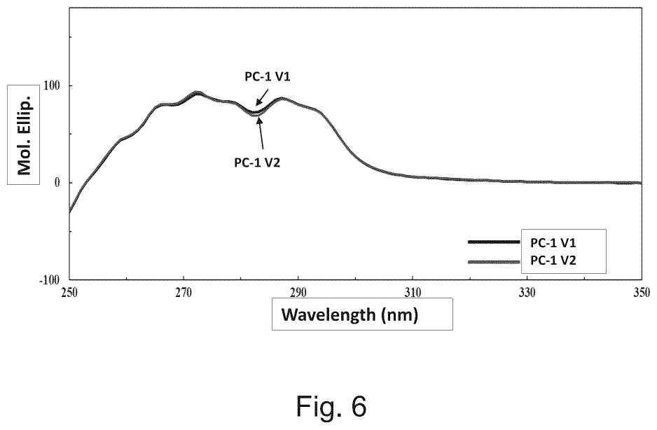

The CD spectra of PC-1 in the near-UV region (250-350 nm) is shown in FIG. 6. The Spectra similarity/structure consistency analysis was achieved by calculating the correlation coefficient of two spectra with “Quick Compare” tool of OPUS Spectroscopy Software.

### Example 6: Thermal Stability Analysis by DSC

Differential scanning calorimetry (DSC) is an experimental technique to obtain thermal transition profile of materials. It is widely used in the investigation of folding/unfolding transitions of proteins under programmed temperature increase in protein characterization. The melting onset (Tₒₙₛₑₜ) and mid-point (Tm) temperatures of the thermal transition (Tm) are commonly used as indicators of thermal stability, and the DSC thermogram reveals the thermal transition profile.

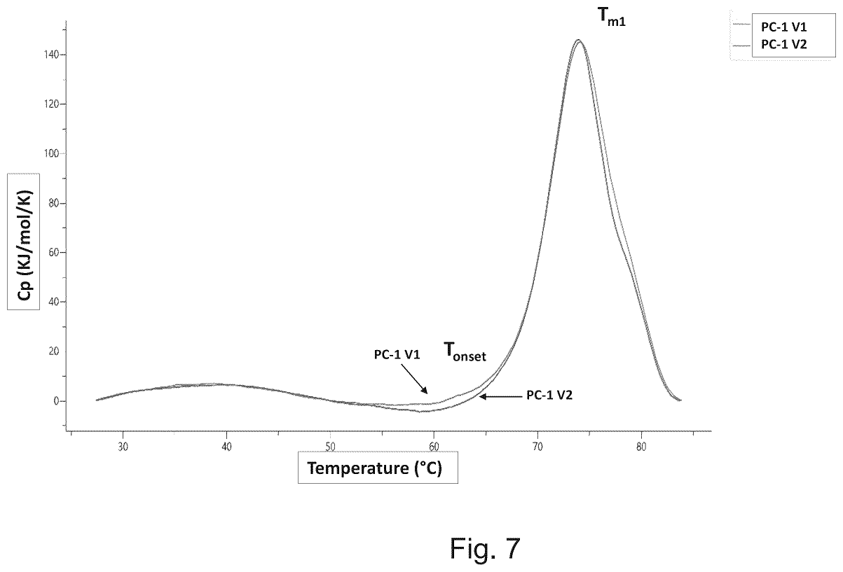

PC-1 was analyzed with MicroCal DSC from Malvern. The protein sample was diluted to 1 mg/mL with 10 mM histidine, 8% (w/v) sucrose, 0.01% (w/v) PS20 pH 5.3 before analysis. 400 microliters (μL) corresponding formulation buffer was added to a 96-well plate as the reference and 400 μL protein sample was added. The samples were heated from 10° C. to 95° C. at a heating rate of 90° C./h in the capillary DSC system. The DSC data were analyzed and fitted with MicroCal PEAQ-DSC Software 1.51. The results are shown in FIG. 7. For PC-1, the T_{Onset} was determined as 62.5° C. and the two transition mid-point temperature (Tm1) were determined as 73.8° C.

### Example 7: Molar Mass and Size Analysis by SEC-MALS

Size exclusion chromatography coupled with multi angle light scattering (SEC-MALS) detector separates proteins based on their sizes and then measure the molecular weight of the separated components via MALS detector. The smaller proteins elute from the SEC column later while larger proteins elute at earlier retention time and results in a separation between the proteins based on their size differences. The separated components including monomer, high molecular weight species (HMWS), and low molecular weight species (LMWS) are quantified via UV detector. The absolute molar mass and size of the molecules in solution is calculated using the intensity and the angular dependence of the scattered light signal from MALS detector. The static multi-angle light scattering method characterizes the absolute molecular weight of proteins based on the principle of static light scattering, expressed in Zimm's equation. The intensity of laser scattering is directly proportional to the molecular weight and protein concentration for proteins larger than 10 nm. Therefore, those protein molecular weight can be calculated according to the relationship between scattered light intensity and angle P (θ), protein size Rg, as well as protein concentration c.

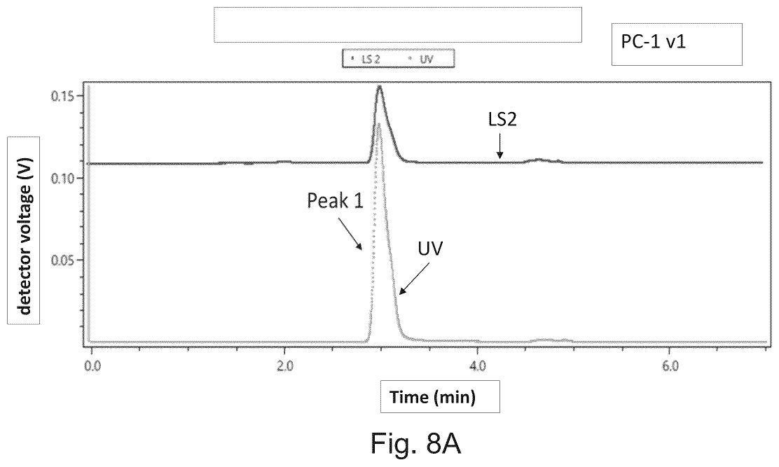

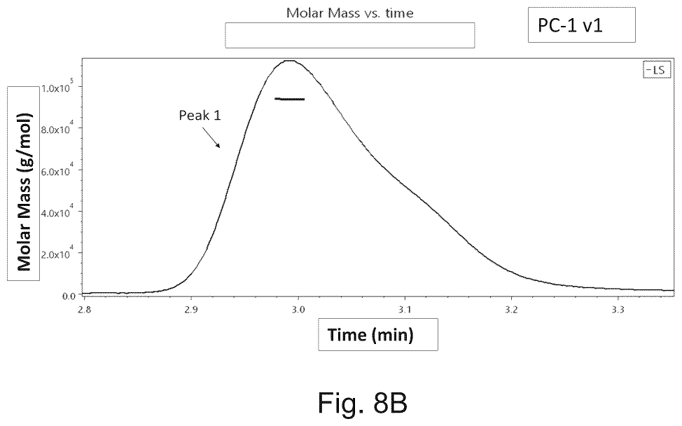

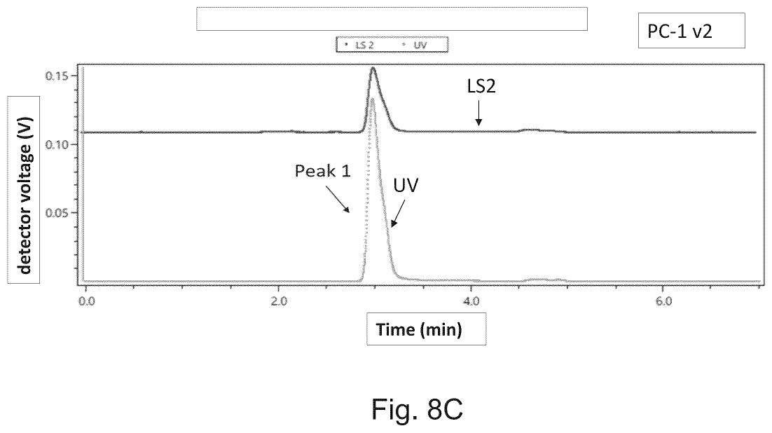

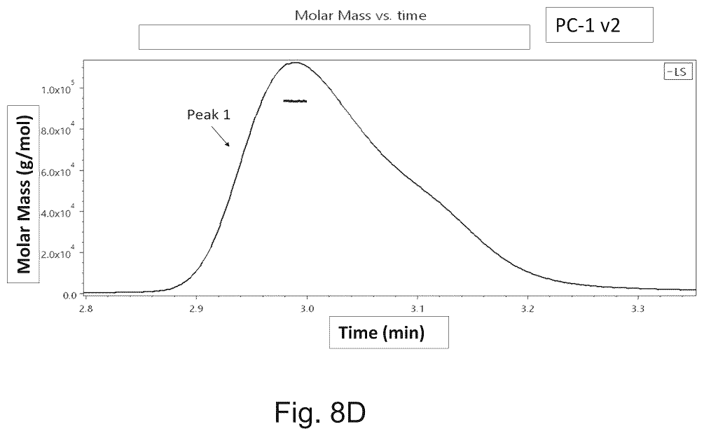

The SEC-MALS chromatograms of PC-1 are shown in FIG. 8A-8D. The molecular weight of the main peak (peak 1) was 90.2 kDa (RT=3.111 min) which corresponds to the monomer.

### Example 8: Protein Size Analysis by Dynamic Light Scattering

Dynamic Light Scattering (DLS) analysis is used to measure the particle size by illuminating the particles with a laser and analyzing the intensity fluctuations in the scattered light. DLS analysis for PC-1 samples were performed using a Malvern Dynamic Light Scattering instrument, model Zen3600. The protein samples in formulation buffer were transferred to a DLS disposable cuvette. Each sample was sampled once and tested for three times. Data was auto-analyzed by Zetasizer Nano software and rendered particle size (Z-average size) and Polydispersity Index (PDI). These results demonstrate that PC-1 samples show no obvious differences in particle size and distributions.

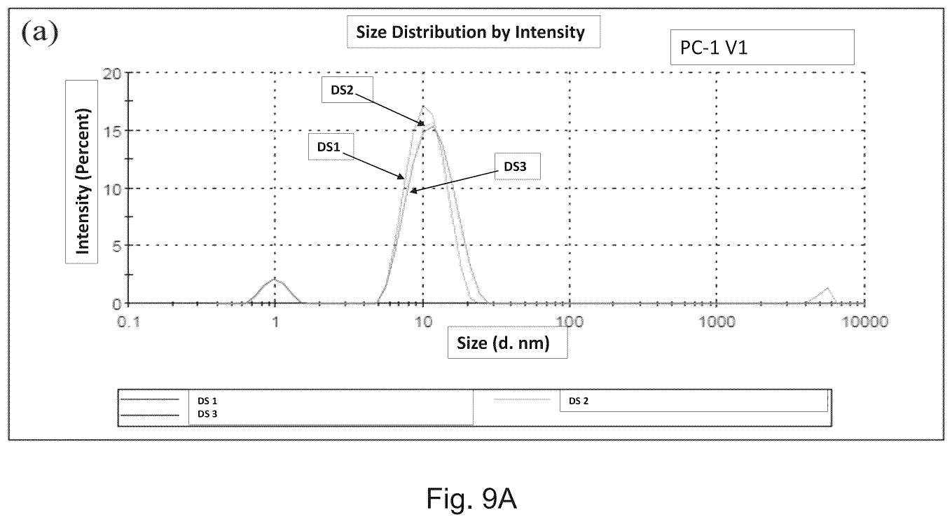

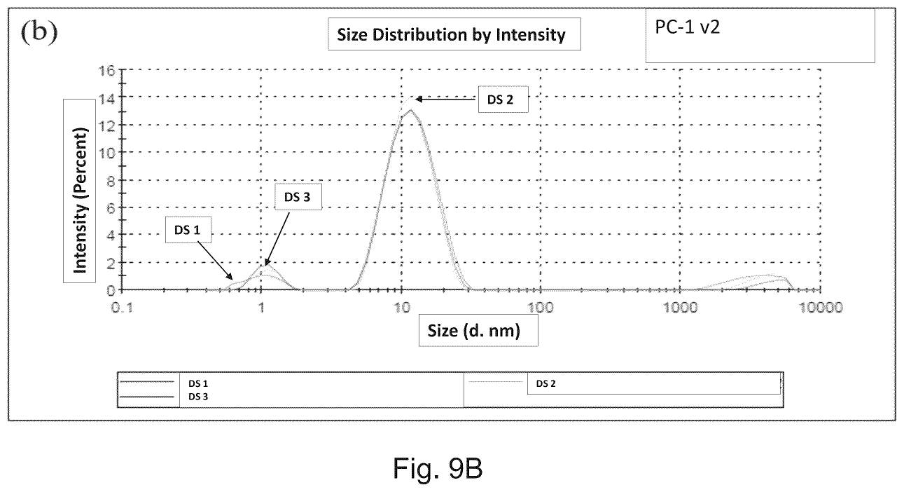

The DLS results are shown in FIGS. 9A-9B and Table 6. The Z-Average is 14.8 nm for PC-1v2, and 14.6 nm for PC-1v1. The Polydispersity Index (PDI) is 0.03 for PC1-v2, and 0.02 for PC1-v1.

TABLE 6
DLS Results
(b)

| | Mean Size | Polydispersity | Peak 1 | | Peak 2 | | Peak 3 | |
|---|---|---|---|---|---|---|---|---|
| Lot | (Z-Average/ | Index | Size | Intensity | Size | Size | Intensity | Size |
| No. | nm) ^{#1} | (PDI)^{#2} | (nm) | (%) | (nm) | (nm) | (%) | (nm) |
| PC-1v1 | 9.4 | 0.23 | 11.6 | 92.1 | 1.0 | 7.1 | 1728.3 | 0.8 |
| PC-1v2 | 10.5 | 0.29 | 12.3 | 88.1 | 1.1 | 6.1 | 4001.7 | 5.7 |

^{#1} Z-average represents the diameter of protein particles.
^{#2}PDI is indicative of uniformity of protein particles, the smaller PDI, the more uniform of protein particles.

### Example 9: Activity Against Tumors with Resistance Mutations

The isolated recombinant antibody was evaluated in a functional in vitro tumor cell killing assay using the EGFR positive tumor cell lines with resistant mutations: HEK293, HeLa, A459, A549 EGFR knockout, H1650, HCT116, HT29, H1975, PC-9, FaDu, mutant PC-9, mutant H1975, Cal27, and A431 as shown in Table 7. Polypeptide complexes were evaluated in a functional in vitro tumor cell killing assay using EGFR positive tumor cell lines. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight on a 96 well E-Plate. The following day polypeptide complexes titrated in human serum supplemented medium along with 30,000 CD8+ T cells were added to the wells. Cell index measurements were taken every 10 minutes for an additional 72 hours. The cell index times number of hours (tumor cell growth kinetics) was then plotted versus concentration of polypeptide complex where the concentration required to reduce the tumor growth 50% (IC50) was calculated using Graphpad Prism software.

TABLE 7
Activity against tumors with resistance mutations

| | | | | | | TCE | |
|---|---|---|---|---|---|---|---|
| | | | | | | cytotox | |
| Cell- | EGFR | Mutation | | | | EC50 | TRACTr |
| Line | density | EGFR | KRAS | BRAF | PIK3CA | (pM) | Shift |
| A549 | none | — | G12S | — | — | inactive | inactive |
| (EGFR | detected | | | | | | |
| KO) | | | | | | | |
| HEK293 | 3,500 | — | — | — | — | 0.7 | 81,200x |
| HeLa | 17,000 | — | — | — | — | 0.2 | 76,000x |
| HT29 | 18,000 | — | — | V600E | P449T | 0.5 | 35,000x |
| A549 | 20,000 | — | G12S | — | — | 1.2 | 43,000x |
| H1650 | 25,000 | Ex19Del | — | — | — | 0.4 | 5,300x |
| HCT116 | 30,000 | — | G13D | — | H1047R | 0.4 | 8,500x |
| H1975 | 34,000 | T790M/ | — | — | G118D | 0.4 | 13,500x |
| | | L858R | | | | | |
| PC-9 | 43,000 | Ex19Del | — | — | — | 0.5 | 32,000x |
| FaDu | 75,000 | — | — | — | — | 0.2 | 2,300x |
| PC-9 | 77,000 | Ex19Del/ | — | — | — | 0.5 | 8,000x |
| mut | | T790M/ | | | | | |
| | | L858R | | | | | |
| H1975 | 82,000 | T790M/ | — | — | G118D | 0.4 | 7,500x |
| mut | | L858R/ | | | | | |
| | | C797S | | | | | |
| Cal27 | 170,000 | — | — | — | — | 0.1 | 1,700x |
| A431 | ≥500,000 | — | — | — | — | 0.2 | 330x |

### Example 10: Clinical Effects of Polypeptide Complex 1 (PC-1)

This Example illustrates the clinical effects of a polypeptide complex 1(PC-1) disclosed herein.

### Summary

### 1.1 PC-1

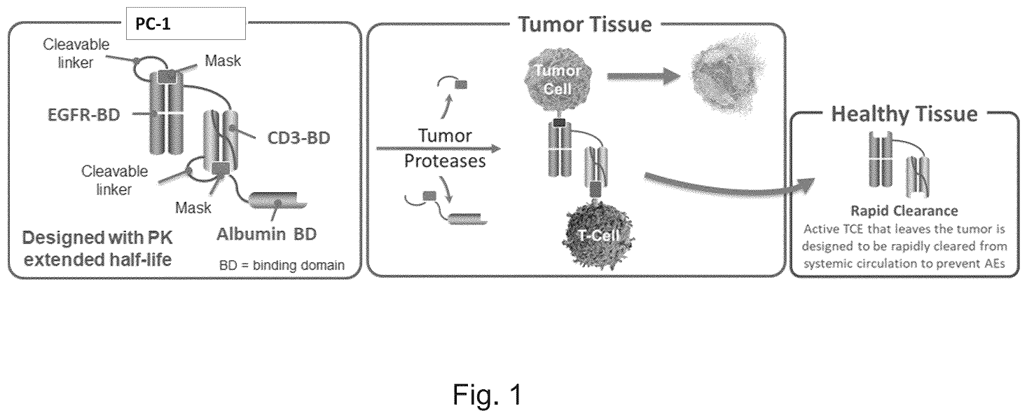

As illustrated in FIG. 1, PC-1 is a tumor activated T cell engager (TRACTr) comprising a humanized tri-specific protein that incorporates epidermal growth factor receptor (EGFR) and cluster of differentiation 3 (CD3) binding domains, an albumin-binding domain to extend circulating half-life, and two separate peptide masks. The peptide masks are fused to the molecule through tumor protease cleavable linkers. One peptide mask inhibits EGFR engagement on target cells, and the other peptide mask inhibits CD3 engagement on T cells.

### 1.2 Pharmacology

Nonclinical pharmacology in vitro studies with PC-1 examined the following:
1. PC-1 binding to EGFR, CD3, and albumin antigens from mouse, rat, cynomolgus monkey, and human
2. PC-1 stability in serum of healthy human donors, and colorectal cancer (CRC), squamous cell carcinoma of head and neck (SCCHN), and non-small cell lung cancer (NSCLC) patients, and cynomolgus monkeys
3. The ability of PC-1 to induce T cell-mediated anti-tumor cytotoxic activity
4. The ability of PC-1 to induce cytokine production

Results from the binding study concluded that PC-1 bound human and cynomolgus monkey EGFR, CD3, and albumin with low nanomolar affinity, while exhibiting minimal binding to mouse or rat antigens. Cleavage-dependent unmasking of PC-1 led to sub-nanomolar affinity to human and cynomolgus EGFR and CD3. Serum stability studies showed that PC-1 was more susceptible to cleavage in cynomolgus monkey serum compared to human serum or serum from CRC, SCCHN, and NSCLC patients. The cleavage rate of both masks was about 1% per day in healthy human serum and 2% in serum from CRC, SCCHN, and NSCLC patients, whereas cleavage of PC-1 in cynomolgus monkey serum was 11.5% for the EGFR mask and 6.6% for the CD3 mask.

Cellular cytotoxicity studies showed that PC-1 induced tumor cell killing is cleavage- and dose-dependent. PC-1 exhibited much lower potency compared to cleaved forms (PC-1 serine protease [SP]-cleaved and PC-1 matrix metalloprotease [MMP]-cleaved) and non-masked, PC-1-T cell engager (TCE). Activity correlated with the density of EGFR expression on the surface of target tumor cells, with activity being lowest (˜8,000 to 12,000× relative to SP cleaved PC-1) in the A549 cell line. Estimated decrease in tumor killing was ˜5,500× and ˜450× in HCT116 and CAL27 cell lines, respectively. The activity of PC-1, ie, the ability to induce T cell-mediated tumor cell killing, depended on the expression level of EGFR on the surface of target tumor cells. None of the test articles induced cytotoxicity against a control EGFR-deficient A549 cell line. In in vitro cytokine release assays, cleaved (PC-1-SP and PC-1-MMP cleaved) and non-masked (PC-1-TCE) test articles exhibited potent, dose-dependent induction of interferon gamma (IFNγ), tumor necrosis factor (TNF), and interleukin (IL)-6 release in the presence of EGFR positive cell lines HCT116 and A549, while dose-dependent production of IFNγ and TNF, but not IL-6, was observed in the presence of the EGFR positive Cal27 cell line. Masked PC-1 showed a decreased ability to induce cytokine release in presence of all cell lines compared to PC-1-TCE.

A research version of PC-1 with a histidine tag (PC-1-Histag) demonstrated dose- and cleavage-dependent anti-tumor activity in a mouse model of CRC. This preclinical proof of mechanism supports the requirement of protease activity in a human tumor to enable cleavage and anti-tumor activity of PC-1 in vivo. The nonclinical toxicity profile of PC-1 was investigated in non-human primates. There were no effects on the central nervous system (CNS) or respiratory systems based on clinical observation of the animals and detailed weekly examinations after administration of ≤0.6 mg/kg/dose of PC-1. There were no effects of PC-1 on qualitative electrocardiogram (ECG) parameters nor on QRS or heart rate-corrected QT intervals at PC-1 doses of ≤0.6 mg/kg/week. There was an increased incidence of animals with minimally increased heart rate and associated shortening of mean RR, PR, and QT intervals at ≥0.2 mg/kg/dose at 1 to 3 hours post-end of infusion (EOI) on Days 1 and 22. These changes were not observed following a 4-week recovery period and were likely secondary to cytokine release and not considered to be a direct physiologic effect of PC-1.

### 1.3 Pharmacokinetics

The pharmacokinetic (PK) properties of PC-1 were evaluated following single- and repeat-dose studies in cynomolgus monkeys. The PK characteristics of PC-1 were investigated as part of both nonclinical PK and toxicokinetic (TK) studies in cynomolgus monkeys. A total of 3 studies were performed: two single-dose, non-Good Laboratory Practice (GLP) PK studies in cynomolgus monkeys (one at doses of 0.1, 0.3, and 1 mg/kg and the other at 0.05, 0.2, and 0.6 mg/kg) and a repeat-dose GLP toxicology study in cynomolgus monkeys that examined weekly doses (4 weeks; total 5 doses) of PC-1 at 0.05, 0.2, and 0.6 mg/kg/dose. When present, anti-drug antibodies (ADA) impacted the PK profile of PC-1 in cynomolgus monkeys. In the first single-dose non-GLP PK study, following a single intravenous (IV) bolus administration of PC-1 to cynomolgus monkeys at doses of 0.1, 0.3, and 1 mg/kg, concentrations of PC-1 increased with increased dose. Mean Cmax increased in a more than dose-proportional manner between doses of 0.1 and 0.3 mg/kg and was dose-proportional between 0.3 and 1 mg/kg. Mean AUC0-216h was more than dose proportional between doses of 0.1 and 0.3 mg/kg and was less than dose-proportional between doses of 0.3 and 1 mg/kg. Mean t_{1/2} of PC-1 following a single dose ranged from 88.8 to 101 hours across all tested dose levels. The animal that was sacrificed after 24 hours had the highest observed Cmax and AUC0-24h in this group. PC-1-TCE was not detectable in any samples. In the second single-dose non-GLP PK study, following a single 30-minute IV infusion of PC-1 to cynomolgus monkeys at doses of 0.05, 0.2, and 0.6 mg/kg, mean Cmax values for PC-1 increased in an approximately dose-proportional manner. Mean AUC0-168h increased in a less than dose-proportional manner from 0.05 to 0.2 mg/kg and increased in an approximately dose-proportional manner from 0.2 to 0.6 mg/kg. Mean PC-1 t1_{/2} values were 81.3, 68.2, and 96.5 hours at 0.05, 0.2, and 0.6 mg/kg, respectively. PC-1-TCE was not detectable in any samples. Following five doses of weekly 30-minute IV infusions of PC-1 to cynomolgus monkeys at doses of 0.05, 0.2, and 0.6 mg/kg/dose, mean Cmax, mean AUC0-24 hr, and mean AUC₀₋₁₆₈ₕᵣ after Day 1 dose increased in a nearly dose-proportional manner. However, mean Cmax, mean AUC₀₋₂₄ₕᵣ, and/or mean AUC₀₋₁₆₈ₕᵣ after Days 22 and 29 dose increased in a less than dose-proportional manner due to ADA presence in 3 out of 6 animals at 0.05 mg/kg dose, 6 out of 6 animals in 0.2 mg/kg dose, and 10 out of 10 animals at 0.6 mg/kg dose. Accumulation was not observed after multiple doses and could not be assessed fully with Groups 2, 3, and 4 due to the confirmed ADA presence. No marked accumulation was observed in ADA-negative animals at 0.05 mg/kg/dose (Group 2) and at 0.2 mg/kg/dose (Group 3). One of the ADA-positive animals in Group 2 (2503) and two of ADA-positive animals in Group 3 still attained substantial exposure after Days 22 and/or 29 doses with slightly lower exposure to Day 1 dose, as measured by Cmax, AUC₀₋₂₄ₕ, and AUC₀₋₁₆₈ₕ. In addition, five of the ADA-positive animals in Group 4 also attained some exposure as measured by AUC0-24h, AUC0-168h, and Cmax after Day 22 dose, however, with substantially lower exposure compared to Day 1 dose. PC-1-TCE was not detectable in the majority of samples, except one sample with measurable concentration that was close to the detection limit of the assay.

No pharmacokinetic drug interaction studies have been performed for PC-1. In general, molecules such as PC-1 are not metabolized by cytochrome P450 (CYP) enzymes or transported by P-glycoprotein (Pgp) or related adenosine triphosphate-binding cassette membrane transporters. Cytokines produced by activated lymphocytes may impact the levels of Pgp and the activity of CYP enzymes (Harvey and Morgan, 2014). The clinical relevance of PC-1 causing immune modulation and potential cytokine production that could impact Pgp and CYP is unknown, but a clinically relevant drug-drug interaction effect is considered highly unlikely. Thus, in accordance with guidelines and scientific evidence (FDA August 2020; Huang et al, 2010; Seitz and Zhou, 2007), no PK drug-drug interactions studies were conducted with PC-1.

### 1.4 Toxicology

In the 4-week repeat-dose toxicity study in monkeys, transient clinical signs considered secondary to cytokine release were observed after PC-1 administration on Day 1 at 0.6 mg/kg/dose. PC-1-related clinical chemistry and hematology changes associated with an acute phase response were observed at ≥0.05 mg/kg/dose. Increases in IL-10 at ≥0.2 mg/kg/dose and IL-6 and IFNγ at 0.6 mg/kg/dose were observed after PC-1 on Day 1. Most changes noted during the study were observed post the first dose and returned to the pre-dose levels before the next dose. Following a 4-week recovery period, any remaining minor changes were fully reversible. The no observed adverse effect level (NOAEL) was considered to be 0.6 mg/kg/dose, the highest dose tested. PC-1-induced concentration- or dose-dependent cytokine release (ie, IL-6, IFNγ, and/or TNF) was observed in vitro in the presence of tumor cells and in vivo in normal monkeys with no tumors. In vivo, cytokine release was primarily observed after the first dose and correlated with clinical signs and clinical chemistry changes indicative of cytokine release syndrome.

### 1.5 Phase 1 Clinical Study Design

The study is a first-in-human (FIH), Phase 1/lb, open-label, multicenter dose escalation and dose expansion study to assess the safety, tolerability, PK, pharmacodynamic (PD), and preliminary anti-tumor activity of PC-1 in adult subjects with histologically confirmed advanced or metastatic CRC, NSCLC, renal cell carcinoma (RCC), and SCCHN. The study will be conducted in 3 parts: Dose Escalation (Part 1) with approximately 40 to 50 subjects, Cohort Backfill Expansion (Part 2) with up to approximately 40 subjects enrolled across 4 dose levels, and Dose Expansion (Part 3) with up to approximately 40 subjects enrolled at the recommended Phase 2 dose (RP2D). Dose Escalation (Part 1) will assess the safety, tolerability, PK, PD, and preliminary efficacy of PC-1 administered by IV infusion. Cohort Backfill Enrichment (Part 2) will allow for further characterization of safety and activity of dose levels. Dose Expansion (Part 3) will determine additional safety, tolerability, PK, PD, and preliminary clinical activity data with PC-1 at a dose and schedule to be determined by the Safety Review Committee after reviewing all available safety, PK, PD, and preliminary efficacy data. Depending on the data, randomization may be integrated for either two different RP2D doses or two different treatment intervals.

### Cancer

The following is a non-limiting list of cancers that can be treated with PC-1.
CRC: colorectal cancer, adenocarcinoma of rectum, adenocarcinoma of colon.
SCCHN: squamous cell carcinoma of primary tumor location of oral cavity, oropharynx, hypopharynx, or larynx.
RCC: renal cell carcinoma with clear cell or papillary cell type (not chromophobe,
hereditary cancer syndrome or other types).
NSCLC: non-small cell lung cancer; squamous cell carcinoma, and adenocarcinoma.
Breast Cancer, Pancreatic Cancer, Ovarian Cancer, Prostate Cancer, Brain Cancer (glioblastoma multiforme)

### 2.2 PC-1 Design, Structure, and Mechanism of Action

PC-1 is a TRACTr comprising a humanized tri-specific protein that incorporates EGFR and cluster of differentiation 3 (CD3)-binding domains, an albumin-binding domain to extend circulating half-life, and two separate peptide masks. The peptide masks are fused to the molecule through tumor protease cleavable linkers. One peptide mask inhibits EGFR engagement on target cells, and the other peptide mask inhibits CD3 engagement on T cells (FIG. 1). TRACTr target engagement requires proteolysis of its two cleavable linkers by proteases present in the tumor microenvironment (TME). Once the cleavage sequences undergo proteolysis, the EGFR mask and the tandem CD3 mask plus albumin-binding domain are released, which enables optimal EGFR and CD3 target engagement. This tumor-restricted binding and subsequent T cell activation by the EGFR x CD3 bispecific components of PC-1 promote T cell-mediated killing of EGFR-expressing cancer cells. In addition, loss of the albumin-binding domain ensures that any cleaved PC-1 that migrates out of the tumor will be cleared from the blood compartment rapidly to minimize its accumulation in healthy tissues that can contribute to long-term safety risks. PC-1 is being developed for the treatment of advanced or metastatic tumors known to overexpress EGFR, including metastatic CRC, NSCLC, SCCHN, and RCC in adults.

The conditional masking and half-life extension of TRACTrs are protease cleavage-dependent. Published work describes the upregulation of many proteases in tumors relative to healthy tissue, including MMPs and SPs. In addition, several protease-activated biologics and imaging agents have been clinically validated across a broad spectrum of tumor types. By design, TRACTr molecules are highly sensitive to tumor-selective proteases. Once the TRACTr reaches the TME, proteases cleave the specific substrates (one SP substrate and one MMP substrate) within the cleavable linker, releasing the CD3 mask and albumin-binding domain. The result of protease cleavage is the conversion of the TRACTr to its active form, a TCE.

Notably, the TCE form of PC-1 (PC-1-TCE) has a very short serum half-life, such that cleaved forms of PC-1 that escape the TME are predicted to be cleared from the body before they can generate significant off-tumor toxicity. Preclinical data indicate a TRACTr, via unmasking by proteases at the tumor site, can drive potent anti-tumor responses while producing 25-fold less systemic IL-6 (a key marker of cytokine release syndrome [CRS]) at a 10× higher dose level relative to a non-masked TCE. We anticipate that the TRACTr approach will allow us to demonstrate differentiated safety and efficacy profiles in patients with metastatic and advanced NSCLC, SCCHN, and CRC. Accordingly, a FIH, Phase 1, multicenter, open-label study is planned to determine the safety, PK, RP2D, and preliminary anti-tumor activity of PC-1 administered as a single agent in adult subjects with metastatic or advanced NSCLC, SCCHN, CRC, and RCC

### 2.3 Rationale for PC-1

Therefore, there is a significant unmet need to optimally leverage T cell mediated cytotoxicity in targeting tumor cells. Products that can selectively activate within the TME may have a significant advantage in developing a favorable risk-to-benefit profile. TRACTr-based approach is designed to offer a more focused way to activate T cells in the tumor, minimize systemic activation, enable higher dosing, and thereby increase anti-tumor efficacy. A TRACTr molecule (PC-1) was designed to improve the therapeutic profile of EGFR-targeted TCEs in patients with tumors known to overexpress EGFR, including metastatic CRC, NCSLC, SCCHN, and RCC. PC-1 consists of a core bispecific TCE that recognizes EGFR and CD3 on T cells that is modified by adding tumor protease cleavable linkers connected to peptide masks that specifically inhibit (1) the CD3 binding domain and (2) the EGFR binding domain of PC-1 (FIG. 1). The CD3 mask is designed to limit activity outside the TME by inhibiting CD3 binding in peripheral blood, therefore helping mitigate broad T cell activation that contributes to CRS. Similarly, the EGFR mask is designed to limit on-target, off-tumor EGFR binding and associated toxicity. In addition, PC-1 exhibits an extended half-life in plasma via incorporation of an albumin-binding domain, fused to the CD3 mask. The conditional masking and half-life extension of TRACTrs are protease cleavage-dependent. TRACTr molecules, by design, are highly sensitive to tumor-selective proteases. Once the TRACTr reaches the TME, two types of proteases can cleave the linkers (the linkers contain both an SP and an MMP substrate sequence), releasing the CD3 mask and albumin-binding domain as well as the EGFR mask. The result of protease cleavage is the conversion of the TRACTr to its active form, a TCE.

### 3 Physical, Chemical, and Pharmaceutical Properties and Formulation

### 3.1 Drug Substance

PC-1 is a 97.1 kDa humanized tri-specific glycosylated protein comprised of:
Anti-EGFR antigen-binding fragment (Fab)
Anti-CD3 single-chain variable fragment (scFv)
Anti-albumin single domain antibody (SDA/sdAb)
A peptide mask that inhibits the anti-EGFR Fab from binding EGFR and is integrated into the molecule via a tumor protease cleavable amino acid linker
A second peptide mask that inhibits the anti-CD3 scFv from binding CD3 and is integrated into the molecule via a tumor protease cleavable amino acid linker

The light chain of the anti-CD3 scFv is fused to the N-terminal heavy chain of the anti-EGFR Fab via a short flexible linker. The EGFR inhibitory peptide mask is fused to the amino terminus of the anti-EGFR Fab light chain via a protease cleavable linker. Tandem albumin-binding sdAb and CD3 inhibitory peptide mask are fused to the amino terminus of the anti-CD3 scFv via a tumor protease cleavable linker. The albumin-binding SDA is connected to the amino terminus of the CD3 inhibitory peptide mask via a short flexible linker (FIG. 1).

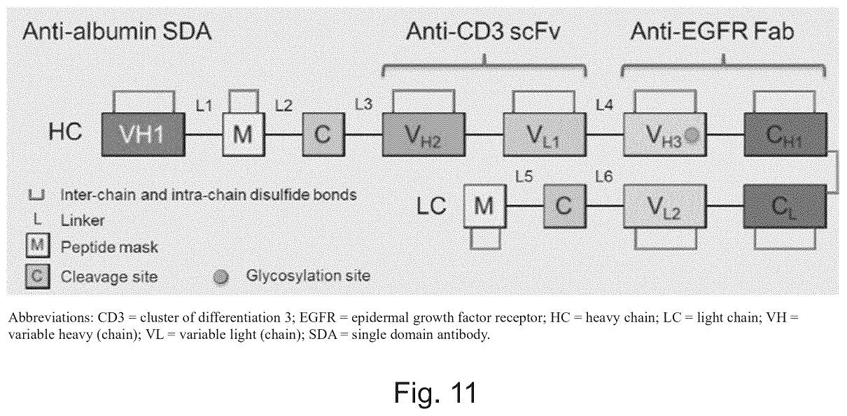

The molecular formula for PC-1 is C₄₁₈₁H₆₄₃₇N₁₁₄₇O₁₃₃₉S₂₆. The predicted average molecular weight of PC-1 without glycosylation is 95,027 Da. PC-1 TRACTr consists of 2 protein chains connected by a single intermolecular disulfide bond between the light chain (LC) and heavy chain (HC) of the TRACTr, and 9 intramolecular disulfide bonds. Each peptide mask contains a single internal disulfide bond. The LC and HC arrangement of PC-1 TRACTr is provided in FIG. 11.

### 3.2 Drug Product

A PC-1 solution for injection (e.g., IV infusion (referred to as PC-1 drug product [DP])), will be provided for clinical investigational as a sterile aqueous solution formulated at a nominal concentration of 2 mg/mL. The solution is formulated in 10 mM histidine, 8% (w/v) sucrose, and 0.01% (w/v) polysorbate 20, and has an approximate pH of 5.3. The formulation of the PC-1 DP is outlined in Table 8. Abbreviations: cGMP=Current good manufacturing practice; BP=British Pharmacopoeia; Ch.P=Chinese Pharmacopoeia; JP=Japanese Pharmacopoeia; Ph Eur=European Pharmacopoeia; Q.S.=quantity sufficient; USP/NF=US Pharmacopeia/National Formulary.

TABLE 8
Composition of PC-1 Drug Product per Vial

| | Amount | | |
|---|---|---|---|
| Component | (mg) | Function | Quality Standard |
| PC-1 | 2.46 | Active pharmaceutical | cGMP |
| | | ingredient | |
| L-Histidine | 0.30 | Buffer | USP, Ph. Eur., JP |
| L-Histidine | 2.17 | Buffer | Ph Eur, BP, JP |
| hydrochloride | | | |
| monohydrate | | | |
| Sucrose | 98.4 | Stabilizer and | NF, Ph. Eur., |
| | | tonicity agent | JP, ChP |
| Polysorbate 20 | 0.12 | Surfactant | USP/NF, Ph. |
| | | | Eur., JP, ChP |
| Water for | Q.S. | Diluent | USP |
| injection | | | |

cGMP = Current good manufacturing practice; BP = British Pharmacopoeia; Ch.P = Chinese Pharmacopoeia; JP = Japanese Pharmacopoeia; Ph Eur = European Pharmacopoeia; Q.S. = quantity sufficient; USP/NF = US Pharmacopeia/National Formulary.

The PC-1 DP comprises the drug substance filled at a target volume of 1.23 mL to enable an extractable volume of ≥1.0 mL in a single dose 2R, Type 1 borosilicate glass vial and sealed with a polypropylene nested cap that contains an embedded elastomeric stopper.

The physical and chemical properties of PC-1 are summarized in Table 9.

TABLE 9
Physical and Chemical Properties of PC-1 Drug Product

| Property | Description | |
|---|---|---|
| Appearance (color, clarity) | Colorless 1.0 NTU | |
| Molecular weight¹ | 97.1 | kDa |
| Melting point Tm by DSC | 73.8° | C. |
| Osmolality | 276 | mOsmol/kg |

| | |
|---|---|
| Solution pH | 5.3 |
| Isoelectric point | 9.1 |
| Potency by ELISA binding assay after cleavage | 102% relative potency |
| of CD3 mask² | |
| Potency by ELISA binding assay after cleavage | 103% relative potency |
| of EGFR mask² | |
| Extinction coefficient | 1.77 (mg/mL)⁻¹ cm⁻¹ |

CD3 = cluster of differentiation 3; DSC = differential scanning calorimetry; ELISA = enzyme-linked immunosorbent assay; NTU = nephelometric turbidity unit; Tm = melting temperature.
¹Molecular weight measured by intact mass for the most abundant glycospecies (G2FS1)
²Relative potency of one batch compared to a second batch

### 3.3 Storage and Handling

The PC-1 DP vials will be stored and shipped frozen (at −20±5° C.). The vial contents will be diluted into an infusion solution that will be chosen based on the results of in-use compatibility studies.

### Nonclinical Studies

### 4.1 Introduction

The nonclinical studies for PC-1 DP are designed to support a Phase 1 clinical program in subjects diagnosed with advanced or metastatic colorectal cancer (CRC), non-small cell lung cancer (NSCLC), renal cell carcinoma (RCC), and squamous cell carcinoma of head and neck (SCCHN). Nonclinical pharmacology studies were performed in in vitro, ex vivo, and in vivo model systems. The in vitro studies examined the following:
PC-1 binding to EGFR, CD3, and albumin antigens from mouse, rat, cynomolgus monkey, and human;
PC-1 stability in serum from healthy human donors, CRC, SCCHN, and NSCLC patients, and cynomolgus monkeys;
The ability of PC-1 to induce T cell mediated anti-tumor cytotoxic activity; and
The ability of PC-1 to induce cytokine production.

Nonclinical pharmacology demonstrated that:
PC-1 exhibits cleavage-dependent binding to human and cynomolgus monkey antigens EGFR, CD3, and albumin.
Masking of EGFR- and CD3-binding domains of PC-1 decreases the affinity of the molecule for EGFR and CD3 antigens. Affinity of PC-1 for human EGFR and CD3 is reduced >300× and >1000× relative to non-masked PC-1-TCE, respectively. Cleaved PC-1 affinity to both antigens reaches non-masked levels upon protease treatment (PC-1-SP cleaved and PC-1-MMP cleaved).
PC-1 is highly stable in human serum from healthy pooled or individual CRC, SCCHN, or NSCLC donors. PC-1 is also stable in cynomolgus monkey serum but to a lesser extent relative to human serum.
PC-1 demonstrated decreased cytotoxic activity against CRC, NSCLC, and SCCHN cell lines relative to PC-1-TCE. Upon proteolytic cleavage, PC-1-SP and/or PC-1-MMP cleaved demonstrated potent T cell-mediated killing of CRC, NSCLC and SCCHN cell lines with the similar potency to PC-1-TCE.
PC-1 demonstrated substantively reduced proinflammatory cytokine production (interferon gamma [IFNγ] and TNF) by peripheral blood mononuclear cells (PBMCs) in the presence of CRC, NSCLC, and SCCHN cells. Upon proteolytic cleavage, PC-1-SP and/or PC-1 MMP cleaved demonstrated potent T cell activation and cytokine release with similar potency as PC-1-TCE.
PC-1-Histag demonstrated dose- and cleavage-dependent anti-tumor activity in a mouse model of CRC.

In vivo pharmacokinetic (PK) and toxicology studies included two non-GLP, single-dose PK studies and one GLP repeat-dose, 4-week toxicity study in cynomolgus monkeys. Nonclinical PK data provided the rationale for a proposed once-weekly dosing schedule in a Phase 1 study.

Nonclinical PK data:

PC-1 was evaluated in cynomolgus monkeys in two non-GLP, single-dose PK and PD studies. Animals were administered PC-1 via IV injection in both studies. Overall, the systemic exposure (examined via area under the plasma concentration versus time curve [AUC] and Cₘₐₓ parameters) increased with increase in dose, generally in a dose-proportional manner. PC-1 exhibited an extended mean t_{1/2} of 79.0 to 101 hours following a single dose between 0.05 to 1.0 mg/kg. The above-mentioned cynomolgus monkey results support the proposed once-weekly dosing regimen in the Phase 1 study.

### Nonclinical Toxicology Data:

PC-1 was evaluated in a definitive 4-week, once-weekly, repeat-dose, IV toxicity study in cynomolgus monkeys (with dosing on Days 1, 8, 15, 22, and 29), transient clinical signs considered secondary to cytokine release were observed after PC-1 administration on Day 1 at 0.6 mg/kg/dose (the highest dosing level). PC-1-related clinical chemistry and hematology changes associated with an acute phase response were observed at ≥0.05 mg/kg/dose. Increases in IL-10 at ≥0.2 mg/kg/dose and IL-6 and IFNγ at 0.6 mg/kg/dose were observed after PC-1 administration on Day 1. Most changes noted during the study were observed post the first dose and returned to the pre-dose level before the next dose. Following a 4-week recovery period, any remaining minor changes were fully reversible. Results from the GLP repeat-dose toxicity study established the no-observed-adverse-effect level (NOAEL) at 0.6 mg/kg/dose, the highest dose tested. Systemic exposure (analyzed through Cₘₐₓ and AUC₀₋₁₆₈ₕ) to PC-1 on Day 1 at the NOAEL was 16100 ng/mL and 775000 hr*ng/mL, respectively, sexes combined.

### 4.2 Nonclinical Pharmacology

The studies were carried out with PC-1 (referred to as uncleaved or Intact PC-1), as well as various MMP or SP cleaved metabolites, the active, non-masked molecule PC-1-TCE, and a non-cleavable (NC) PC-1-NC are shown in Table 10 and Table 11. PC-1-SP cleaved and PC-1-MMP cleaved used in the in vitro pharmacology studies were derived from PC-1 by enzymatic treatment with recombinant human matriptase (MTSP1) and recombinant human matrix metalloprotease 9 (MMP9), respectively.

TABLE 10
Description of PC-1 and Related Molecules

| Name | Description |
|---|---|
| PC-1 | Intact tri-specific TRACTr |
| PC-1-MMP cleaved | Matrix metalloprotease cleaved PC-1 |
| PC-1-SP cleaved | Serine protease cleaved PC-1 |
| PC-1-TCE | Non-masked EGFR × CD3 bispecific TCE |
| PC-1-Histag | PC-1 with Histag |
| PC-1-NC | Non-cleavable PC-1-Histag that lacks protease |
| | substrate sequences |

Abbreviations: CD3 = cluster of differentiation 3; EGFR = epidermal growth factor receptor; MMP = matrix metalloprotease; NC = non-cleavable; SP = serine protease; TCE = T cell engager; TRACTr = tumor activated T cell engager.

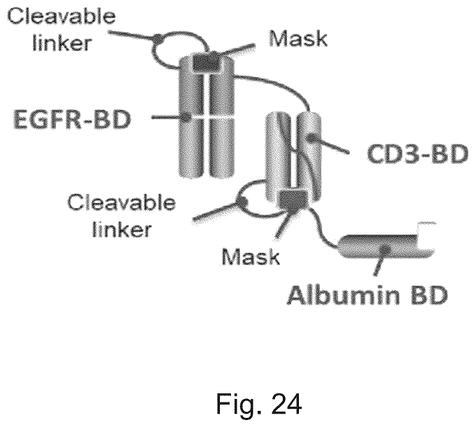

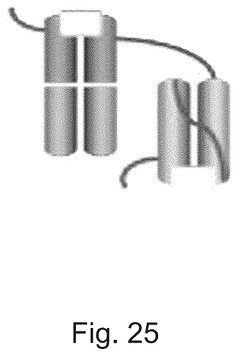

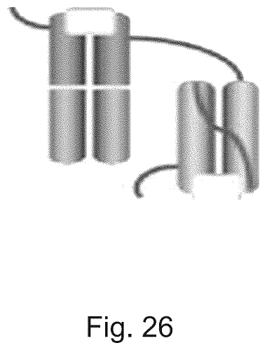

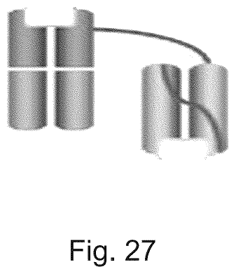

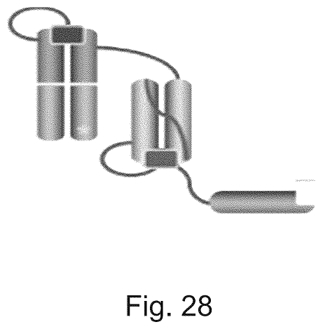

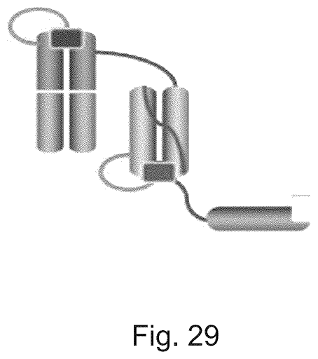

TABLE 11
Summary Description of PC-1 and Related Molecules

| Name | Description | Structure |
|---|---|---|
| PC-1 | Intact tri-specific TRACTr | See FIG. 24 |
| PC-1-SP | PC-1 that has been treated with | See FIG. 25 |
| cleaved | active human recombinant | |
| | MTSP1 enzyme | |
| PC-1-MMP cleaved | PC-1 that has been treated with active | See FIG. 26 |
| | human recombinant MMP9 enzyme | |
| PC-1-TCE | Recombinantly produced EGFR × | See FIG. 27 |
| | CD3 bispecific protein | |
| PC-1-Histag | PC-1 that incorporates a histidine tag | See FIG. 28 |
| | (*) on the C-terminal heavy chain | |
| PC-1-NC | PC-1 that lacks protease substrate | See FIG. 29 |
| | sequences | |

Abbreviations: CD3 = cluster of differentiation 3; EGFR = epidermal growth factor receptor; MMP = matrix metalloprotease; NC = non-cleavable; SP = serine protease; TCE = T cell engager; TRACTr = tumor activated T cell engager. BD = binding domain.;
(*) indicates location of histidine tag.

### 4.2.1 In Vitro Primary Pharmacodynamics

### 4.2.1.1 EGFR Expression in Normal Tissues

EGFR plays a vital role in normal human cellular processes such as proliferation, differentiation, and development. It is heterogeneously expressed in several normal tissues of epithelial, mesenchymal, and neuronal origin, such as skin, heart, lungs, kidney, liver, pancreas, gastrointestinal tract, skeletal muscles, ovaries, testis, brain, etc. Compared to humans, a very similar pattern of EGFR expression at the mRNA level is observed in cynomolgus monkeys.

### 4.2.1.2 PC-1 Binding Affinities for EGFR, CD3 and Albumin

PC-1 is a tri-specific molecule comprising a Fab that binds EGFR and a scFv that binds CD3 and is linked to an SDA/sdAb that binds albumin.

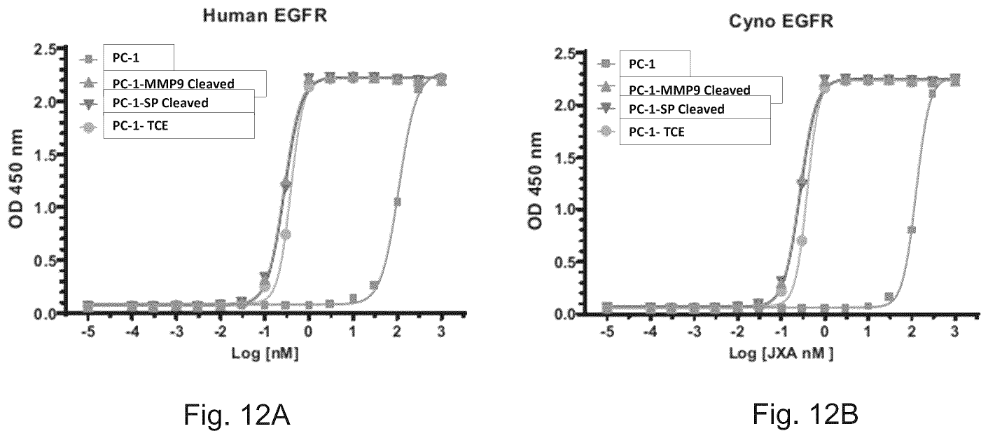

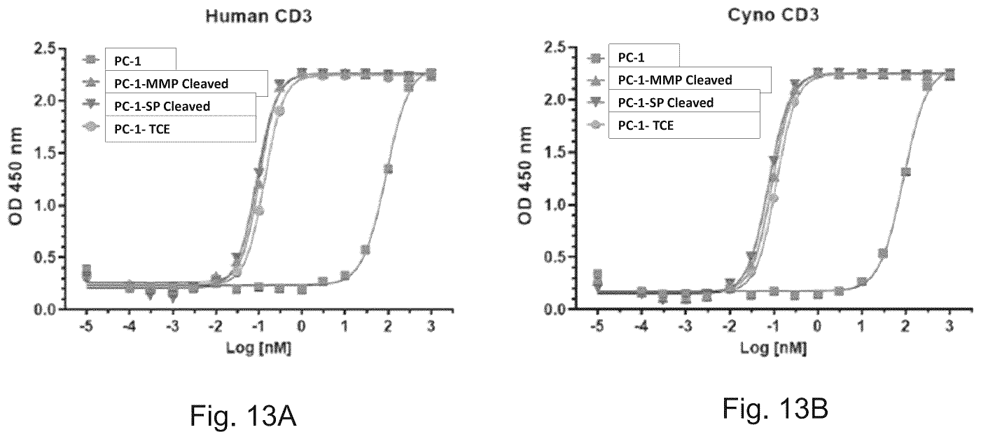

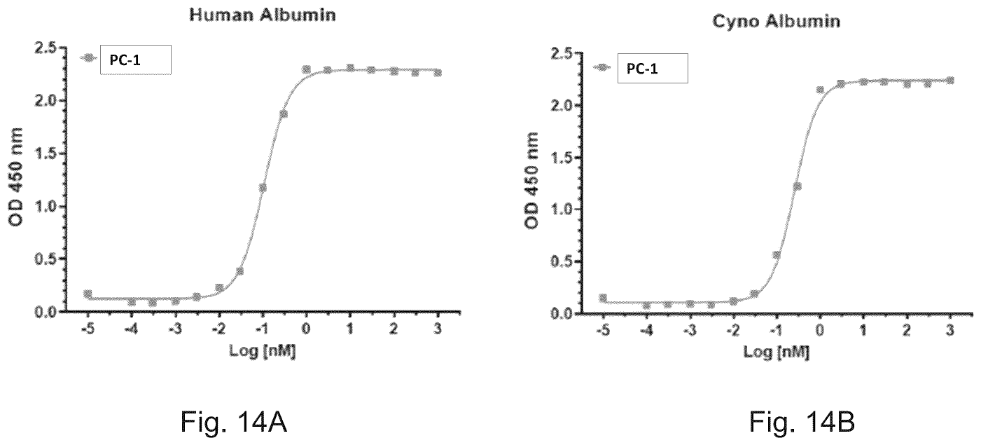

PC-1 was evaluated for its ability to bind human, cynomolgus monkey, mouse and rat antigens in a standard enzyme-linked immunosorbent assay (ELISA) format. PC-1, PC-1-SP cleaved and PC-1-MMP cleaved binding of EGFR or CD3 were measured. FIGS. 12A-12B depicts the binding activity of PC-1, PC-1-MMP9 cleaved, PC-1-SP cleaved, and PC-1-T cell engager (TCE) to epidermal growth factor receptor. FIG. 12A depicts binding of PC-1, PC-1-MMP9 cleaved, PC-1-SP cleaved, and PC-1-T cell engager (TCE) to human EGFR. FIG. 12B depicts binding of PC-1, PC-1-MMP9 cleaved, PC-1-SP cleaved, and PC-1-T cell engager (TCE) to cynomolgus monkey EGFR. FIGS. 13A-13B illustrate the binding activity of PC-1, PC-1-MMP9 cleaved, PC-1-SP cleaved, and PC-1-T cell engager (TCE) to CD3. FIG. 13A depicts binding of PC-1, PC-1-MMP9 cleaved, PC-1-SP cleaved, and PC-1-T cell engager (TCE) to human CD3. FIG. 13B depicts binding of PC-1, PC-1-MMP9 cleaved, PC-1-SP cleaved, and PC-1-T cell engager (TCE) to cynomolgus monkey CD3. FIGS. 14A-14B illustrate the binding activity of isolated recombinant polypeptide complexes to albumin. FIG. 14A depicts binding of PC-1, PC-1-MMP9 cleaved, PC-1-SP cleaved, and PC-1-T cell engager (TCE) to human albumin. FIG. 14B depicts binding of PC-1, PC-1-MMP9 cleaved, PC-1-SP cleaved, and PC-1-T cell engager (TCE) to cynomolgus monkey albumin.

PC-1-TCE was used as a positive control in all ELISAs. The concentration of titrated test articles required to achieve 50% maximal signal (EC50) was calculated for the ELISA. Data are shown in Table 12.

TABLE 12
Binding Affinities of PC-1 and Related Molecules for EGFR, CD3, and Albumin

| | | | Cynomolgus | | |
|---|---|---|---|---|---|
| | | Human | Monkey | Mouse | Rat |
| | Binding Domain | Antigens | Antigens | Antigens | Antigens |
| PC-1 | EGFR EC50 (nM) | 109 | 126 | >1,000 | >1,000 |
| | CD3 EC50 (nM) | 89 | 87 | >1,000 | >1,000 |
| | Albumin EC50 (nM) | 0.1 | 0.3 | >1,000 | >1,000 |
| PC-1-SP | EGFR EC50 (nM) | 0.28 | 0.27 | 29 | 26 |
| cleaved | CD3 EC50 (nM) | 0.08 | 0.08 | >1,000 | 542 |
| (MTSP1 | Albumin EC50 (nM) | NA | NA | NA | NA |
| treated) | | | | | |
| PC-1-MMP | EGFR EC50 (nM) | 0.27 | 0.26 | 20 | 18 |
| cleaved | CD3 EC50 (nM) | 0.09 | 0.09 | >1,000 | 415 |
| (MMP9 | Albumin_{EC50} (nM) | NA | NA | NA | NA |
| treated) | | | | | |
| PC-1-TCE | EGFR_{EC50} (nM) | 0.39 | 0.39 | 295 | 234 |
| | CD3_{EC50} (nM) | 0.13 | 0.11 | 767 | 20 |
| | Albumin EC50 (nM) | NA | NA | NA | NA |

Abbreviations: CD3 = cluster of differentiation 3; EC50 = half-maximal effective concentration; EGFR = epidermal growth factor receptor; MMP = matrix metalloprotease; NA = not applicable; SP = serine protease; TCE = T cell engager.

PC-1, PC-1-SP cleaved, PC-1-MMP cleaved and PC-1-TCE exhibit nanomolar binding affinity to human and cynomolgus monkey EGFR. In contrast, the affinity of PC-1 binding to CD3 and EGFR is cleavage-dependent. While PC-1-SP cleaved, PC-1-MMP cleaved and PC-1-TCE exhibit binding to human and cynomolgus monkey EGFR and CD3, PC-1 exhibits orders of magnitude weaker binding to EGFR and CD3 due to masking of the EGFR- and CD3-binding domains. PC-1 binds to human and cynomolgus monkey albumin with similar potencies. In contrast, PC-1 demonstrates orders of magnitude weaker binding to mouse or rat antigens relative to human and cynomolgus antigens.

4.2.1.3 Stability of PC-1 in Serum from Cynomolgus Monkeys, Healthy Human Donors, and CRC, SCCHN, and NSCLC Cancer Patients

While proteolytic cleavage of PC-1 in the TME is expected to drive anti-tumor activity, a critical safety feature of PC-1 is its stability in the blood compartment, where maintenance of masking is expected to mitigate the safety risks associated with healthy tissue toxicity and cytokine release syndrome (CRS). Maintaining masking and cleavable linker stability of PC-1 was characterized in human and cynomolgus monkey serum. Serum is considered proteolytically rich due to the activation of protease-driven clotting pathways during the conversion of whole blood to serum. With the activation of serum proteases during clotting, serum is likely a more stringent test of PC-1 stability relative to whole blood or plasma.

PC-1 stability in human and cynomolgus monkey serum was evaluated using kinetic binding assays. Briefly, PC-1 or PC-1-TCE was incubated in normal pooled human serum, individual CRC or SCCHN or NSCLC human serum, or normal pooled cynomolgus monkey serum for 0, 24, 48, 72, or 168 hours. Samples at the indicated time points were then tested for their ability to bind EGFR and CD3 using an Octet bio-layer interferometry instrument. The initial slope of the EGFR and CD3 kinetic binding curves were used to calculate the relative concentration of cleaved PC-1 (mixture of PC-1-MMP cleaved and PC-1-SP cleaved) in each sample. Using the relative concentration of cleaved PC-1 over time, a first-order linear regression was used to calculate the rate of de-masking in each of the tested serum matrices.

Results indicated that PC-1 is more susceptible to cleavage in cynomolgus monkey serum compared to human serum or serum from CRC, SCCHN, and NSCLC patients. The cleavage rate of the EGFR mask of PC-1 in cynomolgus monkey serum was 11.5% per day and for the CD3 mask of PC-1 was 6.6%. In pooled healthy human serum, the cleavage rate of both masks within PC-1 was 1% per day. As shown in Table 13, PC-1 exhibited similar cleavage rates in serum from CRC, SCCHN, and NSCLC patients with a cleavage rate of less than 2% per day for either mask. Accordingly, PC-1 appears to be stable in serum derived from human blood.

TABLE 13
Serum Stability of PC-1

| | PC-1% cleavage/day | |
|---|---|---|
| Matrix | EGFR mask | CD3 mask |
| Normal pooled human serum | 1.0% | 1.0% |
| Normal pooled cynomolgus monkey serum | 11.5% | 6.6% |
| CRC donor serum (N = 6) | 1.1% | 1.3% |
| SCCHN donor serum (N = 6) | 1.9% | 1.9% |
| NSCLC donor serum (N = 6) | 1.4% | 1.6% |

Note:
Results are reported as the average percent cleavage per day.
Abbreviations: CD3 = cluster of differentiation 3; CRC = colorectal cancer; EGFR = epidermal growth factor receptor; NSCLC = non-small cell lung cancer; SCCHN = squamous cell carcinoma of head and neck.

### 4.2.1.4 PC-1-Induced In Vitro T Cell-Mediated Killing of Target Tumor Cells Expressing EGFR

The ability of PC-1 to induce T cell-mediated killing of target tumor cells in a functional in vitro assay was compared to that of the cleaved PC-1 (PC-1-SP cleaved, PC-1-MMP cleaved) and non-masked (PC-1-TCE) forms. Three different EGFR-expressing cell lines were used, i) HCT116 cells, ii) Cal27 cells, and iii) EGFR-deficient, A549 EGFR knockout cells. The ability of PC-1 to induce T cell-mediated cytotoxicity was compared to that of cleaved (PC-1-SP cleaved, PC-1-MMP cleaved) and non-masked (PC-1-TCE) forms in these cell lines. PBMCs and selected tumor cells were co-cultured in the presence of increasing concentrations of test articles for 72 hours. Tumor cell growth kinetics were then plotted against the concentration of the test article, and the concentration required to reduce the tumor cell growth by 50% (EC₅₀) was calculated.

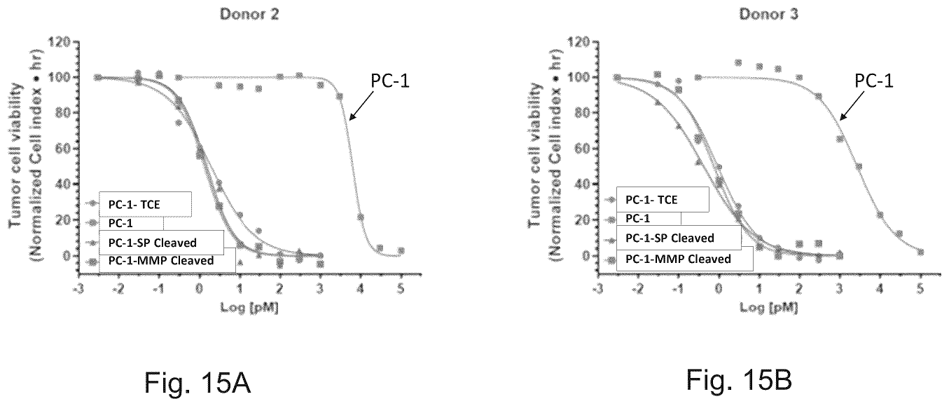

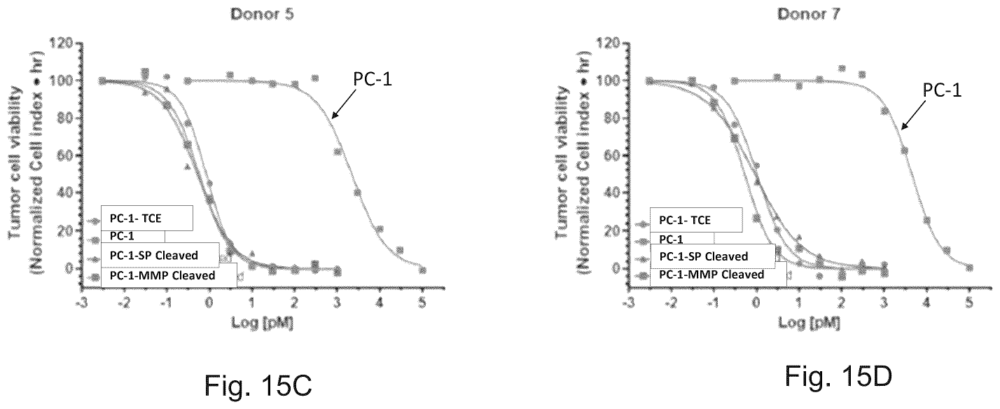

FIGS. 15A-15D illustrate tumor cell killing of HCT116 tumor cells from four donors after administration of the isolated recombinant polypeptide complexes. HCT116 cells were from a CRC-derived cell line, KRAS and PIK3CA mutant, 35,000 EGFR copies/cell. The data from this assay are shown in Table 14.

TABLE 14
Tumor Killing EC50 of EGFR Positive CRC Cell Line HCT116

| HCT116 | | | PC-1-SP | | PC-1-MMP | | PC-1-TCE, | |
|---|---|---|---|---|---|---|---|---|
| Cytotoxicity | PC-1, EC50 | | cleaved, EC50 | | cleaved, EC50 | | EC50 | |
| E:T of 5:1 | pM | ng/mL | pM | ng/mL | pM | ng/mL | pM | ng/mL |
| Average | 5,634 | 535 | 1.2 | 0.09 | 1 | 0.08 | 1.8 | 0.13 |
| (N = 8 donors) | | | | | | | | |

Abbreviations: CRC = colorectal cancer; E = effector; EC50 = half-maximal effective concentration; EGFR = epidermal growth factor receptor; MMP = matrix metalloprotease; PBMC = peripheral blood mononuclear cells; SP = serine protease; T = target; TCE = T cell engager.

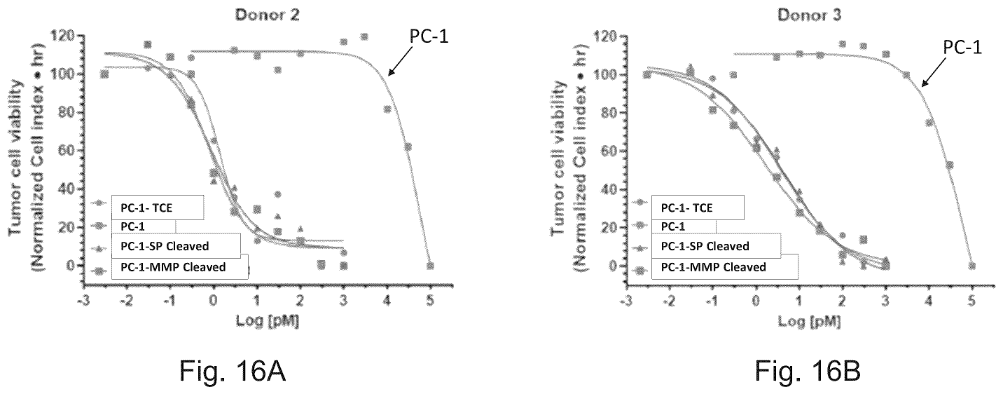

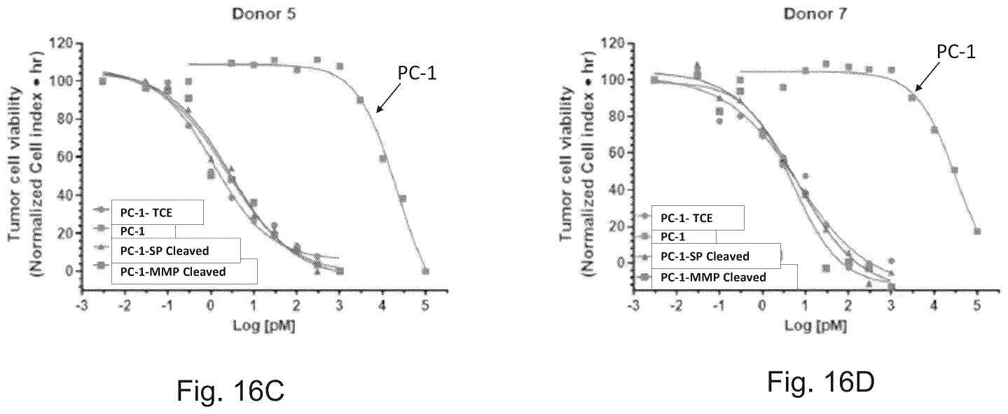

FIGS. 16A-16D illustrate tumor cell killing of A549 tumor cells from four donors after administration of isolated recombinant polypeptide complexes. A549 cells were from a NSCLC-derived cell line, KRAS mutant, 25,000 EGFR copies/cell. The data from this assay are shown in Table 15.

TABLE 15
Tumor Killing EC50 of EGFR Positive NSCLC Cell Line A549

| A549 | | | PC-1-SP | | PC-1-MMP | | PC-1-TCE, | |
|---|---|---|---|---|---|---|---|---|
| Cytotoxicity | PC-1, EC50 | | cleaved, EC50 | | cleaved, EC50 | | EC50 | |
| E:T of 5:1 | pM | ng/mL | pM | ng/mL | pM | ng/mL | pM | ng/mL |
| Average | 38,173 | 3,628 | 4.2 | 0.32 | 3.2 | 0.24 | 4.6 | 0.34 |
| (N = 8 donors) | | | | | | | | |

Abbreviations: E = effector; EC50 = half-maximal effective concentration; EGFR = epidermal growth factor receptor; MMP = matrix metalloprotease; NSCLC = non-small cell lung cancer; PBMC = peripheral blood mononuclear cells; SP = serine protease; T = target; TCE = T cell engager.

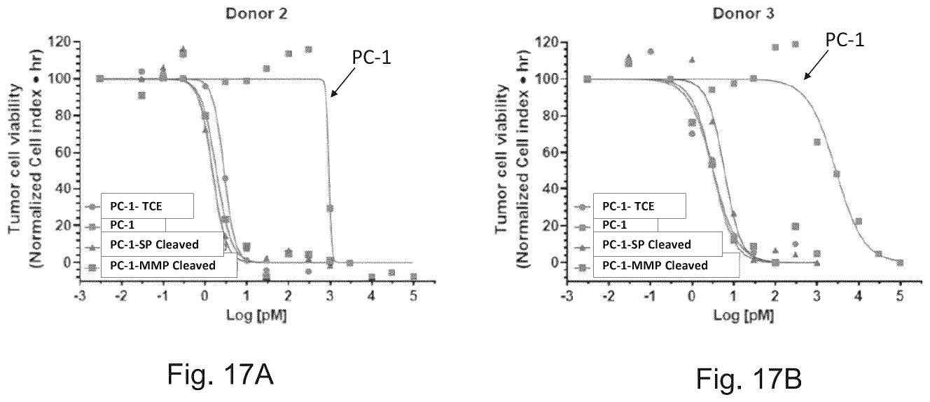

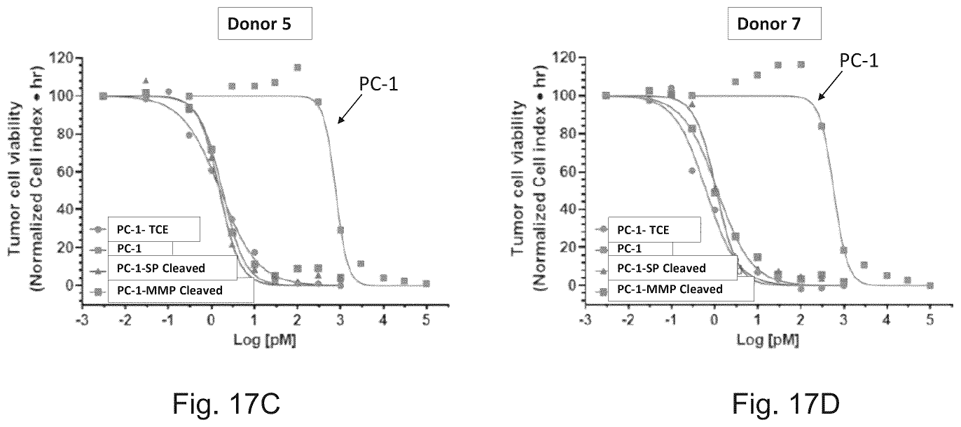

FIGS. 17A-17D illustrate tumor cell killing of Cal27 tumor cells from four donors after administration of isolated recombinant polypeptide complexes. Cal27 cells were from a SCCHN-derived cell line, 170,000 EGFR copies/cell. The data from this assay are shown in Table 16.

TABLE 16
Tumor Killing EC50 of EGFR Positive SCCHN Cell Line Cal27

| Cal27 | | | PC-1-SP | | PC-1-MMP | | PC-1-TCE, | |
|---|---|---|---|---|---|---|---|---|
| Cytotoxicity | PC-1, EC50 | | cleaved, EC50 | | cleaved, EC50 | | EC50 | |
| E:T of 5:1 | pM | ng/mL | pM | ng/mL | pM | ng/mL | pM | ng/mL |
| Average | 1,489 | 142 | 3.33 | 0.25 | 3.05 | 0.23 | 2.66 | 0.2 |
| (N = 8 donors) | | | | | | | | |

Abbreviations: E = effector; EC50 = half-maximal effective concentration; EGFR = epidermal growth factor receptor; MMP = matrix metalloprotease; NSCLC = non-small cell lung cancer; PBMC = peripheral blood mononuclear cells; SP = serine protease; T = target; TCE = T cell engager.

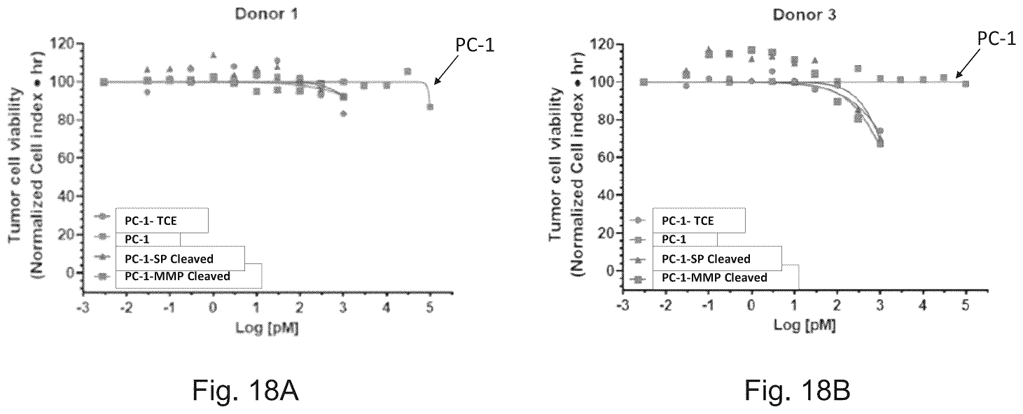

As a negative control, an EGFR-deficient, A549 EGFR knockout (A549 EGFR-KO) cell line was used. FIGS. 18A-18B illustrate the tumor cell killing of the A549 EGFR-KO cells two donors after administration of isolated recombinant polypeptide complexes. The data from this assay are shown in Table 17.

TABLE 17
Tumor Killing EC50 of EGFR-Deficient NSCLC Cell Line A549 EGFR-KO

| A549 EGFR- | | | | | PC-1- | | | |
|---|---|---|---|---|---|---|---|---|
| KO | | | PC-1-SP | | MMP cleaved, | | PC-1-TCE, | |
| Cytotoxicity | PC-1-1, EC50 | | cleaved, EC50 | | EC50 | | EC50 | |
| E:T of 5:1 | pM | ng/mL | pM | ng/mL | pM | ng/mL | pM | ng/mL |
| Average | >100,000 | >9,503 | >1,000 | >76.2 | >1,000 | >74.9 | >1,000 | >73.7 |
| (N = 4 donors) | | | | | | | | |

Abbreviations: E = effector; EC50 = half-maximal effective concentration; EGFR = epidermal growth factor receptor; KO = knockout; MMP = matrix metalloprotease; ND = not determined; NSCLC = non-small cell lung cancer; PBMC = peripheral blood mononuclear cells; SP = serine protease; T = target; TCE = T cell engager.

PC-1-induced tumor cell killing was cleavage- and concentration-dependent. PC-1 exhibited much lower potency than cleaved forms (PC-1 SP-cleaved and PC-1 MMP-cleaved) and non-masked PC-1-TCE. For example, PC-1 exhibits ˜8,000-12,000× decreased ability to induce A549 (˜20,000 EGFR copies/cell) tumor cell killing relative to PC-1 SP cleaved. The decrease in PC-1 ability to induce tumor cell killing relative to its cleaved counterparts was ˜5,500 and ˜450 fold lower in culture systems containing HCT116 (˜30,000 EGFR copies/cell) and Cal27 (170,000 EGFR copies/cell) cells, respectively.

The ability of PC-1 to induce tumor cell killing depended on the expression level of EGFR on the surface of target tumor cells. None of the test articles induced cytotoxicity against a control EGFR-deficient A549 cell line, A549 EGFR-KO (no detectable EGFR expression). Lack of activity against the EGFR-deficient A549 cell line suggests activity of PC-1 and its cleaved forms is EGFR-specific and requires EGFR expression on target cells.

### 4.2.1.5 Cytokine Release Assays

4.2.1.5.1 PC-1-Induced Cytokine Production by T Cells Co-Cultured with Target Tumor Cells Expressing EGFR

The ability of PC-1, PC-1-SP cleaved, PC-1-MMP cleaved, and PC-1-TCE to induce cytokine (e.g., IFNγ, TNF, and IL-6) release was assessed after co-culturing of human PBMCs and tumor cells in the presence of the test articles. Human PBMCs were stimulated with increasing concentrations of test articles in the presence of EGFR-expressing (HCT116, A549, and Cal27) or EGFR-deficient (A549 EGFR-KO) tumor cell lines. The assay was similar to that described in Section 4.2.1.4 (titled “PC-1-Induced In Vitro T Cell-Mediated Killing of Target Tumor Cells Expressing EGFR”). PBMCs were incubated with test articles and tumor cells for 72 hours. Soluble IFNγ, TNF, and IL-6 were measured in cell culture supernatants using an immunoassay. Cytokine concentrations were plotted against test article concentrations and the concentration required to induce 50% (EC50) of maximum cytokine release was calculated.

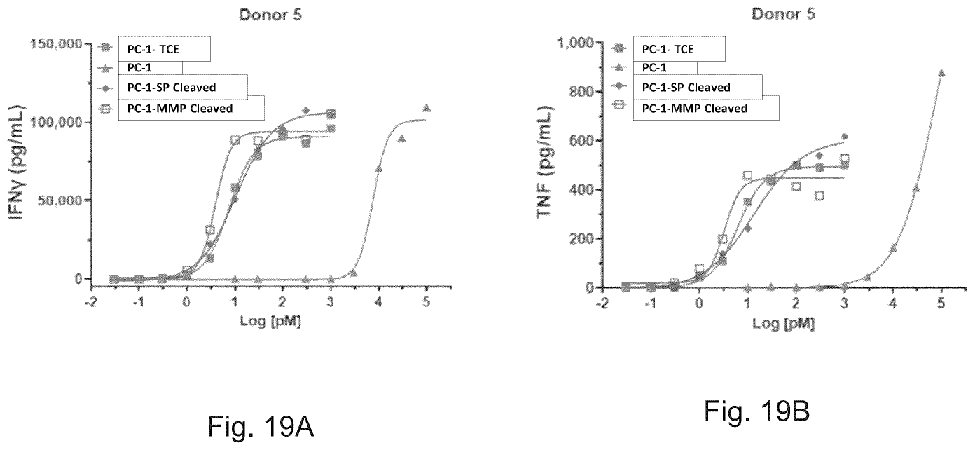

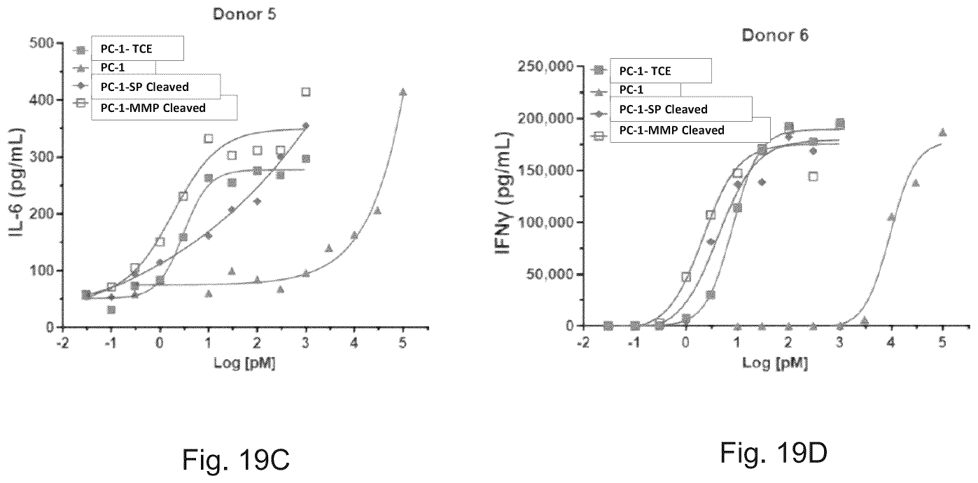

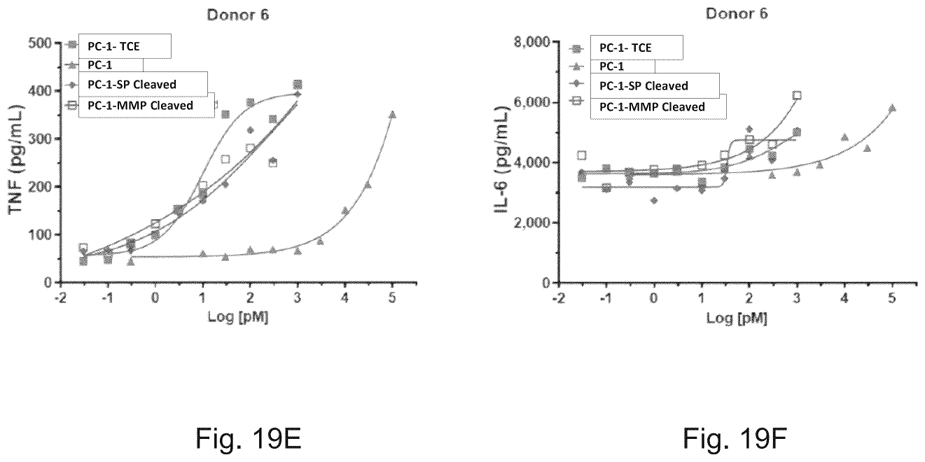

FIGS. 19A-19F illustrate the effect of PC-1, PC-1-SP cleaved, PC-1-MMP cleaved, and PC-1-TCE on cytokine concentrations (e.g., concentrations of IFNγ, TNF, and IL-6) from healthy donor peripheral blood mononuclear cells (PBMCs) in the presence of HCT116 cells. FIG. 19A demonstrates the effects of PC-1, PC-1-SP cleaved, PC-1-MMP cleaved, and PC-1-TCE on concentrations of IFNγ using Donor 5 PBMCs. FIG. 19B demonstrates the effects of PC-1, PC-1-SP cleaved, PC-1-MMP cleaved, and PC-1-TCE on concentrations of TNF using Donor 5 PBMCs. FIG. 19C demonstrates the effects of PC-1, PC-1-SP cleaved, PC-1-MMP cleaved, and PC-1-TCE on concentrations of IL-6 using Donor 5 PBMCs. FIG. 19D demonstrates the effects of PC-1, PC-1-SP cleaved, PC-1-MMP cleaved, and PC-1-TCE on concentrations of IFNγ using Donor 6 PBMCs. FIG. 19E demonstrates the effects of administering PC-1, PC-1-SP cleaved, PC-1-MMP cleaved, and PC-1-TCE on concentrations of TNF using Donor 6 PBMCs. FIG. 19F demonstrates the effects of PC-1, PC-1-SP cleaved, PC-1-MMP cleaved, and PC-1-TCE on concentrations of IL-6 using Donor 6 PBMCs. Data from this assay are shown in Table 18.

TABLE 18
Test Article-Induced Release of IFNγ, TNF, and
IL-6 by Healthy Donor PBMCs Cultured with HCT116 Cells

| HCT116 + | | | | | | | | | | | | |
|---|---|---|---|---|---|---|---|---|---|---|---|---|
| PBMCs | | | | PC-1-SP | | | PC-1-MMP | | | PC-1-TCE, | | |
| cytokine | | | | cleaved,EC50 | | | cleaved, EC50 | | | EC50 | | |
| release | PC-1,_{EC50} (ng/mL) | | | (ng/mL) | | | (ng/mL) | | | (ng/mL) | | |
| E:T of 5:1 | IFNγ | TNF | IL-6 | IFNγ | TNF | IL-6 | IFNγ | TNF | IL-6 | IFNγ | TNF | IL-6 |
| Average | 2116.5 | 2015.4 | 1692.7 | 5.1 | 2.4 | 13.8 | 2.6 | 1.3 | 6.8 | 2.3 | 1.6 | 5.3 |
| (N = 8 | | | | | | | | | | | | |
| donors) | | | | | | | | | | | | |

Abbreviations: E = effector; EC50 = half-maximal effective concentration; EGFR = epidermal growth factor receptor; IFNγ = interferon gamma; IL-6 = interleukin 6; MMP = matrix metalloprotease; ND = not determined; NSCLC = non-small cell lung cancer; PBMC = peripheral blood mononuclear cells; SP = serine protease; T = target; TCE = T cell engager; TNF = tumor necrosis factor.

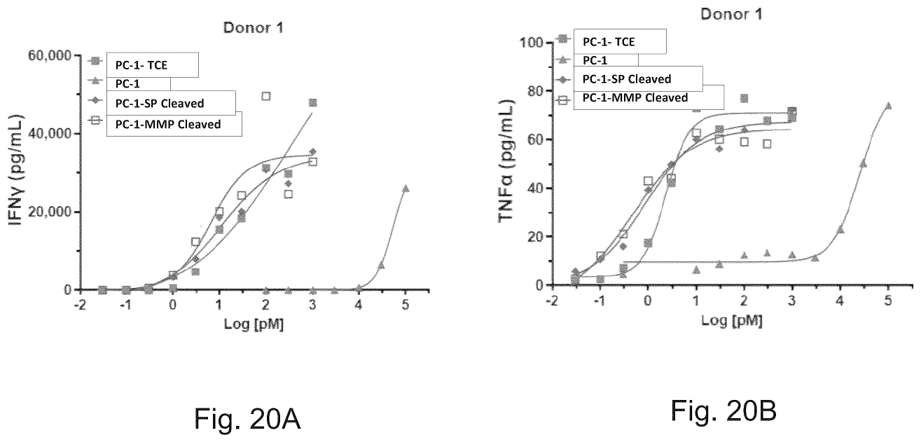

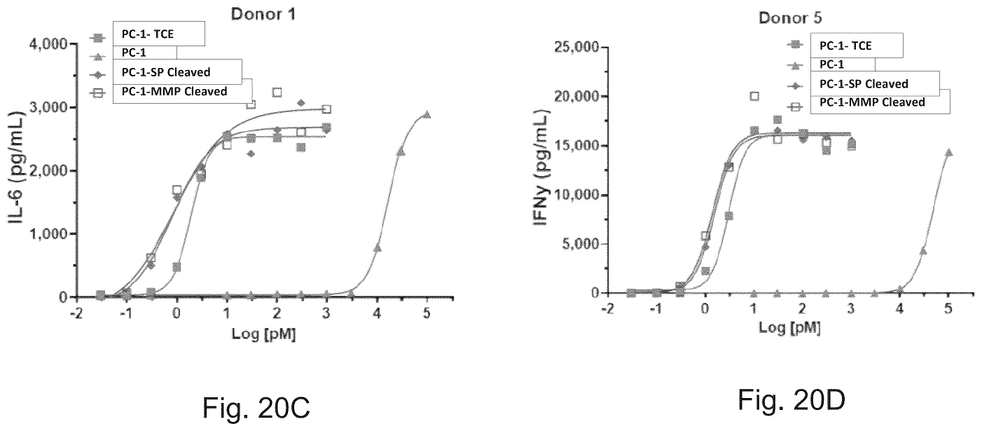

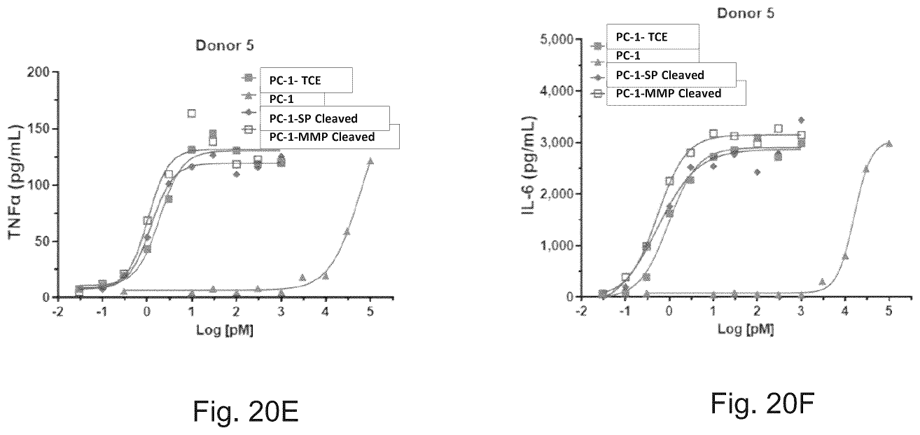

FIGS. 20A-20F illustrate the effect of PC-1, PC-1-SP cleaved, PC-1-MMP cleaved, and PC-1-TCE on cytokine concentrations (e.g., concentrations of IFNγ, TNF, and IL-6) from healthy donor peripheral blood mononuclear cells (PBMCs) in the presence of A549 cells. FIG. 20A demonstrates the effects of PC-1, PC-1-SP cleaved, PC-1-MMP cleaved, and PC-1-TCE on concentrations of IFNγ in Donor 1 PBMCs. FIG. 20B demonstrates the effects of PC-1, PC-1-SP cleaved, PC-1-MMP cleaved, and PC-1-TCE on concentrations of TNF in Donor 1 PBMCs. FIG. 20C demonstrates the effects of PC-1, PC-1-SP cleaved, PC-1-MMP cleaved, and PC-1-TCE on concentrations of IL-6 in Donor 1 PBMCs. FIG. 20D demonstrates the effects of PC-1, PC-1-SP cleaved, PC-1-MMP cleaved, and PC-1-TCE on concentrations of IFNγ in Donor 5 PBMCs. FIG. 20E demonstrates the effects of PC-1, PC-1-SP cleaved, PC-1-MMP cleaved, and PC-1-TCE on concentrations of TNF in Donor 5 PBMCs. FIG. 20F demonstrates the effects of PC-1, PC-1-SP cleaved, PC-1-MMP cleaved, and PC-1-TCE on concentrations of IL-6 in Donor 5 PBMCs. Data from this assay are shown in Table 19.

TABLE 19
Ability of Test Articles to Induce Release of IFNγ, TNF,
and IL-6 by Healthy Donor PBMCs Cultured with A549 Cells

| A549 + | | | | | | | | | | | | |
|---|---|---|---|---|---|---|---|---|---|---|---|---|
| PBMCs | | | | PC-1-SP | | | PC-1-MMP | | | PC-1-TCE, | | |
| cytokine | PC-1,_{EC50} | | | cleaved, EC50 | | | cleaved, EC50 | | | EC50 | | |
| release | (ng/mL) | | | (ng/mL) | | | (ng/mL) | | | (ng/mL) | | |
| E:T of 5:1 | IFNγ | TNF | IL-6 | IFNγ | TNF | IL-6 | IFNγ | TNF | IL-6 | IFNγ | TNF | IL-6 |
| Average | 3627.6 | 1801.3 | 1170.8 | 1.58 | 0.20 | 0.47 | 1.21 | 0.28 | 0.09 | 3.73 | 0.11 | 0.22 |
| (N = 8) | | | | | | | | | | | | |

Abbreviations: E = effector; EC50 = half-maximal effective concentration; EGFR = epidermal growth factor receptor; IFNγ = interferon gamma; IL-6 = interleukin 6; MMP = matrix metalloprotease; NSCLC = non-small cell lung cancer; PBMC = peripheral blood mononuclear cells; SP = serine protease; T = target; TCE = T cell engager; TNF = tumor necrosis factor.

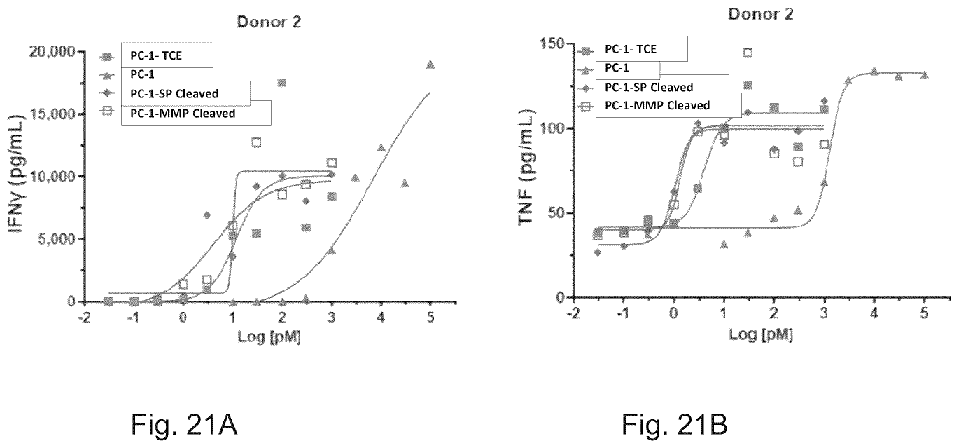

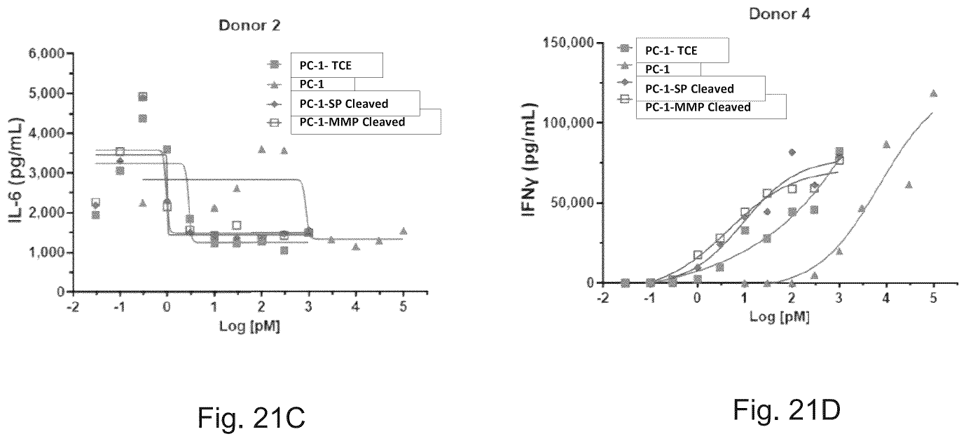

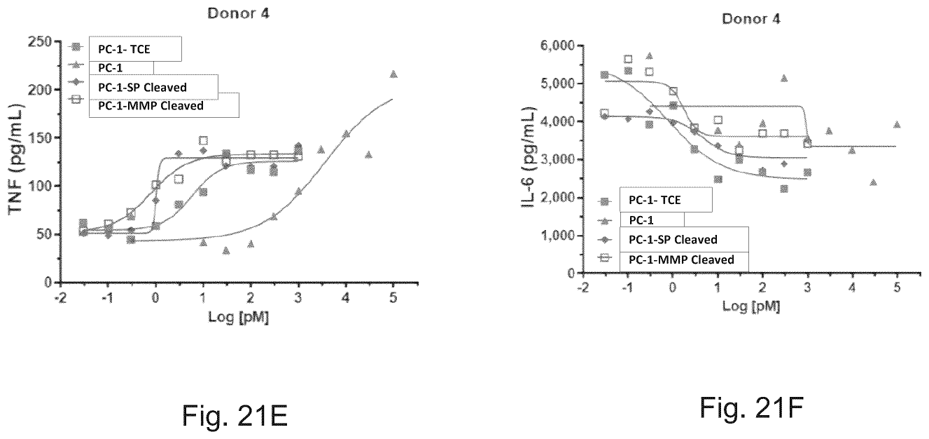

FIGS. 21A-21F illustrate the effect of PC-1, PC-1-SP cleaved, PC-1-MMP cleaved, and PC-1-TCE on cytokine concentrations (e.g., concentrations of IFNγ, TNF, and IL-6) from healthy donor peripheral blood mononuclear cells (PBMCs) in the presence of Cal27 cells. FIG. 21A demonstrates the effects of PC-1, PC-1-SP cleaved, PC-1-MMP cleaved, and PC-1-TCE on concentrations of IFNγ in Donor 2 PBMCs. FIG. 21B demonstrates the effects of PC-1, PC-1-SP cleaved, PC-1-MMP cleaved, and PC-1-TCE on concentrations of TNF in Donor 2 PBMCs. FIG. 21C demonstrates the effects of PC-1, PC-1-SP cleaved, PC-1-MMP cleaved, and PC-1-TCE on concentrations of IL-6 in Donor 2 PBMCs. FIG. 21D demonstrates the effects of PC-1, PC-1-SP cleaved, PC-1-MMP cleaved, and PC-1-TCE on concentrations of IFNγ in Donor 4 PBMCs. FIG. 21E demonstrates the effects of PC-1, PC-1-SP cleaved, PC-1-MMP cleaved, and PC-1-TCE on concentrations of TNF in Donor 4 PBMCs. FIG. 21F demonstrates the effects of PC-1, PC-1-SP cleaved, PC-1-MMP cleaved, and PC-1-TCE on concentrations of IL-6 in Donor 4 PBMCs. Data from this assay are shown in Table 20.

TABLE 20
Ability of Test Articles to Induce Release of IFNγ, TNF,
and IL-6 by Healthy Donor PBMCs Cultured with Cal27 Cells

| Cal27 + | | | | | | | | | | | | |
|---|---|---|---|---|---|---|---|---|---|---|---|---|
| PBMCs | | | | PC-1-SP cleaved, | | | PC-1-MMP | | | PC-1-TCE, | | |
| cytokine | PC-1,_{EC50} | | | EC50 | | | cleaved, EC50 | | | EC50 | | |
| release | (ng/mL) | | | (ng/mL) | | | (ng/mL) | | | (ng/mL) | | |
| E:T of 5:1 | IFNγ | TNF | IL-6 | IFNγ | TNF | IL-6 | IFNγ | TNF | IL-6 | IFNγ | TNF | IL-6 |
| Average | 761.2 | 324.7 | ND | 0.36 | 0.10 | ND | 0.61 | 0.08 | ND | 1.95 | 0.32 | ND |
| (N = 8 donors) | | | | | | | | | | | | |

Abbreviations: E = effector; EC50 = half-maximal effective concentration; EGFR = epidermal growth factor receptor; IFNγ = interferon gamma; IL-6 = interleukin 6; MMP = matrix metalloprotease; ND = not determined; NSCLC = non-small cell lung cancer; PBMC = peripheral blood mononuclear cells; SP = serine protease; T = target; TCE = T cell engager; TNF = tumor necrosis factor.

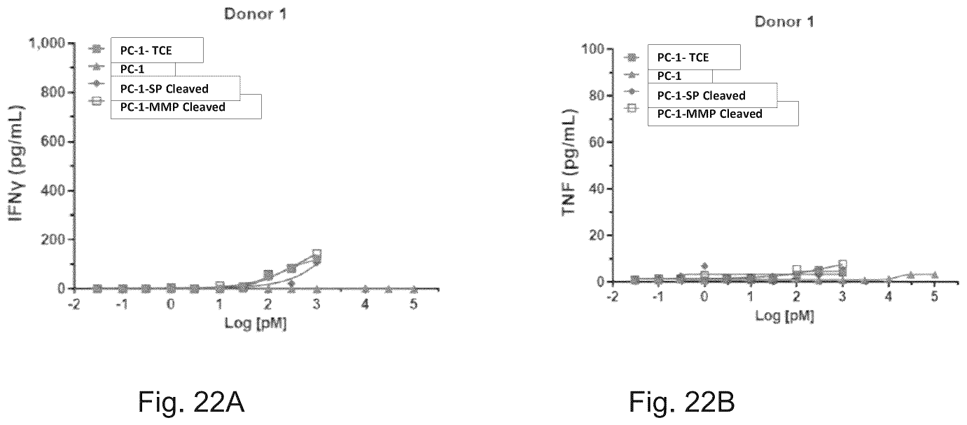

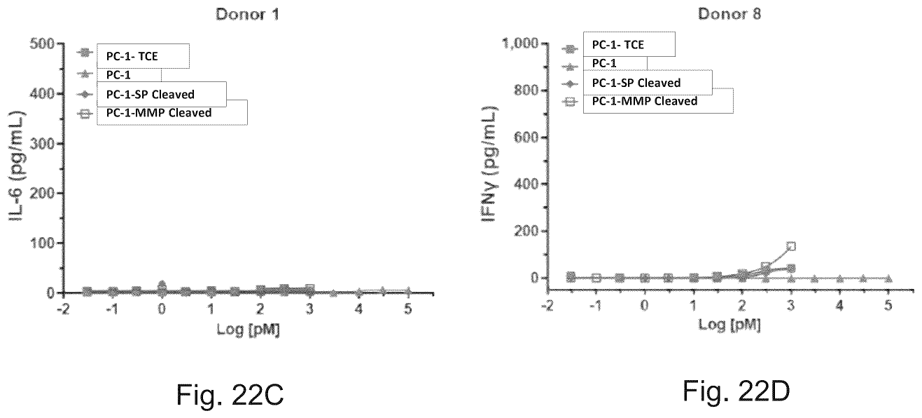

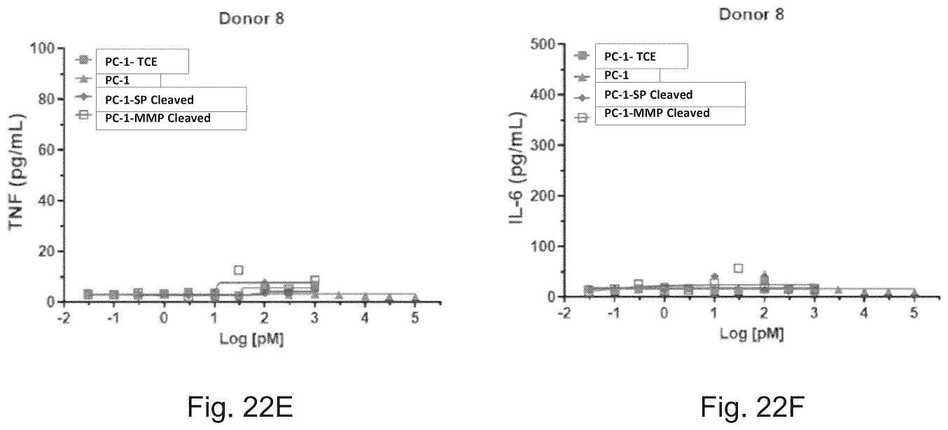

FIGS. 22A-22F illustrate the effect of PC-1, PC-1-SP cleaved, PC-1-MMP cleaved, and PC-1-TCE on cytokine concentrations (e.g., concentrations of IFNγ, TNF, and IL-6) from healthy donor peripheral blood mononuclear cells (“PBMCs”) in the presence of A549 EGFR-KO cells. FIG. 22A demonstrates the effects of PC-1, PC-1-SP cleaved, PC-1-MMP cleaved, and PC-1-TCE on concentrations of IFNγ in Donor 1 PBMCs. FIG. 22B demonstrates the effects of PC-1, PC-1-SP cleaved, PC-1-MMP cleaved, and PC-1-TCE on concentrations of TNF in Donor 1 PBMCs. FIG. 22C demonstrates the effects of PC-1, PC-1-SP cleaved, PC-1-MMP cleaved, and PC-1-TCE on concentrations of IL-6 in Donor 1 PBMCs. FIG. 22D demonstrates the effects of administering PC-1, PC-1-SP cleaved, PC-1-MMP cleaved, and PC-1-TCE on concentrations of IFNγ in Donor 8. FIG. 22E demonstrates the effects of PC-1, PC-1-SP cleaved, PC-1-MMP cleaved, and PC-1-TCE on concentrations of TNF in Donor 8 PBMCs. FIG. 22F demonstrates the effects of PC-1, PC-1-SP cleaved, PC-1-MMP cleaved, and PC-1-TCE on concentrations of IL-6 in Donor 8 PBMCs.

Concentration-dependent production of IFNγ, TNF, and IL-6 by PBMCs co-cultured with HCT116 and A549 cells was observed. Concentration-dependent production of IFNγ and TNF, but not IL-6, was observed in the presence of the Cal27 cell line. Although variable, levels of cytokines produced by PBMCs from different donors followed the same pattern.

Cleaved (PC-1-SP cleaved and PC-1-MMP cleaved) and non-masked (PC-1-TCE) test articles induced concentration-dependent cytokine release. PC-1 showed a decreased ability to induce cytokine release in the presence of all cell lines compared to its non-masked (PC-1-TCE) and cleaved (PC-1-SP cleaved and PC-1-MMP cleaved) counterparts.

While concentration-dependent cytokine release was apparent upon stimulation of PBMCs in the presence of EGFR-expressing cell lines, the magnitude of cytokine production was reduced in co-cultures with A549 EGFR-KO. Low-level cytokine release upon stimulation with cleaved (PC-1-SP and PC-1-MMP cleaved) and non-masked (PC-1-TCE) molecules and a complete absence of cytokine release upon treatment with PC-1 was observed.

4.2.1.5.2 Cytokine Release from Healthy Human Whole Blood in Soluble and Wet-Coated Plate Formats

The ability of PC-1 and PC-1-SP cleaved to induce cytokine release by human immune cells was assessed ex vivo. Whole blood samples from healthy human donors (N=10) were stimulated with test articles in soluble and wet-coated (plate-bound) formats in the absence of tumor cells.

For most whole blood donors, no cytokine (e.g., IL-2, IL-6, IL-10, TNF, and IFNγ) release above untreated controls (in both formats) was observed upon PC-1-SP cleaved or PC-1 in vitro treatment. Immune cells from the whole blood of a single donor released IL-6 in response to the highest concentration of PC-1-SP cleaved and the second-highest concentration of PC-1 in the wet-coated plate format but not in the soluble format. Another donor showed the release of IL-6, IL-10, and TNF in response to the highest concentration(s) of PC-1 tested in the soluble format but not in the wet-coated plate format. Although cytokine induction was observed in the blood samples from two donors at the highest concentration tested (100 nM), this concentration is approximately 600× higher than the projected maximum observed concentration (Cₘₐₓ) at the proposed FIH starting dose (see, Section 4.5 [titled “First-in-Human Dose Justification”).

### 4.2.2 In Vivo Pharmacology

### 4.2.2.1 Anti-Tumor Activity in a Mouse Model of CRC

Anti-tumor activity of PC-1 was evaluated in a mouse model of human CRC. Immunocompromised mice bearing HCT116 human colorectal tumor and engrafted with human PBMCs were treated with PC-1-Histag, PC-1-NC, or PC-1-T-cell engager (TCE). The in vivo tumor growth inhibition data highlights that PC-1-Histag anti-tumor activity is dose- and cleavage-dependent, whereas its non-cleavable version (PC-1-NC) was inactive. This preclinical data supports the requirement of protease activity in a human tumor to enable cleavage and anti-tumor activity of PC-1 in vivo.

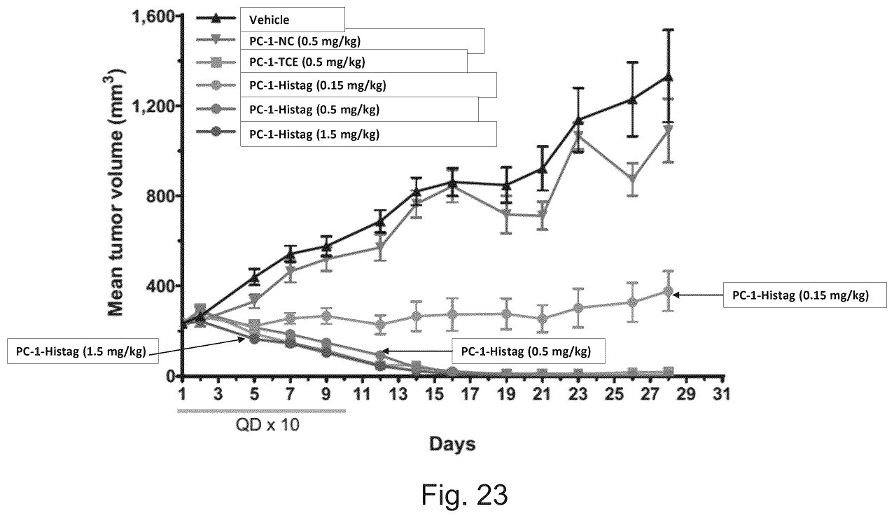

FIG. 23 illustrates the mean tumor volume in HCT116 tumor-bearing mice that were co-engrafted with human PBMCs and administered Vehicle, PC-1-NC at a dose of 0.5 mg/kg, PC-1-TCE at a dose of 0.5 mg/kg, and PC-1-Histag at doses of 0.15 mg/kg, 0.5 mg/kg, and 1.5 mg/kg. Tumor activated T cell engager (TRACTr), PC-1-Histag, and non-masked PC-1-TCE demonstrate comparable anti-tumor activity, while the non-cleavable TRACTr, PC-1-NC, is inactive. Abbreviations: NC=non-cleavable; PBMC=peripheral blood mononuclear cells; QD=once daily; TCE=T cell engager; TRACTr=tumor activated T cell engager.

### 4.2.3 Safety Pharmacology

Cardiovascular, CNS, and respiratory safety pharmacology endpoints were incorporated into a 4-week, repeat-dose, IV toxicity study in cynomolgus monkeys (see, Section 4.4.2).

### 4.2.3.1 Central Nervous System

There were no functional effects on the CNS in the repeat-dose toxicity study at PC-1 doses of ≤0.6 mg/kg/week based on daily clinical observations and detailed weekly examinations (Section 4.4.2).

### 4.2.3.2 Respiratory System

There were no functional effects on the CNS in the repeat-dose toxicity study at PC-1 doses of ≤0.6 mg/kg/week based on daily clinical observations and detailed weekly examinations (Section 4.4.2).

### 4.2.3.3 Cardiovascular System

There were no effects of PC-1 on qualitative ECG parameters nor on QRS or heart rate corrected QT intervals at PC-1 doses of ≤0.6 mg/kg/week (Section 4.4.2). No abnormalities in rhythm or waveform morphology were observed.

There was increased incidence of animals with minimally increased heart rate (derived from RR intervals) at ≥0.2 mg/kg/dose when compared to individual pretreatment baselines and mean control values at 1 to 3 hr post-end of infusion (EOI) on Days 1 and 22. The increased heart rate was generally associated with minimally shortened mean RR, PR, and QT intervals at ≥0.2 mg/kg/dose. These changes were not observed following a 4-week recovery period. There were no PC-1-related changes in the QRS or heart rate-corrected QT intervals. The minimally increased heart rate on Day 1 was likely secondary to cytokine release and not considered a direct electrophysiologic effect of PC-1. The minimal QT interval shortening was observed contemporaneously with decreased RR interval (increased heart rate) and likely represented a normal adaptive physiologic response and not a direct electrophysiologic effect of PC-1. Consistent with this, there was no change in heart rate-corrected QT interval.

The QRS interval was decreased at 1 to 3 hours post-EOI on Day 22 at 0.05 mg/kg/dose when compared to controls. This change was not considered PC-1-related due to the low magnitude and the lack of similar changes at the higher dose groups.

### 4.2.3.4 Drug Interaction Studies

### 4.3 Pharmacokinetics and Product Metabolism in Animals

PK and PD of PC-1 was evaluated in a series of nonclinical in vivo studies in cynomolgus monkeys following single or repeated IV administration. Relevant studies completed in the PC-1 nonclinical PK/TK program are summarized in Table 21 and below.

TABLE 21
Overview of PC-1 Pharmacokinetic
Studies in Cynomolgus Monkeys

| | Route of | |
|---|---|---|
| | Adminis- | Dose |
| Study | tration | (mg/kg) |
| A Single Dose Intravenous Pharmacokinetic | IV bolus | 0.1, 0.3, 1.0 |
| and Pharmacodynamic Study of PC-1 with a | | mg/kg, |
| 28-Day Observation Period in Cynomolgus | | single dose |
| Monkeys | | |
| A Single Dose Intravenous Pharmacokinetic | 30 min IV | 0.05, 0.2, and |
| and Pharmacodynamic Study of PC-1with a | Infusion | 0.6 mg/kg |
| 21-Day Observation Period in Cynomolgus | | single dose |
| Monkeys | | |
| A 4-Week Toxicity Study of PC-1 by | 30 min IV | 0.05, 0.2, and |
| Intravenous Infusion in Cynomolgus | Infusion | 0.6 mg/kg/dose |
| Monkeys with a 4-Week Recovery Period | | QW × 5 |

IV = intravenous; QW = once weekly.

### 4.3.1 Pharmacokinetics and Pharmacodynamics Studies in Cynomolgus Monkeys

4.3.1.1 Single Dose Intravenous Pharmacokinetic and Pharmacodynamic Study of PC-1 with a 28-Day Observation Period in Cynomolgus Monkeys

In cynomolgus monkeys, the pharmacokinetic profile of PC-1 was determined after administration of a single IV dose at dose levels of 0.1, 0.3, and 1 mg/kg (3 animals/dose level, total of 9 male animals). Blood samples were collected, and plasma extracted for evaluation of the pharmacokinetic parameters at the following time points: pre-dose (0), 0.083, 0.5, 1, 2, 4, 8, 12, 24, 48, 120, 168, 216, 336, 408, 552, and 672 hours post-dose. Two analytes, PC-1 and PC-1-TCE, were measured. However, individual animal PC-1-TCE plasma concentrations were all below the lower limit of quantitation.

Following a single IV bolus dose, mean Cₘₐₓ and AUC₀₋₂₁₆ₕ values increased with increase in dose. Mean Cₘₐₓ increased in a more than dose-proportional manner between doses of 0.1 and 0.3 mg/kg and was dose-proportional between 0.3 and 1 mg/kg. Mean AUC₀₋₂₁₆ₕ was more than dose-proportional between doses of 0.1 and 0.3 mg/kg and was less than dose-proportional between doses of 0.3 and 1 mg/kg.

The mean Cₘₐₓ of PC-1 following a single IV injection to cynomolgus monkeys was 1620, 8150, and 27200 ng/mL at doses of 0.1, 0.3, and 1 mg/kg, respectively. The mean half-life of PC-1 was estimated to be 88.8, 101, and 79.0 hours at doses of 0.1, 0.3, and 1 mg/kg, respectively. Mean clearance for PC-1 ranged from 2.5 to 4.47 mL/hour after a single administration of PC-1. Mean volume of distribution values ranged from 372 to 576 mL for PC-1.

In this study, one animal (animal 3002) at the 1 mg/kg dose level was sacrificed early (after 24 hours). The animal that was sacrificed early (24 hours) in the 1 mg/kg group had the highest observed Cₘₐₓ as well as AUC₀₋₂₄ₕ.

4.3.1.2 Single Dose Intravenous Pharmacokinetic and Pharmacodynamic Study of PC-1 with a 21-Day Observation Period in Cynomolgus Monkeys

In female cynomolgus monkeys, the pharmacokinetic profile of PC-1 was determined after administration of a single IV dose at dose levels of 0.05, 0.2, and 0.6 mg/kg (3 animals/dose level, 9 animals total). Blood samples were collected, and plasma was extracted for evaluation of the pharmacokinetic parameters at the following time points: pre-dose (0), 0.083, 1, 2, 4, 12, 24, 48, 72, 96, 168, 336, and 504 hours post-dose. Two analytes, PC-1 and PC-1-TCE, were measured. However, individual animal PC-1-TCE plasma concentrations were all below the lower limit of quantitation.

Following a single 30-minute IV infusion of PC-1, mean Cₘₐₓ, AUClast, AUC₀₋₁₆₈ₕ, and AUCinf values for PC-1 increased with increasing dose. Mean Cₘₐₓ of PC-1 increased with increasing dose in an approximately dose-proportional manner. Mean AUC₀₋₁₆₈ₕ increased with increasing dose in a less than dose-proportional manner from 0.05 to 0.2 mg/kg and was approximately dose-proportional from 0.2 to 0.6 mg/kg. PC-1 was quantifiable up to 168 or 336 hours post-EOI at 0.05 mg/kg, up to 336 hours post-EOI at 0.2 mg/kg, and up to 336 or 504 hours post-EOI at 0.6 mg/kg.

The mean maximum observed concentration (Cₘₐₓ) of PC-1 following a single IV injection to cynomolgus monkeys was 1170, 5950, and 17300 ng/mL at doses of 0.05, 0.2, and 0.6 mg/kg, respectively. The mean PC-1 clearance values were 0.504, 0.762, and 0.650 mL/hr/kg at 0.05, 0.2, and 0.6 mg/kg, respectively. Mean PC-1 volume of distribution (Vz) values were 58.8, 75.7, and 88.9 mL/kg at 0.05, 0.2, and 0.6 mg/kg, respectively. Mean PC-1 t1/2 values were 81.3, 68.2, and 96.5 hours at 0.05, 0.2, and 0.6 mg/kg, respectively.

4.3.1.3 A 4-Week Toxicity Study of PC-1 by Intravenous Infusion in Cynomolgus Monkeys with a 4-Week Recovery Period

A total of 32 (16/sex) cynomolgus monkeys were randomly assigned into one control group (5/sex, Group 1) and 3 test article-treated groups (3/sex/group for Groups 2 and 3, 5/sex for Group 4) in this study. Animals in the test article-treated groups were administered PC-1 by 30 minutes IV infusion once weekly at 0.05, 0.2, or 0.6 mg/kg/dose for a total of 5 doses (Days 1, 8, 15, 22, and 29). Animals in the control group were dosed for a total of 5 doses once weekly with the vehicle only. In control and 0.6 mg/kg/dose, 2 animals/sex from each group were assessed for a 4-week recovery period.

After once-weekly IV infusions of PC-1 at 0.05, 0.2, or 0.6 mg/kg/dose to male and female monkeys for a total of 5 doses, the toxicokinetic profiles of PC-1 and PC-1-TCE were measured. All measured PC-1-TCE levels were below the lower limit of quantification with the exception of one sample with measurable concentration close to the detection limit of the assay.

No marked gender differences in systemic exposure were observed at 0.05, 0.2, and 0.6 mg/kg/dose on Day 1. On Day 22 and Day 29, exposures could not be compared between the two genders due to the presence of ADA and its impact on the PC-1 TK profile. Following five weekly 30-minute IV infusions of PC-1 to cynomolgus monkeys at doses of 0.05, 0.2, and 0.6 mg/kg, mean Cₘₐₓ, mean AUC₀₋₂₄ₕ, and mean AUC₀₋₁₆₈ₕ after Day 1 dose increased in a nearly dose-proportional manner. However, mean Cₘₐₓ, mean AUC₀₋₂₄ₕ, and/or mean AUC₀₋₁₆₈ₕ of Days 22 and 29 dose increased in less than dose-proportional manner due to ADA presence in 3 out of 6 animals at 0.05 mg/kg dose, 6 out of 6 animals in 0.2 mg/kg dose (5 out of 6 animals on Day 22), and 10 out of 10 animals at 0.6 mg/kg dose. No marked accumulation was observed in ADA-negative animals at 0.05 mg/kg/dose (Group 2) and at 0.2 mg/kg/dose (Group 3). The mean Cₘₐₓ of PC-1 following the Day 1 IV infusion to cynomolgus monkeys was 1870, 6180, and 16100 ng/mL at doses of 0.05, 0.2, and 0.6 mg/kg, respectively. The mean PC-1 (sexes combined) AUC₀₋₁₆₈ₕ were 106000, 325000, and 775000 hr*ng/mL at 0.05, 0.2, and 0.6 mg/kg, respectively.

### 4.3.3 Metabolism

Classical drug metabolic elimination does not represent an important clearance mechanism for monoclonal antibodies. Antibodies such as PC-1 are generally catabolized into small peptides, carbohydrates, and amino acids, which are returned to the nutrient pool or excreted via the kidneys without any biological effects.

### 4.3.4 Excretion

Renal elimination is relatively unimportant for monoclonal antibodies, as their large size limits the extent of their glomerular filtration.

### 4.3.5 Pharmacokinetic Drug Interactions

In general, antibodies such as PC-1 are not metabolized by CYP enzymes or transported by Pgp or related adenosine triphosphate-binding cassette membrane transporters. Cytokines produced by activated lymphocytes may impact the levels of Pgp and the activity of CYP enzymes. The clinical relevance of PC-1 immune modulation and potential cytokine production that could impact Pgp and CYP is unknown, but a clinically relevant drug-drug interaction effect is considered highly unlikely.

### 4.4 Toxicology

PC-1 was evaluated in a 4-week once-weekly repeat-dose IV toxicity study in cynomolgus monkeys (dosing at days 1, 8, 15, 22, and 29) as summarized in Table 22. Consistent with the intended clinical route of administration, the toxicity study was conducted using the IV route of administration. The cynomolgus monkey was selected as the pharmacologically relevant species because nearly-equivalent binding of PC-1 to the target antigens (EGFR, CD3, and albumin) in cynomolgus monkeys and humans was observed (Section 4.2.1.2). However, minimal to no binding to mouse and rat antigens was observed (Section 4.2.1.2). The weekly dosing regimen used in the definitive 4-week repeat-dose monkey toxicity study was selected based on the half-life of PC-1 in monkeys and was designed to have a similar or more intensive dosing regimen than the clinical dosing regimen.

PC-1 was also evaluated for cytokine release in vitro (Sections 4.2.1.5.1 and 4.2.1.5.2) and in vivo (Section 4.2.2.1) and for serum stability (Section 4.2.1.3). The IV route of exposure was selected for the in vivo studies since it is the intended route of clinical exposure.

TABLE 22
Overview of the PC-1 Toxicology Program

| | |
|---|---|
| Study | Concentrations or Doses |

Repeat-Dose Toxicity

| | |
|---|---|
| A 4-week toxicity study of PC-1 by | 0, 0.05, 0.2, 0.6 |
| intravenous infusion in cynomolgus | mg/kg/dose |
| monkeys with a 4-week recovery period | |

IV = intravenous.

The NOAEL in the definitive 4-week repeat-dose monkey study was determined to be 0.6 mg/kg/dose (the highest dose tested). Transient clinical signs considered secondary to cytokine release were observed after PC-1 administration on Day 1 at 0.6 mg/kg/dose. PC-1-related clinical chemistry and hematology changes associated with an acute phase response were observed at ≥0.05 mg/kg/dose. IL-10 increases at³ 0.2 mg/kg/dose and IL-6 and IFNγ increases at 0.6 mg/kg/dose were observed after PC-1 administration on Day 1. Transient decreases in the absolute count of T cells and natural killer cells were observed at ≥0.05 mg/kg/dose. Most changes noted during the study were observed post the first dose and returned to pre-dose levels before the next dose. Following a 4-week recovery period, any remaining minor changes were fully reversible. None of the findings were considered adverse due to the transient nature of the changes, minor severity, and/or the lack of correlative microscopic changes. The NOAEL was determined to be 0.6 mg/kg/dose, the highest dose tested. Systemic exposure (Cₘₐₓ and AUC₀₋₁₆₈ₕ) to PC-1 on Day 1 at the NOAEL was 16100 ng/mL and 775000 hr*ng/mL, respectively, sexes combined.

PC-1 induced concentration- and dose-dependent cytokine release in vitro in the presence of tumor cells and in vivo in cynomolgus monkeys. In vivo, cytokine release was primarily observed after the first dose and correlated with clinical signs and clinical chemistry changes.

The stability of PC-1 was assessed in the serum from cynomolgus monkeys, healthy humans, and cancer patients. PC-1 was shown to be stable in human serum, with minimal cleavage. An increase in in vitro cleavage rate per day was observed in monkey serum, but the cleavage rate was still considered low overall in this species (Section 4.2.1.3).

### 4.4.1 Single-Dose Tolerability

In the first study, monkeys (3 males/group) were administered a single IV bolus of PC-1 at 0.1, 0.3, or 1 mg/kg.

One male animal (Animal 3002) administered 1 mg/kg was euthanized in extremis on Dosing Phase Day 2 (approximately 24 hours post-Day 1 dose). The animal did not exhibit any significant clinical signs on the day of dosing. However, by approximately 21 to 22 hours after dose administration, the animal was observed to have moderate to severe clinical signs, including decreased activity, reduced response to stimulation, somnolence (excessive drowsiness or sleeping), hunched posture, and recumbent position. Following veterinary consultation, additional observations included an extremely low body temperature and dehydration. Based on the clinical signs, the animal was euthanized for humane reasons. PC-1-related microscopic findings included minimal or mild lymphocyte necrosis and/or decreased cellularity in the germinal centers were noted in the thymus, spleen, and mesenteric lymph node and may have been due to a direct effect of PC-1 administration or secondary to a stress response. Mild acute hepatocyte necrosis in the subcapsular region was suggestive of an indirect secondary effect related to morbidity because direct test article-related liver toxicity usually has a zonal pattern. Lung/bronchus findings of minimal alveolar edema were likely secondary to the moribund condition (e.g., cardiovascular collapse/shock). Animal 3002 was observed with increases in IL-6, IL-2, IL-5, IL-10, and IFNγ with peaks between 4 hours and 24 hours post-dose. This animal showed high concentrations of IL-6 as early as 4 hours post-dose that remained high through 24 hours post-dose before being euthanized. In addition, the euthanized animal had the highest observed Cₘₐₓ and AUC₀₋₂₄ₕ for PC-1 in this dose group. PC-1-TCE levels in this animal were below the lower limit of quantification. The cause of the moribund condition was not determined from necropsy or histopathology findings; however, increased serum cytokine concentrations were suggestive of cytokine release syndrome as a potential cause.

A PC-1-related decrease in qualitative food consumption in all animals at ≥0.1 mg/kg was observed between Dosing Phase Days 1 and 4. However, there were no PC-1-related effects on body weights at ≥0.1 mg/kg. One of the surviving animals from the 1.0 mg/kg dose group was observed with mild reduced activity at 24 hours and 72 hours post-dose but recovered the following day without any medical treatment.

In the second study, monkeys (3 females/group) were administered a single 30-minute IV infusion of PC-1 at 0.05, 0.2, and 0.6 mg/kg.

All animals survived to the scheduled release from the study on Day 22. PC-1-related effects included acute, reversible clinical observations, increases in C-reactive protein (CRP) levels and/or cytokines at all dose levels. PC-1-related clinical observations at all dose levels included hunched posture, decreased activity, and elevated body temperature. The clinical signs were likely related to the observed cytokine release. One animal in each dose group was administered dexamethasone due to the severity of the clinical signs. Clinical signs resolved with or without dexamethasone treatment by 24 hours post-EOI. On Day 5 post-EOI, one animal administered 0.6 mg/kg PC-1 was observed with moderately inflamed mammary glands, purulent discharge and bleeding, and mild peri-anal ulceration. The animal was treated with diphenhydramine, Baytril, dermal gel, and gentamicin spray. The symptoms persisted until the end of the study; it is unclear if the changes in this animal observed were PC-1 treatment-related.

Increased CRP was observed 24 hours post-dose at all dose levels and were attributed to an acute phase response/inflammation that correlated with the clinical signs and cytokine induction. PC-1-related, dose-dependent increases in IL-10 were observed at ≥0.2 mg/kg between 2 and 24 hours post-EOI. Dose-dependent increases in IL-6 were also observed at 0.6 mg/kg between 2 and 8 hours post-EOI. Increases in IL-10 and IL-6 were transient, peaked between 2 and 8 hours post-EOI, and returned to baseline levels between 24 and 48 hours post-EOI.

### 4.4.2 Repeat-Dose Toxicity

A GLP repeat-dose toxicity study of 4 weeks duration (once-weekly dosing) was conducted with PC-1 in cynomolgus monkeys. PC-1 was administered by 30-minute IV infusion to male and female monkeys (3/sex/group) at doses of 0 (vehicle control), 0.05, 0.2, and 0.6 mg/kg/dose. Additional animals (2/sex/group) treated at 0 and 0.6 mg/kg/dose were assessed after a 4-week recovery period for the reversibility of any PC-1-related effects. Toxicokinetic and ADA data for this study are reported in Section 4.3.1.

All animals survived until the scheduled necropsy. There were no PC-1-related changes in body weights, ophthalmology, qualitative electrocardiology evaluation (rhythm, waveform morphology, or apparent functional changes), QRS or heart rate-corrected QT intervals, body temperature, coagulation or urinalysis parameters, organ weights, or macroscopic or microscopic examinations.

### Clinical Signs

PC-1-related, non-adverse clinical signs included red skin (facial/generalized) observed between Days 2 and 22 at ≥0.2 mg/kg/dose. PC-1-related findings at 0.6 mg/kg/dose included emesis, liquid feces, dehydration, reduced appetite, hunched posture, decreased activity, weakness, pale skin, low blood glucose, and/or increased incidence of animals with minimally increased heart rate (also noted at 0.2 mg/kg/dose) that were generally observed after the first dose and were likely related to the observed cytokine release. Generalized dry skin was observed in two animals between Days 7 and 14 at 0.6 mg/kg/dose.

### Clinical Pathology

PC-1-related non-adverse changes in hematology parameters included minimally to mildly decreased red blood cell mass, minimally decreased reticulocytes and platelets, and changes in leukocytes (decreased lymphocytes, monocytes, basophils, and large unstained cells; and increased eosinophils) at ≥0.05 mg/kg/dose. The decreases in reticulocytes, platelets, and leukocytes were observed on Day 2, 24 hours post the first dose infusion, and at subsequent time points (Days 8, 15, and 31), values approximated or exceeded control and/or pre-study values. PC-1-related changes in clinical chemistry parameters included an acute phase response consisting of minimal to moderate decreases in albumin and cholesterol and minimal to moderate increases in CRP and globulins at ≥0.05 mg/kg/dose from Days 2 to 31, minimal increases in total bilirubin at 0.6 mg/kg/dose on Day 2, and minimally increased urea nitrogen and creatinine and minimally decreased sodium and chloride in 2 individual animals at 0.6 mg/kg/dose on Day 2.

### Cytokine Level

PC-1-related dose-dependent increases in IL-6 and IFNγ concentrations were observed at 0.6 mg/kg/dose and peaked at 4 to 8 hours post the first dose. IL-6 and IFNγ concentrations returned to the baseline level 24 hours post-dose. Dose-dependent increases in IL-10 concentrations were observed at ≥0.2 mg/kg/dose and peaked between 2 and 8 hours post the first dose. IL-10 concentrations returned to baseline 24 hours post-first dose. The increases in IL-10, IL-6, and IFNγ concentrations were transient and mainly observed after the first dose. There were no PC-1-related changes in IL-2 or TNF concentrations.

### T/B/Natural Killer Cell Immunophenotyping

Immunophenotyping changes consisted of transient, generally dose-dependent decreases in the absolute counts of CD8+ and CD4+ T lymphocytes and dose-independent decreases in the absolute counts of natural killer cells and B lymphocytes that were observed at 24 hours post each dose at³ 0.05 mg/kg/dose. In general, the absolute count of these immune subsets trended toward pre-dose levels prior to the subsequent doses and the changes were lower in magnitude after subsequent doses. Transient changes in absolute counts and/or percentages of CD25+ and Ki-67+ natural killer cells and T lymphocytes were also observed at ≥0.2 mg/kg/dose. Absolute counts and percentages of CD25+ cell subsets and Ki67+ natural killer cell subsets returned to the pre-dose levels prior to the next dose and/or by 24 hours post the Day 29 dose. Absolute counts and percentages of Ki67+ T lymphocytes trended toward pre-dose levels but remained slightly elevated through 24 hours post the Day 29 dose.

### Recovery

Following a 4-week recovery period, any remaining changes observed in measured study parameters at 0.6 mg/kg/dose were fully reversible.

### Impact of Anti-Drug Antibodies

Anti-PC-1 antibodies were detected in all PC-1 dosing groups, and the incidence of ADA was dose-dependent (3/6 animals at 0.05 mg/kg/dose, 6/6 animals at 0.2 mg/kg/dose, and 10/10 animals at 0.6 mg/kg/dose) (Section 4.3.1.3). Due to ADA, many animals at ≥0.2 mg/kg/week did not maintain exposure through the end of the dosing phase, with most animals showing a substantive exposure loss by the third or fourth dose. However, one of the three ADA-positive animals in the 0.05 mg/kg dose group sustained substantial exposure after Days 22 and 29 doses with slightly lower Cₘₐₓ, AUC₀₋₂₄ₕ, or AUC₀₋₁₆₈ₕ when compared to Day 1 dose exposure. Two of the six ADA-positive animals in the 0.2 mg/kg dose group sustained substantial exposure after Day 22 dose with slightly lower AUC₀₋₂₄ₕ, AUC₀₋₁₆₈ₕ, and Cₘₐₓ compared to Day 1 dose exposure. In addition, 5 of the 10 ADA-positive animals in the 0.6 mg/kg dose group also sustained some exposure as measured by AUC₀₋₂₄ₕ, AUC₀₋₁₆₈ₕ, and Cₘₐₓ after Day 22; however, exposures were substantially lower compared to Day 1. There were no differences in toxicity findings in animals that maintained exposure at ≥0.2 mg/kg/dose versus animals that did not maintain exposure. Therefore, the assessment of potential toxicity in this study at all dose levels was considered valid.

### CONCLUSIONS

In conclusion, administration of PC-1 by once-weekly intravenous infusion for 4 weeks was tolerated in cynomolgus monkeys at 0.05, 0.2, and 0.6 mg/kg/dose. Transient clinical signs considered secondary to cytokine release were observed after PC-1 administration on Day 1 at 0.6 mg/kg/dose. PC-1-related clinical chemistry and hematology changes associated with an acute phase response were observed at ≥0.05 mg/kg/dose. IL-10 increase at ≥0.2 mg/kg/dose and IL-6 and IFNγ increase at 0.6 mg/kg/dose were observed after PC-1 administration on Day 1. Transient decreases in the absolute count of T cells and natural killer cells were observed at ≥0.05 mg/kg/dose. Most changes noted during the study were observed post the first dose and returned to the pre-dose level before the next dose. Following a 4-week recovery period, any remaining minor changes were fully reversible. None of the findings were considered adverse due to the transient nature of the changes, minor severity, and/or the lack of correlative microscopic changes. The NOAEL for PC-1 at 0.6 mg/kg/dose, the highest dose tested. Systemic exposure (Cₘₐₓ and AUC₀₋₁₆₈ₕ) to total PC-1 on Day 1 at the NOAEL was 16100 ng/mL and 775000 hr*ng/mL, respectively, sexes combined.

### 4.4.3 Genotoxicity and Carcinogenicity

In accordance with ICH S6(R1) and ICH S9 guidelines, genotoxicity and carcinogenicity studies have not been conducted with PC-1 and are not planned.

### 4.4.4 Reproductive and Developmental Toxicity

Reproductive and developmental toxicity studies, including juvenile animal studies, have not been conducted with PC-1.

There were no organ weight, macroscopic, or microscopic findings observed in the male or female reproductive organs of monkeys after administration of ≤0.6 mg/kg/dose PC-1 (Section 4.4.2).

The full potential for PC-1-related reproductive effects could not be fully assessed because the animals were not sexually mature (males were all sexually immature and females were mostly peripubertal).

### 4.4.5 Local Tolerance

No formal studies evaluating local tolerance have been performed.

Procedure-related findings were observed at the infusion site at terminal necropsy and consisted of minimal to mild hemorrhage and mixed cell inflammation in the 4-week repeat-dose toxicity study in monkeys (Section 4.4.2). The inflammation was characterized by perivascular to interstitial infiltrates of lymphocytes, plasma cells, and histiocytes with fewer neutrophils amid small bands of plump fibroblasts and edema. These findings occurred in control and treated animals and fully reversed by the end of the 4-week recovery period.

### 4.4.6 Cytokine Release

PC-1 induced concentration- or dose-dependent cytokine release (i.e., IL-6, IL-10, and/or IFNγ) in vitro in the presence of tumor cells and in vivo in normal monkeys with no tumors (Sections 4.2.1.5.1, 4.2.1.5.2, and 4.2.2.1). In vivo, cytokine release was primarily observed after the first dose and correlated with clinical signs and clinical chemistry changes. In vitro and in vivo cytokine release data were taken into account when determining the proposed clinical starting dose, as discussed in Section 4.2.1.5.2.

### 4.4.7 Serum Stability

The stability of PC-1 was assessed in the serum from cynomolgus monkeys, healthy humans, and cancer patients. Results from these assays are discussed in Section 4.2.1.3. PC-1 was shown to be stable in human serum with minimal cleavage. An increase in in vitro cleavage rate per day was observed in monkey serum, but the cleavage rate was still considered low overall in this species.

### 4.5 First-in-Human Dose Justification

The proposed starting dose in the FIH study is 50 μg, administered once weekly. The selection of the starting dose and regimen was based on a minimally anticipated biologic effect level (MABEL) approach integrating pharmacokinetic and pharmacodynamic data, including in vitro activity and in vivo safety data (Saber et al, 2017). The starting dose was calculated based on the expected Cₘₐₓ of PC-1 in humans translated from cynomolgus monkey PK studies and the most conservative PC-1 half-maximal effective concentration (EC50) derived from an in vitro cytotoxicity assay using an EGFR-expressing SCCHN tumor cell line co-cultured with human PBMCs. This PK-guided approach was used to compare the predicted human doses whose Cₘₐₓ matched the PC-1 EC50 from in vitro cytotoxicity studies as well as the Cₘₐₓ from the cynomolgus monkey GLP toxicity study (Table 16). Compared to the 2 rig/mL PC-1 Cₘₐₓ from the low dose (0.05 mg/kg) group in the GLP toxicity study where minimal to no cytokines were induced, the 142 ng/mL in vitro cytotoxicity PC-1 EC50 was more conservative. The proposed FIH dose includes an additional 10× safety factor to the 500 ug dose, which is based on the most conservative in vitro cytotoxicity assay, to further ensure safety. Using the additional 10× safety factor, the calculated FIH dose for PC-1 is 50 μg.

To ensure that PK, pharmacodynamic, and safety data are fully characterized, a starting dose of 50 μg once-weekly administration is selected, which is 10 times lower than MABEL EC50-based dose.

TABLE 23
Maximum Concentrations of PC-1 in Repeat-dose NHP GLP Toxicity Study
and in Human In Vitro Assays with Projected Human Equivalent Doses

| | | | | | Approximate | | |
|---|---|---|---|---|---|---|---|
| | | | | | Human Equivalent | | |
| | Cₘₐₓ Concentration | | | | Dose (Flat Dose) | | |
| | | | Weight/ | | Needed to Achieve | | |
| Assay/Study | Molar | | Volume | | Cₘₐₓ | | Comment |
| Repeat-dose | 161 | nM | 16 | μg/ml \hat{ } | 56 | mg | NOAEL from |
| cynomolgus monkey | | | | | | | cynomolgus monkey |
| GLP toxicity study: | | | | | | | GLP toxicity study |
| NOAEL, 0.6 mg/kg | | | | | | | |
| Repeat-dose | 63 | nM | 6 | μg/ml | 21 | mg | |
| cynomolgus monkey | | | | | | | |
| GLP toxicity study: | | | | | | | |
| Minimal to no cytokines | | | | | | | |
| induced, 0.2 mg/kg | | | | | | | |
| Repeat-dose | 20 | nM | 2 | μg/ml | 7 | mg | Dose associated with |
| cynomolgus monkey | | | | | | | minimal to no |
| GLP toxicity study: | | | | | | | cytokines |
| Minimal to no cytokines | | | | | | | |
| induced, 0.05 mg/kg | | | | | | | |
| TRACTr in vitro | 1.49 | nM | 142 | ng/ml | 500 | μg | MABEL based FIH |
| MABEL (EC₅₀)* | | | | | | | dose (EC50) |
| Most sensitive cell line | | | | | | | |
| tested | | | | | | | |
| 10x below MABEL | 149 | pM | 14 | ng/ml | 50 | μg | Proposed FIH dose |
| EC50 | | | | | | | |

Abbreviations: Cₘₐₓ = maximum drug concentration; EC50 = half-maximal effective concentration; FIH = first-in-human; GLP = Good Laboratory Practice; MABEL = minimal anticipated biological effect level; NHP = nonhuman primate; NOAEL = no observed adverse effect level; PBMC = peripheral blood mononuclear cell; SCCHN = squamous cell carcinoma of head and neck; TRACTr = tumor activated T cell engager.
*MABEL EC50 based on in vitro cytotoxicity using the most sensitive SCCHN tumor cell line tested co-cultured with human PBMCs.
\hat{ }Average observed Cₘₐₓ from the first dose of GLP tox study

### First-In-Human Study of PC-1

### 5-Effects in Humans

### 5.1 Introduction

There is no clinical experience with PC-1. The current study will be the FIH Phase 1 clinical trial of PC-1. For complete patient eligibility criteria, please see the clinical study protocol. A brief summary of a planned clinical study is provided below.

### 5.1.1 Study Design

Aa first-in-human (FIH), Phase 1/lb, open-label, multicenter dose escalation and dose expansion study to assess the safety, tolerability, PK, PD, and preliminary anti-tumor activity of PC-1 in adult subjects with histologically confirmed advanced or metastatic CRC, NSCLC, SCCHN and RCC will be conducted.

The study will be conducted in 3 parts: Dose Escalation (Part 1) with approximately 40 to 50 subjects, Cohort Backfill Expansion (Part 2) with up to approximately 40 subjects enrolled across 4 dose levels, and Dose Expansion (Part 3) with up to approximately 40 subjects enrolled at the RP2D.

Dose Escalation (Part 1) will assess the safety, tolerability, PK, PD, and preliminary efficacy of PC-1 administered by IV infusion.

Cohort Backfill Enrichment (Part 2) will allow for further characterization of safety and activity of dose levels. Dose Expansion (Part 3) will determine additional safety, tolerability, PK, PD, and preliminary clinical activity data with PC-1 at a dose and schedule to be determined by the Safety Review Committee after reviewing all available safety, PK, PD, and preliminary efficacy data. Depending on the data, randomization may be integrated for either 2 different PR2D doses or 2 different treatment intervals.

### 6-Summary of Data and Guidance

### 6.1 Indication

PC-1 is in development for the treatment of advanced or metastatic solid tumors that are unresponsive to currently available therapies. PC-1 is currently not approved for any indication.

### 6.2 Dosage and Administration

In the FIH study, the starting dose will be 50 μg, to be followed by dose escalation. PC-1 will be administered IV on Days 1, 8, and 15 of 21-day cycles. Different dosing intervals may be considered based on evolving PK, PD, safety, and efficacy data.

### 6.3 Dosage Forms and Strengths

The PC-1 DP will be provided as a solution for injection for IV administration at a single strength presentation of 2 mg/mL.

### 6.4 Contraindications

PC-1 should not be administered to patients who have had allergic or anaphylactic reactions to any component of PC-1. No other contraindications for PC-1 are currently known.

### 6.5 Warnings and Precautions

There has been no prior clinical experience with PC-1. PC-1 is an experimental drug that should be administered only to patients within the context of a clinical study.

PC-1 is an antibody fragment based bispecific protein construct. Like other molecules in this class, it is highly specific for its targets. Although antibody therapeutics are well-tolerated, they are ‘foreign’ proteins, and some patients may experience infusion-related reactions or develop an immune response against them.

### 6.6 Potential Adverse Reactions

The adverse reaction profile of PC-1 is unknown. However, safety measures should be considered based on nonclinical data and the mechanism of action.

Experience with other protein based biological therapies indicates that pyrexia, immunogenicity reactions (i.e., formation of ADAs), and/or hypersensitivity reactions may be observed. These reactions can be both serious and systemic (e.g., anaphylaxis) and may occur acutely or be delayed.

Because PC-1 is a T cell redirecting bispecific antibody, activation of T cell may induce CRS, neurotoxicity, and/or tumor lysis syndrome may occur.

Because PC-1 targets EGFR, adverse events reported with other EGFR targeting agents, such as cetuximab or panitumumab, may be observed. These risks include cardiac toxicity, pulmonary fibrosis or interstitial lung disease, dermatologic and soft tissue toxicities, photosensitivity, and ocular toxicity (ERBITUX® USPI; VECTIBIX® USPI).

### 6.6.1 Infusion-Related Reactions

PC-1 is a recombinant protein based therapeutic, and administration of therapeutic proteins has been associated with infusion reactions with symptoms and signs including fever, rigors, rash, urticaria, dyspnea, hypotension, and/or nausea. To minimize the risk of infusion reactions, premedication with acetaminophen (or paracetamol) and an anti-histamine regimen should be administered during Cycle 1 and per standard institutional practice prior to administration of each dose of PC-1 afterwards as tolerated.

### 6.6.2 Cytokine Release Syndrome

The identified risks of treatment with TCEs are primarily related to cytokine release and CRS. PC-1 is designed to reduce the risk of CRS by requiring protease cleavage for activation, focusing molecular activity to the TME where proteases are overexpressed, dysregulated, and activated. This approach has been shown to markedly reduce systemic cytokine exposure in preclinical models. However, CRS is a potential adverse reaction based on the mechanism of action of PC-1.

Symptoms associated with CRS vary greatly and may be difficult to distinguish from other conditions. The more common symptoms include fever, tachycardia, hypotension, hypoxia, fatigue, nausea, headache, dyspnea, rigors, myalgia/arthralgia, and anorexia. The severity of symptoms can be mild to life-threatening and thus, there should be a high suspicion for CRS if these symptoms occur. Grade 1 CRS according to the American Society for Transplantation and Cellular Therapy consensus grading scale does not require any intervention, but subjects should be monitored closely. Grade ≥2 CRS requires PC-1 dosing interruption and prompt symptomatic treatment per local standard institutional practice.

Preventive measures of cytokine release and CRS will include glucocorticoid premedication, IV pre-hydration, and holding of anti-hypertensive medication on day of the first infusion. Priming and step dosing will be initiated if CRS is seen during dose escalation.

### 6.6.3 Other Potential Toxicities

### 6.6.3.1 Tumor Lysis Syndrome

Subjects with a high disease burden may be at risk for developing tumor lysis syndrome (TLS) with PC-1 treatment. Prophylactic treatment/measures are strongly recommended for subjects considered to be at risk for TLS, per institutional or clinical standards. Subjects should be closely monitored for laboratory evidence of TLS (hyperuricemia, hyperkalemia, hyperphosphatemia, and hypocalcemia).

In the case of evidence of TLS associated with PC-1, subjects will be admitted to the hospital, as clinically indicated. Standard management will include vigorous IV hydration, hypouricemic agents, and correction of acidosis, if present. Renal function, serum uric acid, calcium, phosphorus, and electrolytes should be closely monitored.

Subjects with Grade 3 to 4 TLS during Week 1 or Cycle 1 may also be hospitalized for ≥24 hours after the end of the administration of the subsequent dose, with considerations for dose reduction as described in the study protocol.

6.6.3.2 Neurological Events Administration of solid tumor-targeted bispecifics has shown less potential for severe neurologic toxicity (compared with chimeric antigen receptor T cell therapy or anti-CD19 BiTE format antibodies such as blinatumomab) in early-stage clinical studies. Although PC-1 at active doses has not shown a propensity for induction of cytokine levels that may be associated with neurologic toxicity in in vitro human cell cultures or in vivo cynomolgus monkey studies, the risk of neurotoxicity is unknown and caution is warranted.

Neurological events associated with cytokine release may include tremor, mental status changes, confusion, speech difficulties, and potentially seizures. Monitor subjects for neurological events and exclude other causes for neurological symptoms. Neurological events should be graded according to the National Cancer Institute Common Terminology Criteria for Adverse Events (CTCAE) version 5.0. Mild disorientation or expressive aphasia (trouble word-finding) may be the earliest and most specific signs. Provide supportive care as needed for any neurological events. Workup may include head magnetic resonance imaging and electroencephalogram and may require corticosteroids, or anti-seizure medications, if severe. The medical monitor should be contacted if there is any potentially treatment-related neurological toxicity.

### 6.6.3.3 Infections

As seen with other TCE-based immunotherapies, serious infections, including fatal bacterial, fungal, and new or reactivated viral infections, may occur during and/or following the completion of PC-1-based therapy. New or reactivated viral infections may include cytomegalovirus, herpes simplex virus, parvovirus B19, varicella zoster virus, West Nile virus, hepatitis B virus (HBV), and hepatitis C virus. PC-1 should be discontinued if serious infections develop, and appropriate anti-infective therapy instituted. PC-1 is not recommended for use in subjects with severe, active infections.

### 6.6.3.4 Hepatitis B Reactivation

Hepatitis B virus reactivation can occur in patients treated with drugs classified as TCE. Cases have been reported in patients who are hepatitis B surface antigen (HBsAg)-negative but are hepatitis B core antibody (anti-HBc)-positive.

HBV reactivation is defined as an abrupt increase in HBV replication manifesting as a rapid increase in serum HBV DNA levels or detection of HBsAg in a person who was previously HBsAg-negative and anti-HBc-positive. Reactivation of HBV replication is often followed by hepatitis (i.e., increase in transaminase levels). In severe cases, increase in bilirubin levels, liver failure, and death can occur.

It is recommended to monitor subjects with evidence of prior HBV infection (anti-HBc-positive) with HBV DNA testing monthly and for clinical and laboratory signs of hepatitis during and for several months following PC-1 therapy.

### 6.6.3.5 Immunization

The safety of immunization with live viral vaccines during or following PC-1 therapy has not been studied. Vaccination with live virus vaccines is not recommended for ≥2 weeks prior to the start of PC-1 treatment, during treatment, and until immune recovery following the last cycle of PC-1.

### 6.6.4 Toxicities for Compounds Targeting EGFR

While not observed in preclinical studies with PC-1, cardiac, pulmonary, skin, and ocular toxicities have been observed with other compounds targeting EGFR. Subjects should be informed of these toxicities observed with other compounds targeting EGFR.

### 6.6.4.1 Cardiac Toxicity

Cardiac toxicity, including cardiac arrest, has been observed with other compounds targeting EGFR. Periodic echocardiograms and electrocardiograms will be conducted together with monitoring troponin I and brain natriuretic peptide as early signs of toxicity. In addition, electrolyte abnormalities should be carefully monitored.

### 6.6.4.2 Pulmonary Toxicity

Events of interstitial lung disease or pulmonary fibrosis have been observed with other compounds targeting EGFR. Subjects should be advised to report any suggestive symptoms, such as dyspnea or cough.

### 6.6.4.3 Skin Toxicity

Clinical manifestations of dermatologic and soft skin tissue toxicity, including but not limited to, acneiform dermatitis, pruritus, erythema, rash, skin exfoliation, paronychia, dry skin, and skin fissure, have been observed with other compounds targeting EGFR. Subjects should be monitored not only for infusion site reactions but also any skin lesions. Dermatological toxicity may be exacerbated by exposure to sunlight. Subjects should be advised to wear sunscreen and hats to limit sun exposure.

### 6.6.4.4 Ocular Toxicity

Clinical manifestations of ocular toxicity including eye inflammation, lacrimation, light sensitivity, blurred vision, eye pain and/or red eye have been observed with other compounds targeting EGFR. Subjects should be informed about potential ocular toxicity and should be further examined at the first sign of possible ocular toxicity.

### 6.7 Drug Interactions

The drug interaction profile of PC-1 is unknown, however, in general, antibodies such as PC-1 are not metabolized by CYP enzymes or transported by Pgp or related adenosine triphosphate-binding cassette membrane transporters. Cytokines produced by activated lymphocytes may impact the levels of Pgp and the activity of CYP enzymes. The clinical relevance of PC-1 immune modulation and potential cytokine production that could impact Pgp and CYP is unknown, but a clinically relevant drug-drug interaction effect is considered highly unlikely. Caution needs to be paid where subjects are receiving substrates of CYP enzymes with narrow therapeutic index window. Subject should be monitored closely and have doses for the concomitant treatments adjusted as necessary.

### 6.8 Use in Specific Populations

### 6.8.1 Fertility, Pregnancy, and Lactation

Nonclinical studies evaluating the effect of PC-1 on embryo-fetal development or reproductive parameters have not been conducted. Therefore, pregnant women and those breastfeeding will be excluded. Men and women who are biologically capable of having children must agree to commit to the use of highly effective methods of contraception as outlined in the study protocol.

### 6.8.2 Geriatric Use

No studies in geriatric patients have been conducted. Elderly patients will be included in the initial clinical trials of PC-1. Elderly patients should, however, be carefully monitored.

### 6.8.3 Renal Impairment

PK/functional alterations due to renal impairment are not anticipated.

### 6.10 Clinical Studies

No prior clinical studies of PC-1 have been conducted.

### 6.11 Reference Safety Information for Assessment of Expectedness of Serious Adverse Reactions

For regulatory reporting purposes, all adverse events (AEs) will be assessed as being unexpected at this stage of the development program. Therefore, AEs that are serious and possibly related to PC-1 will be reported to the health authorities per applicable regulations.

While certain embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EMBODIMENTS

Embodiment 1 An isolated recombinant polypeptide complex comprising a first chain with an amino acid sequence having at least 70% sequence identity to SEQ ID NO: 1 and a second chain with an amino acid sequence having at least 70% sequence identity to SEQ ID NO: 2 wherein the isolated recombinant polypeptide complex comprises at least one of the following characteristics:
(a) at least one N-glycan moiety;
(b) at least one disulfide bond;
(c) a melting onset temperature (T_{Onset}) between about 60° C. to about 65° C. and a transition mid-point temperature (Tₘₗ) between about 70° C. and about 75° C. when the isolated recombinant polypeptide complex is formulated at a concentration of about 1.0 mg/mL in about 10 mM histidine, about 8% (w/v) sucrose, about 0.01% (w/v) polysorbate 20 (PS20) pH of about 5.3, wherein the T_{Onset} and the Tₘₗ are measured using differential scanning calorimetry (DSC);
(d) a far UV circular dichroism dip at a wavelength between 210 nm and 230 nm when the isolated recombinant polypeptide complex is formulated at a concentration of about 0.1 mg/mL in water; or
(e) a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

Embodiment 2 The isolated recombinant polypeptide complex of embodiment 1, wherein the polypeptide complex comprises at least two of the characteristics.

Embodiment 3 The isolated recombinant polypeptide complex of embodiment 1, wherein the polypeptide complex comprises at least three of the characteristics.

Embodiment 4 The isolated recombinant polypeptide complex of embodiment 1, wherein the polypeptide complex comprises at least four of the characteristics.

Embodiment 5 The isolated recombinant polypeptide complex of embodiment 1, wherein the polypeptide complex comprises at least five of the characteristics.

Embodiment 6 The isolated recombinant polypeptide complex according to any one of embodiments 1-5, wherein the first chain comprises at least 75% sequence identity to SEQ ID NO: 1.

Embodiment 7 The isolated recombinant polypeptide complex according to any one of embodiments 1-5, wherein the first chain comprises at least 80% sequence identity to SEQ ID NO: 1.

Embodiment 8 The isolated recombinant polypeptide complex according to any one of embodiments 1-5, wherein the first chain comprises at least 85% sequence identity to SEQ ID NO: 1.

Embodiment 9 The isolated recombinant polypeptide complex according to any one of embodiments 1-5, wherein the first chain comprises at least 90% sequence identity to SEQ ID NO: 1.

Embodiment 10 The isolated recombinant polypeptide complex according to any one of embodiments 1-5, wherein the first chain comprises at least 95% sequence identity to SEQ ID NO: 1.

Embodiment 11 The isolated recombinant polypeptide complex according to any one of embodiments 1-5, wherein the first chain comprises at least 99% sequence identity to SEQ ID NOs: 1.

Embodiment 12 The isolated recombinant polypeptide complex according to any one of embodiments 1-5, wherein the first chain comprises the amino acid sequence according to SEQ ID NO: 1.

Embodiment 13 The isolated recombinant polypeptide complex according to any one of embodiments 1-5, wherein the second chain comprises at least 75% sequence identity to SEQ ID NO: 2.

Embodiment 14 The isolated recombinant polypeptide complex according to any one of embodiments 1-5, wherein the second chain comprises at least 80% sequence identity to SEQ ID NO: 2.

Embodiment 15 The isolated recombinant polypeptide complex according to any one of embodiments 1-5, wherein the second chain comprises at least 85% sequence identity to SEQ ID NO: 2.

Embodiment 16 The isolated recombinant polypeptide complex according to any one of embodiments 1-5, wherein the second chain comprises at least 90% sequence identity to SEQ ID NO: 2.

Embodiment 17 The isolated recombinant polypeptide complex according to any one of embodiments 1-5, wherein the second chain comprises at least 95% sequence identity to SEQ ID NO: 2.

Embodiment 18 The isolated recombinant polypeptide complex according to any one of embodiments 1-5, wherein the second chain comprises at least 99% sequence identity to SEQ ID NO: 2.

Embodiment 19 The isolated recombinant polypeptide complex according to any one of embodiments 1-5, wherein the second chain comprises the amino acid sequence according to SEQ ID NO: 2.

Embodiment 20 The isolated recombinant polypeptide complex of embodiment 1, wherein the at least one N-glycan moiety is located on the first chain.

Embodiment 21 The isolated recombinant polypeptide complex of embodiment 1, wherein the at least one N-glycan moiety is located on the second chain.

Embodiment 22 The isolated recombinant polypeptide complex of embodiment 1, wherein the at least one N-glycan moiety comprises G2F, G2FS1, or G2FS2.

Embodiment 23 The isolated recombinant polypeptide complex of embodiment 1, wherein the at least one N-glycan moiety comprises G2F.

Embodiment 24 The isolated recombinant polypeptide complex of embodiment 1, wherein the at least one N-glycan moiety comprises G2FS1.

Embodiment 25 The isolated recombinant polypeptide complex of embodiment 1, wherein the at least one N-glycan moiety comprises G2FS2.

Embodiment 26 The isolated recombinant polypeptide complex of embodiment 1, wherein at least one asparagine deamidation moiety is located at Asparagine 83 of SEQ ID NO: 1.

Embodiment 27 The isolated recombinant polypeptide complex of embodiment 1, wherein the at least one N-glycan moiety is located at Asparagine 519 of SEQ ID NO: 2.

Embodiment 28 The isolated recombinant polypeptide complex of embodiment 1, wherein the isolated recombinant polypeptide complex further comprises O-xylosylation, asparagine deamidation, or succinimide formation.

Embodiment 29 The isolated recombinant polypeptide complex of embodiment 26 or 28, wherein the succinimide formation is located at Asparagine 83 of SEQ ID NO: 1.

Embodiment 30 The isolated recombinant polypeptide complex according to any one of the above embodiments, wherein the polypeptide complex comprises at least two disulfide bonds formed by pairs of cysteine residues.

Embodiment 31 The isolated recombinant polypeptide complex according to any one of the above embodiments, wherein the polypeptide complex comprises at least three disulfide bonds formed by pairs of cysteine residues.

Embodiment 32 The isolated recombinant polypeptide complex according to any one of the above embodiments, wherein the polypeptide complex comprises at least four disulfide bonds formed by pairs of cysteine residues.

Embodiment 33 The isolated recombinant polypeptide complex according to any one of the above embodiments, wherein the polypeptide complex comprises at least five disulfide bonds formed by pairs of cysteine residues.

Embodiment 34 The isolated recombinant polypeptide complex according to any one of the above embodiments, wherein the polypeptide complex comprises at least six disulfide bonds formed by pairs of cysteine residues.

Embodiment 35 The isolated recombinant polypeptide complex according to any one of the above embodiments, wherein the polypeptide complex comprises at least seven disulfide bonds formed by pairs of cysteine residues.

Embodiment 36 The isolated recombinant polypeptide complex according to any one of the above embodiments, wherein the polypeptide complex comprises at least eight disulfide bonds formed by pairs of cysteine residues.

Embodiment 37 The isolated recombinant polypeptide complex according to any one of the above embodiments, wherein the polypeptide complex comprises at least nine disulfide bonds formed by pairs of cysteine residues.

Embodiment 38 The isolated recombinant polypeptide complex according to any one of the above embodiments, wherein the polypeptide complex comprises at least ten disulfide bonds formed by pairs of cysteine residues.

Embodiment 39 The isolated recombinant polypeptide complex according to any one of the above embodiments, wherein the pair of cysteine residues comprises Cysteine 4 and Cysteine 15 of SEQ ID NO: 1.

Embodiment 40 The isolated recombinant polypeptide complex according to any one of the above embodiments, wherein the pair of cysteine residues comprises Cysteine 65 and Cysteine 130 of SEQ ID NO: 1.

Embodiment 41 The isolated recombinant polypeptide complex according to any one of the above embodiments, wherein the pair of cysteine residues comprises Cysteine 176 and Cysteine 236 of SEQ ID NO: 1.

Embodiment 42 The isolated recombinant polypeptide complex according to any one of the above embodiments, wherein the pair of cysteine residues comprises Cysteine 256 of SEQ ID NO: 1, and Cysteine 653 of SEQ ID NO: 2.

Embodiment 43 The isolated recombinant polypeptide complex according to any one of the above embodiments, wherein the pair of cysteine residues comprises Cysteine 22 and Cysteine 96 of SEQ ID NO: 2.

Embodiment 44 The isolated recombinant polypeptide complex according to any one of the above embodiments, wherein the pair of cysteine residues comprises Cysteine 138 and Cysteine 148 of SEQ ID NO: 2.

Embodiment 45 The isolated recombinant polypeptide complex according to any one of the above embodiments, wherein the pair of cysteine residues comprises Cysteine 199 and Cysteine 275 of SEQ ID NO: 2.

Embodiment 46 The isolated recombinant polypeptide complex according to any one of the above embodiments, wherein the pair of cysteine residues comprises Cysteine 339 and Cysteine 407 of SEQ ID NO: 2.

Embodiment 47 The isolated recombinant polypeptide complex according to any one of the above embodiments, wherein the pair of cysteine residues comprises Cysteine 453 and Cysteine 526 of SEQ ID NO: 2.

Embodiment 48 The isolated recombinant polypeptide complex according to any one of the above embodiments, wherein the pair of cysteine residues comprises Cysteine 577 and Cysteine 633 of SEQ ID NO: 2.

Embodiment 49 The isolated recombinant polypeptide complex according to any one of the above embodiments, wherein the polypeptide complex comprises 1 inter-chain disulfide bonds between the first chain and the second chain and 9 intra-chain disulfide bonds.

Embodiment 50 The isolated recombinant polypeptide complex according to any one of the above embodiments, wherein the polypeptide complex comprises 1 inter-chain disulfide bonds between the first chain and the second chain and the second chain comprises 6 intra-chain disulfide bonds and the first chain comprises 3 intra-chain disulfide bonds.

Embodiment 51 The isolated recombinant polypeptide complex according to any one of the above embodiments, having a T_{Onset} between about 61° C. to about 64.5° C.

Embodiment 52 The isolated recombinant polypeptide complex according to any one of the above embodiments, having a T_{Onset} between about 62° C. to about 64° C.

Embodiment 53 The isolated recombinant polypeptide complex according to any one of the above embodiments, having a T_{Onset} of about 62.5° C.

Embodiment 54 The isolated recombinant polypeptide complex according to any one of the above embodiments, having a Tₒₙₛₑₜ of about 63.2° C.

Embodiment 55 The isolated recombinant polypeptide complex according to any one of the above embodiments, having a Tₘₗ between about 71° C. to about 75° C.

Embodiment 56 The isolated recombinant polypeptide complex according to any one of the above embodiments, having a Tₘₗ between about 72.5° C. to about 74.5° C.

Embodiment 57 The isolated recombinant polypeptide complex according to any one of the above embodiments, having a Tₘₗ of about 73.8° C.

Embodiment 58 The isolated recombinant polypeptide complex according to any one of the above embodiments, having a Tₘₗ of about 74.0° C.

Embodiment 59 The isolated recombinant polypeptide complex according to any one of the above embodiments wherein the secondary structure composition comprises a β-sheet and a random coil.

Embodiment 60 The isolated recombinant polypeptide complex according to any one of the above embodiments, having a far UV circular dichroism dip at a wavelength between 215 nm and 225 nm.

Embodiment 61 The isolated recombinant polypeptide complex according to any one of the above embodiments, having a far UV circular dichroism dip at a wavelength between 215 nm and 220 nm.

Embodiment 62 The isolated recombinant polypeptide complex according to any one of the above embodiments, having a near UV circular dichroism dip at a wavelength between 280 nm and 290 nm.

Embodiment 63 The isolated recombinant polypeptide complex according to any one of the above embodiments, having a near UV circular dichroism dip at a wavelength between 280 nm and 285 nm.

Embodiment 64 The isolated recombinant polypeptide complex according to any one of the above embodiments, having a near UV circular dichroism peak at a wavelength between 270 nm and 275 nm.

Embodiment 65 The isolated recombinant polypeptide complex according to any one of the above embodiments, having a near UV circular dichroism peak at a wavelength between 285 nm and 290 nm.

Embodiment 66 The isolated recombinant polypeptide complex according to any one of the above embodiments, wherein the first chain comprises the amino acid sequence according to SEQ ID NO: 1, and the second chain comprises the amino acid sequence according to SEQ ID NO: 2, and the at least one N-glycan moiety comprises G2F, G2FS1, or G2FS2, and the recombinant polypeptide complex comprises disulfide bonds formed by pairs of cysteine residues Cysteine 4 and Cysteine 15 of SEQ ID NO: 1, Cysteine 65 and Cysteine 130 of SEQ ID NO: 1, Cysteine 176 and Cysteine 236 of SEQ ID NO: 1, Cysteine 256 of SEQ ID NO: 1 and Cysteine 653 of SEQ ID NO: 2, Cysteine 138 and Cysteine 148 of SEQ ID NO: 2, Cysteine 22 and Cysteine 96 of SEQ ID NO: 2, Cysteine 199 and Cysteine 275 of SEQ ID NO: 2, Cysteine 339 and Cysteine 407 of SEQ ID NO: 2, Cysteine 453 and Cysteine 526 of SEQ ID NO: 2, and Cysteine 577 and Cysteine 633 of SEQ ID NO: 2, and the Tₒₙₛₑₜ is between about 60° C. to about 65° C. and a transition mid-point temperature (Tₘₗ) between about 70° C. and about 75° C., wherein the T_{Onset} and the Tₘₗ are measured using differential scanning calorimetry (DSC), when the isolated recombinant polypeptide complex is formulated at a concentration of about 1.0 mg/mL in about 10 mM histidine, about 8% (w/v) sucrose, about 0.01% (w/v) PS20 pH of about 5.3, and a far UV circular dichroism dip at a wavelength between 210 nm and 230 nm when the isolated recombinant polypeptide complex is formulated at a concentration of 0.1 mg/mL in water, and a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

Embodiment 67 An isolated recombinant polypeptide complex comprising a first chain with an amino acid sequence having at least 80% sequence identity to SEQ ID NO: 1 and a second chain with an amino acid sequence having at least 80% sequence identity to SEQ ID NO: 2 wherein the isolated recombinant polypeptide complex comprises the following characteristics:
(a) at least one N-glycan moiety;
(b) at least one disulfide bond;
(c) a melting onset temperature (T_{Onset}) between about 60° C. to about 65° C. and a transition mid-point temperature (Tₘₗ) between about 70° C. and about 75° C., wherein the T_{Onset} and the Tₘₗ are measured using differential scanning calorimetry (DSC), when the isolated recombinant polypeptide complex is formulated at a concentration of about 1.0 mg/mL in about 10 mM histidine, about 8% (w/v) sucrose, about 0.01% (w/v) PS20 pH of about 5.3;
(d) a far UV circular dichroism dip at a wavelength between 210 nm and 230 nm when the isolated recombinant polypeptide complex is formulated at a concentration of about 0.1 mg/mL in water; or
(e) a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

Embodiment 68 A pharmaceutical composition comprising:
(a) the isolated recombinant polypeptide complex according to any one of embodiments 1-67; and
(b) a pharmaceutically acceptable carrier.

Embodiment 69 The pharmaceutical composition of embodiment 68, wherein the pharmaceutically acceptable carrier comprises a buffer, a stabilizing agent, a tonicity agent, a surfactant, or combinations thereof.

Embodiment 70 The pharmaceutical composition of embodiment 68, wherein the buffer comprises an amino acid or a derivative thereof.

Embodiment 71 The pharmaceutical composition of embodiment 70, wherein the amino acid or derivative thereof comprises L-histidine, L-histidine hydrochloride monohydrate, or a combination thereof.

Embodiment 72 The pharmaceutical composition of embodiment 68, wherein the surfactant is polysorbate 20.

Embodiment 73 The pharmaceutical composition of embodiment 68, wherein the stabilizing agent is sucrose.

Embodiment 74 The pharmaceutical composition of embodiment 68, having a pH less than 6.0.

Embodiment 75 A method of treating cancer comprising administering to a subject in need thereof the isolated recombinant polypeptide complex or pharmaceutical composition of any one of the above embodiments.

Embodiment 76 The method of embodiment 75, wherein the isolated recombinant polypeptide or pharmaceutical composition is administered once weekly.

Embodiment 77 The method of embodiment 76, wherein the isolated recombinant polypeptide or pharmaceutical composition is administered as a continuous infusion.

Embodiment 78 The method of embodiment 75, wherein the isolated recombinant polypeptide or pharmaceutical composition is administered over a period of no more than 60 minutes.

Embodiment 79 The method of embodiment 75, wherein the isolated recombinant polypeptide or pharmaceutical composition is administered over a period of no more than 30 minutes.

Embodiment 80 The method of embodiment 75, wherein the isolated recombinant polypeptide or pharmaceutical composition is administered over a period of no more than 10 minutes.

Embodiment 81 The method of embodiment 75, wherein the isolated recombinant polypeptide or pharmaceutical composition is administered by intravenous, intramuscular, intralesional, topical, subcutaneous, infusion or oral administration.

Embodiment 82 The method of embodiment 75, wherein the isolated recombinant polypeptide or pharmaceutical composition is administered once weekly as a bolus injection, an IV infusion over 10 minutes to 120 minutes, or a subcutaneous administration.

Embodiment 83 The isolated recombinant polypeptide complex of any one of embodiments 1-67, wherein the isolated recombinant polypeptide complex provides a maximum plasma concentration (Cmax) in a subject after a single intravenous bolus administration to the subject of a dose of about 0.1 milligram per kilogram of the body weight (mg/kg) to about 1 mg/kg, wherein the Cmax increases when the dose increases.

Embodiment 84 The isolated recombinant polypeptide complex of embodiment 83, wherein an increase of the Cmax is proportional to an increase of the dose.

Embodiment 85 The isolated recombinant polypeptide complex of embodiment 83, wherein an increase of the Cmax is more than a value that is proportional to an increase of the dose.

Embodiment 86 The isolated recombinant polypeptide complex of embodiment 83, wherein an increase of the area under the drug concentration versus time curve between 0 hour (h) and 216 h after the administration (AUC0₋₂₁₆ₕ) is more than a value that is proportional to an increase of the dose.

Embodiment 87 The isolated recombinant polypeptide complex of embodiment 83, wherein an increase of AUC₀₋₂₁₆ₕ is less than a value that is proportional to an increase of the dose being administered.

Embodiment 88 The isolated recombinant polypeptide complex of embodiment 83, wherein the subject has the highest Cmax within 24 h after the administration.

Embodiment 89 The isolated recombinant polypeptide complex of embodiment 83, wherein the subject has the highest AUC₀₋₂₄ₕ at 24 h after the administration.

Embodiment 90 The isolated recombinant polypeptide complex of embodiment 83, wherein the dose is about 0.1 mg/kg.

Embodiment 91 The isolated recombinant polypeptide complex of embodiment 83, wherein the dose is about 0.3 mg/kg.

Embodiment 92 The isolated recombinant polypeptide complex of embodiment 83, wherein the dose is about 1 mg/kg.

Embodiment 93 The isolated recombinant polypeptide complex of embodiment 84, wherein the dose is about 0.3 mg/kg to about 1 mg/kg.

Embodiment 94 The isolated recombinant polypeptide complex of embodiment 85, wherein the dose is about 0.1 mg/kg to about 0.3 mg/kg.

Embodiment 95 The isolated recombinant polypeptide complex of embodiment 86, wherein the dose is about 0.1 mg/kg to about 0.3 mg/kg.

Embodiment 96 The isolated recombinant polypeptide complex of embodiment 87, wherein the dose is about 0.3 mg/kg to about 1 mg/kg.

Embodiment 97 The isolated recombinant polypeptide complex of any one of embodiments 83-96, wherein the isolated recombinant polypeptide complex provides a half maximum plasma concentration (T_{1/2}) in the subject at about 88.8 h to about 101 h.

Embodiment 98 The isolated recombinant polypeptide complex of any one of embodiments 1-67, wherein the isolated recombinant polypeptide complex is administered to a subject through a single intravenous infusion over about 30 minutes (min).

Embodiment 99 The isolated recombinant polypeptide complex of embodiment 98, wherein an increase of the Cmax is proportional to an increase of the dose.

Embodiment 100 The isolated recombinant polypeptide complex of embodiment 98, wherein an increase of the area under the curve between 0 h and 168 h after the administration (AUC0-₁₆₈ₕ) is less than a value that is proportional to an increase of the dose.

Embodiment 101 The isolated recombinant polypeptide complex of embodiment 98, wherein an increase of AUC₀₋₁₆₈ₕ is proportional to an increase of the dose.

Embodiment 102 The isolated recombinant polypeptide complex of embodiment 98, wherein the dose is about 0.05 mg/kg.

Embodiment 103 The isolated recombinant polypeptide complex of embodiment 98, wherein the dose is about 0.2 mg/kg.

Embodiment 104 The isolated recombinant polypeptide complex of embodiment 98, wherein the dose is about 0.6 mg/kg.

Embodiment 105 The isolated recombinant polypeptide complex of embodiment 100, wherein the dose is about 0.05 mg/kg to about 0.2 mg/kg.

Embodiment 106 The isolated recombinant polypeptide complex of embodiment 101, wherein the dose is about 0.2 mg/kg to about 0.6 mg/kg.

Embodiment 107 The isolated recombinant polypeptide complex of any one of embodiments 98-106, wherein the isolated recombinant polypeptide complex provides a T_{1/2} at about 68.2 h to about 96.5 h.

Embodiment 108 The isolated recombinant polypeptide complex of any one of embodiments 98-106, wherein the isolated recombinant polypeptide complex provides a T_{1/2} at about 81.3 h at a dose of about 0.05 mg/kg.

Embodiment 109 The isolated recombinant polypeptide complex of any one of embodiments 98-105, wherein the isolated recombinant polypeptide complex provides a T1_{/2} at about 68.2 h at a dose of about 0.2 mg/kg being administered.

Embodiment 110 The isolated recombinant polypeptide complex of any one of embodiments 98-105, wherein the isolated recombinant polypeptide complex provides a T1/2 at about 96.5 h at a dose of about 0.6 mg/kg.

Embodiment 111 The isolated recombinant polypeptide complex of embodiment 98, wherein the isolated recombinant polypeptide complex is administered once weekly.

Embodiment 112 The isolated recombinant polypeptide complex of embodiment 111, wherein the isolated recombinant polypeptide complex is administered for at least 1 week, 2 weeks, 3 weeks, 4 weeks, 5 weeks, 6 weeks, 7 weeks, 8 weeks, 9 weeks, or 10 weeks.

Embodiment 113 The isolated recombinant polypeptide complex of embodiment 111 or 112, wherein an increase of the Cmax is proportional to an increase of the dose after day 1, wherein day 1 is the day of the administration.

Embodiment 114 The isolated recombinant polypeptide complex of embodiment 111 or 112, wherein an increase of the AUC₀₋₂₄ₕ is proportional to an increase of the dose after day 1, wherein day 1 is the day of the administration.

Embodiment 115 The isolated recombinant polypeptide complex of embodiment 111 or 112, wherein an increase of the AUC₀₋₁₆₈ₕ is proportional to an increase of the dose after day 1, wherein day 1 is the day of the administration.

Embodiment 116 The isolated recombinant polypeptide complex of any one of embodiments 111-115, wherein the dose is about 0.05 mg/kg.

Embodiment 117 The isolated recombinant polypeptide complex of any one of embodiments 111-115, wherein the dose is about 0.2 mg/kg.

Embodiment 118 The isolated recombinant polypeptide complex of any one of embodiments 111-115, wherein the dose is about 0.6 mg/kg.

Embodiment 119 The isolated recombinant polypeptide complex of any one of embodiments 1-67, wherein the isolated recombinant polypeptide complex is not metabolized by a cytochrome P450 (CYP) enzyme.

Embodiment 120 The isolated recombinant polypeptide complex of any one of embodiments 1-67, wherein the isolated recombinant polypeptide complex is not transported by P-glycoprotein (Pgp) or a related adenosine triphosphate-binding cassette membrane transporter.

Embodiment 121 The isolated recombinant polypeptide complex of any one of embodiments 83-120, wherein the isolated recombinant polypeptide complex results in release of cytokine in the subject.

Embodiment 122 The isolated recombinant polypeptide complex of embodiment 121, wherein the cytokine is interleukin 6 (IL-6), interleukin 10 (IL-10), interferon γ (IFNγ), tumor necrosis factor (TNF), or a combination thereof.

Embodiment 123 The isolated recombinant polypeptide complex of embodiment 122, wherein the cytokine release is correlated with the dose.

Embodiment 124 The isolated recombinant polypeptide complex of embodiment 123, wherein IL-10 release occurs at a dose no less than 0.2 mg/kg.

Embodiment 125 The isolated recombinant polypeptide complex of embodiment 123, wherein IL-6 release occurs at a dose no less than 0.6 mg/kg.

Embodiment 126 The isolated recombinant polypeptide complex of embodiment 123, wherein IFNγ release occurs at a dose no less than 0.6 mg/kg.

Embodiment 127 The isolated recombinant polypeptide complex of any one of embodiments 1-67, wherein the isolated recombinant polypeptide complex provides a T1/2 of about 79 h to about 101 h after administration of a single dose at about 0.05 mg/kg to about 1 mg/kg.

Embodiment 128 The isolated recombinant polypeptide complex of embodiment 127, wherein the isolated recombinant polypeptide complex is administered to a subject through a single intravenous bolus administration.

Embodiment 129 The isolated recombinant polypeptide complex of embodiment 128, wherein the isolated recombinant polypeptide complex provides a Cmax, wherein an increase of the Cmax is proportional to an increase of the dose.

Embodiment 130 The isolated recombinant polypeptide complex of embodiment 128, wherein the isolated recombinant polypeptide complex provides an AUC, wherein an increase of the AUC is proportional to an increase of the dose.

Embodiment 131 The isolated recombinant polypeptide complex of embodiment 111, wherein the isolated recombinant polypeptide complex is administered for at least 4 weeks.

Embodiment 132 The isolated recombinant polypeptide complex of embodiment 131, wherein the no observed adverse effect level (NOAEL) is about 0.6 mg/kg.

Embodiment 133 The isolated recombinant polypeptide complex of embodiment 132, wherein the isolated recombinant polypeptide complex provides a Cmax of about 16100 ng/ml at day 1 at a dose of about 0.6 mg/kg, wherein day 1 is the day of the administration.

Embodiment 134 The isolated recombinant polypeptide complex of embodiment 132, wherein the isolated recombinant polypeptide complex provides an AUC₀₋₁₆₈ₕ of about 775000 hr*ng/ml at a dose of about 0.6 mg/kg administered on day 1.

Embodiment 135 The isolated recombinant polypeptide complex of embodiment 111, wherein the isolated recombinant polypeptide complex is administered for at least 4 weeks.

Embodiment 136 The isolated recombinant polypeptide complex of embodiment 135, wherein the dose is about 0.05 mg/kg, about 0.2 mg/kg, or about 0.6 mg/kg.

Embodiment 137 The isolated recombinant polypeptide complex of embodiment 135 or 136, wherein the dose is administered at days 1, 8, 15, 22, and 29.

Embodiment 138 The isolated recombinant polypeptide complex of embodiment 136 or 137, wherein the isolated recombinant polypeptide complex provides a Cmax of about 1870 ng/ml at a dose of about 0.05 mg/kg administered on day 1.

Embodiment 139 The isolated recombinant polypeptide complex of embodiment 136 or 137, wherein the isolated recombinant polypeptide complex provides a Cmax of about 6180 ng/ml at a dose of about 0.2 mg/kg administered on day 1.

Embodiment 140 The isolated recombinant polypeptide complex of embodiment 136 or 137, wherein the isolated recombinant polypeptide complex provides a Cmax of about 16100 ng/ml at a dose of about 0.6 mg/kg administered on day 1.

Embodiment 141 The isolated recombinant polypeptide complex of embodiment 136 or 137, wherein the isolated recombinant polypeptide complex provides an AUC0-168h of about 106000 hr*ng/ml at a dose of about 0.05 mg/kg administered on day 1.

Embodiment 142 The isolated recombinant polypeptide complex of embodiment 136 or 137, wherein the isolated recombinant polypeptide complex provides an AUC0-168h of about 325000 hr*ng/ml at a dose of about 0.2 mg/kg administered on day 1.

Embodiment 143 The isolated recombinant polypeptide complex of embodiment 136 or 137, wherein the isolated recombinant polypeptide complex provides an AUC0-168h of about 775000 hr*ng/ml at a dose of about 0.6 mg/kg administered on day 1.

Embodiment 144 The isolated recombinant polypeptide complex of embodiment 136 or 137, wherein the isolated recombinant polypeptide complex provides an AUC at a dose administered on day 22 that is similar to or same as an AUC at the same dose administered on day 1, wherein, after the administering on day 22, the subject exhibits no detectable level of an anti-drug antibody.

Embodiment 145 The isolated recombinant polypeptide complex of embodiment 136 or 137, wherein the isolated recombinant polypeptide complex provides an AUC at a dose administered on day 29 that is similar to or same as an AUC at the same dose administered on day 1, wherein, after the administering on day 29, the subject exhibits no detectable level of an anti-drug antibody.

Embodiment 146 The isolated recombinant polypeptide complex of embodiment 144 or 145, wherein the AUC is AUC₀₋₂₄ₕ or AUC0_{-68h.}

Embodiment 147 The isolated recombinant polypeptide complex of embodiment 136 or 137, wherein the isolated recombinant polypeptide complex provides a Cmax at a dose administered on day 22 that is similar to or same as a Cmax at the same dose administered on day 1, wherein, after the administering on day 22, the subject exhibits no detectable level of an anti-drug antibody.

Embodiment 148 The isolated recombinant polypeptide complex of embodiment 136 or 137, wherein the isolated recombinant polypeptide complex provides a Cmax at a dose administered on day 29 that is similar to or same as a Cmax at the same dose administered on day 1, wherein, after the administering on day 29, the subject exhibits no detectable level of an anti-drug antibody.

Embodiment 149 The isolated recombinant polypeptide complex of any one of embodiments 1-67, wherein the isolated recombinant polypeptide complex provides a Cmax of about 1620 ng/ml after a single intravenous bolus administration of a dose of about 0.1 mg/kg.

Embodiment 150 The isolated recombinant polypeptide complex of any one of embodiments 1-67, wherein the isolated recombinant polypeptide complex provides a Cmax of about 8150 ng/ml after a single intravenous bolus administration of a dose of about 0.3 mg/kg.

Embodiment 151 The isolated recombinant polypeptide complex of any one of embodiments 1-67, wherein the isolated recombinant polypeptide complex provides a Cmax of about 27200 ng/ml after a single intravenous bolus administration of a dose of about 1 mg/kg.

Embodiment 152 The isolated recombinant polypeptide complex of any one of embodiments 1-67, wherein the isolated recombinant polypeptide complex provides a T1/2 of about 88.8 h after a single intravenous bolus administration of a dose of about 0.1 mg/kg.

Embodiment 153 The isolated recombinant polypeptide complex of any one of embodiments 1-67, wherein the isolated recombinant polypeptide complex provides a T1/2 of about 101 h after a single intravenous bolus administration of a dose of about 0.3 mg/kg.

Embodiment 154 The isolated recombinant polypeptide complex of any one of embodiments 1-67, wherein the isolated recombinant polypeptide complex provides a T1/2 of about 79 h after a single intravenous bolus administration of a dose of about 1 mg/kg.

Embodiment 155 The isolated recombinant polypeptide complex of any one of embodiments 1-67, wherein the isolated recombinant polypeptide complex provides a clearance of about 2.5 ml/h to about 4.47 ml/h after a single intravenous bolus administration.

Embodiment 156 The isolated recombinant polypeptide complex of any one of embodiments 1-67, wherein the isolated recombinant polypeptide complex provides a volume of distribution of about 372 ml to about 576 ml after a single intravenous bolus administration.

Embodiment 157 The isolated recombinant polypeptide complex of any one of embodiments 1-67, wherein the isolated recombinant polypeptide complex provides a Cmax of about 1170 ng/ml after a single intravenous infusion of a dose of about 0.05 mg/kg over about 30 min.

Embodiment 158 The isolated recombinant polypeptide complex of any one of embodiments 1-67, wherein the isolated recombinant polypeptide complex provides a Cmax of about 5950 ng/ml after a single intravenous infusion of a dose of about 0.2 mg/kg over about 30 min.

Embodiment 159 The isolated recombinant polypeptide complex of any one of embodiments 1-67, wherein the isolated recombinant polypeptide complex provides a Cmax of about 17300 ng/ml after a single intravenous infusion of a dose of about 0.6 mg/kg over about 30 min.

Embodiment 160 The isolated recombinant polypeptide complex of any one of embodiments 1-67, wherein the isolated recombinant polypeptide complex provides a clearance of about 0.504 ml/hr/kg after a single intravenous infusion of a dose of about 0.05 mg/kg over about 30 min.

Embodiment 161 The isolated recombinant polypeptide complex of any one of embodiments 1-67, wherein the isolated recombinant polypeptide complex provides a clearance of about 0.762 ml/hr/kg after a single intravenous infusion of a dose of about 0.2 mg/kg over about 30 min.

Embodiment 162 The isolated recombinant polypeptide complex of any one of embodiments 1-67, wherein the isolated recombinant polypeptide complex provides a clearance of about 0.650 ml/hr/kg after a single intravenous infusion of a dose of about 0.6 mg/kg over about 30 min.

Embodiment 163 The isolated recombinant polypeptide complex of any one of embodiments 1-67, wherein the isolated recombinant polypeptide complex provides a volume of distribution of about 58.8 ml/kg after a single intravenous infusion of a dose of about 0.05 mg/kg over about 30 min.

Embodiment 164 The isolated recombinant polypeptide complex of any one of embodiments 1-67, wherein the isolated recombinant polypeptide complex provides a volume of distribution of about 75.7 ml/kg after a single intravenous infusion of a dose of about 0.2 mg/kg over about 30 min.

Embodiment 165 The isolated recombinant polypeptide complex of any one of embodiments 1-67, wherein the isolated recombinant polypeptide complex provides a volume of distribution of about 88.9 ml/kg after a single intravenous infusion of a dose of about 0.6 mg/kg over about 30 min.

Embodiment 166 The isolated recombinant polypeptide complex of any one of embodiments 1-67, wherein the isolated recombinant polypeptide complex provides a T_{1/2} of about 81.3 h after a single intravenous infusion of a dose of about 0.05 mg/kg over about 30 min.

Embodiment 167 The isolated recombinant polypeptide complex of any one of embodiments 1-67, wherein the isolated recombinant polypeptide complex provides a T_{1/2} of about 68.2 h after a single intravenous infusion of a dose of about 0.05 mg/kg over about 30 min.

Embodiment 168 The isolated recombinant polypeptide complex of any one of embodiments 1-67, wherein the isolated recombinant polypeptide complex provides a T_{1/2} of about 96.5 h after a single intravenous infusion of a dose of about 0.05 mg/kg over about 30 min.

Embodiment 169 The isolated recombinant polypeptide complex of any one of embodiments 83-168, wherein the subject is a primate.

Embodiment 170 The isolated recombinant polypeptide complex of any one of embodiments 83-168, wherein the subject is a non-human primate.

Embodiment 171 The isolated recombinant polypeptide complex of any one of embodiments 83-168, wherein the subject is a monkey.

Embodiment 172 The isolated recombinant polypeptide complex of any one of embodiments 83-168, wherein the subject is a cynomolgus monkey.

Embodiment 173 The isolated recombinant polypeptide complex of any one of embodiments 83-168, wherein the subject is a human.

Embodiment 174 The isolated recombinant polypeptide complex of any one of embodiments 1-67, wherein the isolated recombinant polypeptide complex degrades in vitro at a rate of about 1% per day in the serum of a subject.

Embodiment 175 The isolated recombinant polypeptide complex of any one of embodiments 1-67, wherein the isolated recombinant polypeptide complex degrades in vitro at a rate of about 2% per day in the serum of a subject.

Embodiment 176 The isolated recombinant polypeptide complex of any one of embodiments 1-67, wherein the isolated recombinant polypeptide complex degrades in vitro at a rate of about 6.6% to about 11.5% per day in the serum of a subject.

Embodiment 177 The isolated recombinant polypeptide complex of embodiment 174 or 175, wherein the subject is human.

Embodiment 178 The isolated recombinant polypeptide complex of embodiment 177, wherein the subject is healthy.

Embodiment 179 The isolated recombinant polypeptide complex of embodiment 177, wherein the subject has cancer.

Embodiment 180 The isolated recombinant polypeptide complex of embodiment 179, wherein the cancer comprises colorectal cancer (CRC), squamous cell carcinoma of head and neck (SCCHN), or non-small cell lung cancer (NSCLC).

Embodiment 181 The isolated recombinant polypeptide complex of embodiment 176, wherein the subject is cynomolgus monkey.

Embodiment 182 A method for treating cancer comprising administering to a subject in need thereof an effective amount of a pharmaceutical composition comprising:
(a) a dose of the isolated recombinant polypeptide complex of any one of embodiments 1-67; and
(b) a pharmaceutically acceptable excipient.

Embodiment 183 The method of embodiment 182, wherein the cancer comprises a cancer cell expressing epidermal growth factor receptor (EGFR).

Embodiment 184 The method of embodiment 182 or 183, wherein the cancer comprises a cancer cell overexpressing EGFR.

Embodiment 185 The method of any one of embodiments 182-184, wherein the cancer comprises CRC, SCCHN, NSCLC, renal cell carcinoma (RCC), breast cancer, pancreatic cancer, ovarian cancer, prostate cancer, brain cancer, glioblastoma multiforme, or papillary carcinoma.

Embodiment 186 The method of any one of embodiments 182-185, wherein the cancer is metastatic, refractory, or relapsed.

Embodiment 187 The method of any one of embodiments 182-186, wherein the cancer is advanced or non-advanced.

Embodiment 188 The method of any one of embodiments 182-187, wherein the cancer comprises advanced, non-advanced, refractory, relapsed or metastatic CRC, SCCHN, NSCLC, RCC, breast cancer, pancreatic cancer, ovarian cancer, prostate cancer, brain cancer, glioblastoma multiforme, or papillary carcinoma.

Embodiment 189 The method of any one of embodiments 182-188, wherein the dose is at least about 25 μg, at least about 50 μg, at least about 100 μg, at least about 150 μg, or at least about 200 ug.

Embodiment 190 The method of any one of embodiments 182-189, wherein the dose is about 25 μg, about 50 μg, about 100 μg, about 150 μg, or about 200 μg.

Embodiment 191 The method of any one of embodiments 182-190, wherein the dose comprises at least 2 doses.

Embodiment 192 The method of any one of embodiments 182-190, wherein the dose comprises at least 3 doses.

Embodiment 193 The method of any one of embodiments 182-192, wherein the dose comprises a first dose and a second dose, wherein the second dose is equal to or higher than the first dose, wherein the first dose is at least about 25 μg.

Embodiment 194 The method of any one of embodiments 182-193, wherein the dose comprises a first dose, a second dose, and a third dose, wherein the second dose is equal to or higher than the first dose, wherein the third dose is equal to or higher than the second dose, wherein the first dose is at least about 25 μg.

Embodiment 195 The method of any one of embodiments 182-194, wherein a dose of the isolated recombinant polypeptide complex is administered at least once weekly.

Embodiment 196 The method of any one of embodiments 182-194, wherein a dose of the isolated recombinant polypeptide complex is administered at least twice weekly.

Embodiment 197 The method of any one of embodiments 182-194, wherein a dose of the isolated recombinant polypeptide complex is administered at least once every two weeks.

Embodiment 198 The method of any one of embodiments 182-194, wherein a dose of the isolated recombinant polypeptide complex is administered at least once every three weeks.

Embodiment 199 The method of any one of embodiments 182-194, wherein a dose of the isolated recombinant polypeptide complex is administered at least once every four weeks.

Embodiment 200 The method of any one of embodiments 182-194, wherein a dose of the isolated recombinant polypeptide complex is administered at least once every five or more weeks.

Embodiment 201 The method of any one of embodiments 182-200, wherein the method comprises at least one 21-day treatment course.

Embodiment 202 The method of any one of embodiments 182-201, further comprising at least one 28-day treatment course.

Embodiment 203 The method of any one of embodiments 182-202, further comprising at least one 21-day treatment course and at least one 28-day treatment course.

Embodiment 204 The method of any one of embodiments 201-203, further comprising at least two, three, four, five, six, seven, eight, nine, ten, or more than ten of the 21-day treatment course.

Embodiment 205 The method of any one of embodiments 202-204, further comprising at least two, three, four, five, six, seven, eight, nine, ten, or more than ten of the 28-day treatment course.

Embodiment 206 The method of any one of embodiments 201-205, further comprising at least one of the 21-day treatment course and at least one of the 28-day treatment course.

Embodiment 207 The method of any one of embodiments 201-206, further comprising at least two of the 21-day treatment course and at least two of the 28-day treatment course.

Embodiment 208 The method of any one of embodiments 201-207, wherein a first dose is administered to the subject in the first week of the treatment course, a second dose is administered to the subject in the second week of the treatment course, and a third dose is administered to the subject in the third week of the treatment course, wherein the second dose is equal to or higher than the first dose and the third dose is equal to or higher than the second dose, wherein the first dose is at least about 25 μg.

Embodiment 209 The method of any one of embodiments 201-208, wherein a first dose and a second dose are each administered to the subject biweekly during the treatment course, wherein the second dose is equal to or higher than the first dose, wherein the first dose is at least about 25 μg.

Embodiment 210 The method of any one of embodiments 201-209, wherein a first dose and a second dose are administered to the subject in the first week of the treatment course, and a third dose is administered to the subject in the second week of the treatment course, wherein the second dose is equal to or higher than the first dose and the third dose is equal to or higher than the second dose, wherein the first dose is at least about 25 μg.

Embodiment 211 The method of any one of embodiments 201-210, wherein the first dose is administered on day 1 of the treatment course, the second dose is administered on day 8 of the treatment course, and the third dose is administered on day 15 of the treatment course.

Embodiment 212 The method of any one of embodiments 201-210, wherein the first dose is administered on day 1 of the treatment course and the second dose is administered on day 15 of the treatment course.

Embodiment 213 The method of any one of embodiments 201-210, wherein the first dose is administered on day 1 of the treatment course, the second dose is administered on day 4 of the treatment course, and the third dose is administered on day 8 of the treatment course.

Embodiment 214 The method of any one of embodiments 201-213, wherein the method comprises a first 21-day treatment course and a second 21-day treatment course, wherein the first 21-day treatment course predates the second 21-day treatment course, wherein a first dose of the second 21-day treatment course is equal to or higher than a first dose of the first 21-day treatment course, a second dose of the second 21-day treatment course is equal to or higher than a second dose of the first 21-day treatment course, and a third dose of the second 21-day treatment course is equal to or higher than a third dose of the 21-day first treatment course, wherein the first dose of the first 21-day treatment course is at least 50 μg.

Embodiment 215 The method of any one of embodiments 201-214, wherein the method comprises a first 28-day treatment course and a second 28-day treatment course, wherein the first 28-day treatment course predates the second 28-day treatment course, wherein a first dose of the second 28-day treatment course is equal to or higher than a first dose of the first 28-day treatment course, a second dose of the second 28-day treatment course is equal to or higher than a second dose of the first 28-day treatment course, wherein the first dose of the first 21-day treatment course is at least 50 μg.

Embodiment 216 The method of any one of embodiments 201-215, wherein the method comprises at least one 21-day treatment course and at least one 28-day treatment course, wherein a dose is administered on day 1, day 8, and day 15 of the 21-day treatment course, and on day 1 and day 15 of the 28-day treatment course.

Embodiment 217 The method of any one of embodiments 201-216, wherein the method comprises a first 28-day treatment course and a second 28-day treatment course, wherein a dose is administered on day 1, day 8, and day 15 of the first 28-day treatment course, and on day 1 and day 15 of the second 28-day treatment course.

Embodiment 218 The method of any one of embodiments 201-217, wherein the method comprises at least one 21-day treatment course and at least one 28-day treatment course, wherein a dose is administered on day 1, day 4, and day 8 of the 21-day treatment course, and on day 1 and day 15 of the 28-day treatment course.

Embodiment 219 The method of any one of embodiments 201-218, wherein the method comprises at least two of the treatment course, wherein the administering is suspended or paused for a period of time between two of the treatment course.

Embodiment 220 The method of any one of embodiments 182-219, wherein the administering comprises administering through intravenous infusion.

Embodiment 221 The method of embodiments 220, wherein the administering comprises administering through intravenous infusion over 30 min to 2 h.

Embodiment 222 The method of embodiments 221, wherein the administering comprises administering a mixture comprising the pharmaceutical composition and dextrose, wherein the mixture comprises about 5% (w/v) dextrose.

Embodiment 223 The method of any one of embodiments 182-222, wherein the pharmaceutical composition comprises the isolated recombinant polypeptide complex at a concentration of about 2 mg/ml.

Embodiment 224 The method of any one of embodiments 182-223, wherein the pharmaceutically acceptable excipient comprises a buffer, a stabilizing agent, a tonicity agent, a surfactant, or combinations thereof.

Embodiment 225 The method of embodiment 224, wherein the buffer comprises an amino acid or a derivative thereof.

Embodiment 226 The method of embodiment 225, wherein the amino acid or the derivative thereof comprises L-histidine, L-histidine monohydrochloride monohydrate, or combinations thereof.

Embodiment 227 The method of any one of embodiments 224-226, wherein the stabilizing agent comprises sugar.

Embodiment 228 The method of embodiment 227, wherein the sugar comprises sucrose.

Embodiment 229 The method of any one of embodiments 224-228, wherein the tonicity agent comprises sugar.

Embodiment 230 The method of embodiment 229, wherein the sugar comprises sucrose.

Embodiment 231 The method of any one of embodiments 224-230, wherein the surfactant comprises a polysorbate (PS).

Embodiment 232 The method of any one of embodiments 224-231, wherein the surfactant comprises polysorbate 20 (PS20).

Embodiment 233 The method of any one of embodiments 182-232, wherein the pharmaceutical composition comprises about 10 millimolar (mM) L-histidine in the form of L-histidine and L-histidine monohydrochloride monohydrate.

Embodiment 234 The method of any one of embodiments 182-2339, wherein the pharmaceutical composition comprises about 8% weight/volume (w/v) sucrose.

Embodiment 235 The method of any one of embodiments 182-234, wherein the pharmaceutical composition comprises about 0.01% (w/v) polysorbate 20.

Embodiment 236 The method of any one of embodiments 182-235, wherein the pharmaceutical composition comprises about 10 mM L-histidine in the form of L-histidine and L-histidine monohydrochloride monohydrate, about 8% (w/v) sucrose, and about 0.01% (w/v) polysorbate 20.

Embodiment 237 The method of any one of embodiments 182-236, wherein the pharmaceutical composition comprises a pH of about 5.3.

Embodiment 238 The method of any one of embodiments 182-237, wherein the pharmaceutical composition comprises an osmolality of about 276 mOsmol/kg.

Embodiment 239 The method of any one of embodiments 182-238, further comprising treating the subject with a therapy for an infusion-related reaction before or after the administering.

Embodiment 240 The method of any one of embodiments 182-239, further comprising treating the subject with a therapy for an infusion-related reaction before the administering of each dose of the isolated recombinant polypeptide complex.

Embodiment 241 The method of embodiment 239 or 240, wherein the therapy for an infusion-related reaction comprises acetaminophen, paracetamol, or an antihistamine drug.

Embodiment 242 The method of any one of embodiments 239-241, wherein the therapy for an infusion-related reaction comprises acetaminophen or paracetamol, and an antihistamine drug.

Embodiment 243 The method of any one of embodiments 239-242, wherein the therapy for an infusion-related reaction comprises a therapy for treating fever, rigors, rash, urticaria, dyspnea, hypotension, or nausea.

Embodiment 244 The method of any one of embodiments 182-243, further comprising treating the subject with a therapy for cytokine release syndrome (CRS) before or after the administering.

Embodiment 245 The method of embodiment 244, wherein the therapy for CRS comprises a glucocorticoid, an intravenous pre-hydration, or suspension of anti-hypertensive medication before the administering of the first dose in the method.

Embodiment 246 The method of embodiment 244 or 245, wherein the therapy for CRS comprises a therapy for fever, tachycardia, hypotension, hypoxia, fatigue, nausea, headache, dyspnea, rigors, myalgia/arthralgia, or anorexia.

Embodiment 247 The method of any one of embodiments 182-246, further comprising treating the subject with a therapy for tumor lysis syndrome (TLS) before or after the administering.

Embodiment 248 The method of embodiment 247, wherein the therapy for TLS comprises an intravenous hydration, a hypouricemic agent, or correction of acidosis, before or after the administering.

Embodiment 249 The method of embodiment 247 or 248, wherein the therapy for TLS comprises a therapy for hyperuricemia, hyperkalemia, hyperphosphatemia, or hypocalcemia.

Embodiment 250 The method of any one of embodiments 182-249, wherein the isolated recombinant polypeptide complex is cleaved by a protease to generate an enzymatic product of the isolated recombinant polypeptide complex after the administering.

Embodiment 251 The method of embodiment 250, wherein the isolated recombinant polypeptide complex is cleaved by a tumor specific protease to generate the enzymatic product of the isolated recombinant polypeptide complex after the administering.

Embodiment 252 The method of embodiment 251, wherein the tumor specific protease comprises two or more proteases.

Embodiment 253 The method of embodiment 252, wherein the isolated recombinant polypeptide complex is cleaved by a first protease of the two or more proteases to generate a first metabolic product of the isolated recombinant polypeptide complex.

Embodiment 254 The method of embodiment 252 or 253, wherein the isolated recombinant polypeptide complex is cleaved by a second protease of the two or more proteases to generate a second metabolic product of the isolated recombinant polypeptide complex.

Embodiment 255 The method of embodiment 253 or 254, wherein the first protease comprises a serine protease.

Embodiment 256 The method of embodiment 254 or 255, wherein the second protease comprises a matrix metalloprotease.

Embodiment 257 The method of embodiment 255, wherein the serine protease comprises human matriptase (MTSP1).

Embodiment 258 The method of embodiment 256, wherein the matrix metalloprotease comprises human matrix metalloprotease 9 (MMP9).

Embodiment 259 The method of any one of embodiments 253-258, wherein the first metabolic product comprises a first amino acid sequence with at least 90% sequence identity to the amino acid sequence of SEQ ID NO: 4, a second amino acid sequence with at least 90% sequence identity to the amino acid sequence of SEQ ID NO: 6, or both.

Embodiment 260 The method of any one of embodiments 253-259, wherein the second metabolic product comprises a first amino acid sequence with at least 90% sequence identity to the amino acid sequence of SEQ ID NO: 3, a second amino acid sequence with at least 90% sequence identity to the amino acid sequence of SEQ ID NO: 5, or both.

Embodiment 261 The method of any one of embodiments 250-260, wherein the isolated recombinant polypeptide complex exhibits a cancer cell killing activity that is dependent on the dose, wherein an increase in the dose of the isolated recombinant polypeptide complex results in an increase in the cancer cell killing activity.

Embodiment 262 The method of embodiment 261, wherein the isolated recombinant polypeptide complex exhibits a cancer cell killing activity that is dependent on the amount of the enzymatic product, wherein an increase in the amount of the enzymatic product results in an increase in the cancer cell killing activity.

Embodiment 263 The method of any one of embodiments 183-262, wherein the isolated recombinant polypeptide complex exhibits a cancer cell killing activity that correlates with the expression level of EGFR on the cancer cell, wherein an increase in the expression level of EGFR on the cancer cell results in an increase in the cancer cell killing activity.

Embodiment 264 The method of any one of embodiments 250-263, wherein the enzymatic product has a binding affinity with EGFR that is more than 300-fold of the binding affinity of the isolated recombinant polypeptide complex with EGFR.

Embodiment 265 The method of any one of embodiments 250-264, wherein the enzymatic product has a binding affinity with cluster of differentiation 3 (CD3) that is more than 1000-fold of the binding affinity of the isolated recombinant polypeptide complex with CD3.

Embodiment 266 The method of any one of embodiments 250-265, wherein the isolated recombinant polypeptide complex binds with:
(g) a human EGFR at a half-maximal effective concentration (EC₅₀) of about 109 nM;
(h) a human CD3 at an EC₅₀ of about 89 nM;
(i) a human albumin at an EC₅₀ of about 0.1 nM;
(j) a cynomolgus monkey EGFR at an EC₅₀ of about 126 nM;
(k) a cynomolgus monkey CD3 at an EC₅₀ of about 87 nM; and/or
(l) a cynomolgus monkey albumin at an EC₅₀ of about 0.3 nM.

Embodiment 267 The method of embodiment 266, wherein the isolated recombinant polypeptide complex binds with a mouse or rat EGFR, CD3, or albumin at an EC₅₀ that is more than 1000 fold higher than the EC₅₀ of the human or cynomolgus monkey EGFR, CD3, or albumin.

Embodiment 268 The method of embodiment 255, wherein the first metabolic product of the isolated recombinant polypeptide complex binds with:
(e) a human EGFR at an EC₅₀ of about 0.28 nM;
(f) a human CD3 at an EC₅₀ of about 0.08 nM;
(g) a cynomolgus monkey EGFR at an EC₅₀ of about 0.27 nM; and/or
(h) a cynomolgus monkey CD3 at an EC₅₀ of about 0.08 nM.

Embodiment 269 The method of embodiment 268, wherein the first metabolic product of the isolated recombinant polypeptide complex:
(f) does not bind with an albumin derived from human, cynomolgus monkey, mouse, or rat;
(g) binds with mouse EGFR at an EC₅₀ of about 29 nM;
(h) binds with mouse CD3 at an EC₅₀ that is more than 1000-fold higher than the EC₅₀ of the human or cynomolgus monkey CD3;
(i) binds with rat EGFR at an EC₅₀ of about 26 nM; and/or
(j) binds with rat CD3 at an EC₅₀ of about 542 nM.

Embodiment 270 The method of embodiment 256, wherein the second metabolic product of the isolated recombinant polypeptide complex binds with:
(e) a human EGFR at an EC₅₀ of about 0.27 nM;
(f) a human CD3 at an EC₅₀ of about 0.09 nM;
(g) a cynomolgus monkey EGFR at an EC₅₀ of about 0.26 nM; and/or
(h) a cynomolgus monkey CD3 at an EC₅₀ of about 0.09 nM.

Embodiment 271 The method of embodiment 270, wherein the second metabolic product of the isolated recombinant polypeptide complex:
(f) does not bind with an albumin derived from human, cynomolgus monkey, mouse, or rat;
(g) binds with mouse EGFR at an EC₅₀ of about 20 nM;
(h) binds with mouse CD3 at an EC₅₀ that is more than 1000-fold higher than the EC₅₀ of the human or cynomolgus monkey CD3;
(i) binds with rat EGFR at an EC₅₀ of about 18 nM; and/or
(j) binds with rat CD3 at an EC₅₀ of about 415 nM.

Embodiment 272 The method of any one of embodiments 250-271, wherein the isolated recombinant polypeptide complex exhibits a cancer cell killing activity in an in vitro assay that is at least 100 fold weaker than the enzymatic product of the isolated recombinant polypeptide complex.

Embodiment 273 The method of any one of embodiments 250-272, wherein the isolated recombinant polypeptide complex induces cytokine release from an immune cell in an in vitro assay at an EC₅₀ that is at least 100 fold higher than the enzymatic product of the isolated recombinant polypeptide complex.

Embodiment 274 The method of embodiment 272, wherein the cancer cell killing activity is measured in the presence of a cancer cell and an immune cell.

Embodiment 275 The method of embodiment 273, wherein the cytokine release is measured in the presence of a cancer cell and an immune cell.

Embodiment 276 The method of embodiment 274 or 275, wherein the immune cell is a human peripheral blood mononuclear cell (PBMC).

Embodiment 277 The method of embodiment 273 or 275, wherein the cytokine is IFNγ, TNF, or IL-6.

Embodiment 278 The method of any one of embodiments 182-277, wherein the subject is human.

Embodiment 279 An isolated polypeptide comprising an amino acid sequence with at least 90% sequence identity to the amino acid sequence of SEQ ID NO: 3 and wherein the isolated polypeptide is 221 amino acids in length.

Embodiment 280 An isolated polypeptide comprising an amino acid sequence with at least 90% sequence identity to the amino acid sequence of SEQ ID NO: 4 and wherein the isolated polypeptide is 229 amino acids in length.

Embodiment 281 An isolated polypeptide comprising an amino acid sequence with at least 90% sequence identity to the amino acid sequence of SEQ ID NO: 5 and wherein the isolated polypeptide is 484 amino acids in length.

Embodiment 282 An isolated polypeptide comprising an amino acid sequence with at least 90% sequence identity to the amino acid sequence of SEQ ID NO: 6 and wherein the isolated polypeptide is 492 amino acids in length.

Embodiment 283 An isolated polypeptide comprising a first amino acid sequence having at least 90% sequence identity to the amino acid sequence of SEQ ID NO: 3 and 221 amino acids in length and a second amino acid sequence having at least 90% sequence identity to the amino acid sequence of SEQ ID NO: 2 and 653 amino acids in length.

Embodiment 284 An isolated polypeptide comprising a first amino acid sequence having at least 90% sequence identity to the amino acid sequence of SEQ ID NO: 3 and 221 amino acids in length and a second amino acid sequence having at least 90% sequence identity to the amino acid sequence of SEQ ID NO: 5 and 484 amino acids in length.

Embodiment 285 An isolated polypeptide comprising a first amino acid sequence having at least 90% sequence identity to the amino acid sequence of SEQ ID NO: 3 and 221 amino acids in length and a second amino acid sequence having at least 90% sequence identity to the amino acid sequence of SEQ ID NO: 6 and 492 amino acids in length.

Embodiment 286 An isolated polypeptide comprising a first amino acid sequence having at least 90% sequence identity to the amino acid sequence of SEQ ID NO: 4 and 229 amino acids in length and a second amino acid sequence having at least 90% sequence identity to the amino acid sequence of SEQ ID NO: 2 and 653 amino acids in length.

Embodiment 287 An isolated polypeptide comprising a first amino acid sequence having at least 90% sequence identity to the amino acid sequence of SEQ ID NO: 4 and 229 amino acids in length and a second amino acid sequence having at least 90% sequence identity to the amino acid sequence of SEQ ID NO: 5 and 484 amino acids in length.

Embodiment 288 An isolated polypeptide comprising a first amino acid sequence having at least 90% sequence identity to the amino acid sequence of SEQ ID NO: 4 and 229 amino acids in length and a second amino acid sequence having at least 90% sequence identity to the amino acid sequence of SEQ ID NO: 6 and 492 amino acids in length.

Embodiment 289 An isolated polypeptide comprising a first amino acid sequence having at least 90% sequence identity to the amino acid sequence of SEQ ID NO: 1 and 256 amino acids in length and a second amino acid sequence having at least 90% sequence identity to the amino acid sequence of SEQ ID NO: 5 and 484 amino acids in length.

Embodiment 290 An isolated polypeptide comprising a first amino acid sequence having at least 90% sequence identity to the amino acid sequence of SEQ ID NO: 1 and 256 amino acids in length and a second amino acid sequence having at least 90% sequence identity to the amino acid sequence of SEQ ID NO: 6 and 492 amino acids in length.

Embodiment 291 A pharmaceutical composition comprising the isolated polypeptide of any one of embodiments 279-290, and a pharmaceutically acceptable excipient.

Embodiment 292 A method for treating cancer comprising administering to a subject in need thereof an effective amount of the pharmaceutical composition of embodiment 291.

Embodiment 293 A pharmaceutical composition comprising:
(a) an isolated recombinant polypeptide complex comprising a first chain with an amino acid sequence having at least 70% sequence identity to SEQ ID NO: 1 and a second chain with an amino acid sequence having at least 70% sequence identity to SEQ ID NO: 2; and
(b) a pharmaceutically acceptable excipient.

Embodiment 294 The pharmaceutical composition of embodiment 293, wherein the pharmaceutically acceptable excipient comprises a buffer, a stabilizing agent, a tonicity agent, a surfactant, or combinations thereof.

Embodiment 295 The pharmaceutical composition of embodiment 294, wherein the buffer comprises an amino acid or a derivative thereof.

Embodiment 296 The pharmaceutical composition of embodiment 295, wherein the amino acid or the derivative thereof comprises L-histidine, L-histidine monohydrochloride monohydrate, or combinations thereof.

Embodiment 297 The pharmaceutical composition of any one of embodiments 294-296, wherein the stabilizing agent comprises sugar.

Embodiment 298 The pharmaceutical composition of embodiment 297, wherein the sugar comprises sucrose.

Embodiment 299 The pharmaceutical composition of any one of embodiments 294-298, wherein the tonicity agent comprises sugar.

Embodiment 300 The pharmaceutical composition of embodiment 299, wherein the sugar comprises sucrose.

Embodiment 301 The pharmaceutical composition of any one of embodiments 294-300, wherein the surfactant comprises a polysorbate (PS).

Embodiment 302 The pharmaceutical composition of any one of embodiments 294-301, wherein the surfactant comprises polysorbate 20 (PS20).

Embodiment 303 The pharmaceutical composition of any one of embodiments 293-301, wherein the pharmaceutical composition comprises about 10 millimolar (mM) L-histidine in the form of L-histidine and L-histidine monohydrochloride monohydrate.

Embodiment 304 The pharmaceutical composition of any one of embodiments 293-302, wherein the pharmaceutical composition comprises about 8% weight/volume (w/v) sucrose.

Embodiment 305 The pharmaceutical composition of any one of embodiments 293-303, wherein the pharmaceutical composition comprises about 0.01% (w/v) polysorbate 20 (PS20).

Embodiment 306 The pharmaceutical composition of any one of embodiments 293-304, wherein the pharmaceutical composition comprises about 10 mM L-histidine in the form of L-histidine and L-histidine monohydrochloride monohydrate, about 8% (w/v) sucrose, and about 0.01% (w/v) polysorbate 20.

Embodiment 307 The pharmaceutical composition of any one of embodiments 293-305, wherein the pharmaceutical composition comprises a pH of about 5.3.

Embodiment 308 The pharmaceutical composition of any one of embodiments 293-306, wherein the pharmaceutical composition comprises an osmolality of about 276 mOsmol/kg.

What is claimed is:

1. An isolated recombinant polypeptide complex comprising a first chain with an amino acid sequence having at least 70% sequence identity to SEQ ID NO: 1 and a second chain with an amino acid sequence having at least 70% sequence identity to SEQ ID NO: 2 wherein the isolated recombinant polypeptide complex comprises at least one of the following characteristics:
(a) at least one N-glycan moiety;
(b) at least one disulfide bond;
(c) a melting onset temperature (T_{Onset}) between about 60° C. to about 65° C. and a transition mid-point temperature (Tₘₗ) between about 70° C. and about 75° C. when the isolated recombinant polypeptide complex is formulated at a concentration of about 1.0 mg/mL in about 10 mM histidine, about 8% (w/v) sucrose, about 0.01% (w/v) polysorbate 20 (PS20) pH of about 5.3, wherein the T_{Onset} and the Tₘₗ are measured using differential scanning calorimetry (DSC);
(d) a far UV circular dichroism dip at a wavelength between 210 nm and 230 nm when the isolated recombinant polypeptide complex is formulated at a concentration of about 0.1 mg/mL in water; or
(e) a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

2. The isolated recombinant polypeptide complex of claim 1, wherein the polypeptide complex comprises at least two of the characteristics.

3. The isolated recombinant polypeptide complex of claim 1, wherein the polypeptide complex comprises at least three of the characteristics.

4. The isolated recombinant polypeptide complex of claim 1, wherein the polypeptide complex comprises at least four of the characteristics.

5. The isolated recombinant polypeptide complex of claim 1, wherein the polypeptide complex comprises at least five of the characteristics.

6. The isolated recombinant polypeptide complex of claim 1, wherein the first chain comprises at least 75% sequence identity to SEQ ID NO: 1.

7. The isolated recombinant polypeptide complex of claim 1, wherein the first chain comprises at least 80% sequence identity to SEQ ID NO: 1.

8. The isolated recombinant polypeptide complex of claim 1, wherein the first chain comprises at least 85% sequence identity to SEQ ID NO: 1.

9. The isolated recombinant polypeptide complex of claim 1, wherein the first chain comprises at least 90% sequence identity to SEQ ID NO: 1.

10. The isolated recombinant polypeptide complex of claim 1, wherein the first chain comprises at least 95% sequence identity to SEQ ID NO: 1.

11. The isolated recombinant polypeptide complex of claim 1, wherein the first chain comprises at least 99% sequence identity to SEQ ID NOs: 1.

12. The isolated recombinant polypeptide complex of claim 1, wherein the first chain comprises the amino acid sequence according to SEQ ID NO: 1.

13. The isolated recombinant polypeptide complex of claim 1, wherein the second chain comprises at least 75% sequence identity to SEQ ID NO: 2.

14. The isolated recombinant polypeptide complex of claim 1, wherein the second chain comprises at least 80% sequence identity to SEQ ID NO: 2.

15. The isolated recombinant polypeptide complex of claim 1, wherein the second chain comprises at least 85% sequence identity to SEQ ID NO: 2.

16. The isolated recombinant polypeptide complex of claim 1, wherein the second chain comprises at least 90% sequence identity to SEQ ID NO: 2.

17. The isolated recombinant polypeptide complex of claim 1, wherein the second chain comprises at least 95% sequence identity to SEQ ID NO: 2.

18. The isolated recombinant polypeptide complex of claim 1, wherein the second chain comprises at least 99% sequence identity to SEQ ID NO: 2.

19. The isolated recombinant polypeptide complex of claim 1, wherein the second chain comprises the amino acid sequence according to SEQ ID NO: 2.

20. The isolated recombinant polypeptide complex of claim 1, wherein the at least one N-glycan moiety is located on the first chain.

21. The isolated recombinant polypeptide complex of claim 1, wherein the at least one N-glycan moiety is located on the second chain.

22. The isolated recombinant polypeptide complex of claim 1, wherein the at least one N-glycan moiety comprises G2F, G2FS1, or G2FS2.

23. The isolated recombinant polypeptide complex of claim 1, wherein the at least one N-glycan moiety comprises G2F.

24. The isolated recombinant polypeptide complex of claim 1, wherein the at least one N-glycan moiety comprises G2FS1.

25. The isolated recombinant polypeptide complex of claim 1, wherein the at least one N-glycan moiety comprises G2FS2.

26. The isolated recombinant polypeptide complex of claim 1, wherein at least one asparagine deamidation moiety is located at Asparagine 83 of SEQ ID NO: 1.

27. The isolated recombinant polypeptide complex of claim 1, wherein the at least one N-glycan moiety is located at Asparagine 519 of SEQ ID NO: 2.

28. The isolated recombinant polypeptide complex of claim 1, wherein the isolated recombinant polypeptide complex further comprises O-xylosylation, asparagine deamidation, or succinimide formation.

29. The isolated recombinant polypeptide complex of claim 26, wherein the succinimide formation is located at Asparagine 83 of SEQ ID NO: 1.

30. The isolated recombinant polypeptide complex of claim 1, wherein the polypeptide complex comprises at least two disulfide bonds formed by pairs of cysteine residues.

31. The isolated recombinant polypeptide complex of claim 1, wherein the polypeptide complex comprises at least three disulfide bonds formed by pairs of cysteine residues.

32. The isolated recombinant polypeptide complex of claim 1, wherein the polypeptide complex comprises at least four disulfide bonds formed by pairs of cysteine residues.

33. The isolated recombinant polypeptide complex of claim 1, wherein the polypeptide complex comprises at least five disulfide bonds formed by pairs of cysteine residues.

34. The isolated recombinant polypeptide complex of claim 1, wherein the polypeptide complex comprises at least six disulfide bonds formed by pairs of cysteine residues.

35. The isolated recombinant polypeptide complex of claim 1, wherein the polypeptide complex comprises at least seven disulfide bonds formed by pairs of cysteine residues.

36. The isolated recombinant polypeptide complex of claim 1, wherein the polypeptide complex comprises at least eight disulfide bonds formed by pairs of cysteine residues.

37. The isolated recombinant polypeptide complex of claim 1, wherein the polypeptide complex comprises at least nine disulfide bonds formed by pairs of cysteine residues.

38. The isolated recombinant polypeptide complex of claim 1, wherein the polypeptide complex comprises at least ten disulfide bonds formed by pairs of cysteine residues.

39. The isolated recombinant polypeptide complex of claim 1, wherein the pair of cysteine residues comprises Cysteine 4 and Cysteine 15 of SEQ ID NO: 1.

40. The isolated recombinant polypeptide complex of claim 1, wherein the pair of cysteine residues comprises Cysteine 65 and Cysteine 130 of SEQ ID NO: 1.

41. The isolated recombinant polypeptide complex of claim 1, wherein the pair of cysteine residues comprises Cysteine 176 and Cysteine 236 of SEQ ID NO: 1.

42. The isolated recombinant polypeptide complex of claim 1, wherein the pair of cysteine residues comprises Cysteine 256 of SEQ ID NO: 1, and Cysteine 653 of SEQ ID NO: 2.

43. The isolated recombinant polypeptide complex of claim 1, wherein the pair of cysteine residues comprises Cysteine 22 and Cysteine 96 of SEQ ID NO: 2.

44. The isolated recombinant polypeptide complex of claim 1, wherein the pair of cysteine residues comprises Cysteine 138 and Cysteine 148 of SEQ ID NO: 2.

45. The isolated recombinant polypeptide complex of claim 1, wherein the pair of cysteine residues comprises Cysteine 199 and Cysteine 275 of SEQ ID NO: 2.

46. The isolated recombinant polypeptide complex of claim 1, wherein the pair of cysteine residues comprises Cysteine 339 and Cysteine 407 of SEQ ID NO: 2.

47. The isolated recombinant polypeptide complex of claim 1, wherein the pair of cysteine residues comprises Cysteine 453 and Cysteine 526 of SEQ ID NO: 2.

48. The isolated recombinant polypeptide complex of claim 1, wherein the pair of cysteine residues comprises Cysteine 577 and Cysteine 633 of SEQ ID NO: 2.

49. The isolated recombinant polypeptide complex of claim 1, wherein the polypeptide complex comprises 1 inter-chain disulfide bonds between the first chain and the second chain and 9 intra-chain disulfide bonds.

50. The isolated recombinant polypeptide complex of claim 1, wherein the polypeptide complex comprises 1 inter-chain disulfide bonds between the first chain and the second chain and the second chain comprises 6 intra-chain disulfide bonds and the first chain comprises 3 intra-chain disulfide bonds.

51. The isolated recombinant polypeptide complex of claim 1, having a T_{Onset} between about 61° C. to about 64.5° C.

52. The isolated recombinant polypeptide complex of claim 1, having a T_{Onset} between about 62° C. to about 64° C.

53. The isolated recombinant polypeptide complex of claim 1, having a T_{Onset} of about 62.5° C.

54. The isolated recombinant polypeptide complex of claim 1, having a T_{Onset} of about 63.2° C.

55. The isolated recombinant polypeptide complex of claim 1, having a Tₘₗ between about 71° C. to about 75° C.

56. The isolated recombinant polypeptide complex of claim 1, having a Tₘₗ between about 72.5° C. to about 74.5° C.

57. The isolated recombinant polypeptide complex of claim 1, having a Tₘₗ of about 73.8° C.

58. The isolated recombinant polypeptide complex of claim 1, having a Tₘₗ of about 74.0° C.

59. The isolated recombinant polypeptide complex of claim 1, wherein the secondary structure composition comprises a β-sheet and a random coil.

60. The isolated recombinant polypeptide complex of claim 1, having a far UV circular dichroism dip at a wavelength between 215 nm and 225 nm.

61. The isolated recombinant polypeptide complex of claim 1, having a far UV circular dichroism dip at a wavelength between 215 nm and 220 nm.

62. The isolated recombinant polypeptide complex of claim 1, having a near UV circular dichroism dip at a wavelength between 280 nm and 290 nm.

63. The isolated recombinant polypeptide complex of claim 1, having a near UV circular dichroism dip at a wavelength between 280 nm and 285 nm.

64. The isolated recombinant polypeptide complex of claim 1, having a near UV circular dichroism peak at a wavelength between 270 nm and 275 nm.

65. The isolated recombinant polypeptide complex of claim 1, having a near UV circular dichroism peak at a wavelength between 285 nm and 290 nm.

66. The isolated recombinant polypeptide complex of claim 1, wherein the first chain comprises the amino acid sequence according to SEQ ID NO: 1, and the second chain comprises the amino acid sequence according to SEQ ID NO: 2, and the at least one N-glycan moiety comprises G2F, G2FS1, or G2FS2, and the recombinant polypeptide complex comprises disulfide bonds formed by pairs of cysteine residues Cysteine 4 and Cysteine 15 of SEQ ID NO: 1, Cysteine 65 and Cysteine 130 of SEQ ID NO: 1, Cysteine 176 and Cysteine 236 of SEQ ID NO: 1, Cysteine 256 of SEQ ID NO: 1 and Cysteine 653 of SEQ ID NO: 2, Cysteine 138 and Cysteine 148 of SEQ ID NO: 2, Cysteine 22 and Cysteine 96 of SEQ ID NO: 2, Cysteine 199 and Cysteine 275 of SEQ ID NO: 2, Cysteine 339 and Cysteine 407 of SEQ ID NO: 2, Cysteine 453 and Cysteine 526 of SEQ ID NO: 2, and Cysteine 577 and Cysteine 633 of SEQ ID NO: 2, and the T_{Onset} is between about 60° C. to about 65° C. and a transition mid-point temperature (Tₘₗ) between about 70° C. and about 75° C., wherein the T_{Onset} and the Tₘₗ are measured using differential scanning calorimetry (DSC), when the isolated recombinant polypeptide complex is formulated at a concentration of about 1.0 mg/mL in about 10 mM histidine, about 8% (w/v) sucrose, about 0.01% (w/v) PS20 pH of about 5.3, and a far UV circular dichroism dip at a wavelength between 210 nm and 230 nm when the isolated recombinant polypeptide complex is formulated at a concentration of about 0.1 mg/mL in water, and a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

67. An isolated recombinant polypeptide complex comprising a first chain with an amino acid sequence having at least 80% sequence identity to SEQ ID NO: 1 and a second chain with an amino acid sequence having at least 80% sequence identity to SEQ ID NO: 2 wherein the isolated recombinant polypeptide complex comprises the following characteristics:
(a) at least one N-glycan moiety;
(b) at least one disulfide bond;
(c) a melting onset temperature (T_{Onset}) between about 60° C. to about 65° C. and a transition mid-point temperature (Tₘₗ) between about 70° C. and about 75° C., wherein the T_{Onset} and the Tₘₗ are measured using differential scanning calorimetry (DSC), when the isolated recombinant polypeptide complex is formulated at a concentration of about 1.0 mg/mL in about 10 mM histidine, about 8% (w/v) sucrose, about 0.01% (w/v) PS20 pH of about 5.3;
(d) a far UV circular dichroism dip at a wavelength between 210 nm and 230 nm when the isolated recombinant polypeptide complex is formulated at a concentration of about 0.1 mg/mL in water; or
(e) a near UV circular dichroism dip at a wavelength between 275 nm and 290 nm when the isolated recombinant polypeptide complex is formulated at a concentration of about 1.0 mg/mL in about 10 mM histidine, about 8% (w/v) sucrose, about 0.01% (w/v) PS20 pH of about 5.3.

68. A pharmaceutical composition comprising:
(a) the isolated recombinant polypeptide complex according to any one of claims 1-67; and
(b) a pharmaceutically acceptable carrier.

69. The pharmaceutical composition of claim 68, wherein the pharmaceutically acceptable carrier comprises a buffer, a stabilizing agent, a tonicity agent, a surfactant, or combinations thereof.

70. The pharmaceutical composition of claim 68, wherein the buffer comprises an amino acid or a derivative thereof.

71. The pharmaceutical composition of claim 70, wherein the amino acid or derivative thereof comprises L-histidine, L-histidine hydrochloride monohydrate, or a combination thereof.

72. The pharmaceutical composition of claim 68, wherein the surfactant is polysorbate 20.

73. The pharmaceutical composition of claim 68, wherein the stabilizing agent is sucrose.

74. The pharmaceutical composition of claim 68, having a pH less than 6.0.
